SEQUENCE LISTING (53 sequences)

SEQ ID NO: 1 - DNA, 8873 bp, Homo sapiens
actgaccatttggcgatccattgagaggagggtttggaaaagtggctcctttgtgacagctctcgccagattggggggctgctgatttgcatctcattagccatgcgggcggccggctgaatataagggcggcaggcgccggcgagagccagatcctctgcgcgcacccgcggagacccgacccggccgagggcagagcgcaggggaacccgggcagccgcggcgcagagcctcctcccacggcccggcccctccggtcctgcgcgtgtgtactggatggcattggctggattcatcggaaagacgcggatctttgctgtgacaccggagatcggagcccggagtgctcccggaacgaccgccgccgccgagtgacaccgggccgcgatccgcaggggccgccgcgcacacccgccgccgccgaccgtcccctcagcgcgcgccgctggccccggattatcgccttgcccgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtaggcgggcaggtgggggcgccgcggccccgcggggtctcacgggtagccggggcgcggggcaggagcgcgcggggaggggcggacagcggcacgggccgcgccagccacggcccggaagatgaatcccgggggcgacgaccccagcgccggccgtgcagcgagcgcgctcggcccctgagcccttccaggctctccgcacaccccccacccaggcctcacgccccctagctcgggcgggacccgcgtcctcacgcccccgccctcccccgtgcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccggtgagtgccgcacctgcgcgcgccgggccggccctgaagctgggcgggctgcaggacgcgctgggatcccgccttgggcgctcggtggcgggacctcggggaccccgcgaggcgcaggtgggcgctgcgatctgcctagcggcggccccaggactccagcccagcagcgcggacacctcgccccggggccccgcggcctgcaggaggggaccgcgctggggcgaggaggagaggccgagcgcgcccgggagatttccgtatccggcctctgtgccaggtctccagtcagaggcgccccttcacgtgggaaggttctggtttcccgactcctagacgcgttggtggcgcgattacccgcgcagcgcgaccgctaccacccggagcgtgcccatcccccaagaaaaatgacaagggccctcgggcctcttccaccccatcctgcctgcattctctctctctctctaattaaaaaaacaacgtaatatcctgtagtacaggctgaaaaaacacgtcaggaaaccactctttaaaaagttcttccatttccttagggaaggtgagagcaggcaggaggtgcgtggagaccctctccagacacgctgccccagacctgcagccttcaggcctctgttgctgacctggctgttaggaatgactgctttttgccgttttcttttcgttacctttctgggttgtctaacgtcttctcccctctctcccagggcaccttctctctgattattgaagctctccacacagattctcctgatgacctcgcaacaggtaaaaacaaaacccaaaccccaaaactgctttccccagttaatagcattggactttgcccacccatcccccagccaaacccggacagctttcattctgcacgtgccccagaaagttcagggtggagcagcttgggcctccttcccgtgctgaatgtctcggcccacccccgctctgtcccgagtcacagggttctcgttcagaaccaaccaggagcatcttctccccgtagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagcgtgagtctctgggaaggcaccgctggctcactcgtccacgaacacggaccgcgcgcagggacggggcttcctgagccacggggggcttgggactgtagagatgttctggtggggaaactgaggcccagaggacagaagtggattgctataagtcacagctcgtcagtgggggggttggggtcaacgcagacattttaacatcccaggctgtgtttatccactatcggaactgcctttcttaatcagggaggattttagagacagggccaggggtcaggaagtaaagccagtgctacccccagggtgtgtgtattagagagggagaggaggaaggaagggaggaacacagagagagcttgtgtgtcaggggcaccatttcaacccgagttcccagtgctggaacagcatcacactgggaaacgttccattttctctctggagctggtgtgcttgacctctctggagcaaacgcctttccggatactccctgtgacacgcactgtctatgctggccagagagcaggctttcactcctgtgggctgctgaggccaggtctccaaggcctgtgtgggcgaggggtgcacagccccgtctggcttgaatgctcaggcagcaccttgtctggagaagcaatgtcttcccaatagtgacagaggctctacctgcctcttattaggtattgatgtgtcaatgtcatggcaggcaggtgactagggcagggttggggccgtgctggctcctggttctggctcatggggacctcaggagccctctctccagctgactgaggcctcgcctgcacgcctggccgtcccagcccattggtaccggatttctctacagctggggattgggtaggtcctggagctgcccagaaactccagggaactgtcattctccttccttggaactggacaaccttggagaggggctctgggaggcccagaacctctggcaggagctgggtagtgcctggggttgagggtgggtcttcccattcactgagtgccttgatgtccttgctccttagcttcccaaattccctccggaacttactgagctccttctaagctttgccttggcctgaactggttctggggaaaaacaaaaaaacaaaaaacaacttgtggagctgcttgttaatgagtttcataaccaggcagcaagagccagctccaagcctcaagcccactgtctactccctgccctgcgggagcctctggccagtctgctgcctcccacccttcctccctgcctctcttcaccacagggtagccagaaacttaaacttttttcttcaaacactgaagtctctccccgcccccagctcgcgcgtgccatagattagatctctccggggataggcgcagggacacccgccggctcccattggcggaaggggtgcgtgtgcgtgtgtgtgtgtgtgtgtgtgtgtacacgcgaggggtgtgtgtgaggaggtggggccgggggcgcgggggaggccggcattgttgcgctggggcagctgccgtggaggacagacaatggagcagctgtcctgccctggcaccctgcataccagctgtccactcttatctgcacacacactttctgggatattaagaggtggagctttgtgcacagaattgggaagtgggggaggaggagggggaagacttctgaccctctcttagaagaaaaggggatagggtgggggtgggggcttccgagagcccttttgtccttgagcccctgtgttaagaagaatgctcatccccagggctgagtcaagtcccaggctactaggcaggggggtcagtcctccacaacctgggaagattaactcagctgggatttgctgactgaagccggcgagttgtgtcctggccccaagggcggcagccctgttgggacgtacttggcgtggggcttgaccctgtttttcctttgcttgtagcgatctgcctgcctggatgtgatgagcagcatggattttgtgacaaaccaggggaatgcaagtaagtctgcacaaggtggtgttttgttttgttgccttttcttgttatcttttcacagctggtgtatttgtaaaaacagccctaggtgatcattcgaaaaactccagtaagattgattgaacagggggccgttttctcatgtttctacttaatcaatgtttggcagcatgtaaggtcatggagttgtcattcgtctaagccccttaacggctatgagaatttacagatagtagtttaaaaagagttggcacaggaaatgatagtatagttcaatggttctcaaatgttgcctcatcctagaatcactcagggagtgatttttgagatgctgacactggtgctgccctaacacccaagaagccagaacctctggtggggcccaggcccaggctgcagctcccaaggtgacccagtgttctgctaatctggagaaccagaggctcactggtgctgcgggaagatggtttctagggtgagaatgtccactgcaaagccagcaacagtcaacgtccatctgagtcttctgcttttctccaaggtgcagagtgggctggcagggccggtactgtgacgagtgtatccgctatccaggctgtctccatggcacctgccagcagccctggcagtgcaactgccaggaaggctgggggggccttttctgcaaccagggtaagccttctctccctgaggcagcctgctccctccagagcagccctggacttccctggctgtttgatcactggaaaaataaagtcttcctgcatttgatgtcgagcttcctatctcctacttttcctgtccccacccttcacagacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggtgagtcggaggctccatggcatctcacccggaagctggggtgccctggtgttgaatggagtgtgtgggctccttggagcaactttggaaagccttttctgacctctccatcgtgtaggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtaagggggcagctggtgattgctcagagactcgggcgagcggtcaatactgaggtggcattaaaaacaagcatttgtgagtgacctcgagtttatgaatcacttttatccagaccgccaggaattctcgatggaaactctatctttgagtctggaaaggcctggggaatgagagaggccagggcatttgttatgaagttctctgtggaaacctagaccaagcagtgaatgacttgctcagggccacaaggtgcttcgggcacctgcggccgcctgaggttcagtaagtgatgcccacaggtgccggccactccagcttgggaggatggcccagctgtgtggccacccagcacagtagttgggggtgtccctgagtgaggacagagagcctcctgctagcagcgaggggctggctgcccaaaggagacacacagcaaggagagctgggccccagatgtgccggagcattccggaatggtcatccttcccctccctccctcccctgttgtcagtgcctgctcctctcacttgctgtgtaactgtgggcaaggacaccctcgttaagcctcagtttccccatctgaaacctgggtcgagtggcacatgctcttgcccggctgttgtggcgactaatgcagccaccagagtgttctgcacagcgcctgtccagatgctggccgtgtggtttctgacttgtagagctagacctggacacctctcgtatttgaggtcctaaaccatgtcaccttgcgctgtggactcattcaggccacagactgtctttggtttgtctggtttctacagtgtcagacagatagatgcttcagagtgactttttggtgaacaaacctacgaggagacacgtgatgttcatgtccctgtgttccaggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcagggggtgcgtgctgcgggccgggcatcaggagggggtacctggggggtgtcttcctggaaccactgctccgtttctcttcccaaatgttctcatgcattcattgtggattttctctattttccttttagtggagaagcatctgaaagaaaaaggccggactcgggctgttcaacttcaaaagacaccaagtaccagtcggtgtacgtcatatccgaggagaaggatgagtgcgtcatagcaactgaggtcagtgcaggcagcagccgctccctcctcctcggcatgggagcacctgaagctggagcacgggaatcggtctcaggctaacttcccatttgtcttgtggccccccaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaa

SEQ ID NO: 2 - DNA, 3779 bp, Homo sapiens
actgaccatttggcgatccattgagaggagggtttggaaaagtggctcctttgtgacagctctcgccagattggggggctgctgatttgcatctcattagccatgcgggcggccggctgaatataagggcggcaggcgccggcgagagccagatcctctgcgcgcacccgcggagacccgacccggccgagggcagagcgcaggggaacccgggcagccgcggcgcagagcctcctcccacggcccggcccctccggtcctgcgcgtgtgtactggatggcattggctggattcatcggaaagacgcggatctttgctgtgacaccggagatcggagcccggagtgctcccggaacgaccgccgccgccgagtgacaccgggccgcgatccgcaggggccgccgcgcacacccgccgccgccgaccgtcccctcagcgcgcgccgctggccccggattatcgccttgcccgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaa

SEQ ID NO: 3 - DNA, 2172 bp, Homo sapiens
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaa

SEQ ID NO: 4 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 5 - protein, 536 aa, Artificial Sequence
  synthetic polypeptide
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKL

SEQ ID NO: 6 - DNA, 9734 bp, Homo sapiens
agtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcaggtaacacgtcccgcgccctctccgtcccctctgccgcgctctgggcctcagccccgggcaccagctgagctgaccggtcccctccctccttccctcggtccctgtgcaatagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccggtgagcacagcctgggcgcactgggaggtcgcagaagccgagagaggaggcgccctgggaccaaagccccctccccagatttccttgtacacacacccccacccccaaaaagcccaggatgcattctttcctggctcttcccgactctctcctgagactgatcccagaaaaggctctcaccagtctccgtcttcccagtttatgtcctcccgtccccagctcttgggacacgattttcattacctaccactctggggcggtaccctaccaccccctcctccagtggctctcccttacactctcccgtctctcaaccctccctctaccgggggttctcctctcgccttccctgctcaagcgctacactgtgcacagccccgttatgttgacccgggcgcagtaactgaatcctgcaattagattaattaaacaggctgccgcaaggcacccccacctctccccgcttgctcatctcgccatctctccgtccccccaccccctttcccagggtaccttctcgctcatcatcgaagcttggcacgcgccaggagacgacctgcggccaggtgagtagctcgctccgccaccacaggggggcgacacggcgcagcgccgaaagagttaatctgttctaggcgggggaagtgcgggcttgggggtgggaggcaggacgcttagcttggcctggagctgcgccccgcgctggacgctcggattccgctcgctgcctggactcagagcacaattgcgtttcctgcgggttatttttggcgtgggaacgcggggagtacggcggtgagaaaggctgaagctgccagcgccgctgacgggccccttcctgtattttacacctttcgcgaattccgctcctttggaaagggaataatggctttgggatgttgttctgacacagaggaaaaggatatttcagcagcacaacaattctcactttgaaaaggaaaaaagaaaaccattacccacctctggaggcagaacccctgaatgggcaccaaaggaccccctgctcccagggtcctctctagcctggggagcttttctttctttttctcttttttccattttgacctcttttcctctttcccctccctatctgcctccaagaccctgggatatcttaacatccttctattgtcccctttttgaatactatcaggccccctgcacatgcacacacgtagggcagctacgtagcggggctttgggtccctctggcctgttcttgctggcaggcgggggtcatctggataactgggctgattggttggctgatcaccatcatcacagccaagaaggacattggccagccgtcactggcacccttggggactggcgacccttccctgacccgaccctctgccccctcagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcgtaagcagtcaagctcccacctgtgtggaaggggagggtcccctgaggaaacacagtggagcttcttggtcacagcttgcctcccttgaagagtgggtctgggcctcctactagctgggcctcagggatgctgagggtgggcttgacctcagacctcctgtctcttcccagtgctcctcccatcatgccaaagcccacaagaaccccatcatgacattccatccagtttggcttctccttccctgtgccattatttcactttaagacactcggggctcctctgggaggccaggagtaggaagagggcccaggagagctaggggatccccagggccagcaggtgagaatggggcttaagagtccttggtatcccagcctcacccagctctgtgttcttcccttagctatctgtctttcgggctgtcatgaacagaatggctactgcagcaagccagcagagtgcctgtgagtaggggacaggaagtggtgagtgggagccctcccttggccaaggcctctcacctcactctgcctctctcttgttccccagctgccgcccaggctggcagggccggctgtgtaacgaatgcatcccccacaatggctgtcgccacggcacctgcagcactccctggcaatgtacttgtgatgagggctggggaggcctgttttgtgaccaaggtgagtcagggtgaagagagggtgcagagggtgcaagagatatggggctggggggtggaaatccgattcgtcacctggatccttcttacttggtgactgcagacttggctttcccatgatcttccaaggatcttgggtcttttaaggatctttacaactggcccagaatgaggcggtgggtccttctccaggtgcggcggcagggggtggtggagccagggtggctgaaaaacccaggggggtgacaaggtcggcagcctggaggttgcactcataaatcctagcaaagccaaagagagagggatggcaggctcagttcctctttcaaccccgtagttacctattaaccccctgagtgtttgcttaccttccagggctgtttgagcagctctcccctaaacagctgtccggtggggtgtgcccaccggccacctgaggctgtgggtgagctgggcctctgggcggagtggcatctaaccgacttttcggtgtgggcacaaacggcctcccctgctcttacctagttaccacctgcctgaacccatgcggtctctacctggtgtttaggggtagtcactctctggctatacaggggcctttcagccccaaccttgggggaggaggaagccttttttcttgcatcctgctagccagctgcagccagctgcagctcccattttcaggatcaaatgggtgcacctgctgcccagagacaccggcgcaggcctgggtagggtgggcagagagcttgccagggtggaaagaaattgcctaggccctgacttgctgtcaacaaggggcttgggattcagtccctgtgttgtgtgtgtgtgtgtgtgtgtgtgtgtgtgtctgtccctttactaccatccccaccccaacactcacacacctggttcctgctcattctcttccctctccaccatatttgctcccaggtgacacagtcatatactcatcatatgcaaacacagcacttgcaggccatatatttactctgtctggttctccctccctgtccttcccaaataaaaaaacaaatacttatatttcaaaatacccttgtaacacctcttcctttaaaaaatgcccgattactgcctatggtggctctcatctctcctctaccatttctacctgttgaaattttatccctccttccaggcttatctcagctgcccctcctccatgaagccttttctgacttcctccccgacatgtggccttgccctctgctcttcttccttatcttcatcctacttgggttggcagtttgtgagtttccctggcaggacgtcttccagttccagttgtgttgtttcacttttggttgactgcactggtcatatgtgattcaaggtgctttaagaaacatgattttcatcctggctaacacagtgaaaccctgtctgtattaaaaatacaaaagttagccaggtgtggtggcaggcacctgtagccccagctgctgggaaggctgaggcaggagaatggcgaagtagagcttgcagtgagccgaggtcgtgccactgcactccagcctgagtgacagagcaagactccgtctcaaaaaaaaaaaaaaaaaaaaaaaaaagaaacatgattttaggctgggtgcgatggcctgtaatcccagcactttgggaggccgaggtaggtggatcacttgaagtcaggagttcgagaccatcctggccatcctggtgaaacccctgtaaaaatacaaatattaatcgggcacagtggcgcatgcctgtaatcccagctacttagaaggttgaggtatgagaatcgcttgaacccggaaggcgaaggttgtagtgagcctatatcacatcactgcactccagcctgggcgacagagtgagactctgttaaaaaaaaaaaaaaaagaaggaaagaaagagaaagagagagaaagaaagaaagaaagagaaagaaaaaagattttattggtggtggaggaaggatgtttgggcctgggagactttgagttgaggtgtctttgagccaaacatgggggcaaacatggactgcaaggagcctggaggtgagtgcattccctggccctgctcagctgcttggttcctgtttctgcagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggtgaggcccagaccagcgcaggaagacagaggtgtcaggtggtgtctgggcatccctaacctaggcagttagtggatgtacagccatggacaggcattgtgggcaggtggagcccagccttcagtcacacatccctgccccccagggtctgactttggcccctttatggtctctctccaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacggtgcgtgctgctgccctgctaacctggtggactggccctggggctgagagagacttctggtgagggagggtcaggagaggagcgaggcattgtctgccactctggccccccatctgctctggagggcgaagagcttgcttgatcagctggggggctgtggaagcggagctggttagttgcacgcaggccttaggagcaggggtggtatgcaccctgcatagcttccattcctattcccatgtcagaaccccgtcctggctggggtggcctctgaccctccccaggaagtcctgagctggagagagggatgttggaggcttcatgtttctcctcaaaggaggcagtgattcagtcagagccctgctcctggaggcctcatcttgccccgtgcccaggtagagcatgaggtagcatgaggcatcttgaatgtttgcaccttttgaggcacaaagcctgttggtaatccttgtctatctggctcccaggtgaccctctgtgaggcaggcaggcaggcagcgctcaggagctggagaggggtgggaagggctgagagggagtctgctctctcactgaagcctctggcactgccatttcttcatcactgaatgggaaactataatacctgtcctctgtccttcatgtggttgtgaagatgaagtaaaacagtcatgattgtacttatccgagcattaactatataccaaacatgggctcttgccttcatgtaccttcccggctatcctatgaaggggctagcattctactccagtctaacaaatggggaaactgaggcttagagacacggttaagcagcaagtgccagatctcaggccacagagtgacagctgaggtcccaactcaagcctatctgtctgattctacgttaaagttctgtaagatgctagtcatttttatacatgagcccactgaggccgagagaatcaaggtcatgctaaactccaggtctcctgactctgtgcagttctctttgtagtgggctctgcaggtggaggtagaagggcccgaacgtgttcctggaatggggctcccaccccctgccccagggagctcccaggctatcactgacttgtgtctcatgcgtcctcacagggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacaggtgagtggcacccagaagcccagggcctggccaccggccccgacatggttctgcctaggctcctcttaggccaggcgggaagcagttaagcagctgaggttttgttactgacaggaagatcctccagtaggatttctgtcaggggtcctttgtccttccctcccattcattcatttgttcattcacacatgtcaagtgtccctagggtgtctcttgtgacttccgtctttccacagtgtggcttgcctctagtggcagcactggctttatgcagggctcagacccttctggtgaggttgggaggcctgtgactctcttaggggccttttcctaagtgcccccctgcagcagcccagcactgggcacgtccagcccctgtgtcttccccaagaaccaccctgcagatgccctttggctctccagggtcctccctccccccaagcctctccccgtccctcccttacacgcctgtcttgtgttccctcagtgaaaagccagagtgtcggatatcagcgatatgctcccccagggactccatgtaccagtctgtgtgtttgatatcagaggagaggaatgaatgtgtcattgccacggaggtgagtgctgggctcgcctttccttctgccttttgtgggagggaaagtggcctggtcactcttgacccatgggccattcctgaagggtaggtcagaaccctgccttggcaggccaagttcagtggactcttgggtccctgctggcctcattgccactaagggtgtgaaacaggaaccatggcggcaagcctggtctggtcctttcctgctgtattggtgctgggttgggcagccacggcactgctggccagcctctgatgggtgagggggcccctcaccccttgtgcccttcctgccccttcccactggcttcctccattgacctcatgagcgcaagctcccaggcccgtgtgtgtgttgggccgaagactggggaggactgccccacctgcccttagcccctgcctgccccatcgccttctcccagggaggcccagggagggcctggagggagtgcgcatgcccagggtaacctgtttccctgccttccgcttgctcccaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaacagatgtgtacagga

SEQ ID NO: 7 - DNA, 3426 bp, Homo sapiens
agtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaacagatgtgtacagga

SEQ ID NO: 8 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 9 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 10 - protein, 526 aa, Artificial Sequence
  synthetic polypeptide
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPS

SEQ ID NO: 11 - DNA, 21 bp, Artificial Sequence
  synthetic oligonucleotide
ctatctggcagtgcgagaatg

SEQ ID NO: 12 - DNA, 21 bp, Artificial Sequence
  synthetic oligonucleotide
agacgtgcaagcaggtctttc

SEQ ID NO: 13 - DNA, 21 bp, Artificial Sequence
  synthetic oligonucleotide
tggatgacttatgcgtgattt

SEQ ID NO: 14 - DNA, 21 bp, Artificial Sequence
  synthetic oligonucleotide
caacagaactttatggtagaa

SEQ ID NO: 15 - DNA, 21 bp, Artificial Sequence
  synthetic oligonucleotide
ccgccgtggtttgtattcatt

SEQ ID NO: 16 - DNA, 21 bp, Artificial Sequence
  synthetic oligonucleotide
gcttcctcttcaacctcaata

SEQ ID NO: 17 - DNA, 21 bp, Artificial Sequence
  synthetic oligonucleotide
ccgccgtggtttgtattcatt

SEQ ID NO: 18 - DNA, 21 bp, Artificial Sequence
  synthetic oligonucleotide
gctcttctttgattacaggta

SEQ ID NO: 19 - DNA, 21 bp, Artificial Sequence
  synthetic oligonucleotide
gctactcggaaaggaaacaaa

SEQ ID NO: 20 - DNA, 128 bp, Artificial Sequence
  synthetic polynucleotide
attacagcaagatggaaataccaaatccccctacctccaaatgtatcacttactggaaaagaaaagtgaaatctgaatacatgcgacttcgacaacttaaacggcttcaggcaaatatgggtgcaaag

SEQ ID NO: 21 - DNA, 20 bp, Artificial Sequence
  synthetic oligonucleotide
tcgacaacttaaacggcttc

SEQ ID NO: 22 - DNA, 20 bp, Artificial Sequence
  synthetic oligonucleotide
tgcgacttcgacaacttaaa

SEQ ID NO: 23 - DNA, 20 bp, Artificial Sequence
  synthetic oligonucleotide
cctccaaatgtatcacttac

SEQ ID NO: 24 - DNA, 20 bp, Artificial Sequence
  synthetic oligonucleotide
taaacggcttcaggcaaata

SEQ ID NO: 25 - DNA, 20 bp, Artificial Sequence
  synthetic oligonucleotide
aaacggcttcaggcaaatat

SEQ ID NO: 26 - DNA, 20 bp, Artificial Sequence
  synthetic oligonucleotide
catttggaggtagggggatt

SEQ ID NO: 27 - DNA, 20 bp, Artificial Sequence
  synthetic oligonucleotide
ccagtaagtgatacatttgg

SEQ ID NO: 28 - DNA, 20 bp, Artificial Sequence
  synthetic oligonucleotide
gtgatacatttggaggtagg

SEQ ID NO: 29 - DNA, 20 bp, Artificial Sequence
  synthetic oligonucleotide
aagtgatacatttggaggta

SEQ ID NO: 30 - DNA, 20 bp, Artificial Sequence
  synthetic oligonucleotide
agtgatacatttggaggtag

SEQ ID NO: 31 - DNA, 20 bp, Artificial Sequence
  synthetic oligonucleotide
tttccagtaagtgatacatt

SEQ ID NO: 32 - DNA, 20 bp, Artificial Sequence
  synthetic oligonucleotide
taagtgatacatttggaggt

SEQ ID NO: 33 - DNA, 129 bp, Artificial Sequence
  synthetic polynucleotide
gctttgtatgtggcaaattttgcaaaggttcaagaaaaaacccagatcctcaatgaagaatggaagaagcttcgtgtccaacctgttcagtcaatgaagcctgtgagtggacacccttttctcaaaaag

SEQ ID NO: 34 - DNA, 20 bp, Artificial Sequence
  synthetic oligonucleotide
gcttcattgactgaacaggt

SEQ ID NO: 35 - DNA, 20 bp, Artificial Sequence
  synthetic oligonucleotide
acaggcttcattgactgaac

SEQ ID NO: 36 - DNA, 20 bp, Artificial Sequence
  synthetic oligonucleotide
agaaaagggtgtccactcac

SEQ ID NO: 37 - DNA, 20 bp, Artificial Sequence
  synthetic oligonucleotide
tccattcttcattgaggatc

SEQ ID NO: 38 - DNA, 20 bp, Artificial Sequence
  synthetic oligonucleotide
ccattcttcattgaggatct

SEQ ID NO: 39 - DNA, 20 bp, Artificial Sequence
  synthetic oligonucleotide
cccagatcctcaatgaagaa

SEQ ID NO: 40 - DNA, 20 bp, Artificial Sequence
  synthetic oligonucleotide
gtatgtggcaaattttgcaa

SEQ ID NO: 41 - DNA, 20 bp, Artificial Sequence
  synthetic oligonucleotide
cagtcaatgaagcctgtgag

SEQ ID NO: 42 - protein, 754 aa, Artificial Sequence
  synthetic polypeptide
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEIEGRMDPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 729 aa, Artificial Sequence
  synthetic polypeptide
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 231 aa, Artificial Sequence
  synthetic polypeptide
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 3702 bp, Homo sapiens
atgcggggtctaccgagagggagggggttgatgcgggcccgggggaggggtcgtgcggcccctccgggcagccgaggccgcggaaggggggggccccacagaggaagaggtaggccccggagcctactctctcttcccagggcccaggcatcctggaccccccaactctctactgggctgaccagccctcctgtcccttgtctcccctcccagggggaggcccccgctgagatgggggcgctgctgctggagaaggaaaccagaggagccaccgagagagttcatggctctttgggggacacccctcgtagtgaagaaaccctgcccaaggccacccccgactccctggagcctgctggcccctcatctccagcctctgtcactgtcactgttggtgatgagggggctgacacccctgtaggggctacaccactcattggggatgaatctgagaatcttgagggagatggggacctccgtgggggccggatcctgctgggccatgccacaaagtcattcccctcttcccccagcaaggggggttcctgtcctagccgggccaagatgtcaatgacaggggcgggaaaatcacctccatctgtccagagtttggctatgaggctactgagtatgccaggagcccagggagctgcagcagcagggtctgaaccccctccagccaccacgagcccagagggacagcccaaggtccaccgagcccgcaaaaccatgtccaaaccaggaaatggacagcccccggtccctgagaagcggccccctgaaatacagcatttccgcatgagtgatgatgtccactcactgggaaaggtgacctcagatctggccaaaaggaggaagctgaactcaggaggtggcctgtcagaggagttaggttctgcccggcgttcaggagaagtgaccctgacgaaaggggaccccgggtccctggaggagtgggagacggtggtgggtgatgacttcagtctctactatgattcctactctgtggatgagcgcgtggactccgacagcaagtctgaagttgaagctctaactgaacaactaagtgaagaggaggaggaggaagaggaggaagaagaagaagaggaagaggaggaggaagaggaagaagaagaggaagatgaggagtcagggaatcagtcagataggagtggttccagtggccggcgcaaggccaagaagaaatggcgaaaagacagcccatgggtgaagccgtctcggaaacggcgcaagcgggagcctccgcgggccaaggagccacgaggggtgtccaatgacacatcttcgctggagacagagcgagggtttgaggagttgcccctgtgcagctgccgcatggaggcacccaagattgaccgcatcagcgagagggcggggcacaagtgcatggccactgagagtgtggacggagagctgtcaggctgcaatgccgccatcctcaagcgggagaccatgaggccatccagccgtgtggccctgatggtgctctgtgagacccaccgcgcccgcatggtcaaacaccactgctgcccgggctgcggctacttctgcacggcgggcaccttcctggagtgccaccctgacttccgtgtggcccaccgcttccacaaggcctgtgtgtctcagctgaatgggatggtcttctgtccccactgtggggaggatgcttctgaagctcaagaggtgaccatcccccggggtgacggggtgaccccaccggccggcactgcagctcctgcacccccacccctgtcccaggatgtccccgggagagcagacacttctcagcccagtgcccggatgcgagggcatggggaaccccggcgcccgccctgcgatcccctggctgacaccattgacagctcagggccctccctgaccctgcccaatgggggctgcctttcagccgtggggctgccactggggccaggccgggaggccctggaaaaggccctggtcatccaggagtcagagaggcggaagaagctccgtttccaccctcggcagttgtacctgtccgtgaagcagggcgagctgcagaaggtgatcctgatgctgttggacaacctggaccccaacttccagagcgaccagcagagcaagcgcacgcccctgcatgcagccgcccagaagggctccgtggagatctgccatgtgctgctgcaggctggagccaacataaatgcagtggacaaacagcagcggacgccactgatggaggccgtggtgaacaaccacctggaggtagcccgttacatggtgcagcgtggtggctgtgtctatagcaaggaggaggacggttccacctgcctccaccacgcagccaaaatcgggaacttggagatggtcagcctgctgctgagcacaggacaggtggacgtcaacgcccaggacagtggggggtggacgcccatcatctgggctgcagagcacaagcacatcgaggtgatccgcatgctactgacgcggggcgccgacgtcaccctcactgacaacgaggagaacatctgcctgcactgggcctccttcacgggcagcgccgccatcgccgaagtccttctgaatgcgcgctgtgacctccatgctgtcaactaccatggggacacccccctgcacatcgcagctcgggagagctaccatgactgcgtgctgttattcctgtcacgtggggccaaccctgagctgcggaacaaagagggggacacagcatgggacctgactcccgagcgctccgacgtgtggtttgcgcttcaactcaaccgcaagctccgacttggggtgggaaatcgggccatccgcacagagaagatcatctgccgggacgtggctcggggctatgagaacgtgcccattccctgtgtcaacggtgtggatggggagccctgccctgaggattacaagtacatctcagagaactgcgagacgtccaccatgaacatcgatcgcaacatcacccacctgcagcactgcacgtgtgtggacgactgctctagctccaactgcctgtgcggccagctcagcatccggtgctggtatgacaaggatgggcgattgctccaggaatttaacaagattgagcctccgctgattttcgagtgtaaccaggcgtgctcatgctggagaaactgcaagaaccgggtcgtacagagtggcatcaaggtgcggctacagctctaccgaacagccaagatgggctggggggtccgcgccctgcagaccatcccacaggggaccttcatctgcgagtatgtcggggagctgatctctgatgctgaggctgatgtgagagaggatgattcttacctcttcgacttagacaacaaggatggagaggtgtactgcatagatgcccgttactatggcaacatcagccgcttcatcaaccacctgtgtgaccccaacatcattcccgtccgggtcttcatgctgcaccaagacctgcgatttccacgcatcgccttcttcagttcccgagacatccggactggggaggagctagggtttgactatggcgaccgcttctgggacatcaaaagcaaatatttcacctgccaatgtggctctgagaagtgcaagcactcagccgaagccattgccctggagcagagccgtctggcccgcctggacccacaccctgagctgctgcccgagctcggctccctgccccctgtcaacacatga

SEQ ID NO: 46 - protein, 1233 aa, Homo sapiens
MRGLPRGRGLMRARGRGRAAPPGSRGRGRGGPHRGRGRPRSLLSLPRAQASWTPQLSTGLTSPPVPCLPSQGEAPAEMGALLLEKETRGATERVHGSLGDTPRSEETLPKATPDSLEPAGPSSPASVTVTVGDEGADTPVGATPLIGDESENLEGDGDLRGGRILLGHATKSFPSSPSKGGSCPSRAKMSMTGAGKSPPSVQSLAMRLLSMPGAQGAAAAGSEPPPATTSPEGQPKVHRARKTMSKPGNGQPPVPEKRPPEIQHFRMSDDVHSLGKVTSDLAKRRKLNSGGGLSEELGSARRSGEVTLTKGDPGSLEEWETVVGDDFSLYYDSYSVDERVDSDSKSEVEALTEQLSEEEEEEEEEEEEEEEEEEEEEEEEDEESGNQSDRSGSSGRRKAKKKWRKDSPWVKPSRKRRKREPPRAKEPRGVSNDTSSLETERGFEELPLCSCRMEAPKIDRISERAGHKCMATESVDGELSGCNAAILKRETMRPSSRVALMVLCETHRARMVKHHCCPGCGYFCTAGTFLECHPDFRVAHRFHKACVSQLNGMVFCPHCGEDASEAQEVTIPRGDGVTPPAGTAAPAPPPLSQDVPGRADTSQPSARMRGHGEPRRPPCDPLADTIDSSGPSLTLPNGGCLSAVGLPLGPGREALEKALVIQESERRKKLRFHPRQLYLSVKQGELQKVILMLLDNLDPNFQSDQQSKRTPLHAAAQKGSVEICHVLLQAGANINAVDKQQRTPLMEAVVNNHLEVARYMVQRGGCVYSKEEDGSTCLHHAAKIGNLEMVSLLLSTGQVDVNAQDSGGWTPIIWAAEHKHIEVIRMLLTRGADVTLTDNEENICLHWASFTGSAAIAEVLLNARCDLHAVNYHGDTPLHIAARESYHDCVLLFLSRGANPELRNKEGDTAWDLTPERSDVWFALQLNRKLRLGVGNRAIRTEKIICRDVARGYENVPIPCVNGVDGEPCPEDYKYISENCETSTMNIDRNITHLQHCTCVDDCSSSNCLCGQLSIRCWYDKDGRLLQEFNKIEPPLIFECNQACSCWRNCKNRVVQSGIKVRLQLYRTAKMGWGVRALQTIPQGTFICEYVGELISDAEADVREDDSYLFDLDNKDGEVYCIDARYYGNISRFINHLCDPNIIPVRVFMLHQDLRFPRIAFFSSRDIRTGEELGFDYGDRFWDIKSKYFTCQCGSEKCKHSAEAIALEQSRLARLDPHPELLPELGSLPPVNT

SEQ ID NO: 47 - DNA, 2427 bp, Homo sapiens
atggccgccgccgatgccgaggcagttccggcgaggggggagcctcagcaggattgctgtgtgaaaaccgagctgctgggagaagagacacctatggctgccgatgaaggctcagcagagaaacaggcaggagaggcccacatggctgcggacggtgagaccaatgggtcttgtgaaaacagcgatgccagcagtcatgcaaatgctgcaaagcacactcaggacagcgcaagggtcaacccccaggatggcaccaacacactaactcggatagcggaaaatggggtttcagaaagagactcagaagcggcgaagcaaaaccacgtcactgccgacgactttgtgcagacttctgtcatcggcagcaacggatacatcttaaataagccggccctacaggcacagcccttgaggactaccagcactctggcctcttcgctgcctggccatgctgcaaaaacccttcctggaggggctggcaaaggcaggactccaagcgcttttccccagacgccagccgccccaccagccacccttggggaggggagtgctgacacagaggacaggaagctcccggcccctggcgccgacgtcaaggtccacagggcacgcaagaccatgccgaagtccgtcgtgggcctgcatgcagccagtaaagatcccagagaagttcgagaagctagagatcataaggaaccaaaagaggagatcaacaaaaacatttctgactttggacgacagcagcttttaccccccttcccatcccttcatcagtcgctacctcagaaccagtgctacatggccaccacaaaatcacagacagcttgcttgccttttgttttagcagctgcagtatctcggaagaaaaaacgaagaatgggaacctatagcctggttcctaagaaaaagaccaaagtattaaaacagaggacggtgattgagatgtttaagagcataactcattccactgtgggttccaagggggagaaggacctgggcgccagcagcctgcacgtgaatggggagagcctggagatggactcggatgaggacgactcagaggagctcgaggaggacgacggccatggtgcagagcaggcggccgcgttccccacagaggacagcaggacttccaaggagagcatgtcggaggctgatcgcgcccagaagatggacggggagtccgaggaggagcaggagtccgtggacaccggggaggaggaggaaggcggtgacgagtctgacctgagttcggaatccagcattaagaagaaatttctcaagaggaaaggaaagaccgacagtccctggatcaagccagccaggaaaaggaggcggagaagtagaaagaagcccagcggtgccctcggttctgagtcgtataagtcatctgcaggaagcgctgagcagacggcaccaggagacagcacagggtacatggaagtttctctggactccctggatctccgagtcaaaggaattctgtcttcacaagcagaagggttggccaacggtccagatgtgctggagacagacggcctccaggaagtgcctctctgcagctgccggatggaaacaccgaagagtcgagagatcaccacactggccaacaaccagtgcatggctacagagagcgtggaccatgaattgggccggtgcacaaacagcgtggtcaagtatgagctgatgcgcccctccaacaaggccccgctcctcgtgctgtgtgaagaccaccggggccgcatggtgaagcaccagtgctgtcctggctgtggctacttctgcacagcgggtaattttatggagtgtcagcccgagagcagcatctctcaccgtttccacaaagactgtgcctctcgagtcaataacgccagctattgtccccactgtggggaggagagctccaaggccaaagaggtgacgatagctaaagcagacaccacctcgaccgtgacaccagtccccgggcaggagaagggctcggccctggagggcagggccgacaccacaacgggcagtgctgccgggccaccactctcggaggacgacaagctgcagggtgcagcctcccacgtgcccgagggctttgatccaacgggacctgctgggcttgggaggccaactcccggcctttcccagggaccagggaaggaaaccttggagagcgctctcatcgccctcgactcggaaaaacccaagaagcttcgcttccacccaaagcagctgtacttctccgccaggcaaggggagcttcagaaggtgctcctcatgctggtggacggaattgaccccaacttcaaaatggagcaccagaataagcgctctccactgcacgccgcggcagaggctggacacgtggacatctgccacatgctggttcagttctgcaggctgggaagcccaaggtcgaggggctgcctttggtga

SEQ ID NO: 48 - protein, 808 aa, Homo sapiens
MAAADAEAVPARGEPQQDCCVKTELLGEETPMAADEGSAEKQAGEAHMAADGETNGSCENSDASSHANAAKHTQDSARVNPQDGTNTLTRIAENGVSERDSEAAKQNHVTADDFVQTSVIGSNGYILNKPALQAQPLRTTSTLASSLPGHAAKTLPGGAGKGRTPSAFPQTPAAPPATLGEGSADTEDRKLPAPGADVKVHRARKTMPKSVVGLHAASKDPREVREARDHKEPKEEINKNISDFGRQQLLPPFPSLHQSLPQNQCYMATTKSQTACLPFVLAAAVSRKKKRRMGTYSLVPKKKTKVLKQRTVIEMFKSITHSTVGSKGEKDLGASSLHVNGESLEMDSDEDDSEELEEDDGHGAEQAAAFPTEDSRTSKESMSEADRAQKMDGESEEEQESVDTGEEEEGGDESDLSSESSIKKKFLKRKGKTDSPWIKPARKRRRRSRKKPSGALGSESYKSSAGSAEQTAPGDSTGYMEVSLDSLDLRVKGILSSQAEGLANGPDVLETDGLQEVPLCSCRMETPKSREITTLANNQCMATESVDHELGRCTNSVVKYELMRPSNKAPLLVLCEDHRGRMVKHQCCPGCGYFCTAGNFMECQPESSISHRFHKDCASRVNNASYCPHCGEESSKAKEVTIAKADTTSTVTPVPGQEKGSALEGRADTTTGSAAGPPLSEDDKLQGAASHVPEGFDPTGPAGLGRPTPGLSQGPGKETLESALIALDSEKPKKLRFHPKQLYFSARQGELQKVLLMLVDGIDPNFKMEHQNKRSPLHAAAEAGHVDICHMLVQFCRLGSPRSRGCLW

SEQ ID NO: 49 - protein, 7 aa, Artificial Sequence
  synthetic oligopeptide
IEGRMDP

SEQ ID NO: 50 - DNA, 20 bp, Artificial Sequence
  synthetic oligonucleotide
aatggccaaaaagcatgtag

SEQ ID NO: 51 - DNA, 20 bp, Artificial Sequence
  synthetic oligonucleotide
ggtgagtgagtaaacaagcc

SEQ ID NO: 52 - DNA, 20 bp, Artificial Sequence
  synthetic oligonucleotide
gtaaacaagcctgggccggg

SEQ ID NO: 53 - DNA, 20 bp, Artificial Sequence
  synthetic oligonucleotide
gtgagtgagtaaacaagcct

(19) United States
(12) Patent Application Publication
(10) Pub. No.: US 2024/0158750 A1
(43) Pub. Date: May 16, 2024

(54) STROMA-FREE NK CELL DIFFERENTIATION FROM HUMAN PLURIPOTENT STEM CELLS

(71) Applicant: The Children's Medical Center Corporation, Boston, MA (US)

(72) Inventors: Ran Jing, Boston, MA (US); Georg Q. Daley, Camebridge, MA (US)

(73) Assignee: The Children's Medical Center Corporation, Boston, MA (US)

(21) Appl. No.: 18/279,336

(22) PCT Filed: Mar. 4, 2022
(86) PCT No.: PCT/US2022/018991

§ 371 Date: Aug. 29, 2023

Related U.S. Application Data
(60) Provisional application No. 63/157,112, filed on Mar. 5, 2021.

(51) Int. Cl. C12N 5/0783

(52) U.S. Cl. CPC C12N 5/0646; C12N 2501/125; C12N 2501/2303; C12N 2501/2307; C12N 2501/2315; C12N 2501/26; C12N 2501/42; C12N 2502/1323; C12N 2506/28; C12N 2506/45; C12N 2510/00

(57) ABSTRACT

The technology described herein is directed to stromal-free methods of NK cell differentiation. Also described herein are cells differentiated using stromal-free methods and compositions comprising such cells. In some embodiments, the cells can be genetically modified. In some embodiments, the cells or compositions comprising said cells can be administered to a patient to treat cancer or as a cellular replacement therapy to treat a condition.

### CROSS-REFERENCE TO RELATED APPLICATIONS

This application claims benefit under 35 U.S.C. § 119(e) of U.S. Provisional Application No. 63/157,112 filed Mar. 5, 2021, the contents of which are incorporated herein by reference in their entirety.

### GOVERNMENT SUPPORT

This invention was made with Government support under Grant No. U01HL134812 awarded by the National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 4, 2022, is named 701039-097920WOPT_SL.txt and is 109,545 bytes in size.

### TECHNICAL FIELD

The technology described herein relates to immune cell differentiation methods.

### BACKGROUND

There is a lack of supply of functional immune cells for the in vivo cellular replacement therapy, therapy for a host of diseases, disorders and conditions, and for the in vitro studies of disease modeling, drug screening, and hematological diseases. Natural Killer (NK) cells are key components of human immune system and have great therapeutic potential. Human induced pluripotent stem cells (iPSCs) represent an ideal source for scalable manufacture of off-the-shelf products for cell therapy. However, the generation of mature and functional NK cells from iPSCs has proven to be difficult. Additionally, the differentiation of iPSC requires co-culture with mouse stromal cells, which limits the translational potential of iPSC-derived NK cells. As such there is a need for high-yield, clinically applicable NK cell differentiation methods.

### SUMMARY

The technology described herein is directed to methods of differentiating cells. In some embodiments, the differentiated cells are immune cells. In some embodiments, the methods are directed to NK cell differentiation. In one aspect, the method described herein is a stroma-free NK cell differentiation method, i.e., a method that does not comprise co-culturing with stromal cells or any other type of supporting cell. Co-culture with stromal cells such as mouse stromal cells limits the translational potential of iPSC-derived NK cells; for example, there can be fears of transplantation rejection due to the presence of stromal cells. Additionally, as described herein, stroma-free NK cell differentiation methods result in increased numbers or percentages of CD56+NK cells and decreased numbers or percentages of CD3+ T cells compared to differentiation methods comprising stromal co-culture. Inhibition of a histone methyltransferase (e.g., EZH1) further increases the yield of CD56+NK cells using the stromal-free differentiation methods described herein. Accordingly, NK cells differentiated with the stromal-free differentiation methods described herein exhibit at least one of the following unexpected benefits compared to stromal co-culture methods: (1) increased potential for transplantation in humans; (2) increased number or percentage of total CD56+NK cells; (3) decreased number or percentage of total CD3+ T cells; (4) increased expression of NK cell receptors; or (5) increased expression of genes that are responsible for lymphoid differentiation/function (see, e.g., Example 1, FIG. 2, FIGS. 3A-3B, Example 3, FIG. 4, FIGS. 5A-5C, FIGS. 6A-6C).

In one aspect, described herein is a method comprising: (a) inhibiting a histone methyltransferase in a population of CD34⁺ hemogenic endothelium; and (b) differentiating the population of CD34⁺ hemogenic endothelium in Natural Killer (NK)-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; (b) inhibiting a histone methyltransferase in the resultant population of CD34⁺ hemogenic endothelium; and (c) differentiating the resultant population of CD34⁺ hemogenic endothelium in Natural Killer (NK)-cell-differentiation media in the presence of a Notch ligand for a sufficient time to promote differentiation into a population of CD56⁺ NK cells.

In one aspect, described herein is a method comprising: (a) inhibiting an epigenetic regulator in a population of CD34⁺ hemogenic endothelium; and (b) differentiating the population of CD34⁺ hemogenic endothelium in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; (b) inhibiting an epigenetic regulator in the resultant population of CD34⁺ hemogenic endothelium; and (c) differentiating the resultant population of CD34⁺ hemogenic endothelium in NK-cell-differentiation media in the presence of a Notch ligand for a sufficient time to promote differentiation into a population of CD56⁺ NK cells.

In one aspect, described herein is a method comprising: (a) inhibiting G9a and/or GLP in a population of CD34⁺ hemogenic endothelium; and (b) differentiating the population of CD34⁺ hemogenic endothelium in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; (b) inhibiting G9a and/or GLP in the resultant population of CD34⁺ hemogenic endothelium; and (c) differentiating the resultant population of CD34⁺ hemogenic endothelium in NK-cell-differentiation media in the presence of a Notch ligand for a sufficient time to promote differentiation into a population of CD56⁺ NK cells.

In one aspect, described herein is a method comprising differentiating a population of CD34⁺ hemogenic endothelium in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media in the presence of a Notch ligand for a sufficient time to promote differentiation into a population of CD56⁺ NK cells.

In some embodiments of any of the aspects, the Notch ligand is attached to a solid substrate.

In some embodiments of any of the aspects, the Notch ligand is attached to a cell culture dish.

In some embodiments of any of the aspects, the Notch ligand is not derived from a stromal cell.

In some embodiments of any of the aspects, differentiating the hemogenic endothelium in the presence of a Notch ligand does not comprise co-culturing with a stromal cell expressing a Notch ligand.

In some embodiments of any of the aspects, differentiating the hemogenic endothelium in the presence of a Notch ligand does not comprise co-culturing with OP9-DLL1 cells or OP9-DLL4 cells.

In some embodiments of any of the aspects, the Notch ligand is selected from the group consisting of Delta-like-1 (DLL1), Delta-like-4 (DLL4), immobilized Delta1^{ext-IgG}, and immobilized Delta4^{ext-IgG}.

In some embodiments of any of the aspects, immobilized Delta1^{ext-IgG} consists of an extracellular domain of human Delta-like-1 fused to the Fc domain of human IgG1.

In some embodiments of any of the aspects, the Notch ligand is DLL4.

In some embodiments of any of the aspects, the Notch ligand is provided at a concentration of at most 5 μg/ml.

In some embodiments of any of the aspects, the sufficient time to promote differentiation into a population of CD56⁺ NK cells is at least 4 weeks.

In some embodiments of any of the aspects, the NK-cell-differentiation media is serum-free.

In some embodiments of any of the aspects, the NK-cell-differentiation media comprises SCF, FLT3, and IL7.

In some embodiments of any of the aspects, the NK-cell-differentiation media comprises 30 ng/ml-100 ng/mL SCF; 15 ng/ml-100 ng/mL FLT3; and 5 ng/ml-20 ng/ml IL7.

In some embodiments of any of the aspects, the NK-cell-differentiation media comprises 30 ng/mL SCF, 15 ng/ml FLT3, and 20 ng/ml IL7.

In some embodiments of any of the aspects, the NK-cell-differentiation media comprises 50 ng/mL SCF, 50 ng/ml FLT3, and 10 ng/ml IL7.

In some embodiments of any of the aspects, the NK-cell-differentiation media comprises 100 ng/mL SCF, 100 ng/ml FLT3, and 5 ng/ml IL7.

In some embodiments of any of the aspects, the NK-cell-differentiation media comprises a composition as described in Table 7 or 8.

In some embodiments of any of the aspects, the NK-cell-differentiation media comprises 30 ng/ml SCF, 20 ng/ml FLT3, and 25 ng/ml IL7.

In some embodiments of any of the aspects, the NK-cell-differentiation media further comprises thrombopoietin (TPO).

In some embodiments of any of the aspects, the NK-cell-differentiation media further comprises 5 ng/mL thrombopoietin (TPO) for at least the first 2 weeks of differentiating in the NK-cell-differentiation media.

In some embodiments of any of the aspects, the NK-cell-differentiation media further comprises interleukin-15 (IL-15).

In some embodiments of any of the aspects, the NK-cell-differentiation media further comprises 10 ng/mL interleukin-15 (IL-15) starting after at least the first 2 weeks of differentiating in the NK-cell-differentiation media.

In some embodiments of any of the aspects, the NK-cell-differentiation media further comprises interleukin-3 (IL-3).

In some embodiments of any of the aspects, the NK-cell-differentiation media further comprises 5 ng/mL interleukin-3 (IL-3) for at least the first week of differentiating in the NK-cell-differentiation media.

In some embodiments of any of the aspects, the method further comprises a step of CD56⁺ NK cell enrichment.

In some embodiments of any of the aspects, the population of pluripotent stem cells comprises induced pluripotent stem cells (iPS cells) or embryonic stem cells (ESC).

In some embodiments of any of the aspects, the induced pluripotent stem cells are produced by introducing into mature cells at least one reprogramming factor selected from the group consisting of: OCT4, SOX2, KLF4, c-MYC, nanog, and LIN28, or any combination thereof (see, e.g., Table 9).

In some embodiments of any of the aspects, the induced pluripotent stem cells are produced by introducing into mature cells combination of at least two reprogramming factor as described in Table 9 herein.

In some embodiments of any of the aspects, the induced pluripotent stem cells are produced by introducing only reprogramming factors OCT4, SOX2, KLF4 and optionally c-MYC or nanog and LIN28 into mature cells.

In some embodiments of any of the aspects, the induced pluripotent stem cells are produced by introducing the reprogramming factors two or more times into the mature cells.

In some embodiments of any of the aspects, the population of pluripotent stem cells is differentiated into a population of CD34⁺ hemogenic endothelium using embryoid bodies or 2D adherent cultures.

In some embodiments of any of the aspects, the sufficient time to promote differentiation into a population of CD34⁺ hemogenic endothelium is at least 8 days.

In some embodiments of any of the aspects, the aggregation media comprises BMP4, SB-431542, CHIR99021, bFGF, VEGF, IL-6, IL-11, IGF-1, SCF, and EPO.

In some embodiments of any of the aspects, the aggregation media comprises: (a) BMP4 for at least days 0 and 2; (b) SB-431542 for at least day 2; (c) CHIR99021 for at least day 2; (d) bFGF for at least day 1, 2, 3, and 6; (e) VEGF for at least days 3 and 6; (f) IL-6 for at least day 6; (g) IL-11 for at least day 6; (h) IGF-1 for at least day 6; (i) SCF for at least day 6; and/or (j) EPO for at least day 6.

In some embodiments of any of the aspects, the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO.

In some embodiments of any of the aspects, BMP4 is at a concentration of about 10 ng/ml in the aggregation media.

In some embodiments of any of the aspects, SB-431542 is at a concentration of about 6 mM in the aggregation media.

In some embodiments of any of the aspects, CHIR99021 is at a concentration of about 3 mM in the aggregation media.

In some embodiments of any of the aspects, bFGF is at a concentration of about 5 ng/ml in the aggregation media.

In some embodiments of any of the aspects, VEGF is at a concentration of about 15 ng/ml in the aggregation media.

In some embodiments of any of the aspects, IL-6 is at a concentration of about 10 ng/ml in the aggregation media.

In some embodiments of any of the aspects, IL-11 is at a concentration of about 5 ng/mL in the aggregation media.

In some embodiments of any of the aspects, IGF-1 is at a concentration of about 25 ng/mL in the aggregation media.

In some embodiments of any of the aspects, SCF is at a concentration of about 50 ng/mL in the aggregation media.

In some embodiments of any of the aspects, EPO is at a concentration of about 2 U/ml in the aggregation media.

In some embodiments of any of the aspects, the method further comprises selecting or isolating the resultant population of CD34⁺ hemogenic endothelium using expression of surface markers on the population of CD34⁺ hemogenic endothelium.

In some embodiments of any of the aspects, the population of CD34⁺ hemogenic endothelium is CD45 negative/low.

In some embodiments of any of the aspects, the population of CD34⁺ hemogenic endothelium is CD38 negative/low.

In some embodiments of any of the aspects, the method further comprises the step of genetically modifying the PSC (e.g., iPSC), the resultant population of CD34⁺ hemogenic endothelium, or the resultant population of CD56⁺ NK cells.

In some embodiments of any of the aspects, the genetic modification is removing an endogenous NK cell receptor and/or expressing a chimeric antigen receptor (CAR).

In some embodiments of any of the aspects, the genetic modification reduces immunogenicity in the cell.

In some embodiments of any of the aspects, the genetic modification that reduces immunogenicity is editing an endogenous HLA.

In some embodiments of any of the aspects, the genetic modification that reduces immunogenicity comprises removing or editing HLA class I or HLA class II.

In some embodiments of any of the aspects, the genetic modification that reduces immunogenicity comprises expressing at least one tolerance-promoting immunomodulatory molecule selected from the group consisting of: HLA-G, HLA-E, CD47, and PD-L1.

In some embodiments of any of the aspects, the genetic modification that reduces immunogenicity comprises expressing at least one immunomodulatory molecule selected from the group consisting of: CCL21, PD-L1, FasL, SERPINB9, H2-M3, CD47, CD200 and MFGE8.

In some embodiments of any of the aspects, the histone methyltransferase catalyzes the addition of methyl group to the histone 3 lysine residue 9 (H3K9) and/or histone 3 lysine residue 27 (H3K27).

In some embodiments of any of the aspects, the histone methyltransferase H3K9 and/or H3K27 is inhibited by a small molecule inhibitor or a nucleic acid inhibitor.

In some embodiments of any of the aspects, the histone methyltransferase H3K9 and/or H3K27 small molecule inhibitor is a heterorganic compound or an organometallic compound.

In some embodiments of any of the aspects, the histone methyltransferase H3K9 and/or H3K27 small molecule inhibitor is selected from the group consisting of BIX-01294, UNC0638, E72, BRD4770, A-366, chaetocin, UNC0224, UNC0631, UNC0646, EPZ005687, EPZ-6438 (E7438), 3-deazaneplanocin A (DZNep), EI1, GSK343, GSK126, and UNC1999.

In some embodiments of any of the aspects, the nucleic acid inhibitor is a nucleic acid targeting the expression of histone methyltransferase.

In some embodiments of any of the aspects, the nucleic acid inhibitor is an RNA interference inhibitor or agent.

In some embodiments of any of the aspects, the nucleic acid inhibitor is an EZH1-specific nucleic acid inhibitor.

In some embodiments of any of the aspects, the nucleic acid inhibitor is an aptamer that binds EZH1.

In some embodiments of any of the aspects, the nucleic acid inhibitor is an EZH1-specific RNA interference agent or a vector encoding an EZH1-specific RNA interference agent, wherein the RNA interference agent comprises one or more of the nucleotide sequences selected from SEQ ID NOS: 11-19.

In some embodiments of any of the aspects, the nucleic acid inhibitor is an EZH1-specific CRISPR guide RNA in combination with a Cas enzyme, or a vector encoding an EZH1-specific CRISPR guide RNA and a Cas enzyme, wherein the CRISPR guide RNA comprises one or more of the nucleotide sequences selected from SEQ ID NOS: 20-41.

In some embodiments of any of the aspects, the nucleic acid inhibitor is an EZH1-specific CRISPRi guide RNA in combination with a dCas enzyme, or a vector encoding an EZH1-specific CRISPRi guide RNA and a dCas enzyme, wherein the CRISPRi guide RNA comprises one or more of the nucleotide sequences selected from SEQ ID NOS: 51-53.

In some embodiments of any of the aspects, the epigenetic regulator is a DNA-methyltransferase (DNMT); a methyl-CpG-binding domain (MBD) protein; a DNA demethylase; a histone methyl transferase (HMT); a methyl-histone binding protein; a histone demethylase; a histone acetyl transferase (HAT); an acetyl-binding protein; or a histone deacetylase (HDAC).

In some embodiments of any of the aspects, the epigenetic regulator is inhibited by a small molecule inhibitor or a nucleic acid inhibitor.

In some embodiments of any of the aspects, the inhibitor of the epigenetic regulator is selected from the group consisting of: UNC0224; MC1568; and CAY10591.

In some embodiments of any of the aspects, the inhibitor of the epigenetic regulator is UNC0224.

In some embodiments of any of the aspects, the inhibitor of the epigenetic regulator is MC1568.

In some embodiments of any of the aspects, the inhibitor of the epigenetic regulator is CAY10591.

In some embodiments of any of the aspects, the inhibitor of the epigenetic regulator is provided at a concentration of at least 500 nM.

In some embodiments of any of the aspects, G9a and/or GLP is inhibited by a small molecule inhibitor.

In some embodiments of any of the aspects, G9a and/or GLP is inhibited by a nucleic acid inhibitor.

In some embodiments of any of the aspects, the G9a and/or GLP inhibitor is selected from the group consisting of: UNC0224; UNC0638; A366; BRD4770; BIX01294; UNC0642; UNC0631; UNC0646; UNC0321; E72; BIX-01338; BRD9539; Chaetocin; and DCG066.

In some embodiments of any of the aspects, the G9a and/or GLP inhibitor is UNC0224.

In some embodiments of any of the aspects, the G9a and/or GLP inhibitor is UNC0638.

In some embodiments of any of the aspects, the G9a and/or GLP inhibitor is A366

In some embodiments of any of the aspects, the G9a and/or GLP inhibitor is BRD4770

In some embodiments of any of the aspects, the G9a and/or GLP inhibitor is BIX01294

In some embodiments of any of the aspects, the G9a and/or GLP inhibitor is UNC0642

In some embodiments of any of the aspects, the G9a and/or GLP inhibitor is UNC0631

In some embodiments of any of the aspects, the G9a and/or GLP inhibitor is UNC0646

In some embodiments of any of the aspects, the G9a and/or GLP inhibitor is UNC0321

In some embodiments of any of the aspects, G9a and/or GLP inhibitor is E72

In some embodiments of any of the aspects, the G9a and/or GLP inhibitor is BIX-01338

In some embodiments of any of the aspects, the G9a and/or GLP inhibitor is BRD9539

In some embodiments of any of the aspects, the G9a and/or GLP inhibitor is Chaetocin

In some embodiments of any of the aspects, the G9a and/or GLP inhibitor is DCG066.

In some embodiments of any of the aspects, the G9a and/or GLP inhibitor is provided at a concentration of 300 nM-5 μM.

In some embodiments of any of the aspects, the G9a inhibitor comprises SEQ ID NO: 50, or a nucleic acid sequence that is at least 95% identical and maintains the same function.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: (a) differentiating a population of pluripotent stem cells in aggregation media for a sufficient time to promote differentiation into a population of CD34⁺ hemogenic endothelium; and differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: differentiating a population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

In one aspect, described herein is a method comprising: differentiating a population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.

In one aspect, described herein is a method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

In one aspect, described herein is a method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: differentiating a population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In one aspect, described herein is a method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In some embodiments of any of the aspects, the method further comprises inhibiting G9a and/or GLP in the population of CD34⁺ hemogenic endothelium.

In some embodiments of any of the aspects, the method further comprises inhibiting EZH1 in the population of CD34⁺ hemogenic endothelium.

In some embodiments of any of the aspects, the method further comprises inhibiting EZH1 and G9a in the population of CD34⁺ hemogenic endothelium.

In some embodiments of any of the aspects, the population of CD56⁺ NK cells comprises an at least 2-fold higher number of CD56+NK cells than the number of CD56+NK cells produced by a NK differentiation method comprising stroma cells.

In some embodiments of any of the aspects, the population of CD56⁺ NK cells comprises an at least 2-fold higher percentage of CD56+NK cells than the percentage of CD56+NK cells produced by a NK differentiation method comprising stroma cells.

In one aspect, described herein is a cell produced by a method as described herein.

In one aspect, described herein is a composition comprising a cell as described herein or population thereof.

In some embodiments of any of the aspects, the composition further comprises a pharmaceutically acceptable carrier.

In one aspect, described herein is a pharmaceutical composition comprising a cell as described herein or population thereof, and a pharmaceutically acceptable carrier.

In some embodiments of any of the aspects, the pharmaceutical composition is for use in cellular replacement therapy in a subject.

In one aspect, described herein is a method of cellular replacement therapy, the method comprising administering a cell as described herein or population thereof, or a composition of as described herein, or a pharmaceutical composition as described herein to a recipient subject in need thereof.

In some embodiments of any of the aspects, the recipient subject has undergone chemotherapy and/or irradiation.

In some embodiments of any of the aspects, the recipient subject has cancer.

In some embodiments of any of the aspects, the recipient subject has deficiencies in immune function and/or immune cell reconstitution.

In some embodiments of any of the aspects, prior to transplanting, the cell or population thereof is treated ex vivo with prostaglandin E2 and/or antioxidant N-acetyl-L-cysteine (NAC) to promote subsequent engraftment in a recipient subject.

In some embodiments of any of the aspects, the cell or population thereof is autologous to the recipient subject.

In some embodiments of any of the aspects, the cell or population thereof is HLA type matched with the recipient subject.

In some embodiments of any of the aspects, the cell or population thereof is hypoimmunogenic. In certain embodiments, the hypoimmunogenecity of the cell is achieved by engineering (e.g., knocking in or knocking out certain genes).

In one aspect, described herein is a method of treating cancer, comprising administering an effective amount of an cell as described herein or population thereof, or a composition as described herein, or a pharmaceutical composition as described herein to a recipient subject in need thereof. In some embodiments of any of the aspects, the cell is a CD56+NK cell.

### BRIEF DESCRIPTION OF THE DRAWINGS

FIG. 1 is a flow cytometry plot showing cells produced from a traditional differentiation protocol. Co-culture with OP9-DLL1 cells yields both NK (CD56+) and T cells (CD3+) at a low efficiency. The culture conditions for NK and T cells included: (1) 5 weeks of co-culture of EB-derived CD34+ iPSC-derived hemogenic endothelium (HE) with OP9-DLL1 stromal cells; and (2) media supplemented with IL-7 (5 ng/ml), SCF (30 ng/ml), and Flt3 (5 ng/ml).

FIG. 2 is a series of flow cytometry plot showing cells produced from a stroma-free differentiation protocol, which can be used to more specifically generate T or NK cells after 5 weeks. The culture conditions for T cells included: (i) EB-derived CD34⁺ hemogenic endothelium (HE) on DLL4 coating (10 μg/ml), and (ii) IL-7 (50 ng/ml), SCF (100 ng/ml), Flt3 (100 ng/ml), and TPO (50 ng/ml, first 2 weeks). For more details concerning the stroma-free T cell differentiation protocol, see, e.g., International PCT publication WO 2021/150919, the content of which is incorporated herein by reference in its entirety. The culture conditions for NK cells included: EB-derived CD34⁺ hemogenic endothelium (HE) on DLL4 coating (5 μg/ml), IL7 (10 ng/ml), SCF (50 ng/ml), and Flt3 (50 ng/ml).

FIGS. 3A-3B are a series of graphs showing stroma-free NK cell differentiation. Human iPSCs were engineered with a CRISPRi system for DOX-inducible EZH1 knockdown (kd). These iPSCs were differentiated into CD34⁺ HE cells via EB formation (see, e.g., Example 2). The CD34⁺ HE cells were then induced to differentiate into NK cells using a stromal-free NK differentiation method as described herein. FIG. 3A is a series of flow cytometry plots showing the expression of NK cell marker CD56 and T cell marker CD3 detected at day 28 of differentiation. In the EZH1 kd group, cells were treated with 1 μg/ml DOX to induce the CRISPRi-mediated EZH1 knockdown for 14 days.

FIG. 3B is a bar graph of the results, showing a significant increase of NK cell differentiation efficiency after EZH1 knockdown, as seen by an increase in CD56+ cells. The experiment was performed on three differentiations of the 1157 iPSC cell line transduced with 3 individual CRISPRi gRNAs (see, e.g., SEQ ID NOs: 51-53).

FIG. 4 is a series of flow cytometry plots showing that NK cells produced according to the differentiation methods described herein expressed higher levels of activating/inhibitory NK cell receptors, as compared to NK cells produced by another method. Flow cytometry results show the expression of activating (NCR1, CD16) and inhibitory (NKG2A) receptors on NK cells derived from iPSCs using a previously published protocol (see, e.g., Li et al, 2018, Cell Stem Cell 3(2):181-192; the content of which is incorporated herein by reference in its entirety) or the stroma-free differentiation protocol described herein (without EZH1 or G9a knockdown). Note that the stroma-free NK cells exhibited 33.3% NCR1+ NKG2A+NK cells compared to 14.0% NCR1+ NKG2A+NK cells in the Kaufman protocol (a 137.9% increase), and the stroma-free NK cells exhibited 37.1% CD16+NK cells compared to 9.41% CD16+NK cells in the previously published protocol (a 294.3% increase).

FIGS. 5A-5C are a series of schematics and graphs showing that G9a (also referred to herein as EHMT2) repression promoted NK cell differentiation. FIG. 5A is a schematic showing the experimental design. FIG. 5B is representative flow cytometry results showing the production of CD5-CD56+NK cells from control iPSCs or iPSCs with EHMT2 (G9a) knockdown by doxycycline (Dox) induced CRISPRi (see, e.g., SEQ ID NO: 50) for 14 days. Note that in these representative flow cytometry plots, G9a knockdown resulted in 64.7% CD5-CD56+NK cells compared to 25.2% CD5-CD56+NK cells in the control. FIG. 5C is a dot pot showing the frequencies of CD5-CD56+NK cells derived from control iPSCs or iPSCs with EHMT2 (G9a) knockdown (n=3, * P<0.05).

FIGS. 6A-6C are a series of schematics and graphs showing that G9a modulates chromatin accessibility of lymphoid genes. FIG. 6A is a schematic showing the experimental design. FIG. 6B is a heatmap showing assay for transposase-accessible chromatin using sequencing (ATAC-seq) peaks enriched in iPSC-derived CD34+CD45+ HSPCs that were treated with control vehicle (“DMSO”), 500 nM of UNC0224 (“UNC low”), or 1 μM of UNC0224 (“UNC high”) during the conversion of CD34+CD45− HE into CD34+CD45+ HSPCs for 5 days; 5 peaks are shown, labeled K1-K5. FIG. 6C is a bar graph showing the GO terms of enriched pathways associated with significantly upregulated peaks in iPSC-HSPCs treated with UNC0224 (K5 peaks).

FIG. 7 is a schematic showing an exemplary NK cell differentiation protocol.

### DETAILED DESCRIPTION

Embodiments of the technology described herein include methods of differentiating cells. In some embodiments, the cells are immune cells. In some embodiments the cells are NK cells. NK cells, also known as large granular lymphocytes (LGL), are a type of cytotoxic lymphocyte critical to the innate immune system. NK cells (belonging to the group of innate lymphoid cells) are one of the three kinds of cells differentiated from the common lymphoid progenitor, the other two being B and T lymphocytes. NK cells are known to differentiate and mature in the bone marrow, lymph nodes, spleen, tonsils, and thymus, where they then enter into the circulation.

The role of NK cells is analogous to that of cytotoxic T cells in the vertebrate adaptive immune response. NK cells provide rapid responses to virus-infected cells, acting at around 3 days after infection, and respond to tumor formation. Immune cells typically detect the major histocompatibility complex (MHC) presented on infected cell surfaces, triggering cytokine release, causing the death of the infected cell by lysis or apoptosis. NK cells also have the ability to recognize and kill stressed cells in the absence of antibodies and MHC, allowing for a faster immune reaction. This role is especially important because harmful cells that are missing MHC I markers (e.g., cancer cells; virally infected cells) cannot be detected and destroyed by other immune cells, such as T lymphocyte cells. In addition to natural killer cells being effectors of innate immunity, both activating and inhibitory NK cell receptors play important functional roles, including self-tolerance and the sustaining of NK cell activity. NK cells also play a role in the adaptive immune response; numerous experiments have demonstrated their ability to readily adjust to the immediate environment and formulate antigen-specific immunological memory, fundamental for responding to secondary infections with the same antigen.

NK cells can be identified by the presence of CD56 and the absence of CD3 (CD56+, CD3−). NK cells do not express T-cell antigen receptors (TCR) or pan T marker CD3 or surface immunoglobulins (Ig) B cell receptors, but they usually express the surface markers CD16 (Fc7RIII) and CD57 in humans, or NK1.1 or NK1.2 in C57BL/6 mice. The NKp46 cell surface marker constitutes, at the moment, another NK cell marker of preference being expressed in both humans, several strains of mice (including BALB/c mice) and in three common monkey species. Non-limiting examples of NK cell maturation markers include: CD3−; CD56+; Fc gamma RIII/CD16+; CD57+; NK1.1+; NK1.2+; CD94+, CD122/IL-2 beta+; CD217/IL-7R alpha−; KIR family receptors+; NKG2A+; NKG2D+; NKp30+(also referred to as NCR3+); NKp44+(also referred to as NCR2+); NKp46+(also referred to as NCR1+); or NKp80+. Non-limiting examples of NK cell markers include TRAIL, IFNg, TNFa, granzyme B, or perforin. Such markers can be used to detect NK cells using methods such as magnetic-activated cell sorting (MACS) or fluorescence-activated cell sorting (FACS), using antibodies directed to at least one of the above markers.

NK cell differentiation methods typically comprise contacting with stromal cells. However, co-culture with stromal cells such as mouse stromal cells limits the translational potential of iPSC-derived NK cells; for example, there can be fears of transplantation rejection due to the presence of stromal cells. In one aspect, the method described herein is a stroma-free NK cell differentiation method, i.e., a method that does not comprise co-culturing with stromal cells or any other type of supporting cell. Stroma-free methods have also been described for T cell differentiation; see, e.g., Example 1 and International PCT publication WO 2021/150919, the content of which is incorporated herein by reference in its entirety.

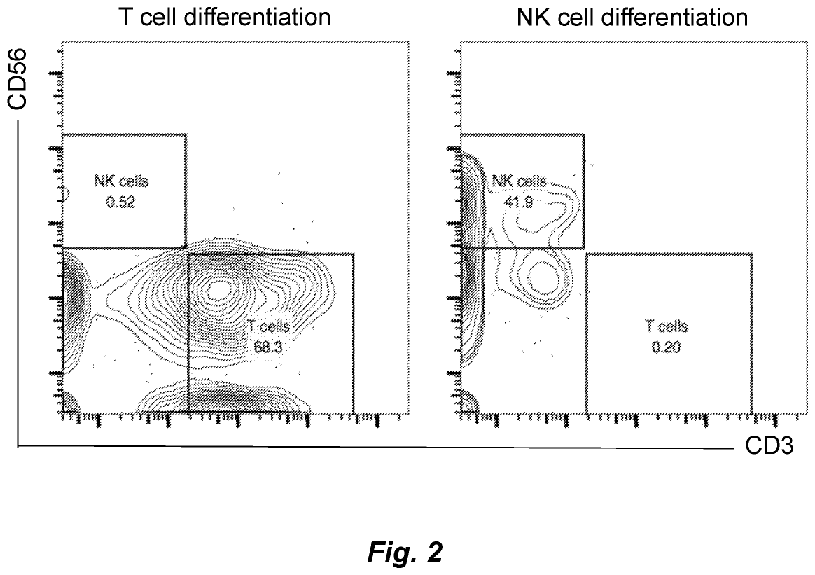

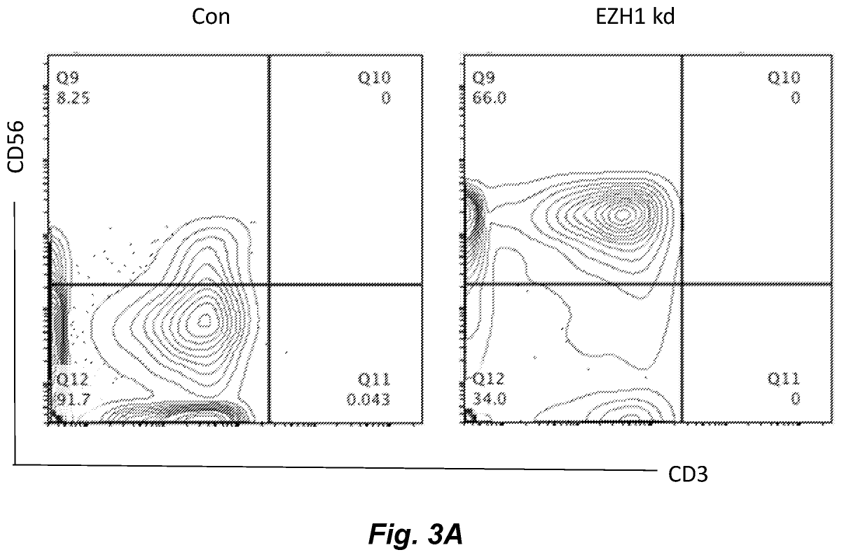

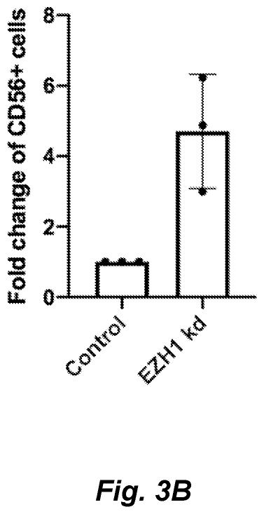

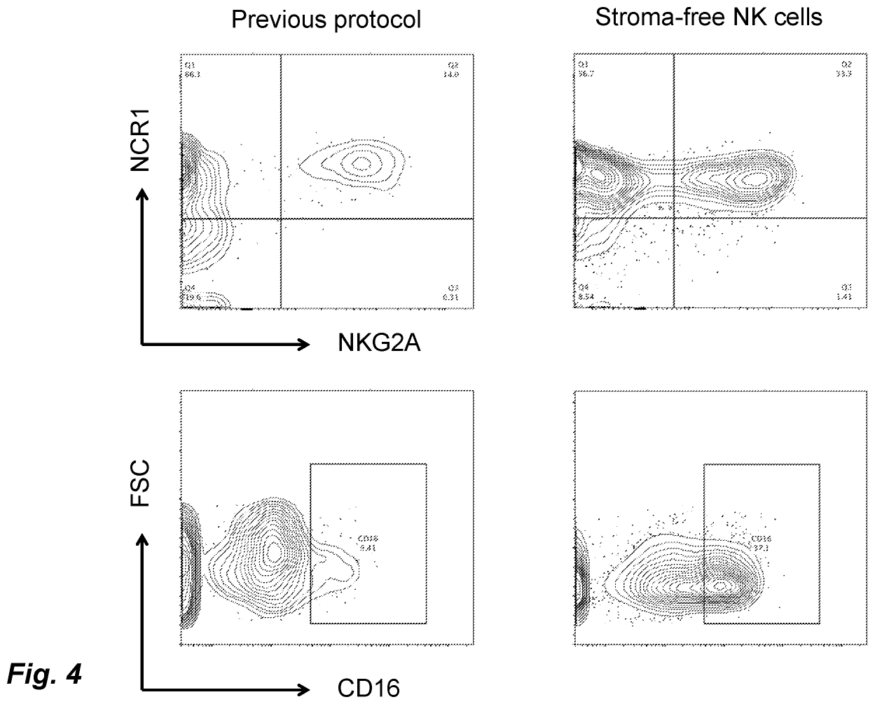

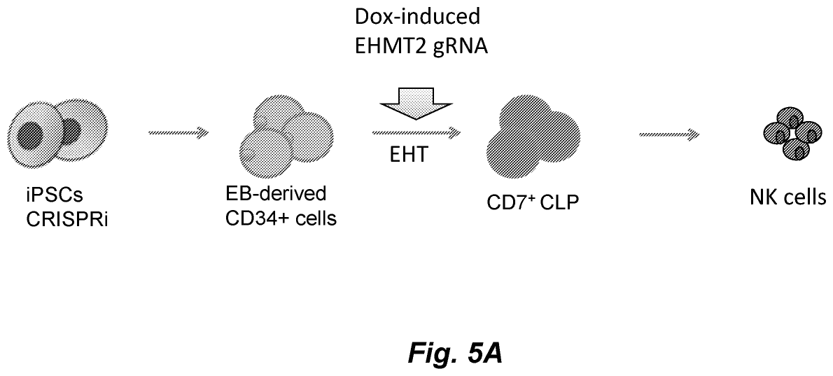

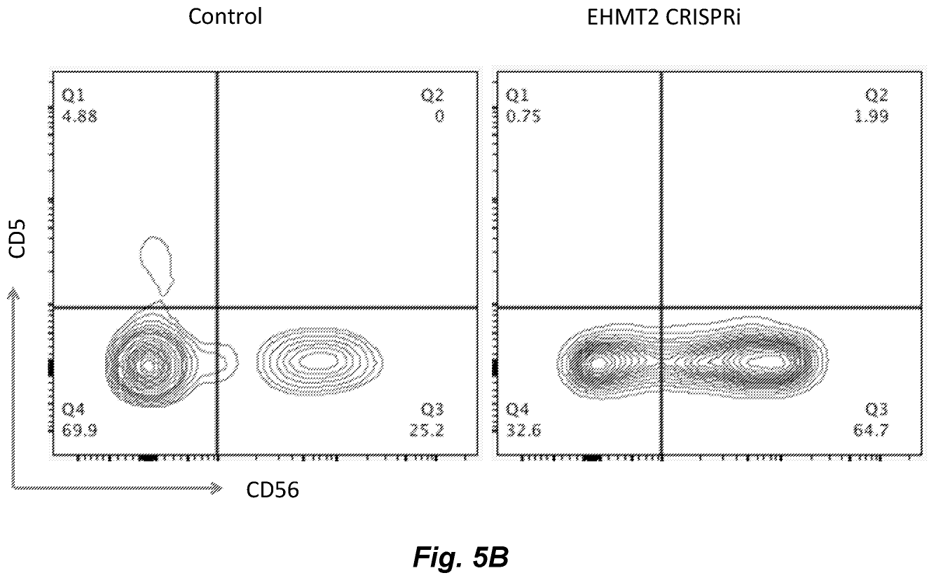

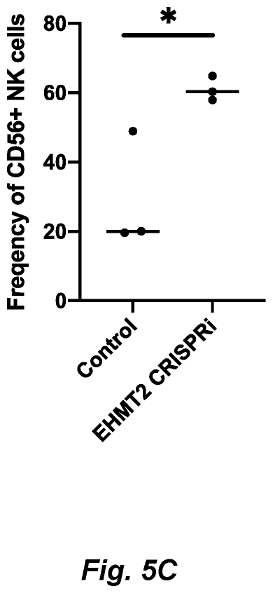

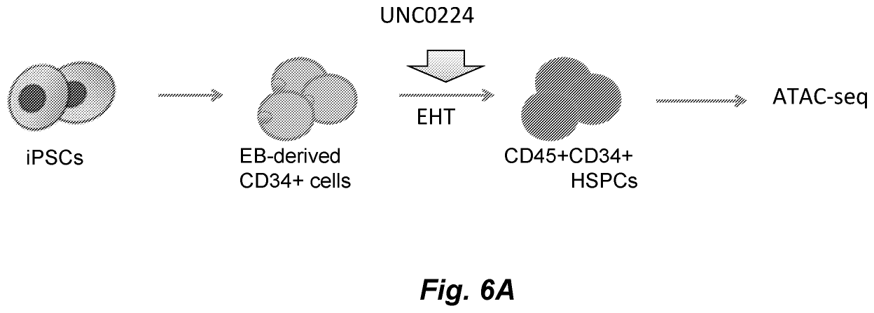

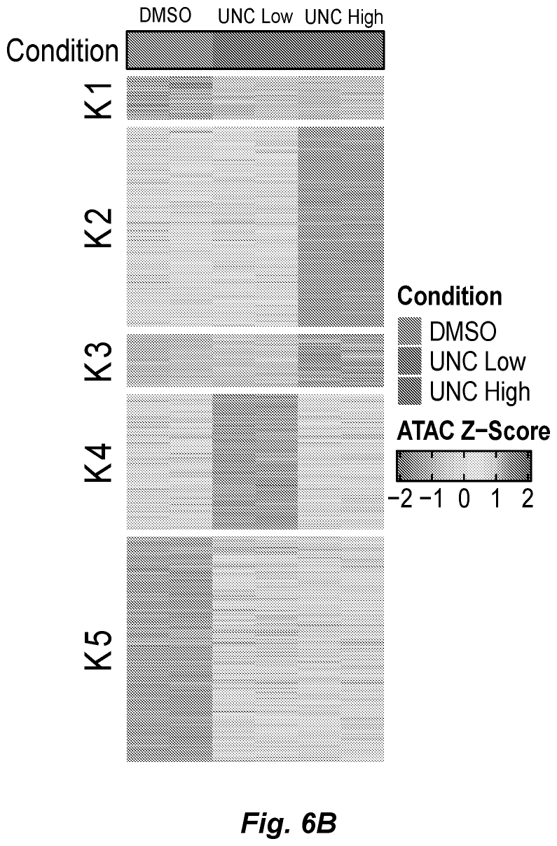

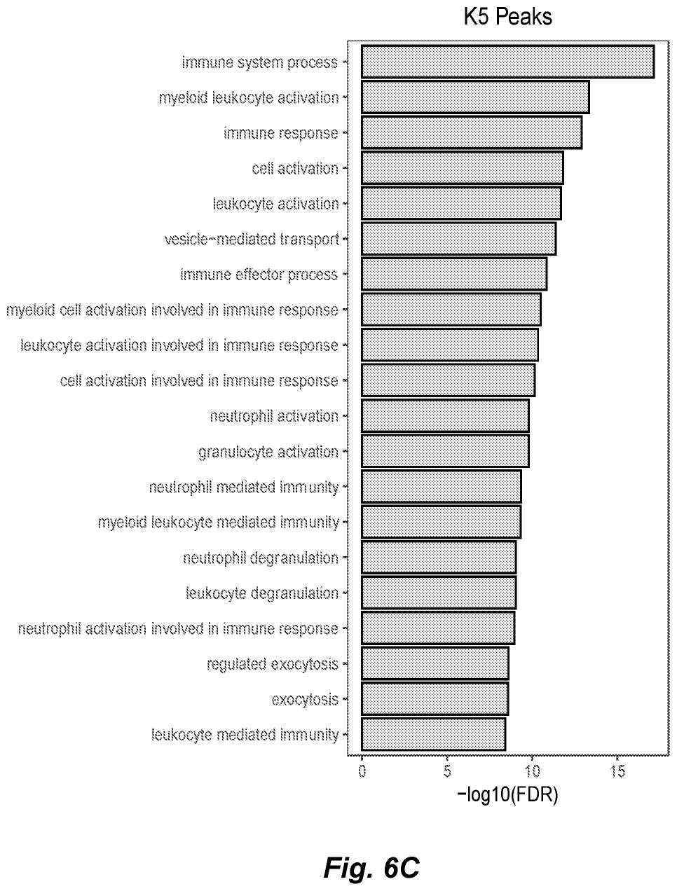

Additionally, as described herein, stroma-free NK cell differentiation methods result in increased numbers or percentages of CD56+NK cells and decreased numbers or percentages of CD3+ T cells compared to differentiation methods comprising stromal co-culture. Inhibition of a histone methyltransferase (e.g., EZH1) further increases the yield of CD56+NK cells using the stromal-free differentiation methods described herein. Accordingly, NK cells differentiated with the stromal-free differentiation methods described herein exhibit at least one of the following unexpected benefits compared to stromal co-culture methods: (1) increased potential for transplantation in humans; (2) increased number or percentage of total CD56+NK cells; (3) decreased number or percentage of total CD3+ T cells; (4) increased expression of NK cell receptors; or (5) increased expression of genes that are responsible for lymphoid differentiation/function (see, e.g., Example 1, FIG. 2, FIGS. 3A-3B, Example 3, FIG. 4, FIGS. 5A-5C, FIGS. 6A-6C).

### Differentiation Methods

In one aspect, described herein is a method comprising: (a) differentiating a population of pluripotent stem cells in an aggregation media for a sufficient time to promote differentiation into a population of CD34+ hemogenic endothelium; and (b) differentiating the resultant population of CD34+ hemogenic endothelium in NK-cell-differentiation media in the presence of a Notch ligand for a sufficient time to promote differentiation into a population of CD56+NK cells.

In some embodiments, the method further comprises inhibiting a histone methyltransferase in the resultant population of CD34+ hemogenic endothelium. Such an inhibition can increase the efficiency of differentiation into NK cells. Accordingly, in one aspect, described herein is a method comprising: (a) differentiating a population of pluripotent stem cells in an aggregation media for a sufficient time to promote differentiation into a population of CD34+ hemogenic endothelium; (b) inhibiting a histone methyltransferase in the resultant population of CD34+ hemogenic endothelium; and (c) differentiating the resultant population of CD34+ hemogenic endothelium in NK-cell-differentiation media in the presence of a Notch ligand for a sufficient time to promote differentiation into a population of CD56+NK cells.

In some aspects, the NK cell differentiation method begins with CD34⁺ hemogenic endothelium. Accordingly, in one aspect, described herein is a method comprising: (a) inhibiting a histone methyltransferase in a population of CD34⁺ hemogenic endothelium; and (b) differentiating the population of CD34⁺ hemogenic endothelium in Natural Killer (NK)-cell-differentiation media in the presence of a Notch ligand for a sufficient time to promote differentiation into a population of CD56⁺ NK cells. In another aspect, described herein is a method comprising differentiating a population of CD34⁺ hemogenic endothelium in NK-cell-differentiation media in the presence of a Notch ligand for a sufficient time to promote differentiation into a population of CD56⁺ NK cells.

In some aspects, the NK cell differentiation method uses or begins with CD34⁺CD45⁺ HSCs. Accordingly, in one aspect, described herein is a method comprising differentiating a population of CD34⁺CD45⁺ HSCs in NK-cell-differentiation media in the presence of a Notch ligand for a sufficient time to promote differentiation into a population of CD56⁺ NK cells.

In one aspect, described herein is a method comprising: (a) inhibiting a histone methyltransferase in a population of CD34⁺CD45⁺ hematopoietic stem cells (HSCs); and (b) differentiating the population of CD34⁺CD45⁺ HSCs in Natural Killer (NK)-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺CD45⁺ hematopoietic stem cells (HSCs); (b) inhibiting a histone methyltransferase in the resultant population of CD34⁺CD45⁺ HSCs; and (c) differentiating the resultant population of CD34⁺CD45⁺ HSCs in Natural Killer (NK)-cell-differentiation media in the presence of a Notch ligand for a sufficient time to promote differentiation into a population of CD56⁺ NK cells.

In one aspect, described herein is a method comprising: (a) inhibiting an epigenetic regulator in a population of CD34⁺CD45⁺ hematopoietic stem cells (HSCs); and (b) differentiating the population of CD34⁺CD45⁺ HSCs in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺CD45⁺ hematopoietic stem cells (HSCs); (b) inhibiting an epigenetic regulator in the resultant population of CD34⁺CD45⁺ HSCs; and (c) differentiating the resultant population of CD34⁺CD45⁺ HSCs in NK-cell-differentiation media in the presence of a Notch ligand for a sufficient time to promote differentiation into a population of CD56⁺ NK cells.

In one aspect, described herein is a method comprising: (a) inhibiting G9a and/or GLP in a population of CD34⁺CD45⁺ hematopoietic stem cells (HSCs); and (b) differentiating the population of CD34⁺CD45⁺ HSCs in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺CD45⁺ hematopoietic stem cells (HSCs); (b) inhibiting G9a and/or GLP in the resultant population of CD34⁺CD45⁺ HSCs; and (c) differentiating the resultant population of CD34⁺CD45⁺ HSCs in NK-cell-differentiation media in the presence of a Notch ligand for a sufficient time to promote differentiation into a population of CD56⁺ NK cells.

In one aspect, described herein is a method comprising: (a) differentiating a population of pluripotent stem cells in aggregation media for a sufficient time to promote differentiation into a population of CD34⁺CD45⁺ hematopoietic stem cells (HSCs); and (b) differentiating the resultant population of CD34⁺CD45⁺ HSCs in NK-cell-differentiation media in the presence of a Notch ligand for a sufficient time to promote differentiation into a population of CD56⁺ NK cells.

In one embodiment, the CD34⁺ hemogenic endothelium population is cultured in NK-cell-differentiation media comprising 100 ng/ml SCF, 100 ng/ml FLT3, and 5 ng/ml IL7 in the presence of 5 μg/mL Notch ligand for at least 4 weeks to promote differentiation into a population of CD56⁺ NK cells.

In one embodiment, the CD34⁺ hemogenic endothelium population is cultured in NK-cell-differentiation media comprising 50 ng/ml SCF, 50 ng/ml FLT3, and 10 ng/ml IL7 in the presence of 5 μg/mL Notch ligand for at least 4 weeks to promote differentiation into a population of CD56⁺ NK cells.

In one embodiment, the CD34⁺ hemogenic endothelium population is cultured in NK-cell-differentiation media comprising 30 ng/ml SCF, 15 ng/ml FLT3, and 20 ng/ml IL7 in the presence of 5 μg/mL Notch ligand for at least 4 weeks to promote differentiation into a population of CD56⁺ NK cells.

In one aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising, 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising, 100 ng/mL SCF, 100 ng/ml FLT3 and 5 ng/ml IL7 in the presence of 5 μg/mL Notch ligand for at least 4 weeks to promote differentiation into a population of CD56⁺ NK cells.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising, 50 ng/mL SCF, 50 ng/ml FLT3 and 10 ng/ml IL7 in the presence of 5 μg/mL Notch ligand for at least 4 weeks to promote differentiation into a population of CD56⁺ NK cells; wherein the NK-cell-differentiation media further comprises a G9a inhibitor for at least the first two weeks.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 100 ng/ml SCF, 100 ng/ml FLT3, and 5 ng/ml IL7 in the presence of 5 μg/mL Notch ligand for at least 4 weeks to promote differentiation into a population of CD56⁺ NK cells; wherein the NK-cell-differentiation media further comprises a G9a/GLP inhibitor for at least the first two weeks.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: (a) differentiating a population of pluripotent stem cells in aggregation media for a sufficient time to promote differentiation into a population of CD34⁺ hemogenic endothelium; and (b) differentiating the resultant population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: (a) differentiating a population of pluripotent stem cells in aggregation media for a sufficient time to promote differentiation into a population of CD34⁺ hemogenic endothelium; and differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: differentiating a population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

In another aspect, described herein is a method comprising: differentiating a population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.

In another aspect, described herein is a method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

In another aspect, described herein is a method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: differentiating a population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In another aspect, described herein is a method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

In some embodiments, the method further comprises inhibiting G9a and/or GLP in the population of CD34⁺ hemogenic endothelium. In some embodiments, the method further comprises inhibiting EZH1 in the population of CD34⁺ hemogenic endothelium. In some embodiments, the method further comprises inhibiting EZH1 and G9a in the population of CD34⁺ hemogenic endothelium.

### Pluripotent Stem Cells

In some embodiments, the stroma-free NK cell differentiation method comprises differentiating a population of pluripotent stem cells. Pluripotent stem cells (PSCs) have the potential to give rise to all the somatic tissues. In one embodiment of any method, cells, or composition described herein, the population of pluripotent stem cells is induced pluripotent stem cells (iPSCs) or embryonic stem cells (ESC). IPSC and ESC can be produced by any method known in the art. In some embodiments, the population of pluripotent stem cells comprises embryonic stem cells (ESC). Embryonic stem cells (ESCs) are stem cells derived from the undifferentiated inner mass cells of a human embryo. In other embodiments, the population of pluripotent stem cells comprises induced pluripotent stem cells (iPSC).

Directed differentiation of PSCs aims to recapitulate embryonic development to generate patient-matched tissues by specifying the three germ layers. A common theme in directed differentiation across all germ layers is the propensity of PSCs to give rise to embryonic- and fetal-like cell types, which poses a problem for integration and function in an adult recipient. This distinction is particularly striking in the hematopoietic system, which emerges in temporally and spatially separated waves at during ontogeny. The earliest “primitive” progenitors emerge in the yolk sac at 8.5 dpc and give rise to a limited repertoire of macrophages, megakaryocytes and nucleated erythrocytes. These early embryonic-like progenitors are generally myeloid-based and cannot functionally repopulate the bone marrow of adult recipients. By contrast, “definitive” cells with hematopoietic stem cell (HSC) potential emerge later in arterial endothelium within the aorta-gonad-mesonephros (AGM) and other anatomical sites. Directed differentiation of PSCs gives rise to hematopoietic progenitors, which resemble those found in the yolk sac of the early embryo. These lack functional reconstitution potential, are biased to myeloid lineages, and express embryonic globins. Thus, understanding key fate determining mechanisms that promote development of either primitive or definitive lineages is critical for specifying HSCs, and other adult-like cell types (e.g., red blood cells) from PSCs.

In some embodiments, the population of pluripotent stem cells (PSCs) comprises induced pluripotent stem cells (iPS cells). In some embodiments, the induced pluripotent stem cells are produced by introducing into mature cells at least one reprogramming factor selected from the group consisting of: OCT4, SOX2, KLF4, c-MYC, nanog, and LIN28. In some embodiments, the induced pluripotent stem cells are produced by introducing into mature cells a combination of reprogramming factors selected from the group consisting of: OCT4, SOX2, KLF4, c-MYC, nanog, and LIN28. Exemplary combinations of reprogramming factors are described in Table 9 herein. In some embodiments, the induced pluripotent stem cells are produced by introducing only reprogramming factors OCT4, SOX2, KLF4 and optionally c-MYC or nanog and LIN28 into mature cells. In some embodiments, the induced pluripotent stem cells are produced by introducing the reprogramming factors two or more times into the mature cells. In some embodiments, the induced pluripotent stem cells are produced by introducing DNA (e.g., vector(s), viral vector(s) such as Sendai virus (SeV)) encoding at least one reprogramming factor into mature cells. In some embodiments, the induced pluripotent stem cells are produced by introducing RNA encoding at least one reprogramming factor into mature cells. In some embodiments, the induced pluripotent stem cells are produced by introducing circular RNA encoding at least one reprogramming factor into mature cells.

TABLE 9
Exemplary combinations of reprogramming factors. In Table 9 herein,
each row indicates a particular combination. “x” indicates
that the given reprogramming factor is included in the combination.

| OCT4 | SOX2 | KLF4 | c-MYC | nanog | LIN28 |
|---|---|---|---|---|---|
| X | | | | | |
| | X | | | | |
| X | X | | | | |
| | | X | | | |
| X | | X | | | |
| | X | X | | | |
| X | X | X | | | |
| | | | X | | |
| X | | | X | | |
| | X | | X | | |
| X | X | | X | | |
| | | X | X | | |
| X | | X | X | | |
| | X | X | X | | |
| X | X | X | X | | |
| | | | | X | |
| X | | | | X | |
| | X | | | X | |
| X | X | | | X | |
| | | X | | X | |
| X | | X | | X | |
| | X | X | | X | |
| X | X | X | | X | |
| | | | X | X | |
| X | | | X | X | |
| | X | | X | X | |
| X | X | | X | X | |
| | | X | X | X | |
| X | | X | X | X | |
| | X | X | X | X | |
| X | X | X | X | X | |
| | | | | | X |
| X | | | | | X |
| | X | | | | X |
| X | X | | | | X |
| | | X | | | X |
| X | | X | | | X |
| | X | X | | | X |
| X | X | X | | | X |
| | | | X | | X |
| X | | | X | | X |
| | X | | X | | X |
| X | X | | X | | X |
| | | X | X | | X |
| X | | X | X | | X |
| | X | X | X | | X |
| X | X | X | X | | X |
| | | | | X | X |
| X | | | | X | X |
| | X | | | X | X |
| X | X | | | X | X |
| | | X | | X | X |
| X | | X | | X | X |
| | X | X | | X | X |
| X | X | X | | X | X |
| | | | X | X | X |
| X | | | X | X | X |
| | X | | X | X | X |
| X | X | | X | X | X |
| | | X | X | X | X |
| X | | X | X | X | X |
| | X | X | X | X | X |
| X | X | X | X | X | X |

In some embodiments, the pluripotent stem cells (PSCs) described herein are induced pluripotent stem cells (iPSCs). An advantage of using iPSCs is that the cells can be derived from the same subject to which the eventual cells would be reintroduced. That is, a somatic cell can be obtained from a subject, reprogrammed to an induced pluripotent stem cell, and then transfected and differentiated into a modified cell to be administered to the subject (e.g., autologous cells). Since the progenitors are essentially derived from an autologous source, the risk of engraftment rejection or allergic responses is reduced compared to the use of cells from another subject or group of subjects. In some embodiments, the cells for generating iPSCs are derived from non-autologous sources. In addition, the use of iPSCs negates the need for cells obtained from an embryonic source. Thus, in one embodiment, the PSCs used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a common myeloid stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not necessarily critical to the methods described. Thus, any method that re-programs a somatic cell to the pluripotent phenotype would be appropriate for use in the methods described herein.

Reprogramming methodologies for generating pluripotent cells using defined combinations of transcription factors have been described to induce pluripotent stem cells from somatic cells. Yamanaka and Takahashi converted mouse somatic cells to ES cell-like cells with expanded developmental potential by the direct transduction of Oct4, Sox2, Klf4, and optionally c-Myc. See U.S. Pat. Nos. 8,058,065 and 9,045,738 to Yamanaka and Takahashi. iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission, and tetraploid complementation.

Subsequent studies have shown that human iPS cells can be obtained using similar transduction methods, and the transcription factor trio, OCT4, SOX2, and NANOG, has been established as the core set of transcription factors that govern pluripotency. The production of iPS cells can be achieved by the introduction of nucleic acid sequences encoding stem cell-associated genes into an adult, somatic cell, using viral vectors.

OCT4, SOX2, KLF4 and c-MYC are the original four transcription factors identified to reprogram mouse fibroblasts into iPSCs. These same four factors were also sufficient to generate human iPSCs. OCT3/4 and SOX2 function as core transcription factors of the pluripotency network by regulating the expression of pluripotency-associated genes. Kruppel-like factor 4 (KLF4) is a downstream target of LIF-STAT3 signaling in mouse ES cells and regulates self-renewal. Human iPSCs can also be generated using four alternative factors; OCT4 and SOX2 are required but KLF4 and c-MYC could be replaced with NANOG, a homeobox protein important for the maintenance of pluripotency in both ES cells and early embryos, and LIN28, an RNA binding protein. The combination of OCT4, SOX2, NANOG and LIN28 reprogramming factors have been reported to be also sufficient to generate human iPSCs.

In one embodiment of any method, cells, or composition described herein, the iPSCs are produced, for example, by introducing exogenous copies of only three reprogramming factors OCT4, SOX2, and KLF4 into mature or somatic cells. In one embodiment of any method, cells, or composition described herein, c-MYC, or nanog and/or LIN28 are further introduced to iPSCs having exogenous gene coding copies of OCT4, SOX2, and KLF4 to differentiate into mature or somatic cells. In one embodiment of any method, cells, or composition described herein, the iPSCs are produced by introducing exogenous copies of reprogramming factors OCT4, SOX2, and KLF4, and optionally with c-MYC or nanog and/or LIN28 to differentiate into mature or somatic cells.

In one embodiment of any method, cells, or composition described herein, the iPSCs are produced by contacting mature cells with at least one vector, wherein the at least one vector carries an exogenous gene coding copy of reprogramming factors OCT4, SOX2, and KLF4, and optionally with c-MYC, or nanog and/or LIN28 to differentiate into mature or somatic cells, and wherein the reprogramming factors are expressed in vivo in the contacted mature or somatic cells. The contacting is in vitro or ex vivo. The reprogramming factors needed for differentiation can all be expressed by one vector (e.g., a vector that carries an exogenous gene coding copy of OCT4, SOX2, KLF4, and c-MYC). Alternatively, the reprogramming factors can be expressed in more than one vector that is each used to contact the iPSCs. For example, an iPSCs can be contacted by a first vector that carries an exogenous gene coding copy of OCT4, SOX2, and a second vector that carries an exogenous gene coding copy KLF4 and c-MYC.

In one embodiment of any disclosed methods, the iPS cell comprises at least an exogenous copy of a nucleic acid sequence encoding a reprogramming factor selected from the group consisting of genes Oct4 (Pou5f1), Sox2, cMyc, Klf4, Nanog, Lin 28 and Glisl. In some embodiments, combinations of reprogramming factors are used. For example, a combination of four reprogramming factors consisting of Oct4, Sox2, cMyc, and Klf4, or a combination of four reprogramming factors consisting of Oct4, Sox2, Nanog, and Lin 28.

In one embodiment of any method, cells, or composition described herein, the iPSCs are produced by introducing the disclosed reprogramming factors, or any combination of the reprograming factors two or more times into the mature or somatic cells. In one embodiment of any method, cells, or composition described herein, the iPSCs are produced by introducing the disclosed reprogramming factors, or any combination of the reprograming factors one or more times into the mature or somatic cells. In one embodiment, the combination of reprograming factors is different when a combination is introduced to the iPSC more than once, for example, the combination of Oct4 (Pou5f1), Sox2, cMyc, Klf4, Nanog is first introduced to the iPSCs, and the combination of Oct4 (Pou5f1), Sox2, cMyc is subsequently introduced to the iPSCs. In one embodiment of any method, cells, or composition described herein, the iPSCs are produced by contacting mature cells with the disclosed vector(s) factors two or more times into the mature/somatic cells. In some embodiments the reprogramming factors are encoded in one vector (e.g., polycistronically) or each in a separate vector.

In some embodiments, the population of pluripotent stem cells (e.g., iPSCs) are not differentiated in the presence of a Notch ligand. In some embodiments, the aggregation media used to promote the differentiation of the population of pluripotent stem cells (e.g., iPSCs) into a population of CD34+ hemogenic endothelium do not comprise a Notch ligand. In some embodiments, the cell culture vessel used during the differentiation of the population of pluripotent stem cells (e.g., iPSCs) into the population of CD34+ hemogenic endothelium does not comprise a Notch ligand.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see, e.g., Warren et al., Cell Stem Cell, 2010 Nov. 5; 7(5):618-30, this reference is incorporated herein by reference in its entirety). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment, the reprogramming is not effected by a method that alters the genome. Thus, in such embodiments, the reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, the contents of each of which are incorporated herein by reference in its entirety. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecatl, Esgl, Eras, Gdf3, Fgf4, Cripto, Daxl, Zpf296, Slc2a3, Rex1, Utf1, and Natl. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Many US patents and patent application Publications teach and describe methods of generating iPSCs and related subject matter. For examples, U.S. Pat. Nos. 8,058,065, 9,347,044, 9,347,042, 9,347,045, 9,340,775, 9,341,625, 9,340,772, 9,250,230, 9,132,152, 9,045,738, 9,005,975, 9,005,976, 8,927,277, 8,993,329, 8,900,871, 8,852,941, 8,802,438, 8,691,574, 8,735,150, 8,765,470, 8,058,065, 8,048,675, and US Patent Publication Nos: 20090227032, 20100210014, 20110250692, 20110201110, 20110200568, 20110223669, 20110306516, 20100021437, 20110256626, 20110044961, 20120276070, 20120214243, 20120263689, 20120128655, 20120100568, 20130295064, 20130029866, 20130059386, 20130183759, 20130189786, 20130295579, 20130130387, 20130157365, 20140234973, 20140227736, 20140093486, 20140301988, 20140170746, 20140178989, 20140349401, 20140065227, and 20150140662, all of which are incorporated herein by reference in their entireties.

In some embodiments, the iPSCs can be derived from somatic cells. Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells. In one embodiment of any method, cells, or composition described herein, the mature cells from which iPS cells are made include any somatic cells such as NK cells, B lymphocytes (B-cells), T lymphocytes, (T-cells), and fibroblasts and keratinocytes.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, skin, immune cells, hepatic, splenic, lung, peripheral circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; beta-III-tubulin; alpha-smooth muscle actin (α-SMA); fibroblast growth factor 4 (Fgf4), Cripto, Daxl; zinc finger protein 296 (Zfp296); N-acetyltransferase-1 (Nati); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sa114; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; 13-catenin, and Bmil. Such cells can also be characterized by the down-regulation of markers characteristic of the somatic cell from which the induced pluripotent stem cell is derived. In one embodiment, the iPSCs are derived from mature, differentiated, somatic cells.

In some embodiments, the population of pluripotent stem cells used in the differentiation methods described herein does not comprise CD34+ HSPCs or multipotent lymphoid progenitors (MLPs) purified from a patient sample. In some embodiments, the population of pluripotent stem cells does not comprise stem cells purified or isolated from cord blood or bone marrow samples. In some embodiments, the population of pluripotent stem cells is not derived from stem cells isolated from a patient sample (e.g., cord blood or bone marrow). In a preferred embodiment, the population of pluripotent stem cells comprise iPSCs, such as those derived from a somatic cell sample from a patient. See, e.g., Tabatabaei-Zavareh et al., J Immunol May 1, 2017, 198 (1 Supplement) 202.9.

### Hemogenic Endothelium

In some embodiments, the methods described herein comprise differentiating a population of pluripotent stem cells (e.g., iPSCs) into a population of cells with hematopoietic potential. In some embodiments, the population of cells with hematopoietic potential comprises hemogenic endothelium and/or hematopoietic stem cells (HSCs). The cells with hematopoietic potential (e.g., hemogenic endothelium, HSCs) can be produced using any method known in the art.

One exemplary approach to generate HSCs from hPSCs is to specify HSCs from its ontogenetic precursors. It is now widely accepted that HSCs originate from hemogenic endothelium (HE) in the aorta-gonad-mesonephros (AGM) and arterial endothelium in other anatomical sites. Recent work on the directed differentiation of HE from hPSCs have provided valuable insights into some of the signaling pathways that control the emergence of primitive or definitive populations; however, the endothelial-to-hematopoietic transition (e.g., HE to HSC) remains incompletely understood in human hematopoietic development.

As used herein, the term “hemogenic endothelium” refers to a unique subset of endothelial cells scattered within blood vessels that can differentiate into haematopoietic cells. In the developing mouse, HSCs arise beginning embryonic day 10.5 from a small population of endothelial cells with hemogenic potential (hemogenic endothelium) located within the aorta-gonad-mesonephros region. In a process known as endothelial to hematopoietic transition (EHT), endothelial cells in the floor of the aorta round up and bud into the extravascular space followed by reentry into the circulation via the underlying vein. In some embodiments, a population of cells comprising the properties of hemogenic endothelium is differentiated in vitro from a population of pluripotent stem cells (e.g., iPSCs). Said “cells comprising the properties of hemogenic endothelium” can also be referred to herein as hemogenic endothelium.

Efforts to derive HSCs from pluripotent stem cells (PSCs) are complicated by the fact that embryonic hematopoiesis consists of two programs, primitive and definitive, but only definitive hematopoiesis generates HSCs and thus the lymphoid lineage. Definitive hematopoiesis, as measured by T-lymphoid potential, emerges after the establishment of the primitive hematopoietic program and develops from a progenitor population that displays characteristics of hemogenic endothelium.

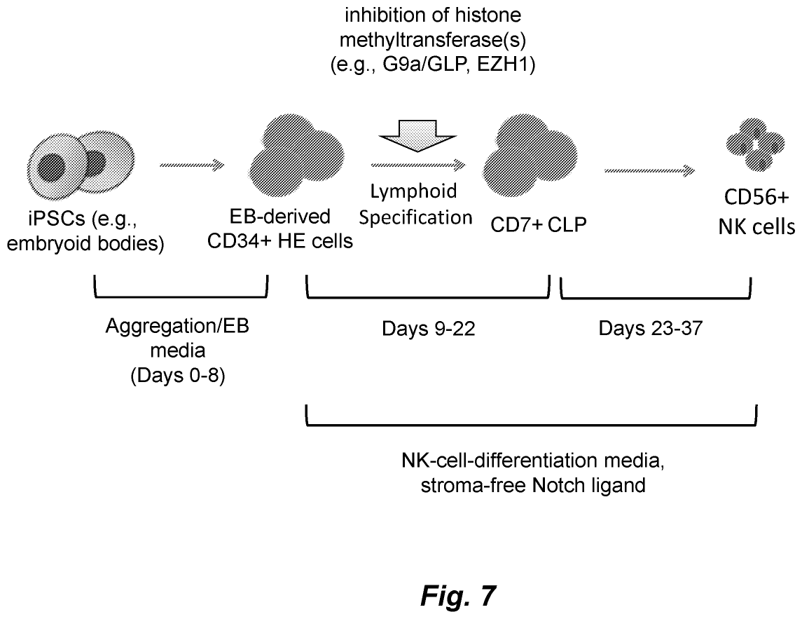

In some embodiments, the stroma-free NK cell differentiation method comprises differentiating a population of pluripotent stem cells in aggregation media for a sufficient time to promote differentiation into a population of CD34+ hemogenic endothelium. In some embodiments, the resultant CD34+ hemogenic endothelium can undergo definitive hematopoiesis and/or exhibits lymphoid potential. In some embodiments, the hemogenic endothelium differentiates or is differentiated into hematopoietic stem cells (HSCs). In some embodiments, HE are CD34+CD43−CD45− cells that can give rise to CD34+CD43+CD45+ HSCs via endothelial-to-haematopoietic transition (EHT). The CD34+CD43+CD45+ cells can comprise progenitor cells and can also be referred to as hematopoietic stem and progenitor cells (HSPCs). In some embodiments, the CD34+CD43−CD45− HE or CD34+CD43+CD45+ HSCs can differentiate into CD7+ common lymphoid progenitors (CLPs), which further differentiate into CD56⁺ NK cells (see, e.g., FIG. 7).

In some embodiments, the aggregation media comprises Iscove's Modified Dulbecco Media (IMDM). In some embodiments, the aggregation media comprises HAM'S F-12. In some embodiments, the aggregation media comprises a combination of Iscove's Modified Dulbecco Media (IMDM) and HAM'S F-12. In some embodiments, the aggregation media (e.g., IMDM and/or HAM's F12) comprises Penicillin-Streptomycin (Pen/Strep); N2 (e.g., LIFETECH 17502-048); B27 (e.g., LIFETECH 17504-044); BSA; L-glutamin; Ascorbic Acid; and/or Holo-Transferrin (see, e.g., Table 10).

In some embodiments, the aggregation media comprises STEMPRO-34 medium. In some embodiments, the aggregation media comprises STEMPRO-34 medium with at least one supplement. In some embodiments, the aggregation media (e.g., STEMPRO-34) comprises Penicillin-Streptomycin (Pen/Strep); L-glutamin; Ascorbic Acid; and/or Holo-Transferrin (see, e.g., Table 11).

In some embodiments, the population of pluripotent stem cells (e.g., iPSCs) is differentiated into a population of CD34+ hemogenic endothelium or CD34+CD45+ HSCs using embryoid bodies (EBs). In some embodiments, the population of pluripotent stem cells (e.g., iPSCs) is differentiated into a population of CD34+ hemogenic endothelium or CD34+CD45+ HSCs using 2D adherent cultures or pluripotent stem cell aggregates; see, e.g., Pineda et al., Differentiation patterns of embryonic stem cells in two versus three dimensional culture, Cells Tissues Organs. 2013; 197(5): 399-410, which is incorporated herein by reference. EBs are three-dimensional aggregates of pluripotent stem cells produced and cultured in vitro in the presence of serum. The EBs can generate a mixture of primitive and definitive hematopoietic progenitor cell types. Primitive progenitors equate to those that arise in vivo naturally in the earliest stages of embryonic development, whereas at later stages of maturation the embryonic populations give rise to definitive progenitor cells, which behave similarly to the cells typical of adult hematopoiesis.

In some embodiments, the sufficient time to promote differentiation into a population of CD34+ hemogenic endothelium is at least 8 days (e.g., at least 8, at least 9, at least 10 days, or more). In some embodiments, the sufficient time to promote differentiation into a population of CD34+ hemogenic endothelium is at most 8 days, at most 9 days, at most 10 days or more.

In some embodiments, the aggregation media comprises BMP4, SB-431542, CHIR99021, bFGF, VEGF, IL-6, IL-11, IGF-1, SCF, and EPO or any combination of the same. In some embodiments, the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/ml IL-11, 25 ng/ml IGF-1, 50 ng/ml SCF, and 2 U/ml EPO; see, e.g., Example 2 and Table 1 presented herein.

In some embodiments, the components of the aggregation media are varied during the differentiation of pluripotent stem cells into hemogenic endothelium. As a non-limiting example, embryoid bodies are differentiated in the presence of BMP4, followed by stage-specific addition of bFGF, VEGF, and hematopoietic cytokines (e.g., IL-6, IL-11, IGF-1, SCF, and EPO). Activin-nodal signaling can be manipulated (e.g., using SB-431542 and CHIR99021) between days 2 and 3. See, e.g., Example 2 herein below; and Sturgeon et al., Wnt signaling controls the specification of definitive and primitive hematopoiesis from human pluripotent stem cells, Nat Biotechnol. 2014 June; 32(6): 554-561, which is incorporated herein by reference.

In some embodiments, the aggregation media comprises BMP (e.g., 10 μg/mL BMP) during days 0, 1, and/or 2 of differentiation. In some embodiments, the aggregation media does not comprise BMP during days 3, 4, 5, 6, 7, or 8 of differentiation.

In some embodiments, the aggregation media comprises SB-431542 (e.g., 6 mM SB-431542) and/or CHIR99021 (e.g., 3 mM CHIR99021) during day 2 of differentiation. SB-431542 is a small-molecule antagonist of activin-nodal signaling CHIR99021 is a GSK-3 inhibitor and a Wnt agonist. Inhibition of activin-nodal signaling and activation of Wnt signaling has been shown to drive PSC differentiation into definitive progenitors (KDR⁺CD235a⁻) with lymphoid potential (see, e.g., Sturgeon 2014, supra, which is incorporated herein by reference). In some embodiments, the aggregation media does not comprise SB-431542 and/or CHIR99021 during days 0, 1, 3, 4, 5, 6, 7, and/or 8 of differentiation.

In some embodiments, the aggregation media comprises bFGF (e.g., 5 ng/ml bFGF) during days 1, 2, 3, 4, 5, 6, 7, and/or 8 of differentiation. In some embodiments, the aggregation media does not comprise bFGF during day 0 of differentiation.

In some embodiments, the aggregation media comprises VEGF (e.g., 15 ng/ml VEGF) during days 3, 4, 5, 6, 7, and/or 8 of differentiation. In some embodiments, the aggregation media does not comprise VEGF during days 0, 1, or 2 of differentiation.

In some embodiments, the aggregation media comprises hematopoietic cytokine(s) during days 6, 7, and/or 8 of differentiation. In some embodiments, the aggregation media does not comprise hematopoietic cytokine(s) during days 0, 1, 2, 3, 4, or 5 of differentiation. In some embodiments, the hematopoietic cytokines are selected from the group consisting of: IL-6 (e.g., 10 ng/ml IL-6), IL-11 (e.g., 5 ng/ml IL-11), IGF-1 (e.g., 25 ng/ml IGF-1), SCF (e.g., 50 ng/ml SCF), and EPO (e.g., 2 U/ml EPO).

In some embodiments, the differentiation method further comprises selecting or isolating the resultant population of CD34+ hemogenic endothelium using expression of surface markers on the population of CD34+ hemogenic endothelium. Non-limiting examples of methods for selecting or isolating hemogenic endothelium include magnetic-activated cell sorting (MACS) and fluorescence-activated cell sorting (FACS). In some embodiments, the surface marker for hemogenic endothelium is CD34 (e.g., high CD34 surface expression).

In some embodiments, additional positive or negative markers for hemogenic endothelium can include, but are not limited to, CD45, CD38, KDR, CD235, and CD43. In some embodiments, the population of CD34+ hemogenic endothelium is CD45 negative/low. In some embodiments, the population of CD34+ hemogenic endothelium is CD38 negative/low. In some embodiments, the population of CD34+ hemogenic endothelium is KDR+. In some embodiments, the population of CD34+ hemogenic endothelium is CD235 negative/low. In some embodiments, the population of CD34+ hemogenic endothelium is CD43 negative/low.

In some embodiments, the hemogenic endothelium and/or HSCs are produced using any method known in the art. As a non-limiting example, the method of differentiating PSCs into hemogenic endothelium can comprise the introduction of transcription factors such as ERG, HOXA5, HOXA9, HOXA10, LCOR, RUNX1, and/or SPI1; see, e.g., International Application No. WO 2018/048828, US Patent Application No. 2019/0225940, Doulatov et al., Cell Stem Cell. 2013 October 3, 13(4); Vo et al., Nature 2018, 553(7689): 506-510; the contents of each of which are incorporated herein by reference in their entireties.

In some embodiments, the hemogenic endothelium is not derived from PSCs but is rather derived directly from endothelial cells. For example, endothelial cells (e.g., from lung, brain, and other tissues) can be directly reprogrammed into hemogenic endothelium by transduction with transcription factors (e.g., Fosb, Gfil, Runxl, and Spil) and co-culture with an immortalized endothelial cell line; the endothelial cells can be further exposed to cell-extrinsic factors (e.g., serum, SB-431542, and/or endothelial mitogen). See, e.g., Lis et al., Nature. 2017 May 25, 545(7655):439-445; Blaser and Zon, Blood. 2018 Sep. 27; 132(13): 1372-1378, which are incorporated herein by reference.

### Inhibition of an Epigenetic Regulator

In some aspects described herein is a NK-cell-differentiation method comprising a step of inhibiting at least one epigenetic regulator. As used herein, the term “epigenetic regulator” refers to a factor, e.g., a polypeptide, e.g., an enzyme, that influences DNA methylation and/or histone modifications (e.g., histone acetylation, histone methylation), and as such affect the transcription levels of genes without an alteration (e.g., substitution or deletion) to the nucleotide sequence of the genome. Non-limiting examples of epigenetic regulators include: DNA-methyltransferase (DNMT; e.g., DNMT1; DNMT3a; DNMT3b); methyl-CpG-binding domain (MBD) protein (e.g., MeCP2; MBD1; MBD2; MCD4; KAISO; ZBTB4; ZBTB38; UHRHRF2); DNA demethylase (e.g., 5′-methylcytokine hydroxylase; TET1; TET2; TET3); histone methyltransferase (HMT; e.g., SUV39s; SET1s; EZH1; EZH2; Set2s; PRDMs; SMYDs; DOT1L; PRMTs; G9a; GLP); methyl-histone binding protein (e.g., HP1; Chdl; BPTF; L3MBTL1; ING2; BHC80; JMJD2A); histone demethylase (e.g., KDMs; e.g., LSDs; JHDMs; JMJDs; JARID; Uts; PHFs); histone acetyltransferase (HAT; e.g., HAT1; GCNS; PCAF; MYSTs; p300; CBP; SRC/p160); acetyl-binding proteins (e.g., BROMO-domain, DPF-domain, or YEATS-domain-containing proteins); histone deacetylase (HDAC; e.g., HDAC1; HDAC2; HDAC3; HDAC4; HDAC5; HDAC6; HDAC7; HDAC8; HDAC9; HDAC10; HDAC11; Sirt1; Sirt2; Sirt3; Sirt4; Sirt5; Sirt6; Sirt7). See, e.g., Cheng et al., Signal Transduction and Targeted Therapy volume 4, Article number: 62 (2019); the content of which is incorporated herein by reference in its entirety.

In some embodiments, the method comprises the step of, after the step of differentiating a population of pluripotent stem cells in aggregation media for a sufficient time to promote differentiation into a population of CD34+ hemogenic endothelium, inhibiting an epigenetic regulator in the resultant population of CD34+ hemogenic endothelium. In some embodiments, the method comprises the step of, prior to the step of differentiating a population of CD34+ hemogenic endothelium in NK-cell-differentiation media in the presence of a Notch ligand for a sufficient time to promote differentiation into a population of CD56+NK cells, inhibiting an epigenetic regulator in the population of CD34+ hemogenic endothelium. In some embodiments, inhibiting an epigenetic regulator can promote differentiation of CD34+ hemogenic endothelium into CD34+CD45+ hematopoietic stem cells (HSCs) and/or CD7+ CLPs. In some embodiments, inhibiting an epigenetic regulator can promote endothelial-to-hematopoietic transition (EHT) and/or lymphoid specification.

Accordingly, in one aspect, described herein is a method comprising: (a) differentiating a population of pluripotent stem cells in aggregation media for a sufficient time to promote differentiation into a population of CD34+ hemogenic endothelium; (b) inhibiting an epigenetic regulator in the resultant population of CD34+ hemogenic endothelium; and (c) differentiating the resultant population of CD34+ hemogenic endothelium in NK-cell-differentiation media in the presence of a Notch ligand for a sufficient time to promote differentiation into a population of CD56+NK cells. In another aspect, described herein is a method comprising: (a) inhibiting an epigenetic regulator in a population of CD34⁺ hemogenic endothelium; and (b) differentiating the population of CD34⁺ hemogenic endothelium in NK-cell-differentiation media in the presence of a Notch ligand for a sufficient time to promote differentiation into a population of CD56⁺ NK cells.

In some embodiment, CD34+ hemogenic endothelium is treated with an inhibitor of an epigenetic regulator. Exemplary inhibitors of an epigenetic regulator include an inhibitor of at least one of the following: DNMT; MBD; DNA demethylase; HMT; methyl-histone binding protein; histone demethylase; HAT; acetyl-binding protein; or HDAC. In some embodiments, the epigenetic regulator is an H3K9 methyltransferase. Methylation of H3K9 in humans relies mostly on members of the Suv39 family, namely EHMT1/GLP, EHMT2/G9a, SUV39H1, SUV39H2, SETDB1 and SETDB2, as well as then non-Suv39 enzymes PRDM2 and ASH1L.

Non-limiting examples of DNMT inhibitors include azacitidine; decitabine; guadecitabine; hydralazine. Non-limiting examples of HMT inhibitors include pinometostat; tazemetostat; GSK2816126; CPI-1205; TCP; ORY-2001; GSK2879552; 4SC-202. Non-limiting examples of HDAC inhibitors include valproic acid, phenylbutyrate; vorinostat; trichostatin A; belinostat; entinostat; panobinostat; mocetinostat; CI-994; romidepsin; nicotinamide; suramin; PRI-724; GSK525762; CPI-0610; R06870810; MK-8628.

In some embodiments, the inhibitor of an epigenetic regulator is selected from Table 3. In some embodiments, the inhibitor of an epigenetic regulator is selected from the group consisting of: SB939 (Pracinostat); 4-iodo-SAHA; Scriptaid; Oxaflatin (i.e., Oxamflatin); s-HDAC-42; UNC0224; Pyroxamide; MC1568; CAY10398; CAY10591; SAHA (Vorinostat) (SIH-359); SGI-1027; and Rucaparib (Rubraca™). In some embodiments, the inhibitor of an epigenetic regulator is selected from the group consisting of: SB939 (Pracinostat); 4-iodo-SAHA; Scriptaid; Oxaflatin (i.e., Oxamflatin); s-HDAC-42; UNC0224; Pyroxamide; MC1568; CAY10398; CAY10591; and SAHA (Vorinostat) (SIH-359); see, e.g., Table 3.

TABLE 3
Small molecule inhibitors that can promote NK cell differentiation (e.g., at 500
nM). All references cited in Table 3 are specifically incorporated herein by reference in their
entireties.

| Small | | |
|---|---|---|
| molecule | Function of small molecule | Structure of small molecule |
| SB939 (Pracinostat) | Pan-HDAC inhibitor (e.g., with IC50 of 40- 140 nM with exception for HDAC6). It has no activity against the class III isoenzyme SIRT1. | 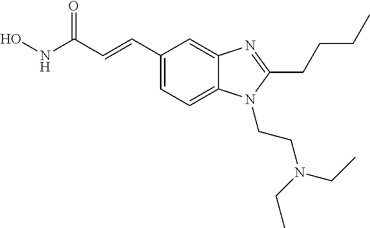 [SMILES: CCCCc1nc2cc(/C=C/C(=O)NO)ccc2n1CCN(CC)CC] |
| 4-iodo- SAHA | 4-iodo-SAHA is a hydrophobic derivative of the class I and class II HDAC (e.g., HDAC1 or HDAC6) inhibitor SAHA. | 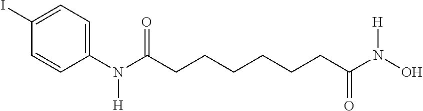 [SMILES: O=C(CCCCCCC(=O)Nc1ccc(I)cc1)NO] |
| Scriptaid | HDAC inhibitor (see, e.g., U.S. Pat. No. 6,544,957). | 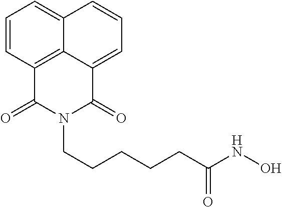 [SMILES: O=C(CCCCCN1C(=O)c2cccc3cccc(c23)C1=O)NO] |
| Oxaflatin (i.e., Oxamflatin) | HDAC inhibitor | 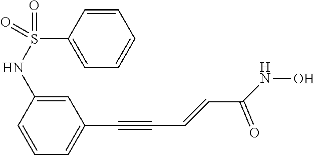 [SMILES: O=C(/C=C/C#Cc1cccc(NS(=O)(=O)c2ccccc2)c1)NO] |
| s-HDAC-42 | HDAC inhibitor | 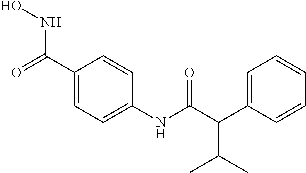 [SMILES: CC(C)C(C(=O)Nc1ccc(C(=O)NO)cc1)c1ccccc1] |
| UNC0224 | G9a and GLP inhibitor (H3K9 methyltransferase) | 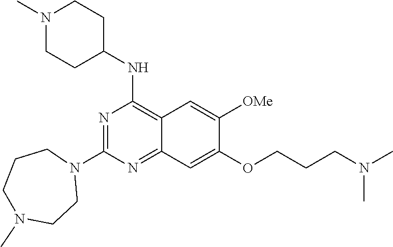 |
| Pyroxamide | HDAC (e.g., HDAC1) inhibitor | 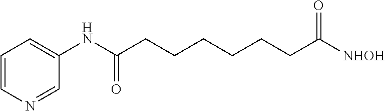 |
| MC1568 | Class II HDAC (e.g., HDAC4 and HDAC5) inhibitor. Displays no inhibition of class I HDAC activity (e.g., HDAC1, HDAC2, HDAC3). | 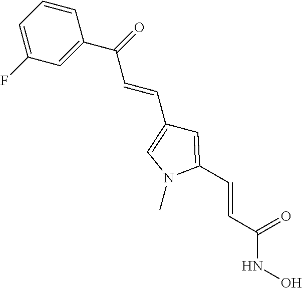 [SMILES: Cn1cc(/C=C/C(=O)c2cccc(F)c2)cc1/C=C/C(=O)NO] |
| CAY10398 | HDAC (e.g., HDAC1) inhibitor | 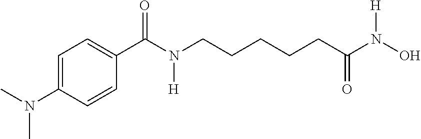 [SMILES: CN(C)c1ccc(C(=O)NCCCCCC(=O)NO)cc1] |
| CAY10591 | SIRT1 activator. Sirtuins (SIRTs) represent a distinct class of trichostatin A-insensitive lysyl-deacetylases (class III HDACs). | 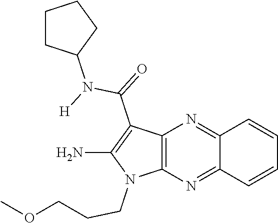 [SMILES: COCCCn1c(N)c(C(=O)NC2CCCC2)c2nc3ccccc3nc21] |
| SAHA (Vorinostat) (SIH-359) | Potent reversible pan-histone deacetylase HDAC inhibitor, including both class I and class II HDACs. | 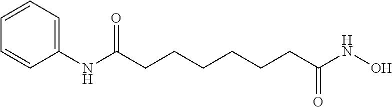 [SMILES: O=C(CCCCCCC(=O)Nc1ccccc1)NO] |
| SGI-1027 | DNA Methyltransferase (DNMT; e.g., DNMT1, DNMT3A, or DNMT3B) inhibitor | 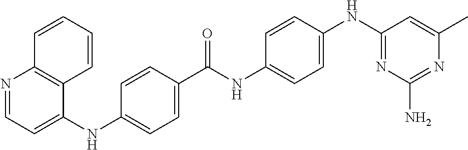 [SMILES: Cc1cc(Nc2ccc(NC(=O)c3ccc(Nc4ccnc5ccccc45)cc3)cc2)nc(N)n1] |
| Rucaparib (Rubraca ™) | poly(ADP-ribose) polymerase (PARP; e.g., PARP1, PARP2, PARP3) inhibitor | 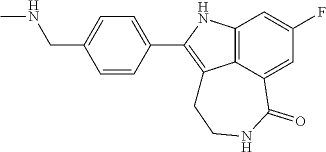 [SMILES: CNCc1ccc(-c2[nH]c3cc(F)cc4c3c2CCNC4=O)cc1] |
| 5-Methyl-2′- deoxycytidine | 5-Methyl-2′-deoxycytidine is a pyrimidine nucleoside that when incorporated into single-stranded DNA can act in cis to signal de novo DNA methylation; see, e.g., Christman et al. Proceedings of the National Academy of Sciences of the United States of America 92(16), 7347- 7351 (1995). | 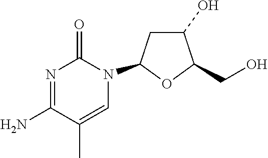 [SMILES: Cc1cn([C@H]2C[C@H](O)[C@@H](CO)O2)c(=O)nc1N] |

In some embodiments, the inhibitor of an epigenetic regulator is selected from the group consisting of: UNC0224; MC1568; 5-Methyl-2′-deoxycytidine; and CAY10591. In some embodiments, the inhibitor of an epigenetic regulator is UNC0224. In some embodiments, the inhibitor of an epigenetic regulator is MC1568. In some embodiments, the inhibitor of an epigenetic regulator is CAY10591.

In some embodiments, the inhibitor of an epigenetic regulator is 5-Methyl-2′-deoxycytidine.

In some embodiments, the inhibitor of an epigenetic regulator is provided at a concentration of at least 500 nM. In some embodiments, the inhibitor of an epigenetic regulator is provided at a concentration of at least 1.0 μM. In some embodiments, the inhibitor of an epigenetic regulator is provided at a concentration of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 at least 1.25 at least 1.5 at least 1.75 at least 2.0 at least 2.5 at least 3 at least 4 at least 5 at least 6 at least 7 at least 8 at least 9 or at least 10 μM. In some embodiments, the inhibitor of an epigenetic regulator is provided at a concentration of 1 nM-10 nM, 10 nM-50 nM, 50 nM-100 nM, 100 nM-500 nM, 500 nM-1 μM, 1 μM-5 μM, or 5 μM-10 μM.

In some embodiments, the cells (e.g., CD34+ hemogenic endothelium) are cultured exposed to an inhibitor of an epigenetic regulator until the development of CD56+NK cells. In some embodiments, the cells (e.g., CD34+ hemogenic endothelium) are cultured exposed to an inhibitor of an epigenetic regulator for about 14 days. In some embodiments, the cells (e.g., CD34+ hemogenic endothelium) are cultured exposed to an inhibitor of an epigenetic regulator for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more.

Inhibition of G9a and/or GLP

In some aspects described herein is a NK-cell-differentiation method comprising a step of inhibiting G9a and/or GLP. In some aspects described herein is a NK-cell-differentiation method comprising a step of inhibiting G9a. G9a can also be referred to interchangeably as Euchromatic Histone Lysine Methyltransferase 2 (EHMT2); Histone H3-K9 Methyltransferase 3; KMT1C; Lysine N-Methyltransferase 1C; BAT8; or NG36. G9a is a methyltransferase that methylates lysine residues of histone H3 (see, e.g., NCBI Gene ID: 10919; e.g., one of SEQ ID NOs: 45-46 or a sequence that is at least 95% identical and maintains the same function, or a functional fragment thereof). In some aspects described herein is a NK-cell-differentiation method comprising a step of inhibiting G9a-like protein (GLP). GLP is also referred to interchangeably as Euchromatic Histone Lysine Methyltransferase 1 (EHMT1); KMT1D; Eu-HMTase1; or Histone-Lysine N-Methyltransferase, H3 Lysine-9 Specific 5 (see, e.g., NCBI Gene ID: 79813; e.g., one of SEQ ID NOs: 47-48 or a sequence that is at least 95% identical and maintains the same function, or a functional fragment thereof).

G9a and GLP exist predominantly as a G9a-GLP heteromeric complex. G9a and GLP are the primary enzymes for mono- and dimethylation at Lys 9 of histone H3 (H3K9me1 and H3K9me2) in euchromatin. H3K9me represents a specific tag for epigenetic transcriptional repression by recruiting HP1 proteins to methylated histones. G9a/GLP also weakly methylates ‘Lys-27’ of histone H3 (H3K27me). G9a/GLP is also required for DNA methylation; the histone methyltransferase activity of G9a/GLP is not required for DNA methylation, suggesting that these two activities function independently. G9a/GLP is probably targeted to histone H3 by different DNA-binding proteins, e.g., E2F6, MGA, MAX and/or DP1. In addition to the histone methyltransferase activity, G9a/GLP also methylates non-histone proteins, e.g., dimethylation of ‘Lys-373’ of p53/TP53.

G9a also mediates monomethylation of ‘Lys-56’ of histone H3 (H3K56me1) in G1 phase, leading to promote interaction between histone H3 and PCNA and regulating DNA replication. G9a is also though to methylate histone H1. G9a also methylates CDYL, WIZ, ACIN1, DNMT1, HDAC1, ERCC6, KLF12, and itself. During GO phase, GLP may contribute to silencing of MYC- and E2F-responsive genes, suggesting a role in GO/G1 transition in cell cycle. In addition to the histone methyltransferase activity, GLP also methylates non-histone proteins: mediates dimethylation of ‘Lys-373’ of p53/TP53.

SEQ ID NO: 45, Homo sapiens euchromatic
histone lysine methyltransferase 2
(EHMT2), transcript variant 1, mRNA,
NCBI Reference Sequence: NM_001289413.1
(region 5-3706), 3702 bp
(SEQ ID NO: 45)
ATGCGGGGTCTACCGAGAGGGAGGGGGTTGATGCGGGCCCGGGGGAGGGG
TCGTGCGGCCCCTCCGGGCAGCCGAGGCCGCGGAAGGGGGGGGCCCCACA
GAGGAAGAGGTAGGCCCCGGAGCCTACTCTCTCTTCCCAGGGCCCAGGCA
TCCTGGACCCCCCAACTCTCTACTGGGCTGACCAGCCCTCCTGTCCCTTG
TCTCCCCTCCCAGGGGGAGGCCCCCGCTGAGATGGGGGCGCTGCTGCTGG
AGAAGGAAACCAGAGGAGCCACCGAGAGAGTTCATGGCTCTTTGGGGGAC
ACCCCTCGTAGTGAAGAAACCCTGCCCAAGGCCACCCCCGACTCCCTGGA
GCCTGCTGGCCCCTCATCTCCAGCCTCTGTCACTGTCACTGTTGGTGATG
AGGGGGCTGACACCCCTGTAGGGGCTACACCACTCATTGGGGATGAATCT
GAGAATCTTGAGGGAGATGGGGACCTCCGTGGGGGCCGGATCCTGCTGGG
CCATGCCACAAAGTCATTCCCCTCTTCCCCCAGCAAGGGGGGTTCCTGTC
CTAGCCGGGCCAAGATGTCAATGACAGGGGGGGGAAAATCACCTCCATCT
GTCCAGAGTTTGGCTATGAGGCTACTGAGTATGCCAGGAGCCCAGGGAGC
TGCAGCAGCAGGGTCTGAACCCCCTCCAGCCACCACGAGCCCAGAGGGAC
AGCCCAAGGTCCACCGAGCCCGCAAAACCATGTCCAAACCAGGAAATGGA
CAGCCCCCGGTCCCTGAGAAGCGGCCCCCTGAAATACAGCATTTCCGCAT
GAGTGATGATGTCCACTCACTGGGAAAGGTGACCTCAGATCTGGCCAAAA
GGAGGAAGCTGAACTCAGGAGGTGGCCTGTCAGAGGAGTTAGGTTCTGCC
CGGCGTTCAGGAGAAGTGACCCTGACGAAAGGGGACCCCGGGTCCCTGGA
GGAGTGGGAGACGGTGGTGGGTGATGACTTCAGTCTCTACTATGATTCCT
ACTCTGTGGATGAGCGCGTGGACTCCGACAGCAAGTCTGAAGTTGAAGCT
CTAACTGAACAACTAAGTGAAGAGGAGGAGGAGGAAGAGGAGGAAGAAGA
AGAAGAGGAAGAGGAGGAGGAAGAGGAAGAAGAAGAGGAAGATGAGGAGT
CAGGGAATCAGTCAGATAGGAGTGGTTCCAGTGGCCGGCGCAAGGCCAAG
AAGAAATGGCGAAAAGACAGCCCATGGGTGAAGCCGTCTCGGAAACGGCG
CAAGCGGGAGCCTCCGCGGGCCAAGGAGCCACGAGGGGTGTCCAATGACA
CATCTTCGCTGGAGACAGAGCGAGGGTTTGAGGAGTTGCCCCTGTGCAGC
TGCCGCATGGAGGCACCCAAGATTGACCGCATCAGCGAGAGGGCGGGGCA
CAAGTGCATGGCCACTGAGAGTGTGGACGGAGAGCTGTCAGGCTGCAATG
CCGCCATCCTCAAGCGGGAGACCATGAGGCCATCCAGCCGTGTGGCCCTG
ATGGTGCTCTGTGAGACCCACCGCGCCCGCATGGTCAAACACCACTGCTG
CCCGGGCTGCGGCTACTTCTGCACGGCGGGCACCTTCCTGGAGTGCCACC
CTGACTTCCGTGTGGCCCACCGCTTCCACAAGGCCTGTGTGTCTCAGCTG
AATGGGATGGTCTTCTGTCCCCACTGTGGGGAGGATGCTTCTGAAGCTCA
AGAGGTGACCATCCCCCGGGGTGACGGGGTGACCCCACCGGCCGGCACTG
CAGCTCCTGCACCCCCACCCCTGTCCCAGGATGTCCCCGGGAGAGCAGAC
ACTTCTCAGCCCAGTGCCCGGATGCGAGGGCATGGGGAACCCCGGCGCCC
GCCCTGCGATCCCCTGGCTGACACCATTGACAGCTCAGGGCCCTCCCTGA
CCCTGCCCAATGGGGGCTGCCTTTCAGCCGTGGGGCTGCCACTGGGGCCA
GGCCGGGAGGCCCTGGAAAAGGCCCTGGTCATCCAGGAGTCAGAGAGGCG
GAAGAAGCTCCGTTTCCACCCTCGGCAGTTGTACCTGTCCGTGAAGCAGG
GCGAGCTGCAGAAGGTGATCCTGATGCTGTTGGACAACCTGGACCCCAAC
TTCCAGAGCGACCAGCAGAGCAAGCGCACGCCCCTGCATGCAGCCGCCCA
GAAGGGCTCCGTGGAGATCTGCCATGTGCTGCTGCAGGCTGGAGCCAACA
TAAATGCAGTGGACAAACAGCAGCGGACGCCACTGATGGAGGCCGTGGTG
AACAACCACCTGGAGGTAGCCCGTTACATGGTGCAGCGTGGTGGCTGTGT
CTATAGCAAGGAGGAGGACGGTTCCACCTGCCTCCACCACGCAGCCAAAA
TCGGGAACTTGGAGATGGTCAGCCTGCTGCTGAGCACAGGACAGGTGGAC
GTCAACGCCCAGGACAGTGGGGGGTGGACGCCCATCATCTGGGCTGCAGA
GCACAAGCACATCGAGGTGATCCGCATGCTACTGACGCGGGGCGCCGACG
TCACCCTCACTGACAACGAGGAGAACATCTGCCTGCACTGGGCCTCCTTC
ACGGGCAGCGCCGCCATCGCCGAAGTCCTTCTGAATGCGCGCTGTGACCT
CCATGCTGTCAACTACCATGGGGACACCCCCCTGCACATCGCAGCTCGGG
AGAGCTACCATGACTGCGTGCTGTTATTCCTGTCACGTGGGGCCAACCCT
GAGCTGCGGAACAAAGAGGGGGACACAGCATGGGACCTGACTCCCGAGCG
CTCCGACGTGTGGTTTGCGCTTCAACTCAACCGCAAGCTCCGACTTGGGG
TGGGAAATCGGGCCATCCGCACAGAGAAGATCATCTGCCGGGACGTGGCT
CGGGGCTATGAGAACGTGCCCATTCCCTGTGTCAACGGTGTGGATGGGGA
GCCCTGCCCTGAGGATTACAAGTACATCTCAGAGAACTGCGAGACGTCCA
CCATGAACATCGATCGCAACATCACCCACCTGCAGCACTGCACGTGTGTG
GACGACTGCTCTAGCTCCAACTGCCTGTGCGGCCAGCTCAGCATCCGGTG
CTGGTATGACAAGGATGGGCGATTGCTCCAGGAATTTAACAAGATTGAGC
CTCCGCTGATTTTCGAGTGTAACCAGGCGTGCTCATGCTGGAGAAACTGC
AAGAACCGGGTCGTACAGAGTGGCATCAAGGTGCGGCTACAGCTCTACCG
AACAGCCAAGATGGGCTGGGGGGTCCGCGCCCTGCAGACCATCCCACAGG
GGACCTTCATCTGCGAGTATGTCGGGGAGCTGATCTCTGATGCTGAGGCT
GATGTGAGAGAGGATGATTCTTACCTCTTCGACTTAGACAACAAGGATGG
AGAGGTGTACTGCATAGATGCCCGTTACTATGGCAACATCAGCCGCTTCA
TCAACCACCTGTGTGACCCCAACATCATTCCCGTCCGGGTCTTCATGCTG
CACCAAGACCTGCGATTTCCACGCATCGCCTTCTTCAGTTCCCGAGACAT
CCGGACTGGGGAGGAGCTAGGGTTTGACTATGGCGACCGCTTCTGGGACA
TCAAAAGCAAATATTTCACCTGCCAATGTGGCTCTGAGAAGTGCAAGCAC
TCAGCCGAAGCCATTGCCCTGGAGCAGAGCCGTCTGGCCCGCCTGGACCC
ACACCCTGAGCTGCTGCCCGAGCTCGGCTCCCTGCCCCCTGTCAACACAT
GA
SEQ ID NO: 46, histone-lysine N-methyl-
transferase EHMT2 isoform c (Homo sapiens),
NCBI Reference Sequence: NP_001276342.1,
1233 aa
(SEQ ID NO: 46)
MRGLPRGRGLMRARGRGRAAPPGSRGRGRGGPHRGRGRPRSLLSLPRAQA
SWTPQLSTGLTSPPVPCLPSQGEAPAEMGALLLEKETRGATERVHGSLGD
TPRSEETLPKATPDSLEPAGPSSPASVTVTVGDEGADTPVGATPLIGDES
ENLEGDGDLRGGRILLGHATKSFPSSPSKGGSCPSRAKMSMTGAGKSPPS
VQSLAMRLLSMPGAQGAAAAGSEPPPATTSPEGQPKVHRARKTMSKPGNG
QPPVPEKRPPEIQHFRMSDDVHSLGKVTSDLAKRRKLNSGGGLSEELGSA
RRSGEVTLTKGDPGSLEEWETVVGDDFSLYYDSYSVDERVDSDSKSEVEA
LTEQLSEEEEEEEEEEEEEEEEEEEEEEEEDEESGNQSDRSGSSGRRKAK
KKWRKDSPWVKPSRKRRKREPPRAKEPRGVSNDTSSLETERGFEELPLCS
CRMEAPKIDRISERAGHKCMATESVDGELSGCNAAILKRETMRPSSRVAL
MVLCETHRARMVKHHCCPGCGYFCTAGTFLECHPDFRVAHRFHKACVSQL
NGMVFCPHCGEDASEAQEVTIPRGDGVTPPAGTAAPAPPPLSQDVPGRAD
TSQPSARMRGHGEPRRPPCDPLADTIDSSGPSLTLPNGGCLSAVGLPLGP
GREALEKALVIQESERRKKLRFHPRQLYLSVKQGELQKVILMLLDNLDPN
FQSDQQSKRTPLHAAAQKGSVEICHVLLQAGANINAVDKQQRTPLMEAVV
NNHLEVARYMVQRGGCVYSKEEDGSTCLHHAAKIGNLEMVSLLLSTGQVD
VNAQDSGGWTPIIWAAEHKHIEVIRMLLTRGADVTLTDNEENICLHWASF
TGSAAIAEVLLNARCDLHAVNYHGDTPLHIAARESYHDCVLLFLSRGANP
ELRNKEGDTAWDLTPERSDVWFALQLNRKLRLGVGNRAIRTEKIICRDVA
RGYENVPIPCVNGVDGEPCPEDYKYISENCETSTMNIDRNITHLQHCTCV
DDCSSSNCLCGQLSIRCWYDKDGRLLQEFNKIEPPLIFECNQACSCWRNC
KNRVVQSGIKVRLQLYRTAKMGWGVRALQTIPQGTFICEYVGELISDAEA
DVREDDSYLFDLDNKDGEVYCIDARYYGNISRFINHLCDPNIIPVRVFML
HQDLRFPRIAFFSSRDIRTGEELGFDYGDRFWDIKSKYFTCQCGSEKCKH
SAEAIALEQSRLARLDPHPELLPELGSLPPVNT
SEQ ID NO: 47, Homo sapiens euchromatic
histone lysine methyltransferase 1 (EHMT1),
transcript variant 2, mRNA, NCBI Reference
Sequence: NM_001145527.2 (region 25-2451),
2427 bp
(SEQ ID NO: 47)
ATGGCCGCCGCCGATGCCGAGGCAGTTCCGGCGAGGGGGGAGCCTCAGCA
GGATTGCTGTGTGAAAACCGAGCTGCTGGGAGAAGAGACACCTATGGCTG
CCGATGAAGGCTCAGCAGAGAAACAGGCAGGAGAGGCCCACATGGCTGCG
GACGGTGAGACCAATGGGTCTTGTGAAAACAGCGATGCCAGCAGTCATGC
AAATGCTGCAAAGCACACTCAGGACAGCGCAAGGGTCAACCCCCAGGATG
GCACCAACACACTAACTCGGATAGCGGAAAATGGGGTTTCAGAAAGAGAC
TCAGAAGCGGCGAAGCAAAACCACGTCACTGCCGACGACTTTGTGCAGAC
TTCTGTCATCGGCAGCAACGGATACATCTTAAATAAGCCGGCCCTACAGG
CACAGCCCTTGAGGACTACCAGCACTCTGGCCTCTTCGCTGCCTGGCCAT
GCTGCAAAAACCCTTCCTGGAGGGGCTGGCAAAGGCAGGACTCCAAGCGC
TTTTCCCCAGACGCCAGCCGCCCCACCAGCCACCCTTGGGGAGGGGAGTG
CTGACACAGAGGACAGGAAGCTCCCGGCCCCTGGCGCCGACGTCAAGGTC
CACAGGGCACGCAAGACCATGCCGAAGTCCGTCGTGGGCCTGCATGCAGC
CAGTAAAGATCCCAGAGAAGTTCGAGAAGCTAGAGATCATAAGGAACCAA
AAGAGGAGATCAACAAAAACATTTCTGACTTTGGACGACAGCAGCTTTTA
CCCCCCTTCCCATCCCTTCATCAGTCGCTACCTCAGAACCAGTGCTACAT
GGCCACCACAAAATCACAGACAGCTTGCTTGCCTTTTGTTTTAGCAGCTG
CAGTATCTCGGAAGAAAAAACGAAGAATGGGAACCTATAGCCTGGTTCCT
AAGAAAAAGACCAAAGTATTAAAACAGAGGACGGTGATTGAGATGTTTAA
GAGCATAACTCATTCCACTGTGGGTTCCAAGGGGGAGAAGGACCTGGGCG
CCAGCAGCCTGCACGTGAATGGGGAGAGCCTGGAGATGGACTCGGATGAG
GACGACTCAGAGGAGCTCGAGGAGGACGACGGCCATGGTGCAGAGCAGGC
GGCCGCGTTCCCCACAGAGGACAGCAGGACTTCCAAGGAGAGCATGTCGG
AGGCTGATCGCGCCCAGAAGATGGACGGGGAGTCCGAGGAGGAGCAGGAG
TCCGTGGACACCGGGGAGGAGGAGGAAGGCGGTGACGAGTCTGACCTGAG
TTCGGAATCCAGCATTAAGAAGAAATTTCTCAAGAGGAAAGGAAAGACCG
ACAGTCCCTGGATCAAGCCAGCCAGGAAAAGGAGGCGGAGAAGTAGAAAG
AAGCCCAGCGGTGCCCTCGGTTCTGAGTCGTATAAGTCATCTGCAGGAAG
CGCTGAGCAGACGGCACCAGGAGACAGCACAGGGTACATGGAAGTTTCTC
TGGACTCCCTGGATCTCCGAGTCAAAGGAATTCTGTCTTCACAAGCAGAA
GGGTTGGCCAACGGTCCAGATGTGCTGGAGACAGACGGCCTCCAGGAAGT
GCCTCTCTGCAGCTGCCGGATGGAAACACCGAAGAGTCGAGAGATCACCA
CACTGGCCAACAACCAGTGCATGGCTACAGAGAGCGTGGACCATGAATTG
GGCCGGTGCACAAACAGCGTGGTCAAGTATGAGCTGATGCGCCCCTCCAA
CAAGGCCCCGCTCCTCGTGCTGTGTGAAGACCACCGGGGCCGCATGGTGA
AGCACCAGTGCTGTCCTGGCTGTGGCTACTTCTGCACAGCGGGTAATTTT
ATGGAGTGTCAGCCCGAGAGCAGCATCTCTCACCGTTTCCACAAAGACTG
TGCCTCTCGAGTCAATAACGCCAGCTATTGTCCCCACTGTGGGGAGGAGA
GCTCCAAGGCCAAAGAGGTGACGATAGCTAAAGCAGACACCACCTCGACC
GTGACACCAGTCCCCGGGCAGGAGAAGGGCTCGGCCCTGGAGGGCAGGGC
CGACACCACAACGGGCAGTGCTGCCGGGCCACCACTCTCGGAGGACGACA
AGCTGCAGGGTGCAGCCTCCCACGTGCCCGAGGGCTTTGATCCAACGGGA
CCTGCTGGGCTTGGGAGGCCAACTCCCGGCCTTTCCCAGGGACCAGGGAA
GGAAACCTTGGAGAGCGCTCTCATCGCCCTCGACTCGGAAAAACCCAAGA
AGCTTCGCTTCCACCCAAAGCAGCTGTACTTCTCCGCCAGGCAAGGGGAG
CTTCAGAAGGTGCTCCTCATGCTGGTGGACGGAATTGACCCCAACTTCAA
AATGGAGCACCAGAATAAGCGCTCTCCACTGCACGCCGCGGCAGAGGCTG
GACACGTGGACATCTGCCACATGCTGGTTCAGTTCTGCAGGCTGGGAAGC
CCAAGGTCGAGGGGCTGCCTTTGGTGA
SEQ ID NO: 48, histone-lysine N-methyl-
transferase EHMTI isoform 2 (Homo sapiens),
NCBI Reference Sequence: NP_001138999.1,
808 aa
(SEQ ID NO: 48)
MAAADAEAVPARGEPQQDCCVKTELLGEETPMAADEGSAEKQAGEAHMAA
DGETNGSCENSDASSHANAAKHTQDSARVNPQDGTNTLTRIAENGVSERD
SEAAKQNHVTADDFVQTSVIGSNGYILNKPALQAQPLRTTSTLASSLPGH
AAKTLPGGAGKGRTPSAFPQTPAAPPATLGEGSADTEDRKLPAPGADVKV
HRARKTMPKSVVGLHAASKDPREVREARDHKEPKEEINKNISDFGRQQLL
PPFPSLHQSLPQNQCYMATTKSQTACLPFVLAAAVSRKKKRRMGTYSLVP
KKKTKVLKQRTVIEMFKSITHSTVGSKGEKDLGASSLHVNGESLEMDSDE
DDSEELEEDDGHGAEQAAAFPTEDSRTSKESMSEADRAQKMDGESEEEQE
SVDTGEEEEGGDESDLSSESSIKKKFLKRKGKTDSPWIKPARKRRRRSRK
KPSGALGSESYKSSAGSAEQTAPGDSTGYMEVSLDSLDLRVKGILSSQAE
GLANGPDVLETDGLQEVPLCSCRMETPKSREITTLANNQCMATESVDHEL
GRCTNSVVKYELMRPSNKAPLLVLCEDHRGRMVKHQCCPGCGYFCTAGNF
MECQPESSISHRFHKDCASRVNNASYCPHCGEESSKAKEVTIAKADTTST
VTPVPGQEKGSALEGRADTTTGSAAGPPLSEDDKLQGAASHVPEGFDPTG
PAGLGRPTPGLSQGPGKETLESALIALDSEKPKKLRFHPKQLYFSARQGE
LQKVLLMLVDGIDPNFKMEHQNKRSPLHAAAEAGHVDICHMLVQFCRLGS
PRSRGCLW

In some embodiments, the method comprises the step of, after the step of differentiating a population of pluripotent stem cells in aggregation media for a sufficient time to promote differentiation into a population of CD34+ hemogenic endothelium, inhibiting G9a and/or GLP in the resultant population of CD34+ hemogenic endothelium. In some embodiments, the method comprises the step of, before the step of differentiating a population of CD34+ hemogenic endothelium in NK-cell-differentiation media in the presence of a Notch ligand for a sufficient time to promote differentiation into a population of CD56+NK cells, inhibiting G9a and/or GLP in the population of CD34+ hemogenic endothelium. In some embodiments of any of the aspects, inhibiting G9a and/or GLP can promote differentiation of CD34⁺ hemogenic endothelium into CD34⁺CD45⁺ hematopoietic stem cells (HSCs) and/or CD7+ CLPs. In some embodiments of any of the aspects, inhibiting G9a and/or GLP promotes endothelial-to-hematopoietic transition (EHT) and/or lymphoid specification.

Accordingly, in one aspect described herein is a method comprising: (a) differentiating a population of pluripotent stem cells in aggregation media for a sufficient time to promote differentiation into a population of CD34⁺ hemogenic endothelium; (b) inhibiting G9a and/or GLP in the resultant population of CD34⁺ hemogenic endothelium; and (c) differentiating the resultant population of CD34⁺ hemogenic endothelium in NK-cell-differentiation media in the presence of a Notch ligand for a sufficient time to promote differentiation into a population of CD56⁺ NK cells. In another aspect, described herein is a method comprising: (a) inhibiting G9a and/or GLP in a population of CD34⁺ hemogenic endothelium; and (b) differentiating the population of CD34⁺ hemogenic endothelium in NK-cell-differentiation media in the presence of a Notch ligand for a sufficient time to promote differentiation into a population of CD56⁺ NK cells.

In one embodiment, the inhibitor is a G9a/GLP inhibitor. In one embodiment, the G9a/GLP inhibitor is a small molecule inhibitor of G9a/GLP. In one embodiment, the G9a/GLP inhibitor is a nucleic acid inhibitor of G9a/GLP. In some embodiments, the method comprises inhibiting G9a, e.g., using a G9a-specific nucleic acid inhibitor (see e., SEQ ID NO: 50) or a G9a-specific small molecule inhibitor (see, e.g., Table 4). In some embodiments, the method comprises inhibiting GLP, e.g., using a GLP-specific nucleic acid inhibitor or a GLP-specific small molecule inhibitor. In some embodiments, the method comprises inhibiting G9a and GLP.

In one embodiment, the G9a/GLP inhibitor is selected from a small molecule listed in Table 4, or a derivative or analog thereof. In one embodiment, the G9a/GLP inhibitor is selected from the group consisting of: UNC0224; UNC0638; A366; BRD4770; BIX01294; UNC0642; UNC0631; UNC0646; UNC0321; E72; BIX-01338; BRD9539; Chaetocin; and DCG066. In one embodiment, the G9a/GLP inhibitor is selected from the group consisting of: UNC0224; UNC0638; A366; BRD4770; BIX01294; and UNC0642. In some embodiments, the G9a/GLP inhibitor is selected from the group consisting of: UNC0224; UNC0638; BRD4770; BIX01294; and UNC0642.

In some embodiments, the G9a/GLP inhibitor is a Type I G9a/GLP inhibitor (e.g., a BIX-01294 derivative) selected from the group consisting of: UNC0224; UNC0638; A366; BIX01294; UNC0642; UNC0631; UNC0646; UNC0321; and E72. In some embodiments, the G9a/GLP inhibitor is a Type II G9a/GLP inhibitor (e.g., a BIX-01338 derivative) selected from the group consisting of: BRD4770; BIX-01338; and BRD9539. In some embodiments, the G9a/GLP inhibitor is a Type III G9a/GLP inhibitor such as Chaetocin. In some embodiments, the G9a/GLP inhibitor is a Type IV G9a/GLP inhibitor selected from the group consisting of: DCG066.

TABLE 4
G9a/GLP inhibitors that can promote NK cell differentiation. All references
cited in Table 4 are specifically incorporated herein by reference in their entireties.

| | | Exemplary | |
|---|---|---|---|
| G9a/GLP | | effective | |
| inhibitor | Exemplary references | dose(s) | Small molecule structure |
| UNC0224 (e.g., Type I G9a inhibitor, BIX-01294 derivative) | See, e.g., Kondengaden et al., Eur J Med Chem. 2016 Oct. 21, 122:382-393 | ~5 nM-200 nM | 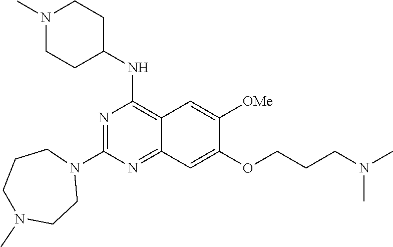 |
| UNC0638 (e.g., Type I G9a inhibitor, BIX-01294 derivative) | See, e.g., Kondengaden et al., Eur J Med Chem. 2016 Oct. 21, 122:382-393 | ~5 nM-200 nM | 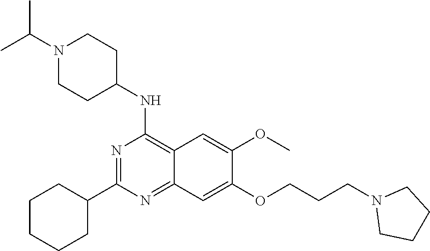 [SMILES: COc1cc2c(NC3CCN(C(C)C)CC3)nc(C3CCCCC3)nc2cc1OCCCN1CCCC1] |
| A366 (e.g., Type I G9a inhibitor, BIX-01294 derivative) | See, e.g., Kondengaden et al., Eur J Med Chem. 2016 Oct. 21, 122:382-393 | ~5 nM-200 nM | 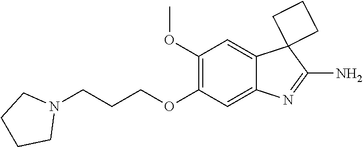 [SMILES: COc1cc2c(cc1OCCCN1CCCC1)N=C(N)C21CCC1] |
| BRD4770 (e.g., Type II G9a inhibitor, BIX-01338 derivative) | See, e.g., Yuan et al. ACS Chem Biol. 2012 Jul. 20; 7(7): 1152-1157 | ~5 nM-200 nM | 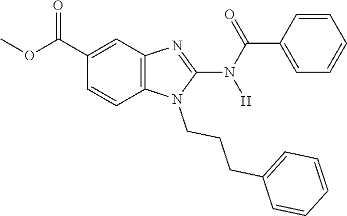 [SMILES: COC(=O)c1ccc2c(c1)nc(NC(=O)c1ccccc1)n2CCCc1ccccc1] |
| BIX01294 (e.g., Type I G9a inhibitor) | See, e.g., Kondengaden et al., Eur J Med Chem. 2016 Oct. 21, 122:382-393 | ~5 nM-200 nM | 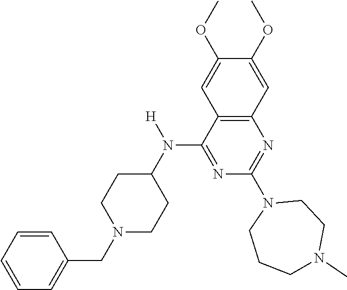 [SMILES: COc1cc2nc(N3CCCN(C)CC3)nc(NC3CCN(Cc4ccccc4)CC3)c2cc1OC] |
| UNC0642 (e.g., Type I G9a inhibitor, BIX-01294 derivative) | See, e.g., Kondengaden et al., Eur J Med Chem. 2016 Oct. 21, 122:382-393 | ~5 nM-200 nM | 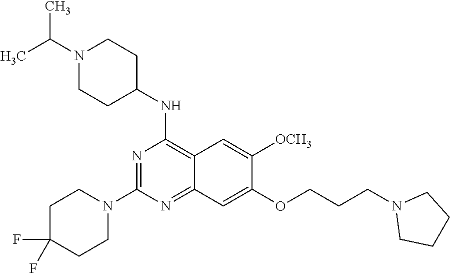 [SMILES: COc1cc2c(NC3CCN(C(C)C)CC3)nc(N3CCC(F)(F)CC3)nc2cc1OCCCN1CCCC1] |
| UNC0631 (e.g., Type I G9a inhibitor, BIX-01294 derivative) | See, e.g., Liu et al. Journal of Medicinal Chemistry 54(17), 6139-6150 (2011). | E.g., in MDA-MB- 231 cells, IC₅₀ = 25 nM | 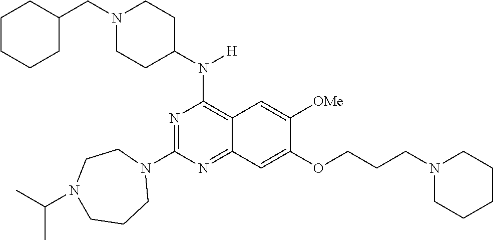 [SMILES: COc1cc2c(NC3CCN(CC4CCCCC4)CC3)nc(N3CCCN(C(C)C)CC3)nc2cc1OCCCN1CCCCC1] |
| UNC0646 (e.g., Type I G9a inhibitor, BIX-01294 derivative) | See, e.g., Liu et al. Journal of Medicinal Chemistry 54(17), 6139-6150 (2011). | E.g., in MDA-MB- 231 cells, IC₅₀ = 26 nM | 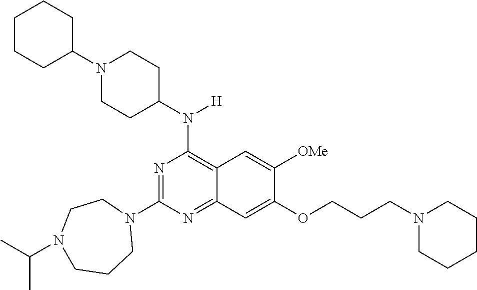 [SMILES: COc1cc2c(NC3CCN(C4CCCCC4)CC3)nc(N3CCCN(C(C)C)CC3)nc2cc1OCCCN1CCCCC1] |
| UNC0321 (e.g., Type I G9a inhibitor, BIX-01294 derivative) | See, e.g., Liu et al. J Med Chem. 2010 Aug. 12; 53(15): 5844-5857; Liu et al. J Med Chem. 2009 Dec. 24; 52(24): 7950-7953. A cell- permeable, quinazoline analog that potently and selectively inhibits PKMT G9a (IC₅₀ = 6 nM and 9 nM in two biochemical assays for CLOT and ECSD, respectively, and Morrison Ki = 63 pM, which is approximately 250-fold | E.g., G9a (IC₅₀ = 6 nM and 9 nM, and Morrison Ki = 63 pM; GLP (e.g., 15 nM) | 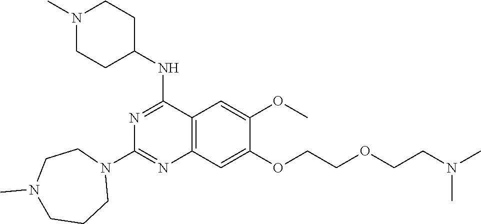 |
| | more potent than a closely- | | |
| | related analog, BIX01294. It | | |
| | inhibits GLP with reduced | | |
| | potency (e.g., 15 nM) and is | | |
| | found to be inactive (IC50 > | | |
| | 40 μM) toward other protein | | |
| | lysine and arginine | | |
| | methyltransferases, such as | | |
| | SET7/9 (aH3K4 PKMT), | | |
| | SET8/PreSET7 (aH4K20 | | |
| | PKMT), and PRMT3, as | | |
| | well as JMJD2E (>1000- | | |
| | fold selectivity) in ECSD | | |
| | enzymatic assays. | | |
| E72 (e.g., Type I G9a inhibitor, BIX-01294 derivative) | See, e.g., Chang et al. J Mol Biol. 2010 Jul. 2; 400(1): 1-7 | E.g., G9a EC₅₀ 100 nM | 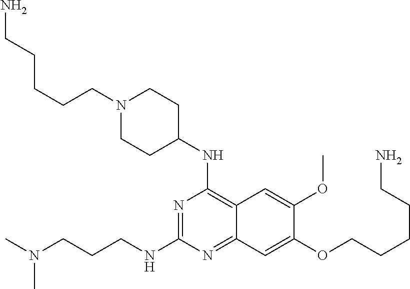 [SMILES: COc1cc2c(NC3CCN(CCCCCN)CC3)nc(NCCCN(C)C)nc2cc1OCCCCCN] |
| BIX-01338 (e.g., Type II G9a inhibitor) | See, e.g., Greiner et al. Nature Chemical Biology 1(3), 143-145 (2005). A cell- permeable amino- benzimidazolo compound that is shown to inhibit a broad-spectrum of histone methyltransferases, including the PRMT1 H4R3 me2 activity, SET7/9 H3K4 me activity, G9a H3K9 me2 activity, as well as the H3K9 me3 activity of SUV39H1 (wild-type and H320R hyperactive mutant) and GLP (effective conc. = 15 μM) in a SAM-competitive manner. | G9a/GLP effective conc. = 15 μM | 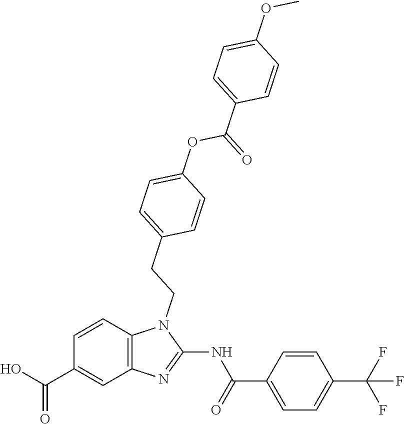 [SMILES: COc1ccc(C(=O)Oc2ccc(CCn3c(NC(=O)c4ccc(C(F)(F)F)cc4)nc4cc(C(=O)O)ccc43)cc2)cc1] |
| BRD9539 (e.g., Type II G9a inhibitor, BIX-01338 derivative) | See, e.g., Yuan et al., ACS Chem Biol. 2012 Jul. 20; 7(7): 1152-1157. BRD9539 is an inhibitor of G9a, with an IC50 value of 6.3 μM. It inhibits polycomb repressive complex 2 (PRC2) to a similar extent with 54 and 43% activity remaining for G9a and PRC2, respectively, when used at a concentration of 10 μM. It is selective for G9a and PRC2 over SU39H1 and NDMT1 up to | E.g., G9a, IC50 value of 6.3 μM | 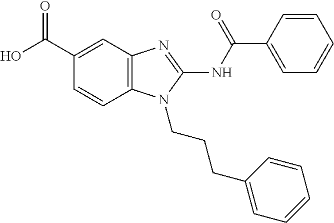 [SMILES: O=C(O)c1ccc2c(c1)nc(NC(=O)c1ccccc1)n2CCCc1ccccc1] |
| | a concentration of 40 μM. It | | |
| | is more potent than | | |
| | BRD4770 in enzyme assays, | | |
| | but may require a higher | | |
| | concentration for cell-based | | |
| | assays. | | |
| Chaetocin (e.g., (+)- Chaetocin; Type III G9a inhibitor) | See, e.g., Greiner et al. Nature Chemical Biology 1(3), 143-145 (2005). Chaetocin is a fungal mycotoxin that inhibits the HMT SU(VAR)3-9 (IC₅₀ = 0.8 μM).1 It inhibits other Lys9-specific HMTs, including G9a (IC₅₀ = 2.5 μM) and DIM5 (IC₅₀ = 3 μM).1 Selectivity for Lys9- HMTs is indicated by higher IC50 values (>90 μM) for Lys20-specific PRSET7, Lys27-specific EZH2, and Lys4-specific SET7/9. | E.g., G9a IC₅₀ = 2.5 μM | 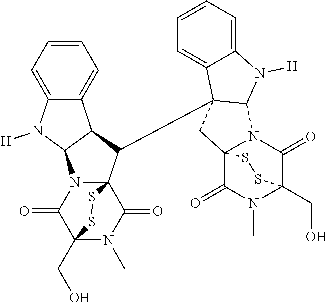 |
| DCG066 (e.g., Type IV G9a inhibitor) | See, e.g., Kondengaden et al., Eur J Med Chem. 2016 Oct. 21;122:382-393. | E.g., G9a EC₅₀ 6.5 μM | 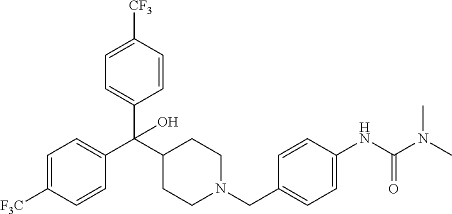 [SMILES: CN(C)C(=O)Nc1ccc(CN2CCC(C(O)(c3ccc(C(F)(F)F)cc3)c3ccc(C(F)(F)F)cc3)CC2)cc1] |

In some embodiments, the G9a/GLP inhibitor is provided at a concentration of at least 500 nM. In some embodiments, the G9a/GLP inhibitor is provided at a concentration of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 μM, at least 1.25 μM, at least 1.5 μM, at least 1.75 μM, at least 2.0 μM, at least 2.5 μM, at least 3 μM, at least 4 μM, at least 5 μM, at least 6 μM, at least 7 μM, at least 8 μM, at least 9 μM, or at least 10 μM. In some embodiments, the G9a/GLP inhibitor is provided at a concentration of 1 nM-10 nM, 10 nM-50 nM, 50 nM-100 nM, 100 nM-500 nM, 500 nM-1 μM, 1 μM-5 μM, or 5 μM-10 μM.

In some embodiments, the G9a/GLP inhibitor (e.g., UNC0224) is provided at a concentration of at least 312 nM, at least 625 nM, at least 1.25 μM, at least 2.5 μM, or at least 5 μM. In some embodiments, the G9a/GLP inhibitor (e.g., UNC0638) is provided at a concentration of at least 8 nM. In some embodiments, the G9a/GLP inhibitor (e.g., BRD4770) is provided at a concentration of at least 200 nM. In some embodiments, the G9a/GLP inhibitor (e.g., BIX01294) is provided at a concentration of at least 200 nM. In some embodiments, the G9a/GLP inhibitor (e.g., UNC0642) is provided at a concentration of at least 40 nM.

In some embodiments, the G9a/GLP inhibitor is a nucleic acid inhibitor. In some embodiments, the G9a/GLP nucleic acid inhibitor is a CRISPRi guide RNA (gRNA). CRISPRi is a variant of CRISPR in which a catalytically dead (d) Cas9 is fused with a transcriptional effector (e.g., inhibitor) to modulate target gene expression. In CRISPRi, when the guide RNA navigates to the genome locus (e.g., promoter region associated with the gene of interest; e.g., the G9a promoter, or the GLP promoter) along with the effector arm, it represses the downstream gene expression instead of activating it. In some embodiments, the nucleic acid inhibitor is inducible (e.g., Dox-inducible). In some embodiments, the G9a nucleic acid inhibitor comprises SEQ ID NO: 50 or a nucleic acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 50, and that maintains the same functions as SEQ ID NO: 50 (knockdown of G9a).

SEQ ID NO: 50, G9a CRISPRi gRNA:
AATGGCCAAAAAGCATGTAG

In some embodiments, the cells (e.g., CD34+ hemogenic endothelium) are cultured exposed to a G9a/GLP inhibitor until the development of CD56+NK cells. In some embodiments, the cells (e.g., CD34+ hemogenic endothelium) are cultured exposed to a G9a/GLP inhibitor for about 14 days. In some embodiments, the cells (e.g., CD34⁺ hemogenic endothelium) are cultured exposed to a G9a/GLP inhibitor for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more.

In some embodiments, culturing cells (e.g., CD34+ hemogenic endothelium) in the presence of a G9a/GLP inhibitor (e.g., a small molecule G9a/GLP inhibitor or nucleic acid G9a/GLP inhibitor) increases the number of resultant CD56+NK cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, culturing cells (e.g., CD34+ hemogenic endothelium) in the presence of a G9a/GLP inhibitor (e.g., a small molecule G9a/GLP inhibitor or nucleic acid G9a/GLP inhibitor) decreases the number of erythroid or myeloid lineage cells (e.g., erythroid cell; macrophage; granulocyte; megakaryocyte) by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, culturing cells (e.g., CD34+ hemogenic endothelium) in the presence of a G9a/GLP inhibitor (e.g., a small molecule G9a/GLP inhibitor or nucleic acid G9a/GLP inhibitor) increases the percentage of resultant CD56+NK cells amongst the total number of differentiated cells by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 600%, at least 700%, at least 800%, at least 900%, or more, or at least 10×, 20×, 30×, 40×, 50×, 60×, 70×, 80×, 90×, 100×, 500×, 1,000×, or more higher compared to cells not cultured in the presence of a G9a/GLP inhibitor.

In some embodiments, a method for differentiating NK cells as described herein (e.g., G9a/GLP inhibition and stromal-free NK cell differentiation) produces a population that comprises at least 1%, at least 5%, at least 10%, at least 15%, at least 20%, at least 30%, at least 40%, at least 50%, at least 60%, at least 70%, at least 80%, at least 90%, at least 95%, or at least 99% CD56+NK cells. In some embodiments, a method for differentiating NK cells as described herein (e.g., G9a/GLP inhibition and stromal-free NK cell differentiation) produces a population that comprises at least 40% CD56+NK cells. In some embodiments, a method for differentiating NK cells as described herein (e.g., G9a/GLP inhibition and stromal-free NK cell differentiation) produces a population that comprises at least 60% CD56+NK cells (see, e.g., FIGS. 5B-5C).

For further details about small molecule inhibitors that can promote NK cell differentiation, including but not limited to G9a/GLP inhibitors, see, e.g., Greiner et al. Nature Chemical Biology 1(3), 143-145 (2005); Liu et al. Journal of Medicinal Chemistry 54(17), 6139-6150 (2011); Liu et al. J Med Chem. 2010 Aug. 12; 53(15): 5844-5857; Liu et al., J Med Chem. 2009 Dec. 24; 52(24): 7950-7953; Kondengaden et al., Eur J Med Chem. 2016 Oct. 21, 122:382-393; Yuan et al. ACS Chem Biol. 2012 Jul. 20; 7(7): 1152-1157; Chang et al. J Mol Biol. 2010 Jul. 2; 400(1): 1-7; Christman et al. Proceedings of the National Academy of Sciences of the United States of America 92(16), 7347-7351 (1995); Cheng et al., Signal Transduction and Targeted Therapy volume 4, Article number: 62 (2019); the contents of each of which are incorporated herein by reference in their entireties.

In some embodiments, the population of cells (e.g., CD45+CD34+ HSPCs; e.g., CD56+NK cells) derived using inhibition of an epigenetic regulator as described herein (e.g., G9 and/or GLP) expresses at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more, gene(s) or gene pathway(s) associated with or responsible for lymphoid differentiation and/or function (see, e.g., FIGS. 6A-6C).

In some embodiments, the population of cells (e.g., CD45+CD34+ HSPCs; e.g., CD56+NK cells) derived using inhibition of an epigenetic regulator as described herein (e.g., G9 and/or GLP) exhibits increased expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more, gene(s) or gene pathway(s) associated with or responsible for lymphoid differentiation and/or function as compared to a population of cells (e.g., CD45+CD34+ HSPCs; e.g., CD56+NK cells) derived without inhibition of the epigenetic regulator (see, e.g., FIGS. 6A-6C).

In some embodiments, the gene pathways up-regulated by G9a-inhibition, and that can be associated with or responsible for lymphoid differentiation and/or function include, but are not limited to: immune system process; myeloid leukocyte activation; immune response; cell activation; leukocyte activation; vesicle-mediated transport; immune effector process; myeloid cell activation involved in immune response; leukocyte cell activation involved in immune response; cell activation involved in immune response; neutrophil activation; granulocyte activation; neutrophil mediated immunity; myeloid leukocyte mediated immunity; neutrophil degranulation; leukocyte degranulation; neutrophil activation in immune response; regulated exocytosis; exocytosis; or leukocyte mediated immunity (see, e.g., FIG. 6C).

In some embodiments, the genes associated with or responsible for lymphoid differentiation and/or function include, but are not limited to: Runt-related transcription factor 3 (RUNX3), CD1A, tumor necrosis factor (TNF), Granzyme A (GZMA), Granulysin (GNLY), and/or IKAROS Family Zinc Finger 5 (IKZF5, also referred to as Pegasus). In some embodiments, G9a inhibition results in increased expression of at least 1, at least 2, at least 3, at least 4, or all of RUNX3, CD1A, TNF, GZMA, GNLY, and/or IKZF5.

In some embodiments, the gene pathways up-regulated by G9a-inhibtion, and that can be associated with or responsible for lymphoid differentiation and/or function include, but are not limited to: antigen processing and presentation (GO:0019882); positive regulation of leukocyte cell-cell adhesion (GO:1903039); positive regulation of T cell activation (GO:0050870); positive regulation of cell-cell adhesion (GO:0022409); T cell differentiation (GO:0030217); positive regulation of cell adhesion (GO:0045785); regulation of leukocyte cell-cell adhesion (GO:1903037); regulation of T cell activation (GO:0050863); leukocyte cell-cell adhesion (GO:0007159); regulation of cell-cell adhesion (GO:0022407); T cell activation (GO:0042110); regulation of cell adhesion (GO:0030155); adaptive immune response based on somatic recombination of immune receptors built from immunoglobulin superfamily domains (GO:0002460); lymphocyte mediated immunity (GO:0002449); production of molecular mediator of immune response (GO:0002440); B cell mediated immunity (GO:0019724); immunoglobulin mediated immune response (GO:0016064); leukocyte mediated immunity (GO:0002443); adaptive immune response (GO:0002250); immune effector process (GO:0002252); positive regulation of lymphocyte activation (GO:0051251); lymphocyte activation (GO:0046649); positive regulation of leukocyte activation (GO:0002696); positive regulation of cell activation (GO:0050867); leukocyte activation (GO:0045321); regulation of lymphocyte activation (GO:0051249); cell activation (GO:0001775); regulation of leukocyte activation (GO:0002694); positive regulation of immune system process (GO:0002684); regulation of cell activation (GO:0050865); cell-cell adhesion (GO:0098609); cellular protein-containing complex assembly (GO:0034622); regulation of immune system process (GO:0002682); protein-containing complex assembly (GO:0065003); protein-containing complex subunit organization (GO:0043933); immune response (GO:0006955); immune system process (GO:0002376); cellular component biogenesis (GO:0044085); cellular component assembly (GO:0022607); positive regulation of cellular process (GO:0048522); positive regulation of biological process (GO:0048518); cellular component organization or biogenesis (GO:0071840); or gene expression (GO:0010467) (see, e.g., Table 6). “GO” refers to Gene Ontology analysis on RNA-seq data.

Inhibition of a Histone Methyltransferase

In some embodiments, the differentiation method can comprise inhibiting a histone methyltransferase. The step of inhibiting a histone methyltransferase (e.g., EZH1 knockdown) can increase differentiation efficiency (e.g., of the NK cells). Accordingly, in some embodiments, the differentiation method comprises inhibiting a histone methyltransferase, e.g., in the resultant population of CD34⁺ hemogenic endothelium. Methods of inhibiting a histone methyltransferase are known in the art; see, e.g., International Application No. WO 2018/048828, US Application No. 2019/0225940, Doulatov et al., Cell Stem Cell. 2013 Oct. 3, 13(4); Vo et al., Nature 2018, 553(7689): 506-510; the contents of each of which are incorporated herein by reference in their entireties.

However, the step of inhibiting a histone methyltransferase (e.g., EZH1 knockdown) is not required. Thus, in some embodiments, the differentiation method does not comprise inhibiting a histone methyltransferase, e.g., in the resultant population of CD34+ hemogenic endothelium.

In the course of these experiments, the inventors discovered that inhibition of specific histone modifying enzymes targeting H3K9 and H3K27 promotes lymphoid potential of hematopoietic progenitors derived from pluripotent stem cells. The histone modifying enzymes are histone lysine methyltransferases. Post-translational modifications of histone proteins regulate chromatin compaction, mediate epigenetic regulation of transcription, and control cellular differentiation in health and disease. Methylation of histone tails is one of the fundamental events of epigenetic signaling. Tri-methylation of lysine 9 of histone H3 (H3K9) mediates chromatin recruitment of HP1, heterochromatin condensation and gene silencing. Similarly, methylation of H3K27 and H4K20 are associated with a repressed state of chromatin, whereas expressed genes are methylated at H3K4, H3K36 and H3K79. Methylation of H3K9 in humans relies mostly on members of the Suv39 family, namely EHMT1/GLP, EHMT2/G9a, SUV39H1, SUV39H2, SETDB1 and SETDB2, as well as then non-Suv39 enzymes PRDM2 and ASH1L (see, e.g., Hong Wu et al., Structural Biology of Human H3K9 Methyltransferases, 2010, PLoS ONE, 5(2): e8570, which is incorporated herein by reference). In contrast, the methylation of H3K27 is carry out by the polycomb repressive complex 2 (PRC2).

Di/trimethylation of H3K9 is mainly catalyzed by the conserved SUV39H1/2 histone methyltransferases, while the polycomb repressive complex 2 (PRC2) ensures di/trimethylation of H3K27 (see, e.g., Rea S, 2000. Nature 406:593-599; Margueron R, and Reinberg D. 2011. Nature 469:343-349). PRC2 comprises the EZH1/2 catalytic subunit, SUZ12, EED, and RBBP7/4 (see, e.g., Margueron R, and Reinberg D, 2011).

It is specifically contemplated herein that inhibiting the histone lysine methyltransferases that target H3K9 and H3K27 relieves transcriptional repression that results from methylation of histone H3, and thereby promotes gene expression which facilitates cell differentiation, specifically NK cell specification.

In one embodiment, the histone methyltransferase catalyzes the addition of methyl group to the histone H3 lysine residue 9 (H3K9) and/or histone H3 lysine residue 27 (H3K27).

In one embodiment, the histone methyltransferase inhibitor inhibits the G9a/GLP heteromeric complex.

G9a (EC 2.1.1.43) (UniProtKB: Q96KQ7) is also known as EHMT2, (Euchromatic Histone-Lysine N-Methyltransferase 2), G9A Histone Methyltransferase and protein G9a.

GLP (EC 2.1.1.43) (UniProtKB: Q9H9B1) is also known as EHMT1 (Euchromatic Histone-Lysine N-Methyltransferase 1), G9a-Like Protein 1 and GLP1.

In one embodiment, the histone methyltransferase inhibitor inhibits EZH1 (Enhancer of Zeste 1 Polycomb Repressive Complex 2 Subunit).

In one embodiment, the H3K27 histone methyltransferase is EZH1 (EC:2.1.1.43) (UniproKB Q92800-1).

In one embodiment, the H3K27 histone methyltransferase is not EZH2 (EC:2.1.1.43) (Unipro Q15910-1).

In one embodiment, the inhibitor of histone methyltransferase inhibits the gene expression or protein catalytic activity of the histone methyltransferase.

In one embodiment, the histone methyltransferase H3K9 and/or H3K27 is inhibited by a small molecule or a nucleic acid or a CRISPR-mediated target genetic interference.

In some embodiments, the histone methyltransferase H3K9 and/or H3K27 is inhibited by a small molecule inhibitor or a nucleic acid inhibitor. In one embodiment of any method, cells, or composition described, the histone methyltransferase small molecule inhibitor is a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the small molecule is a heterorganic compound or an organometallic compound.

In one embodiment, the histone methyltransferase small molecule inhibitor include but are not limited to AMI-1, A-366, BIX-01294, BIX01338, BRD4770, chaetocin, E72, UNC0224, UNC0631, UNC0638, UNC0642, UNC0646, EPZ5676, EPZ005687, GSK343, EPZ-6438 (E7438), 3-deazaneplanocin A (DZNeP) HCl, UNC1999, MM-102, SGC 0946, Entacapone, EPZ015666, UNC0379, EI1, MI-2 (Menin-MLL Inhibitor), MI-3 (Menin-MLL Inhibitor), PFI-2, GSK126, or EPZ004777.

In one embodiment, the histone methyltransferase small molecule inhibitor is selected from the group consisting of UNC0631, BRD4770, UNC1999, CPI-360, and BIX 01294.

In one embodiment, the nucleic acid inhibitor is a nucleic acid targeting the expression of histone methyltransferase. For example, targeting the mRNA or primary transcript of the histone methyltransferase, EZH1, thereby inhibiting protein expression of the enzyme. Histone-lysine N-methyltransferase aka Enhancer of Zeste 1 Polycomb Repressive Complex 2 Subunit (EZH1) or EC 2.1.1.43, is a component of a noncanonical Polycomb repressive complex-2 (PRC2) that mediates methylation of histone H3 (see MIM 602812) lys27 (H3K27) and functions in the maintenance of embryonic stem cell pluripotency and plasticity. The external identification for the human EZH1 gene are as follows: HGNC: 3526; Entrez Gene: 2145; Ensembl: ENSG00000108799; OMIM: 601674; UniProtKB: Q92800; EMBL: AB002386 mRNA and the corresponding mRNA translation: BAA20842.2; GENBANK: BT009782 mRNA and the corresponding mRNA translation: AAP88784.1.

In one embodiment, the nucleic acid inhibitor targets the human EZH1 mRNA.

In one embodiment, the nucleic acid inhibitor is an RNA interference inhibitor or CRISPR-mediated genetic interference inhibitor. The RNA interference inhibitor can be designed using the predictor RNAi softwares found at the Whitehead Institute, MIT, siRNA website, BLOCK-iT™ RNAi Designer at Invitrogen/ThermoFisher, and other online siRNA design tools at The RNAi Web using the mRNA of EZH1 as the target.

Similarly, Crisper guide RNA can be designed using the Broad Institute (MIT) CRISPR software (available on the world-wide web at, for example, portals.broadinstitute.org/gpp/public/analysis-tools/sgrna-design), dna20, Clontech, AddGene, e-crisp, and Innovative Genomic using the mRNA or genomic gene of EZH1 as the target.

CRISPR (Clustered Regularly Interspaced Short Palindromic Repeats) Cas9-mediated gene disruption has been widely used in generating loss-of-function mutations in diverse organisms including mammals (Cong et al., 2013, Science, 339(6121):819-23; reviewed in Hsu et al., 2014, Cell, 157(6):1262-78)). Cas9-based knockout screens have been applied in identifying essential genes and genes involved in drug resistance in various cell lines. With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830, US 2014-0287938, US 2014-0273234, U52014-0273232, US 2014-0273231, US 2014-0256046, US 2014-0248702, US 2014-0242700, US 2014-0242699, US 2014-0242664, US 2014-0234972, US 2014-0227787, US 2014-0189896, US 2014-0186958, US 2014-0186919, US 2014-0186843, US 2014-0179770 and US 2014-0179006, US 2014-0170753; European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and International Application No. WO 2014/093661, all of which are incorporated herein by reference in their entirety.

The CRISPR/Cas system envisaged for use in the context of the invention can make use of any suitable CRISPR enzyme. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell.

As described herein, the CRISPR/Cas system is used to specifically target a multitude of sequences within the continuous genomic region of interest. The targeting typically comprises introducing into each cell of a population of cells a vector system of one or more vectors comprising an engineered, non-naturally occurring CRISPR-Cas system comprising: at least one Cas protein, and one or more guide RNAs of the guide RNA library described herein.

In these methods, the Cas protein and the one or more guide RNAs may be on the same or on different vectors of the system and are integrated into each cell, whereby each guide sequence targets a sequence within the continuous genomic region in each cell in the population of cells. The Cas protein is operably linked to a regulatory element to ensure expression in said cell, more particularly a promoter suitable for expression in the cell of the cell population. In particular embodiments, the promoter is an inducible promoter, such as a doxycycline inducible promoter. When transcribed within the cells of the cell population, the guide RNA comprising the guide sequence directs sequence-specific binding of a CRISPR-Cas system to a target sequence in the continuous genomic region. Typically binding of the CRISPR-Cas system induces cleavage of the continuous genomic region by the Cas protein.

RNA interference (RNAi) mediated by short interfering RNAs (siRNA) or microRNAs (miRNA) is a powerful method for post-transcriptional regulation of gene expression. RNAi has been extensively used for the study of biological processes in mammalian cells and could constitute a therapeutic approach to human diseases in which selective modulation of gene expression would be desirable. Depending on the degree of complementarity between miRNA and target mRNA sequences, loss of gene expression occurs by inducing degradation of the cognate mRNA or by translational attenuation. Endogenous miRNAs are transcribed as primary transcripts and subsequently processed by the RNAse III enzyme Drosha to create a stem loop structure. Nuclear export and cleavage by Dicer generates a mature short double stranded molecule (siRNA) that is separated into guide and passenger strands. The guide strand is loaded into the RNA induced silencing complex (RISC), the effector complex mediating cleavage of target mRNAs with the functional guide strand binding to RISC proteins while the passenger strand is degraded. The loading of guide versus passenger strands into RISC largely depends on the 5′ end stability of the siRNA, with the less stable strand preferentially incorporated into RISC, although the exact regulation in mammalian cells is incompletely understood. The 5′ end of the guide strand contains the “seed region,” which is critical for target identification. Precise cleavage by Drosha and Dicer is critical for the generation of guide RNAs with defined seed regions that mediate efficient binding to the appropriate target mRNAs. Inaccurate processing results in binding to off-target molecules but a shift in cleavage sites also alters the nucleotide composition of duplex ends, which may have a profound effect on strand loading into RISC.

The inhibiting the expression of selected target polypeptides is through the use of RNA interference agents. RNA interference (RNAi) uses small interfering RNA (siRNA) duplexes that target the messenger RNA encoding the target polypeptide for selective degradation. siRNA-dependent post-transcriptional silencing of gene expression involves cleaving the target messenger RNA molecule at a site guided by the siRNA. RNAi is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see, e.g., Coburn, G. and Cullen, B. (2002) J. Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, at least 20%, at least 30%, at least 40%, at least 50%, at least 60%, at least 70%, at least 80%, at least 90%, at least 95%, at least 99%, or more as compared to the expression of a target gene or the activity or level of the protein encoded by a target gene which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. siRNA is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety). The target gene or sequence of the RNA interfering agent may be a cellular gene or genomic sequence, e.g., the G9a/GLP or EZH1 sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target. The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target G9a/GLP or EZH1 mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human G9a/GLP or EZH1 mRNA. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. Preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA. The Examples herein provide specific examples of RNA interfering agents, such as shRNA molecules that effectively target mRNA.

In one embodiment, the nucleic acid is a G9a/GLP or EZH1 specific RNA interference agent or a vector encoding the RNA interference agent. In one embodiment, the EZH1 specific RNA interference agent comprises one or more of the nucleotide sequences selected from the group consisting of CTATCTGGCAGTGCGAGAATG (SEQ ID NO: 11), AGACGTGCAAGCAGGTCTTTC (SEQ ID NO: 12), TGGATGACTTATGCGTGATTT (SEQ ID NO: 13), CAACAGAACTTTATGGTAGAA (SEQ ID NO: 14), CCGCCGTGGTTTGTATTCATT (SEQ ID NO: 15), GCTTCCTCTTCAACCTCAATA (SEQ ID NO: 16), CCGCCGTGGTTTGTATTCATT (SEQ ID NO: 17), GCTCTTCTTTGATTACAGGTA (SEQ ID NO: 18), and GCTACTCGGAAAGGAAACAAA (SEQ ID NO: 19).

In some embodiments, the nucleic acid inhibitor is an EZH1 specific nucleic acid that is selected from the group consisting of an aptamer that binds EZH1, an EZH1 specific RNA interference agent, and a vector encoding an EZH1 specific RNA interference agent, wherein the RNA interference agent comprises one or more of the nucleotide sequences selected from SEQ ID NOS: 11-19.

In one embodiment, the multilineage hematopoietic progenitor cells are contacted with the viral vector or vector carrying a nucleic acid molecule comprising a nucleic acid sequence selected from a group consisting of SEQ ID NOS: 11-19.

In one embodiment, the contacting with the histone methyltransferase inhibitor occurs more than once. For example, after the initial first contacting of the multilineage hematopoietic progenitor cell with the virus or vector carrying a nucleic acid molecule comprising a nucleic acid sequence selected from a group consisting of SEQ ID NOS: 11-19, or contacting with a small molecule inhibitor described herein, the contacted cell is washed to remove that virus or vector, and the washed cell is then contacted for a second time with the same virus or vector used in the first contact.

It is contemplated herein that the Cas9/CRISPR system of genome editing be employed with the methods, cells and compositions described herein. Clustered regularly interspaced short palindromic repeats (CRISPR)/CRISPR-associated (Cas) systems is useful for RNA-programmable genome editing (see, e.g., Jinek, M. et al. Science (2012) 337(6096):816-821).

Trans-activating crRNA (tracrRNA) is a small trans-encoded RNA. It was first discovered in the human pathogen Streptococcus pyogenes. (See Deltcheva E, et al. (2011). Nature 471 (7340): 602-7). In bacteria and archaea, CRISPR/Cas (clustered, regularly interspaced short palindromic repeats/CRISPR-associated proteins) constitute an RNA-mediated defense system which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into effector complexes, where the crRNA guides the complex to the invading nucleic acid and the Cas proteins degrade this nucleic acid. (See, e.g., Terns M P and Terns R M (2011). Curr Opin Microbiol 14 (3): 321-7). There are several pathways of CRISPR activation, one of which requires a tracrRNA which plays a role in the maturation of crRNA. TracrRNA is complementary to and base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid. (See, e.g., Deltcheva E, et al. supra; Jinek M, et al. (2012), Science 337 (6096): 816-21; and Brouns S J (2012), Science 337 (6096): 808-9).

In some embodiments, Cas9/CRISPR system guide RNAs are designed to target the exon 3 of EZH1 gene, which is present in all transcripts of EZH1 known. Exon 3 sequence is

(SEQ ID NO: 20)
ATTACAGCAAGATGGAAATACCAAATCCCCCTACCTCCAAATGTATCACT
TACTGGAAAAGAAAAGTGAAATCTGAATACATGCGACTTCGACAACTTAA
ACGGCTTCAGGCAAATATGGGTGCAAAG.

Non-limiting exemplary gRNAs that target exon 3 of EZH1 are

(SEQ ID NO: 21)
TCGACAACTTAAACGGCTTC,
(SEQ ID NO: 22)
TGCGACTTCGACAACTTAAA,
(SEQ ID NO: 23)
CCTCCAAATGTATCACTTAC,
(SEQ ID NO: 24)
TAAACGGCTTCAGGCAAATA,
(SEQ ID NO: 25)
AAACGGCTTCAGGCAAATAT,
(SEQ ID NO: 26)
CATTTGGAGGTAGGGGGATGT,
(SEQ ID NO: 27)
CCAGTAAGTGATACATTTGG,
(SEQ ID NO: 28)
GTGATACATTTGGAGGTAGG,
(SEQ ID NO: 29)
AAGTGATACATTTGGAGGTA,
(SEQ ID NO: 30)
AGTGATACATTTGGAGGTAG,
(SEQ ID NO: 31)
TTTCCAGTAAGTGATACATT,
and
(SEQ ID NO: 32)
TAAGTGATACATTTGGAGGT

In other embodiments, Cas9/CRISPR system guide RNAs are designed to target the exon 4 of EZH1 gene, which is also present in all transcripts of EZH1 known. Exon 4 sequence of EZH1 is

(SEQ ID NO: 33)
GCTTTGTATGTGGCAAATTTTGCAAAGGTTCAAGAAAAAACCCAGATCCT
CAATGAAGAATGGAAGAAGCTTCGTGTCCAACCTGTTCAGTCAATGAAGC
CTGTGAGTGGACACCCTTTTCTCAAAAAG.

Non-limiting exemplary gRNAs that target exon 4 of EZH1 are

(SEQ ID NO: 34)
GCTTCATTGACTGAACAGGT,
(SEQ ID NO: 35)
ACAGGCTTCATTGACTGAAC,
(SEQ ID NO: 36)
AGAAAAGGGTGTCCACTCAC,
(SEQ ID NO: 37)
TCCATTCTTCATTGAGGATC,
(SEQ ID NO: 38)
CCATTCTTCATTGAGGATCT,
(SEQ ID NO: 39)
CCCAGATCCTCAATGAAGAA,
(SEQ ID NO: 40)
GTATGTGGCAAATTTTGCAA,
and
(SEQ ID NO: 41)
CAGTCAATGAAGCCTGTGAG.

In some embodiments, the EZH1 nucleic acid inhibitor is a CRISPR interference (CRISPRi) guide RNA (gRNA). In some embodiments, the nucleic acid inhibitor is inducible (e.g., Dox-inducible). In some embodiments, the EZH1 nucleic acid inhibitor comprises one of SEQ ID NOS: 51-53 or a nucleic acid sequence that is at least 85%, at least 87%, at least 90%, at least 91%, at least 92%, at least 93%, at least 94%, at least 95%, at least 96%, at least 97%, at least 98%, or at least 99% identical to the sequence of one of SEQ ID NOS: 51-53, and that maintains the same functions as one of SEQ ID NOS: 51-53 (knockdown of EZH1).

TABLE 5
Exemplary EZH1 CRISPRi gRNAs

| | | | | | | |
|---|---|---|---|---|---|---|
| EZH1_KD_1 | CRISPRi | EZH1 | TSS | 1 | GGTGAGTGAGTA | SEQ ID |
| | | | | | AACAAGCC | NO: 51 |
| EZH1_KD_2 | CRISPRi | EZH1 | TSS | 2 | GTAAACAAGCCT | SEQ ID |
| | | | | | GGGCCGGG | NO: 52 |
| EZH1_KD_3 | CRISPRi | EZH1 | TSS | 3 | GTGAGTGAGTAA | SEQ ID |
| | | | | | ACAAGCCT | NO: 53 |

In one embodiment, a vector is used as a transport vehicle to introduce any of the herein described nucleic acid inhibitors of a histone methyltransferase into the target cells selected from the cell populations as described herein (e.g., ESCs; PSCs; iPSCs; hemogenic endothelium; HSCs). In one embodiment, a vector is used as a transport vehicle to introduce any of the herein described nucleic acid comprising the described nucleic acid inhibitors of a histone methyltransferase into the target cells selected from the cell populations as described herein (e.g., ESCs; PSCs; iPSCs; hemogenic endothelium; HSCs). The in vivo expression of the nucleic acid inhibitor is for degrading the mRNA of the targeted histone methyltransferase such as G9a/GLP or EZH1 so as to reduce and inhibit the expression of the respective histone methyltransferase, with the goal being to reduce methylation of the histone H3 in the transfected cells and relief repression of gene expression therein.

In one embodiment, the host cell is an embryonic stem cell, a somatic stem cell, a progenitor cell, a bone marrow cell, a hematopoietic stem cell, a hematopoietic progenitor cell, an immune cell such as a NK cell, T cell or B cell, an erythrocyte, a fibroblast, a keratinocyte, or a myeloid progenitor cell. In one embodiment, the host cell is isolated from a subject. In one embodiment, the host cell is isolated from a subject who has been diagnosed with a hematological disease.

In one embodiment, the vector further comprises a spleen focus-forming virus promoter, a tetracycline-inducible promoter, a Doxycycline (Dox)-inducible, or ar3-globin locus control region and al3-globin promoter. In one embodiment, the promoter provides for targeted expression of the nucleic acid molecule therein. Other examples of promoters include but are not limited to the CMV promoter and EF1-alpha promoters for the various transgenes, and U6 promoter for shRNAs targeting EZH1.

In one embodiment, the vector is a virus or a non-viral vector. Non-limiting examples of viral vectors for gene delivery and expression in cells are retrovirus, adenovirus (types 2 and 5), adeno-associated virus (AAV), Helper-dependent adenoviral vector (HdAd), hybrid adenoviral vectors, herpes virus, pox virus, human foamy virus (HFV), and lentivirus. Exemplary vectors useful in the invention described herein include episomal vectors, integrating vectors, non-integrating vectors, and excisable vectors.

### Stroma-Free NK Cell Differentiation

In some embodiments, the differentiation method comprises differentiating the resultant population of CD34+ hemogenic endothelium in NK-cell-differentiation media in the presence of a Notch ligand for a sufficient time to promote differentiation into a population of CD56+NK cells. The method described herein is a stroma-free NK cell differentiation method. Compared to differentiation with stromal cells expressing a Notch ligand, stroma-free differentiation unexpectedly results in an increased number of differentiated NK cells (see, e.g., Example 1, FIG. 2, FIGS. 3A-3B). Unexpectedly, the inventors found that the stroma-free protocol described herein requires starting with hemogenic endothelium (HE), not iPSC or HE-derived progenitors (e.g., lymphoid progenitor).

In nature, the haematopoietic stem cells (HSCs) in the bone marrow give rise to multipotent progenitors (MPPs) before differentiating into common myeloid progenitors (CMPs) and common lymphoid progenitors (CLPs). CLPs migrate from the bone marrow to the thymus, where thymic epithelial cells that express Delta-like ligand 4 (DLL4) trigger canonical Notch 1 signaling in early NK cell progenitors. This Notch 1 signal is essential for NK cell differentiation. Notch signaling is mediated by the Notch 2 receptor. Notch signaling pathway is highly conserved in both vertebrate and invertebrate species and it regulates many different cell fate decisions. It is important for pattern formation during development such as neurogenesis, angiogenesis or myogenesis and regulates immune cell development and stem cell maintenance. Notch signaling is also involved in cellular processes throughout adulthood. Signaling via Notch occurs between neighboring cells and both the receptor and its ligands are transmembrane proteins. See, e.g., Schmitt T. M., Aliga-Pflücker J. C. (2002) Induction of T cell development from hematopoietic progenitor cells by delta-like-1 in vitro. Immunity 17:749-756; Mohtashami M. (2010) Direct Comparison of Dll1- and Dll4-Mediated Notch Activation Levels Shows Differential Lymphomyeloid Lineage Commitment Outcomes. J Immunol. 185(2):867-76; Ohishi K et al, which are incorporated herein by reference. Delta-1 enhances marrow and thymus repopulating ability of human CD34(⁺) CD38(⁻) cord blood cells. J Clin Invest. 2002 October; 110(8):1165-74; and Dallas M H et al. Density of the Notch ligand Delta1 determines generation of B and T cell precursors from hematopoietic stem cells J Exp Med. 2005 May 2; 201(9): 1361-1366, which are incorporated herein by reference. NOTCH signaling at the late stage of NK cell development promotes maturation and cytotoxic activities. Notch activation facilitates NK cell differentiation in culture. See, e.g., Felices et al., J Immunol. 2014 Oct. 1; 193(7): 3344-3354; Haraguchi et al., J Immunol May 15, 2009, 182 (10) 6168-6178; the contents of each of which are incorporated herein by reference in their entireties.

Accordingly, to initiate differentiation in the lymphoid lineage and NK cell lineage commitment, the hemogenic endothelium is exposed to a Notch ligand to activate the Notch signaling pathway therein. Unexpectedly, the inventors found that the stroma-free protocol described herein, which comprising exposure to a Notch ligand, requires starting with hemogenic endothelium (HE), not iPSC or HE-derived progenitors (e.g., lymphoid progenitor). Accordingly, in some embodiments, iPSC or HE-derived progenitors are not the initial population that is differentiated into NK cells in the presence of a Notch ligand.

Notch ligands are single-pass transmembrane proteins with a DSL (Delta, Serrate, LAG-2)-domain and varying numbers of EGF-like repeats. There are two classes of canonical Notch ligands, the Delta/Delta-like and the Serrate/Jagged class. The later has an additional domain of cysteine rich repeats close to the transmembrane domain. There are 5 canonical Notch ligands in mammals: Jagged-1, Jagged-2, DLL1, DLL3 and DLL4. These can bind to the four Notch receptors Notch 1-4. DLL1, also known as Notch Delta ligand, Delta-like 1, is a protein which interacts with a NOTCH2 receptor. See, e.g., Shimizu K, et al., 2001, J. Biol. Chem. 276 (28): 25753-8; Blaumueller C M, et al., 1997, Cell 90 (2): 281-91; Shimizu K, et al., 2000, Mol. Cell. Biol. 20 (18): 6913-22. DLL1 is a protein that in humans is encoded by the DLL1 gene. DLL1 is a human homolog of the Notch Delta ligand. In some embodiments, the Notch ligand is DLL4.

In some embodiments, the Notch ligand is selected from the group consisting of Delta-like-1 (DLL1, also referred to as DL1), Delta-like-4 (DLL4, also referred to as DL4), immobilized Delta1ext-IgG, and immobilized Delta4ext-IgG. In some embodiments, immobilized Deltalext-IgG consists of an extracellular domain of human Delta-like-1 fused to the Fc domain of human IgG1. “Immobilized Deltalext-IgG” refers to recombinant Notch ligand made by fusing the extracellular domain of Delta-like 1 to the Fc domain of human IgG1 (see, e.g., SEQ ID NO: 42). This is a synthetic way of providing a titratable dose of NOTCH ligand. See, e.g., Varnum-Finney et al., J Cell Sci. 2000 December; 113 Pt 23:4313-8, which is incorporated herein by reference in its entirety. Recombinant Notch ligands and Fc-fusions are commercially available at AdipoGen™. “Immobilized Delta4ext-IgG” refers to recombinant Notch ligand made by fusing the extracellular domain of Delta-like 4 to the Fc domain of human IgG1 (see, e.g., SEQ ID NO: 43).

In some embodiments, the IgG domain of Delta1ext-IgG or Delta4ext-IgG can comprise any known IgG domain in the art. In some embodiments, Deltalext-IgG or Delta4ext-IgG can be immobilized to a solid substrate (e.g., tissue culture plate) by coating the solid substrate with a composition that binds IgG Fc, including but not limited to anti-human IgG antibody, Protein G, or Protein A. In some embodiments, an immobilized Notch ligand (e.g., DLL1 or DLL4) is linked to the Fc domain of IgG1, IgG2, IgG3, or IgG4. In some embodiments, an immobilized Notch ligand (e.g., DLL1 or DLL4) is linked to the Fc domain of human IgG1, IgG2, IgG3, or IgG4.

In some embodiments, the nucleic acid sequence of the Notch ligand (e.g., DLL1) comprises one of SEQ ID NOS: 1-3 or a sequence that is at least 85%, at least 87%, at least 90%, at least 91%, at least 92%, at least 93%, at least 94%, at least 95%, at least 96%, at least 97%, at least 98%, or at least 99%) identical to the sequence of one of SEQ ID NOS: 1-3 that maintains the same functions as one of SEQ ID NOS: 1-3 (e.g., binding and/or activating a Notch receptor).

SEQ ID NO: 1, DLL1 delta like canonical Notch ligand 1 [Homo sapiens (human)], Gene
ID: 28514, NCBI Reference Sequence: NG_027940.1, 8873 bp
(SEQ ID NO: 1)
actgaccatttggcgatccattgagaggagggtttggaaaagtggctcctttgtgacagctctcgccagattggggggctgctgatttgcatc
tcattagccatgcgggcggccggctgaatataagggcggcaggcgccggcgagagccagatcctctgcgcgcacccgcggagacccgacccgg
ccgagggcagagcgcaggggaacccgggcagccgcggcgcagagcctcctcccacggcccggcccctccggtcctgcgcgtgtgtactggatg
gcattggctggattcatcggaaagacgcggatctttgctgtgacaccggagatcggagcccggagtgctcccggaacgaccgccgccgccgag
tgacaccgggccgcgatccgcaggggccgccgcgcacacccgccgccgccgaccgtcccctcagcgcgcgccgctggccccggattatcgcct
tgcccgtgggatttccagaccgcggctttctaatcggctcgggaggaagctctgcagctctcttgggaattaagctcaatctctggactctct
ctctttctctttctccccctccctctcctgcgaagaagctcaagacaaaaccaggaagccggcgaccctcacctcctcgggggctgggaggaa
ggaggaaaacgaaagtcgccgccgccgcgctgtcccccgagagctgcctttcctcgggcatccctggggctgccgcgggacctcgcagggcgg
atataaagaaccgcggccttgggaagaggcggagaccggcttttaaagaaagaagtcctgggtcctgcggtctggggcgaggcaagggcgctt
ttctgcccacgctccccgtggcccatcgatcccccgcgcgtccgccgctgttctaaggagagaagtgggggccccccaggctcgcgcgtggag
cgaagcagcatgggcagtcggtgcgcgctggccctggcggtgctctcggccttgctgtgtcaggtagggggcaggtgggggcgccgcggcccc
gcggggtctcacgggtagccggggcgcggggcaggagcgcgcggggaggggcggacagcggcacgggccgcgccagccacggcccggaagatg
aatcccgggggcgacgaccccagcgccggccgtgcagcgagcgcgctcggcccctgagcccttccaggctctccgcacaccccccacccaggc
ctcacgccccctagctcggggggacccgcgtcctcacgcccccgccctcccccgtgcaggtctggagctctggggtgttcgaactgaagctgc
aggagttcgtcaacaagaaggggctgctggggaaccgcaactgctgccgcgggggcgcggggccaccgccgtgcgcctgccggaccttcttcc
gcgtgtgcctcaagcactaccaggccagcgtgtcccccgagccgccctgcacctacggcagcgccgtcacccccgtgctgggcgtcgactcct
tcagtctgcccgacggcgggggcgccgactccgcgttcagcaaccccatccgcttccccttcggcttcacctggccggtgagtgccgcacctg
cgcgcgccgggccggccctgaagctgggcgggctgcaggacgcgctgggatcccgccttgggcgctcggtggcgggacctcggggaccccgcg
aggcgcaggtgggcgctgcgatctgcctagcggcggccccaggactccagcccagcagcgcggacacctcgccccggggccccgcggcctgca
ggaggggaccgcgctggggcgaggaggagaggccgagcgcgcccgggagatttccgtatccggcctctgtgccaggtctccagtcagaggcgc
cccttcacgtgggaaggttctggtttcccgactcctagacgcgttggtggcgcgattacccgcgcagcgcgaccgctaccacccggagcgtgc
ccatcccccaagaaaaatgacaagggccctcgggcctcttccaccccatcctgcctgcattctctctctctctctaattaaaaaaacaacgta
atatcctgtagtacaggctgaaaaaacacgtcaggaaaccactctttaaaaagttcttccatttccttagggaaggtgagagcaggcaggagg
tgcgtggagaccctctccagacacgctgccccagacctgcagccttcaggcctctgttgctgacctggctgttaggaatgactgctttttgcc
gttttcttttcgttacctttctgggttgtctaacgtcttctcccctctctcccagggcaccttctctctgattattgaagctctccacacaga
ttctcctgatgacctcgcaacaggtaaaaacaaaacccaaaccccaaaactgctttccccagttaatagcattggactttgcccacccatccc
ccagccaaacccggacagctttcattctgcacgtgccccagaaagttcagggtggagcagcttgggcctccttcccgtgctgaatgtctcggc
ccacccccgctctgtcccgagtcacagggttctcgttcagaaccaaccaggagcatcttctccccgtagaaaacccagaaagactcatcagcc
gcctggccacccagaggcacctgacggtgggcgaggagtggtcccaggacctgcacagcagcggccgcacggacctcaagtactcctaccgct
tcgtgtgtgacgaacactactacggagagggctgctccgttttctgccgtccccgggacgatgccttcggccacttcacctgtggggagcgtg
gggagaaagtgtgcaaccctggctggaaagggccctactgcacagagcgtgagtctctgggaaggcaccgctggctcactcgtccacgaacac
ggaccgcgcgcagggacggggcttcctgagccacggggggcttgggactgtagagatgttctggtggggaaactgaggcccagaggacagaag
tggattgctataagtcacagctcgtcagtgggggggttggggtcaacgcagacattttaacatcccaggctgtgtttatccactatcggaact
gcctttcttaatcagggaggattttagagacagggccaggggtcaggaagtaaagccagtgctacccccagggtgtgtgtattagagagggag
aggaggaaggaagggaggaacacagagagagcttgtgtgtcaggggcaccatttcaacccgagttcccagtgctggaacagcatcacactggg
aaacgttccattttctctctggagctggtgtgcttgacctctctggagcaaacgcctttccggatactccctgtgacacgcactgtctatgct
ggccagagagcaggctttcactcctgtgggctgctgaggccaggtctccaaggcctgtgtgggcgaggggtgcacagccccgtctggcttgaa
tgctcaggcagcaccttgtctggagaagcaatgtcttcccaatagtgacagaggctctacctgcctcttattaggtattgatgtgtcaatgtc
atggcaggcaggtgactagggcagggttggggccgtgctggctcctggttctggctcatggggacctcaggagccctctctccagctgactga
ggcctcgcctgcacgcctggccgtcccagcccattggtaccggatttctctacagctggggattgggtaggtcctggagctgcccagaaactc
cagggaactgtcattctccttccttggaactggacaaccttggagaggggctctgggaggcccagaacctctggcaggagctgggtagtgcct
ggggttgagggtgggtcttcccattcactgagtgccttgatgtccttgctccttagcttcccaaattccctccggaacttactgagctccttc
taagctttgccttggcctgaactggttctggggaaaaacaaaaaaacaaaaaacaacttgtggagctgcttgttaatgagtttcataaccagg
cagcaagagccagctccaagcctcaagcccactgtctactccctgccctgcgggagcctctggccagtctgctgcctcccacccttcctccct
gcctctcttcaccacagggtagccagaaacttaaacttttttcttcaaacactgaagtctctccccgcccccagctcgcgcgtgccatagatt
agatctctccggggataggcgcagggacacccgccggctcccattggcggaaggggtgcgtgtgcgtgtgtgtgtgtgtgtgtgtgtgtacac
gcgaggggtgtgtgtgaggaggtggggccgggggcgcgggggaggccggcattgttgcgctggggcagctgccgtggaggacagacaatggag
cagctgtcctgccctggcaccctgcataccagctgtccactcttatctgcacacacactttctgggatattaagaggtggagctttgtgcaca
gaattgggaagtgggggaggaggagggggaagacttctgaccctctcttagaagaaaaggggataggggggggtgggggcttccgagagccct
tttgtccttgagcccctgtgttaagaagaatgctcatccccagggctgagtcaagtcccaggctactaggcaggggggtcagtcctccacaac
ctgggaagattaactcagctgggatttgctgactgaagccggcgagttgtgtcctggccccaagggcggcagccctgttgggacgtacttggc
gtggggcttgaccctgtttttcctttgcttgtagcgatctgcctgcctggatgtgatgagcagcatggattttgtgacaaaccaggggaatgc
aagtaagtctgcacaaggtggtgttttgttttgttgccttttcttgttatcttttcacagctggtgtatttgtaaaaacagccctaggtgatc
attcgaaaaactccagtaagattgattgaacagggggccgttttctcatgtttctacttaatcaatgtttggcagcatgtaaggtcatggagt
tgtcattcgtctaagccccttaacggctatgagaatttacagatagtagtttaaaaagagttggcacaggaaatgatagtatagttcaatggt
tctcaaatgttgcctcatcctagaatcactcagggagtgatttttgagatgctgacactggtgctgccctaacacccaagaagccagaacctc
tggtggggcccaggcccaggctgcagctcccaaggtgacccagtgttctgctaatctggagaaccagaggctcactggtgctgcgggaagatg
gtttctagggtgagaatgtccactgcaaagccagcaacagtcaacgtccatctgagtcttctgcttttctccaaggtgcagagtgggctggca
gggccggtactgtgacgagtgtatccgctatccaggctgtctccatggcacctgccagcagccctggcagtgcaactgccaggaaggctgggg
gggccttttctgcaaccagggtaagccttctctccctgaggcagcctgctccctccagagcagccctggacttccctggctgtttgatcactg
gaaaaataaagtcttcctgcatttgatgtcgagcttcctatctcctacttttcctgtccccacccttcacagacctgaactactgcacacacc
ataagccctgcaagaatggagccacctgcaccaacacgggccaggggagctacacttgctcttgccggcctgggtacacaggtgccacctgcg
agctggggattgacgagtgtgaccccagcccttgtaagaacggagggagctgcacggtgagtcggaggctccatggcatctcacccggaagct
ggggtgccctggtgttgaatggagtgtgtgggctccttggagcaactttggaaagccttttctgacctctccatcgtgtaggatctcgagaac
agctactcctgtacctgcccacccggcttctacggcaaaatctgtgaattgagtgccatgacctgtgcggacggcccttgctttaacgggggt
cggtgctcagacagccccgatggagggtacagctgccgctgccccgtgggctactccggcttcaactgtgagaagaaaattgactactgcagc
tcttcaccctgttctaatggtaagggggcagctggtgattgctcagagactcgggcgagcggtcaatactgaggtggcattaaaaacaagcat
ttgtgagtgacctcgagtttatgaatcacttttatccagaccgccaggaattctcgatggaaactctatctttgagtctggaaaggcctgggg
aatgagagaggccagggcatttgttatgaagttctctgtggaaacctagaccaagcagtgaatgacttgctcagggccacaaggtgcttcggg
cacctgcggccgcctgaggttcagtaagtgatgcccacaggtgccggccactccagcttgggaggatggcccagctgtgtggccacccagcac
agtagttgggggtgtccctgagtgaggacagagagcctcctgctagcagcgaggggctggctgcccaaaggagacacacagcaaggagagctg
ggccccagatgtgccggagcattccggaatggtcatccttcccctccctccctcccctgttgtcagtgcctgctcctctcacttgctgtgtaa
ctgtgggcaaggacaccctcgttaagcctcagtttccccatctgaaacctgggtcgagtggcacatgctcttgcccggctgttgtggcgacta
atgcagccaccagagtgttctgcacagcgcctgtccagatgctggccgtgtggtttctgacttgtagagctagacctggacacctctcgtatt
tgaggtcctaaaccatgtcaccttgcgctgtggactcattcaggccacagactgtctttggtttgtctggtttctacagtgtcagacagatag
atgcttcagagtgactttttggtgaacaaacctacgaggagacacgtgatgttcatgtccctgtgttccaggtgccaagtgtgtggacctcgg
tgatgcctacctgtgccgctgccaggccggcttctcggggaggcactgtgacgacaacgtggacgactgcgcctcctccccgtgcgccaacgg
gggcacctgccgggatggcgtgaacgacttctcctgcacctgcccgcctggctacacgggcaggaactgcagtgcccccgtcagcaggtgcga
gcacgcaccctgccacaatggggccacctgccacgagaggggccaccgctatgtgtgcgagtgtgcccgaggctacgggggtcccaactgcca
gttcctgctccccgagctgcccccgggcccagcggtggtggacctcactgagaagctagagggccaggggggccattcccctgggtggccgtg
tgcgccggggtcatccttgtcctcatgctgctgctgggctgtgccgctgtggtggtctgcgtccggctgaggctgcagaagcaccggccccca
gccgacccctgccggggggagacggagaccatgaacaacctggccaactgccagcgtgagaaggacatctcagtcagcatcatcggggccacg
cagatcaagaacaccaacaagaaggcggacttccacggggaccacagcgccgacaagaatggcttcaaggcccgctacccagcggtggactat
aacctcgtgcaggacctcaagggtgacgacaccgccgtcagggacgcgcacagcaagcgtgacaccaagtgccagccccagggctcctcaggg
gaggagaaggggaccccgaccacactcagggggtgcgtgctgcgggccgggcatcaggagggggtacctggggggtgtcttcctggaaccact
gctccgtttctcttcccaaatgttctcatgcattcattgtggattttctctattttccttttagtggagaagcatctgaaagaaaaaggccgg
actcgggctgttcaacttcaaaagacaccaagtaccagtcggtgtacgtcatatccgaggagaaggatgagtgcgtcatagcaactgaggtca
gtgcaggcagcagccgctccctcctcctcggcatgggagcacctgaagctggagcacgggaatcggtctcaggctaacttcccatttgtcttg
tggccccccaggtgtaaaatggaagtgagatggcaagactcccgtttctcttaaaataagtaaaattccaaggatatatgccccaacgaatgc
tgctgaagaggagggaggcctcgtggactgctgctgagaaaccgagttcagaccgagcaggttctcctcctgaggtcctcgacgcctgccgac
agcctgtcgcggcccggccgcctgcggcactgccttccgtgacgtcgccgttgcactatggacagttgctcttaagagaatatatatttaaat
gggtgaactgaattacgcataagaagcatgcactgcctgagtgtatattttggattcttatgagccagtcttttcttgaattagaaacacaaa
cactgcctttattgtcctttttgatacgaagatgtgctttttctagatggaaaagatgtgtgttattttttggatttgtaaaaatatttttca
tgatatctgtaaagcttgagtattttgtgatgttcgttttttataatttaaattttggtaaatatgtacaaaggcacttcgggtctatgtgac
tatatttttttgtatataaatgtatttatggaatattgtgcaaatgttatttgagttttttactgttttgttaatgaagaaattcctttttaa
aatatttttccaaaataaattttatgaatgacaa
SEQ ID NO: 2 Homo sapiens delta like canonical Notch ligand 1 (DLL1), mRNA, NCBI
Reference Sequence: NM_005618.4, 3779 bp
(SEQ ID NO: 2)
actgaccatttggcgatccattgagaggagggtttggaaaagtggctcctttgtgacagctctcgccagattggggggctgctgatttgcatc
tcattagccatgcgggcggccggctgaatataagggcggcaggcgccggcgagagccagatcctctgcgcgcacccgcggagacccgacccgg
ccgagggcagagcgcaggggaacccgggcagccgcggcgcagagcctcctcccacggcccggcccctccggtcctgcgcgtgtgtactggatg
gcattggctggattcatcggaaagacgcggatctttgctgtgacaccggagatcggagcccggagtgctcccggaacgaccgccgccgccgag
tgacaccgggccgcgatccgcaggggccgccgcgcacacccgccgccgccgaccgtcccctcagcgcgcgccgctggccccggattatcgcct
tgcccgtgggatttccagaccgcggctttctaatcggctcgggaggaagctctgcagctctcttgggaattaagctcaatctctggactctct
ctctttctctttctccccctccctctcctgcgaagaagctcaagacaaaaccaggaagccggcgaccctcacctcctcgggggctgggaggaa
ggaggaaaacgaaagtcgccgccgccgcgctgtcccccgagagctgcctttcctcgggcatccctggggctgccgcgggacctcgcagggcgg
atataaagaaccgcggccttgggaagaggcggagaccggcttttaaagaaagaagtcctgggtcctgcggtctggggcgaggcaagggcgctt
ttctgcccacgctccccgtggcccatcgatcccccgcgcgtccgccgctgttctaaggagagaagtgggggccccccaggctcgcgcgtggag
cgaagcagcatgggcagtcggtgcgcgctggccctggcggtgctctcggccttgctgtgtcaggtctggagctctggggtgttcgaactgaag
ctgcaggagttcgtcaacaagaaggggctgctggggaaccgcaactgctgccgcgggggcgcggggccaccgccgtgcgcctgccggaccttc
ttccgcgtgtgcctcaagcactaccaggccagcgtgtcccccgagccgccctgcacctacggcagcgccgtcacccccgtgctgggcgtcgac
tccttcagtctgcccgacggcgggggcgccgactccgcgttcagcaaccccatccgcttccccttcggcttcacctggccgggcaccttctct
ctgattattgaagctctccacacagattctcctgatgacctcgcaacagaaaacccagaaagactcatcagccgcctggccacccagaggcac
ctgacggtgggcgaggagtggtcccaggacctgcacagcagcggccgcacggacctcaagtactcctaccgcttcgtgtgtgacgaacactac
tacggagagggctgctccgttttctgccgtccccgggacgatgccttcggccacttcacctgtggggagcgtggggagaaagtgtgcaaccct
ggctggaaagggccctactgcacagagccgatctgcctgcctggatgtgatgagcagcatggattttgtgacaaaccaggggaatgcaagtgc
agagtgggctggcagggccggtactgtgacgagtgtatccgctatccaggctgtctccatggcacctgccagcagccctggcagtgcaactgc
caggaaggctgggggggccttttctgcaaccaggacctgaactactgcacacaccataagccctgcaagaatggagccacctgcaccaacacg
ggccaggggagctacacttgctcttgccggcctgggtacacaggtgccacctgcgagctggggattgacgagtgtgaccccagcccttgtaag
aacggagggagctgcacggatctcgagaacagctactcctgtacctgcccacccggcttctacggcaaaatctgtgaattgagtgccatgacc
tgtgcggacggcccttgctttaacgggggtcggtgctcagacagccccgatggagggtacagctgccgctgccccgtgggctactccggcttc
aactgtgagaagaaaattgactactgcagctcttcaccctgttctaatggtgccaagtgtgtggacctcggtgatgcctacctgtgccgctgc
caggccggcttctcggggaggcactgtgacgacaacgtggacgactgcgcctcctccccgtgcgccaacgggggcacctgccgggatggcgtg
aacgacttctcctgcacctgcccgcctggctacacgggcaggaactgcagtgcccccgtcagcaggtgcgagcacgcaccctgccacaatggg
gccacctgccacgagaggggccaccgctatgtgtgcgagtgtgcccgaggctacgggggtcccaactgccagttcctgctccccgagctgccc
ccgggcccagcggtggtggacctcactgagaagctagagggccaggggggccattcccctgggtggccgtgtgcgccggggtcatccttgtcc
tcatgctgctgctgggctgtgccgctgtggtggtctgcgtccggctgaggctgcagaagcaccggcccccagccgacccctgccggggggaga
cggagaccatgaacaacctggccaactgccagcgtgagaaggacatctcagtcagcatcatcggggccacgcagatcaagaacaccaacaaga
aggcggacttccacggggaccacagcgccgacaagaatggcttcaaggcccgctacccagcggtggactataacctcgtgcaggacctcaagg
gtgacgacaccgccgtcagggacgcgcacagcaagcgtgacaccaagtgccagccccagggctcctcaggggaggagaaggggaccccgacca
cactcaggggtggagaagcatctgaaagaaaaaggccggactcgggctgttcaacttcaaaagacaccaagtaccagtcggtgtacgtcatat
ccgaggagaaggatgagtgcgtcatagcaactgaggtgtaaaatggaagtgagatggcaagactcccgtttctcttaaaataagtaaaattcc
aaggatatatgccccaacgaatgctgctgaagaggagggaggcctcgtggactgctgctgagaaaccgagttcagaccgagcaggttctcctc
ctgaggtcctcgacgcctgccgacagcctgtcgcggcccggccgcctgcggcactgccttccgtgacgtcgccgttgcactatggacagttgc
tcttaagagaatatatatttaaatgggtgaactgaattacgcataagaagcatgcactgcctgagtgtatattttggattcttatgagccagt
cttttcttgaattagaaacacaaacactgcctttattgtcctttttgatacgaagatgtgctttttctagatggaaaagatgtgtgttatttt
ttggatttgtaaaaatatttttcatgatatctgtaaagcttgagtattttgtgatgttcgttttttataatttaaattttggtaaatatgtac
aaaggcacttcgggtctatgtgactatatttttttgtatataaatgtatttatggaatattgtgcaaatgttatttgagttttttactgtttt
gttaatgaagaaattcctttttaaaatatttttccaaaataaattttatgaatgacaa
SEQ ID NO: 3 Homo sapiens delta like canonical Notch ligand 1 (DLL1), CDS mRNA,
NCBI Reference Sequence: NM_005618.4, 2172 bp
(SEQ ID NO: 3)
atgggcagtcggtgcgcgctggccctggcggtgctctcggccttgctgtgtcaggtctggagctctggggtgttcgaactgaagctgcaggag
ttcgtcaacaagaaggggctgctggggaaccgcaactgctgccgcgggggcgcggggccaccgccgtgcgcctgccggaccttcttccgcgtg
tgcctcaagcactaccaggccagcgtgtcccccgagccgccctgcacctacggcagcgccgtcacccccgtgctgggcgtcgactccttcagt
ctgcccgacggcgggggcgccgactccgcgttcagcaaccccatccgcttccccttcggcttcacctggccgggcaccttctctctgattatt
gaagctctccacacagattctcctgatgacctcgcaacagaaaacccagaaagactcatcagccgcctggccacccagaggcacctgacggtg
ggcgaggagtggtcccaggacctgcacagcagcggccgcacggacctcaagtactcctaccgcttcgtgtgtgacgaacactactacggagag
ggctgctccgttttctgccgtccccgggacgatgccttcggccacttcacctgtggggagcgtggggagaaagtgtgcaaccctggctggaaa
gggccctactgcacagagccgatctgcctgcctggatgtgatgagcagcatggattttgtgacaaaccaggggaatgcaagtgcagagtgggc
tggcagggccggtactgtgacgagtgtatccgctatccaggctgtctccatggcacctgccagcagccctggcagtgcaactgccaggaaggc
tgggggggccttttctgcaaccaggacctgaactactgcacacaccataagccctgcaagaatggagccacctgcaccaacacgggccagggg
agctacacttgctcttgccggcctgggtacacaggtgccacctgcgagctggggattgacgagtgtgaccccagcccttgtaagaacggaggg
agctgcacggatctcgagaacagctactcctgtacctgcccacccggcttctacggcaaaatctgtgaattgagtgccatgacctgtgcggac
ggcccttgctttaacgggggtcggtgctcagacagccccgatggagggtacagctgccgctgccccgtgggctactccggcttcaactgtgag
aagaaaattgactactgcagctcttcaccctgttctaatggtgccaagtgtgtggacctcggtgatgcctacctgtgccgctgccaggccggc
ttctcggggaggcactgtgacgacaacgtggacgactgcgcctcctccccgtgcgccaacgggggcacctgccgggatggcgtgaacgacttc
tcctgcacctgcccgcctggctacacgggcaggaactgcagtgcccccgtcagcaggtgcgagcacgcaccctgccacaatggggccacctgc
cacgagaggggccaccgctatgtgtgcgagtgtgcccgaggctacgggggtcccaactgccagttcctgctccccgagctgcccccgggccca
gcggtggtggacctcactgagaagctagagggccagggcgggccattcccctgggtggccgtgtgcgccggggtcatccttgtcctcatgctg
ctgctgggctgtgccgctgtggtggtctgcgtccggctgaggctgcagaagcaccggcccccagccgacccctgccggggggagacggagacc
atgaacaacctggccaactgccagcgtgagaaggacatctcagtcagcatcatcggggccacgcagatcaagaacaccaacaagaaggcggac
ttccacggggaccacagcgccgacaagaatggcttcaaggcccgctacccagcggtggactataacctcgtgcaggacctcaagggtgacgac
accgccgtcagggacgcgcacagcaagcgtgacaccaagtgccagccccagggctcctcaggggaggagaaggggaccccgaccacactcagg
ggtggagaagcatctgaaagaaaaaggccggactcgggctgttcaacttcaaaagacaccaagtaccagtcggtgtacgtcatatccgaggag
aaggatgagtgcgtcatagcaactgaggtgtaa

In some embodiments, the amino acid sequence of the Notch ligand (e.g., DLL1) comprises SEQ ID NO: 4 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 4 and that maintains the same functions as SEQ ID NO: 4 (e.g., binding and/or activating a Notch receptor).

SEQ ID NO: 4 delta-like protein 1 precursor
[Homo sapiens], NCBI Reference Sequence:
NP_005609.3, 723 aa
(SEQ ID NO: 4)
MGSRCALALAVLSALLCQVWSSGVFELKLQEFVNKKGLLGNRNCCRGGA
GPPPCACRTFFRVCLKHYQASVSPEPPCTYGSAVTPVLGVDSFSLPDGG
GADSAFSNPIRFPFGFTWPGTFSLIIEALHTDSPDDLATENPERLISRL
ATQRHLTVGEEWSQDLHSSGRTDLKYSYRFVCDEHYYGEGCSVFCRPRD
DAFGHFTCGERGEKVCNPGWKGPYCTEPICLPGCDEQHGFCDKPGECKC
RVGWQGRYCDECIRYPGCLHGTCQQPWQCNCQEGWGGLFCNQDLNYCTH
HKPCKNGATCTNTGQGSYTCSCRPGYTGATCELGIDECDPSPCKNGGSC
TDLENSYSCTCPPGFYGKICELSAMTCADGPCFNGGRCSDSPDGGYSCR
CPVGYSGFNCEKKIDYCSSSPCSNGAKCVDLGDAYLCRCQAGFSGRHCD
DNVDDCASSPCANGGTCRDGVNDFSCTCPPGYTGRNCSAPVSRCEHAPC
HNGATCHERGHRYVCECARGYGGPNCQFLLPELPPGPAVVDLTEKLEGQ
GGPFPWVAVCAGVILVLMLLLGCAAVVVCVRLRLQKHRPPADPCRGETE
TMNNLANCQREKDISVSIIGATQIKNTNKKADFHGDHSADKNGFKARYP
AVDYNLVQDLKGDDTAVRDAHSKRDTKCQPQGSSGEEKGTPTTLRGGEA
SERKRPDSGCSTSKDTKYQSVYVISEEKDECVIATEV

In some embodiments, the Notch ligand (e.g., Delta1ext-IgG) comprises the extracellular domain of human DLL1, which corresponds to approximately amino acids 1-536, or amino acids 22-544, or amino acids 22-537 of DLL1 (see, e.g., SEQ ID NO: 4 for full-length sequence of DLL1). In some embodiments, the extracellular domain of human DLL1 comprises SEQ ID NO: 5, or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 5, and that maintains the same functions as SEQ ID NO: 5 (e.g., binding and/or activating a Notch receptor).

SEQ ID NO: 5, human DLL1 extracellular domain,
536 amino acids
(SEQ ID NO: 5)
MGSRCALALAVLSALLCQVWSSGVFELKLQEFVNKKGLLGNRNCCRGGA
GPPPCACRTFFRVCLKHYQASVSPEPPCTYGSAVTPVLGVDSFSLPDGG
GADSAFSNPIRFPFGFTWPGTFSLIIEALHTDSPDDLATENPERLISRL
ATQRHLTVGEEWSQDLHSSGRTDLKYSYRFVCDEHYYGEGCSVFCRPRD
DAFGHFTCGERGEKVCNPGWKGPYCTEPICLPGCDEQHGFCDKPGECKC
RVGWQGRYCDECIRYPGCLHGTCQQPWQCNCQEGWGGLFCNQDLNYCTH
HKPCKNGATCTNTGQGSYTCSCRPGYTGATCELGIDECDPSPCKNGGSC
TDLENSYSCTCPPGFYGKICELSAMTCADGPCFNGGRCSDSPDGGYSCR
CPVGYSGFNCEKKIDYCSSSPCSNGAKCVDLGDAYLCRCQAGFSGRHCD
DNVDDCASSPCANGGTCRDGVNDFSCTCPPGYTGRNCSAPVSRCEHAPC
HNGATCHERGHRYVCECARGYGGPNCQFLLPELPPGPAVVDLTEKL

In some embodiments, the nucleic acid sequence of the Notch ligand (e.g., DLL4) comprises one of SEQ ID NOS: 6-9 or a sequence that is at least 85%, at least 87%, at least 90%, at least 91%, at least 92%, at least 93%, at least 94%, at least 95%, at least 96%, at least 97%, at least 98%, or at least 99% identical to the sequence of one of SEQ ID NOS: 6-9, and that maintains the same functions as one of SEQ ID NOS: 6-9 (e.g., binding and/or activating a Notch receptor).

SEQ ID NO: 6, DLL4 delta like canonical Notch ligand 4 [Homo sapiens (human)], Gene
ID: 54567, NCBI Reference Sequence: NG_046974.1, 9734 bp
(SEQ ID NO: 6)
agtagcggcgctgcgcgcaggccgggaacacgaggccaagagccgcagccccagccgccttggtgcagcgtacaccggcactagcccgctt
gcagccccaggattagacagaagacgcgtcctcggcgcggtcgccgcccagccgtagtcacctggattacctacagcggcagctgcagcgg
agccagcgagaaggccaaaggggagcagcgtcccgagaggagcgcctcttttcagggaccccgccggctggcggacgcgcgggaaagcggc
gtcgcgaacagagccagattgagggcccgcgggtggagagagcgacgcccgaggggatggcggcagcgtcccggagcgcctctggctgggc
gctactgctgctggtggcactttggcagcaggtaacacgtcccgcgccctctccgtcccctctgccgcgctctgggcctcagccccgggca
ccagctgagctgaccggtcccctccctccttccctcggtccctgtgcaatagcgcgcggccggctccggcgtcttccagctgcagctgcag
gagttcatcaacgagcgcggcgtactggccagtgggcggccttgcgagcccggctgccggactttcttccgcgtctgccttaagcacttcc
aggcggtcgtctcgcccggaccctgcaccttcgggaccgtctccacgccggtattgggcaccaactccttcgctgtccgggacgacagtag
cggcggggggcgcaaccctctccaactgcccttcaatttcacctggccggtgagcacagcctgggcgcactgggaggtcgcagaagccgag
agaggaggcgccctgggaccaaagccccctccccagatttccttgtacacacacccccacccccaaaaagcccaggatgcattctttcctg
gctcttcccgactctctcctgagactgatcccagaaaaggctctcaccagtctccgtcttcccagtttatgtcctcccgtccccagctctt
gggacacgattttcattacctaccactctggggcggtaccctaccaccccctcctccagtggctctcccttacactctcccgtctctcaac
cctccctctaccgggggttctcctctcgccttccctgctcaagcgctacactgtgcacagccccgttatgttgacccgggcgcagtaactg
aatcctgcaattagattaattaaacaggctgccgcaaggcacccccacctctccccgcttgctcatctcgccatctctccgtccccccacc
ccctttcccagggtaccttctcgctcatcatcgaagcttggcacgcgccaggagacgacctgcggccaggtgagtagctcgctccgccacc
acaggggggcgacacggcgcagcgccgaaagagttaatctgttctaggcgggggaagtgcgggcttggggggggaggcaggacgcttagct
tggcctggagctgcgccccgcgctggacgctcggattccgctcgctgcctggactcagagcacaattgcgtttcctgcgggttatttttgg
cgtgggaacgcggggagtacggcggtgagaaaggctgaagctgccagcgccgctgacgggccccttcctgtattttacacctttcgcgaat
tccgctcctttggaaagggaataatggctttgggatgttgttctgacacagaggaaaaggatatttcagcagcacaacaattctcactttg
aaaaggaaaaaagaaaaccattacccacctctggaggcagaacccctgaatgggcaccaaaggaccccctgctcccagggtcctctctagc
ctggggagcttttctttctttttctcttttttccattttgacctcttttcctctttcccctccctatctgcctccaagaccctgggatatc
ttaacatccttctattgtcccctttttgaatactatcaggccccctgcacatgcacacacgtagggcagctacgtagcggggctttgggtc
cctctggcctgttcttgctggcaggcgggggtcatctggataactgggctgattggttggctgatcaccatcatcacagccaagaaggaca
ttggccagccgtcactggcacccttggggactggcgacccttccctgacccgaccctctgccccctcagaggccttgccaccagatgcact
catcagcaagatcgccatccagggctccctagctgtgggtcagaactggttattggatgagcaaaccagcaccctcacaaggctgcgctac
tcttaccgggtcatctgcagtgacaactactatggagacaactgctcccgcctgtgcaagaagcgcaatgaccacttcggccactatgtgt
gccagccagatggcaacttgtcctgcctgcccggttggactggggaatattgccaacagcgtaagcagtcaagctcccacctgtgtggaag
gggagggtcccctgaggaaacacagtggagcttcttggtcacagcttgcctcccttgaagagtgggtctgggcctcctactagctgggcct
cagggatgctgagggtgggcttgacctcagacctcctgtctcttcccagtgctcctcccatcatgccaaagcccacaagaaccccatcatg
acattccatccagtttggcttctccttccctgtgccattatttcactttaagacactcggggctcctctgggaggccaggagtaggaagag
ggcccaggagagctaggggatccccagggccagcaggtgagaatggggcttaagagtccttggtatcccagcctcacccagctctgtgttc
ttcccttagctatctgtctttcgggctgtcatgaacagaatggctactgcagcaagccagcagagtgcctgtgagtaggggacaggaagtg
gtgagtgggagccctcccttggccaaggcctctcacctcactctgcctctctcttgttccccagctgccgcccaggctggcagggccggct
gtgtaacgaatgcatcccccacaatggctgtcgccacggcacctgcagcactccctggcaatgtacttgtgatgagggctggggaggcctg
ttttgtgaccaaggtgagtcagggtgaagagagggtgcagagggtgcaagagatatggggctggggggtggaaatccgattcgtcacctgg
atccttcttacttggtgactgcagacttggctttcccatgatcttccaaggatcttgggtcttttaaggatctttacaactggcccagaat
gaggcggtgggtccttctccaggtgcggcggcagggggtggtggagccagggtggctgaaaaacccaggggggtgacaaggtcggcagcct
ggaggttgcactcataaatcctagcaaagccaaagagagagggatggcaggctcagttcctctttcaaccccgtagttacctattaacccc
ctgagtgtttgcttaccttccagggctgtttgagcagctctcccctaaacagctgtccggtggggtgtgcccaccggccacctgaggctgt
gggtgagctgggcctctgggcggagtggcatctaaccgacttttcggtgtgggcacaaacggcctcccctgctcttacctagttaccacct
gcctgaacccatgcggtctctacctggtgtttaggggtagtcactctctggctatacaggggcctttcagccccaaccttgggggaggagg
aagccttttttcttgcatcctgctagccagctgcagccagctgcagctcccattttcaggatcaaatgggtgcacctgctgcccagagaca
ccggcgcaggcctgggtagggtgggcagagagcttgccagggtggaaagaaattgcctaggccctgacttgctgtcaacaaggggcttggg
attcagtccctgtgttgtgtgtgtgtgtgtgtgtgtgtgtgtgtgtctgtccctttactaccatccccaccccaacactcacacacctggt
tcctgctcattctcttccctctccaccatatttgctcccaggtgacacagtcatatactcatcatatgcaaacacagcacttgcaggccat
atatttactctgtctggttctccctccctgtccttcccaaataaaaaaacaaatacttatatttcaaaatacccttgtaacacctcttcct
ttaaaaaatgcccgattactgcctatggtggctctcatctctcctctaccatttctacctgttgaaattttatccctccttccaggcttat
ctcagctgcccctcctccatgaagccttttctgacttcctccccgacatgtggccttgccctctgctcttcttccttatcttcatcctact
tgggttggcagtttgtgagtttccctggcaggacgtcttccagttccagttgtgttgtttcacttttggttgactgcactggtcatatgtg
attcaaggtgctttaagaaacatgattttcatcctggctaacacagtgaaaccctgtctgtattaaaaatacaaaagttagccaggtgtgg
tggcaggcacctgtagccccagctgctgggaaggctgaggcaggagaatggcgaagtagagcttgcagtgagccgaggtcgtgccactgca
ctccagcctgagtgacagagcaagactccgtctcaaaaaaaaaaaaaaaaaaaaaaaaaagaaacatgattttaggctgggtgcgatggcc
tgtaatcccagcactttgggaggccgaggtaggtggatcacttgaagtcaggagttcgagaccatcctggccatcctggtgaaacccctgt
aaaaatacaaatattaatcgggcacagtggcgcatgcctgtaatcccagctacttagaaggttgaggtatgagaatcgcttgaacccggaa
ggcgaaggttgtagtgagcctatatcacatcactgcactccagcctgggcgacagagtgagactctgttaaaaaaaaaaaaaaaagaagga
aagaaagagaaagagagagaaagaaagaaagaaagagaaagaaaaaagattttattggtggtggaggaaggatgtttgggcctgggagact
ttgagttgaggtgtctttgagccaaacatgggggcaaacatggactgcaaggagcctggaggtgagtgcattccctggccctgctcagctg
cttggttcctgtttctgcagatctcaactactgcacccaccactccccatgcaagaatggggcaacgtgctccaacagtgggcagcgaagc
tacacctgcacctgtcgcccaggctacactggtgtggactgtgagctggagctcagcgagtgtgacagcaacccctgtcgcaatggaggca
gctgtaaggtgaggcccagaccagcgcaggaagacagaggtgtcaggtggtgtctgggcatccctaacctaggcagttagtggatgtacag
ccatggacaggcattgtgggcaggtggagcccagccttcagtcacacatccctgccccccagggtctgactttggcccctttatggtctct
ctccaggaccaggaggatggctaccactgcctgtgtcctccgggctactatggcctgcattgtgaacacagcaccttgagctgcgccgact
ccccctgcttcaatgggggctcctgccgggagcgcaaccagggggccaactatgcttgtgaatgtccccccaacttcaccggctccaactg
cgagaagaaagtggacaggtgcaccagcaacccctgtgccaacggtgcgtgctgctgccctgctaacctggtggactggccctggggctga
gagagacttctggtgagggagggtcaggagaggagcgaggcattgtctgccactctggccccccatctgctctggagggcgaagagcttgc
ttgatcagctggggggctgtggaagcggagctggttagttgcacgcaggccttaggagcaggggtggtatgcaccctgcatagcttccatt
cctattcccatgtcagaaccccgtcctggctggggggcctctgaccctccccaggaagtcctgagctggagagagggatgttggaggcttc
atgtttctcctcaaaggaggcagtgattcagtcagagccctgctcctggaggcctcatcttgccccgtgcccaggtagagcatgaggtagc
atgaggcatcttgaatgtttgcaccttttgaggcacaaagcctgttggtaatccttgtctatctggctcccaggtgaccctctgtgaggca
ggcaggcaggcagcgctcaggagctggagagggggggaagggctgagagggagtctgctctctcactgaagcctctggcactgccatttct
tcatcactgaatgggaaactataatacctgtcctctgtccttcatgtggttgtgaagatgaagtaaaacagtcatgattgtacttatccga
gcattaactatataccaaacatgggctcttgccttcatgtaccttcccggctatcctatgaaggggctagcattctactccagtctaacaa
atggggaaactgaggcttagagacacggttaagcagcaagtgccagatctcaggccacagagtgacagctgaggtcccaactcaagcctat
ctgtctgattctacgttaaagttctgtaagatgctagtcatttttatacatgagcccactgaggccgagagaatcaaggtcatgctaaact
ccaggtctcctgactctgtgcagttctctttgtagtgggctctgcaggtggaggtagaagggcccgaacgtgttcctggaatggggctccc
accccctgccccagggagctcccaggctatcactgacttgtgtctcatgcgtcctcacagggggacagtgcctgaaccgaggtccaagccg
catgtgccgctgccgtcctggattcacgggcacctactgtgaactccacgtcagcgactgtgcccgtaacccttgcgcccacggtggcact
tgccatgacctggagaatgggctcatgtgcacctgccctgccggcttctctggccgacgctgtgaggtgcggacatccatcgatgcctgtg
cctcgagtccctgcttcaacagggccacctgctacaccgacctctccacagacacctttgtgtgcaactgcccttatggctttgtgggcag
ccgctgcgagttccccgtgggcttgccgcccagcttcccctgggtggccgtctcgctgggtgtggggctggcagtgctgctggtactgctg
ggcatggtggcagtggctgtgcggcagctgcggcttcgacggccggacgacggcagcagggaagccatgaacaacttgtcggacttccaga
aggacaacctgattcctgccgcccagcttaaaaacacaaaccagaagaaggagctggaagtggactgtggcctggacaagtccaactgtgg
caaacagcaaaaccacacattggactataatctggccccagggcccctggggcgggggaccatgccaggaaagtttccccacagtgacaag
agcttaggagagaaggcgccactgcggttacacaggtgagtggcacccagaagcccagggcctggccaccggccccgacatggttctgcct
aggctcctcttaggccaggcgggaagcagttaagcagctgaggttttgttactgacaggaagatcctccagtaggatttctgtcaggggtc
ctttgtccttccctcccattcattcatttgttcattcacacatgtcaagtgtccctagggtgtctcttgtgacttccgtctttccacagtg
tggcttgcctctagtggcagcactggctttatgcagggctcagacccttctggtgaggttgggaggcctgtgactctcttaggggcctttt
cctaagtgcccccctgcagcagcccagcactgggcacgtccagcccctgtgtcttccccaagaaccaccctgcagatgccctttggctctc
cagggtcctccctccccccaagcctctccccgtccctcccttacacgcctgtcttgtgttccctcagtgaaaagccagagtgtcggatatc
agcgatatgctcccccagggactccatgtaccagtctgtgtgtttgatatcagaggagaggaatgaatgtgtcattgccacggaggtgagt
gctgggctcgcctttccttctgccttttgtgggagggaaagtggcctggtcactcttgacccatgggccattcctgaagggtaggtcagaa
ccctgccttggcaggccaagttcagtggactcttgggtccctgctggcctcattgccactaagggtgtgaaacaggaaccatggcggcaag
cctggtctggtcctttcctgctgtattggtgctgggttgggcagccacggcactgctggccagcctctgatgggtgagggggcccctcacc
ccttgtgcccttcctgccccttcccactggcttcctccattgacctcatgagcgcaagctcccaggcccgtgtgtgtgttgggccgaagac
tggggaggactgccccacctgcccttagcccctgcctgccccatcgccttctcccagggaggcccagggagggcctggagggagtgcgcat
gcccagggtaacctgtttccctgccttccgcttgctcccaggtataaggcaggagcctacctggacatccctgctcagccccgcggctgga
ccttccttctgcattgtttacattgcatcctggatgggacgtttttcatatgcaacgtgctgctctcaggaggaggagggaatggcaggaa
ccggacagactgtgaacttgccaagagatgcaatacccttccacacctttgggtgtctgtctggcatcagattggcagctgcaccaaccag
aggaacagaagagaagagagatgccactgggcactgccctgccagtagtggccttcagggggctccttccggggctccggcctgttttcca
gagagagtggcagtagccccatggggcccggagctgctgtggcctccactggcatccgtgtttccaaaagtgcctttggcccaggctccac
ggcgacagttgggcccaaatcagaaaggagagagggggccaatgagggcagggcctcctgtgggctggaaaaccactgggtgcgtctcttg
ctggggtttgccctggaggtgaggtgagtgctcgagggaggggagtgctttctgccccatgcctccaactactgtatgcaggcctggctct
ctggtctaggccctttgggcaagaatgtccgtctacccggcttccaccaccctctggccctgggcttctgtaagcagacaggcagagggcc
tgcccctcccaccagccaagggtgccaggcctaactggggcactcagggcagtgtgttggaaattccactgagggggaaatcaggtgctgc
ggccgcctgggccctttcctccctcaagcccatctccacaacctcgagcctgggctctggtccactactgccccagaccaccctcaaagct
ggtcttcagaaatcaataatatgagtttttattttgtttttttttttttttttgtagtttattttggagtctagtatttcaataatttaag
aatcagaagcactgacctttctacattttataacattattttgtatataatgtgtatttataatatgaaacagatgtgtacagga
SEQ ID NO: 7, Homo sapiens delta like canonical Notch ligand 4 (DLL4), mRNA,
NCBI Reference Sequence: NM_019074.4, 3426 bp
(SEQ ID NO: 7)
agtagcggcgctgcgcgcaggccgggaacacgaggccaagagccgcagccccagccgccttggtgcagcgtacaccggcactagcccgctt
gcagccccaggattagacagaagacgcgtcctcggcgcggtcgccgcccagccgtagtcacctggattacctacagcggcagctgcagcgg
agccagcgagaaggccaaaggggagcagcgtcccgagaggagcgcctcttttcagggaccccgccggctggcggacgcgcgggaaagcggc
gtcgcgaacagagccagattgagggcccgcgggtggagagagcgacgcccgaggggatggcggcagcgtcccggagcgcctctggctgggc
gctactgctgctggtggcactttggcagcagcgcgcggccggctccggcgtcttccagctgcagctgcaggagttcatcaacgagcgcggc
gtactggccagtgggcggccttgcgagcccggctgccggactttcttccgcgtctgccttaagcacttccaggcggtcgtctcgcccggac
cctgcaccttcgggaccgtctccacgccggtattgggcaccaactccttcgctgtccgggacgacagtagcggcggggggcgcaaccctct
ccaactgcccttcaatttcacctggccgggtaccttctcgctcatcatcgaagcttggcacgcgccaggagacgacctgcggccagaggcc
ttgccaccagatgcactcatcagcaagatcgccatccagggctccctagctgtgggtcagaactggttattggatgagcaaaccagcaccc
tcacaaggctgcgctactcttaccgggtcatctgcagtgacaactactatggagacaactgctcccgcctgtgcaagaagcgcaatgacca
cttcggccactatgtgtgccagccagatggcaacttgtcctgcctgcccggttggactggggaatattgccaacagcctatctgtctttcg
ggctgtcatgaacagaatggctactgcagcaagccagcagagtgcctctgccgcccaggctggcagggccggctgtgtaacgaatgcatcc
cccacaatggctgtcgccacggcacctgcagcactccctggcaatgtacttgtgatgagggctggggaggcctgttttgtgaccaagatct
caactactgcacccaccactccccatgcaagaatggggcaacgtgctccaacagtgggcagcgaagctacacctgcacctgtcgcccaggc
tacactggtgtggactgtgagctggagctcagcgagtgtgacagcaacccctgtcgcaatggaggcagctgtaaggaccaggaggatggct
accactgcctgtgtcctccgggctactatggcctgcattgtgaacacagcaccttgagctgcgccgactccccctgcttcaatgggggctc
ctgccgggagcgcaaccagggggccaactatgcttgtgaatgtccccccaacttcaccggctccaactgcgagaagaaagtggacaggtgc
accagcaacccctgtgccaacgggggacagtgcctgaaccgaggtccaagccgcatgtgccgctgccgtcctggattcacgggcacctact
gtgaactccacgtcagcgactgtgcccgtaacccttgcgcccacggtggcacttgccatgacctggagaatgggctcatgtgcacctgccc
tgccggcttctctggccgacgctgtgaggtgcggacatccatcgatgcctgtgcctcgagtccctgcttcaacagggccacctgctacacc
gacctctccacagacacctttgtgtgcaactgcccttatggctttgtgggcagccgctgcgagttccccgtgggcttgccgcccagcttcc
cctgggtggccgtctcgctgggtgtggggctggcagtgctgctggtactgctgggcatggtggcagtggctgtgcggcagctgcggcttcg
acggccggacgacggcagcagggaagccatgaacaacttgtcggacttccagaaggacaacctgattcctgccgcccagcttaaaaacaca
aaccagaagaaggagctggaagtggactgtggcctggacaagtccaactgtggcaaacagcaaaaccacacattggactataatctggccc
cagggcccctgggggggggaccatgccaggaaagtttccccacagtgacaagagcttaggagagaaggcgccactgcggttacacagtgaa
aagccagagtgtcggatatcagcgatatgctcccccagggactccatgtaccagtctgtgtgtttgatatcagaggagaggaatgaatgtg
tcattgccacggaggtataaggcaggagcctacctggacatccctgctcagccccgcggctggaccttccttctgcattgtttacattgca
tcctggatgggacgtttttcatatgcaacgtgctgctctcaggaggaggagggaatggcaggaaccggacagactgtgaacttgccaagag
atgcaatacccttccacacctttgggtgtctgtctggcatcagattggcagctgcaccaaccagaggaacagaagagaagagagatgccac
tgggcactgccctgccagtagtggccttcagggggctccttccggggctccggcctgttttccagagagagtggcagtagccccatggggc
ccggagctgctgtggcctccactggcatccgtgtttccaaaagtgcctttggcccaggctccacggcgacagttgggcccaaatcagaaag
gagagagggggccaatgagggcagggcctcctgtgggctggaaaaccactgggtgcgtctcttgctggggtttgccctggaggtgaggtga
gtgctcgagggaggggagtgctttctgccccatgcctccaactactgtatgcaggcctggctctctggtctaggccctttgggcaagaatg
tccgtctacccggcttccaccaccctctggccctgggcttctgtaagcagacaggcagagggcctgcccctcccaccagccaagggtgcca
ggcctaactggggcactcagggcagtgtgttggaaattccactgagggggaaatcaggtgctgcggccgcctgggccctttcctccctcaa
gcccatctccacaacctcgagcctgggctctggtccactactgccccagaccaccctcaaagctggtcttcagaaatcaataatatgagtt
tttattttgtttttttttttttttttgtagtttattttggagtctagtatttcaataatttaagaatcagaagcactgacctttctacatt
ttataacattattttgtatataatgtgtatttataatatgaaacagatgtgtacagga
SEQ ID NO: 8, Homo sapiens delta like canonical Notch ligand 4 (DLL4), CDS mRNA,
NCBI Reference Sequence: NM_019074.4, 2058 bp
(SEQ ID NO: 8)
atggcggcagcgtcccggagcgcctctggctgggcgctactgctgctggtggcactttggcagcagcgcgcggccggctccggcgtcttcc
agctgcagctgcaggagttcatcaacgagcgcggcgtactggccagtgggcggccttgcgagcccggctgccggactttcttccgcgtctg
ccttaagcacttccaggcggtcgtctcgcccggaccctgcaccttcgggaccgtctccacgccggtattgggcaccaactccttcgctgtc
cgggacgacagtagcggggggggcgcaaccctctccaactgcccttcaatttcacctggccgggtaccttctcgctcatcatcgaagcttg
gcacgcgccaggagacgacctgcggccagaggccttgccaccagatgcactcatcagcaagatcgccatccagggctccctagctgtgggt
cagaactggttattggatgagcaaaccagcaccctcacaaggctgcgctactcttaccgggtcatctgcagtgacaactactatggagaca
actgctcccgcctgtgcaagaagcgcaatgaccacttcggccactatgtgtgccagccagatggcaacttgtcctgcctgcccggttggac
tggggaatattgccaacagcctatctgtctttcgggctgtcatgaacagaatggctactgcagcaagccagcagagtgcctctgccgccca
ggctggcagggccggctgtgtaacgaatgcatcccccacaatggctgtcgccacggcacctgcagcactccctggcaatgtacttgtgatg
agggctggggaggcctgttttgtgaccaagatctcaactactgcacccaccactccccatgcaagaatggggcaacgtgctccaacagtgg
gcagcgaagctacacctgcacctgtcgcccaggctacactggtgtggactgtgagctggagctcagcgagtgtgacagcaacccctgtcgc
aatggaggcagctgtaaggaccaggaggatggctaccactgcctgtgtcctccgggctactatggcctgcattgtgaacacagcaccttga
gctgcgccgactccccctgcttcaatgggggctcctgccgggagcgcaaccagggggccaactatgcttgtgaatgtccccccaacttcac
cggctccaactgcgagaagaaagtggacaggtgcaccagcaacccctgtgccaacgggggacagtgcctgaaccgaggtccaagccgcatg
tgccgctgccgtcctggattcacgggcacctactgtgaactccacgtcagcgactgtgcccgtaacccttgcgcccacggtggcacttgcc
atgacctggagaatgggctcatgtgcacctgccctgccggcttctctggccgacgctgtgaggtgcggacatccatcgatgcctgtgcctc
gagtccctgcttcaacagggccacctgctacaccgacctctccacagacacctttgtgtgcaactgcccttatggctttgtgggcagccgc
tgcgagttccccgtgggcttgccgcccagcttcccctgggtggccgtctcgctgggtgtggggctggcagtgctgctggtactgctgggca
tggtggcagtggctgtgcggcagctgcggcttcgacggccggacgacggcagcagggaagccatgaacaacttgtcggacttccagaagga
caacctgattcctgccgcccagcttaaaaacacaaaccagaagaaggagctggaagtggactgtggcctggacaagtccaactgtggcaaa
cagcaaaaccacacattggactataatctggccccagggcccctgggggggggaccatgccaggaaagtttccccacagtgacaagagctt
aggagagaaggcgccactgcggttacacagtgaaaagccagagtgtcggatatcagcgatatgctcccccagggactccatgtaccagtct
gtgtgtttgatatcagaggagaggaatgaatgtgtcattgccacggaggtataa

In some embodiments, the amino acid sequence of the Notch ligand (e.g., DLL4) comprises SEQ ID NO: 9 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 9, and that maintains the same functions as SEQ ID NO: 9 (e.g., binding and/or activating a Notch receptor).

SEQ ID NO: 9, delta-like protein 4 precursor
[Homo sapiens], NCBI Reference Sequence:
NP_061947.1, 685 amino acids
(SEQ ID NO: 9)
MAAASRSASGWALLLLVALWQQRAAGSGVFQLQLQEFINERGVLASGRP
CEPGCRTFFRVCLKHFQAVVSPGPCTFGTVSTPVLGTNSFAVRDDSSGG
GRNPLQLPFNFTWPGTFSLIIEAWHAPGDDLRPEALPPDALISKIAIQG
SLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGW
QGRLCNECIPHNGCRHGTCSTPWQCTCDEGWGGLFCDQDLNYCTHHSPC
KNGATCSNSGQRSYTCTCRPGYTGVDCELELSECDSNPCRNGGSCKDQE
DGYHCLCPPGYYGLHCEHSTLSCADSPCFNGGSCRERNQGANYACECPP
NFTGSNCEKKVDRCTSNPCANGGQCLNRGPSRMCRCRPGFTGTYCELHV
SDCARNPCAHGGTCHDLENGLMCTCPAGFSGRRCEVRTSIDACASSPCF
NRATCYTDLSTDTFVCNCPYGFVGSRCEFPVGLPPSFPWVAVSLGVGLA
VLLVLLGMVAVAVRQLRLRRPDDGSREAMNNLSDFQKDNLIPAAQLKNT
NQKKELEVDCGLDKSNCGKQQNHTLDYNLAPGPLGRGTMPGKFPHSDKS
LGEKAPLRLHSEKPECRISAICSPRDSMYQSVCLISEERNECVIATEV

In some embodiments, the Notch ligand comprises the extracellular domain of human DLL4, which corresponds to amino acids 1-526 of DLL4, or amino acids 1-524 of DLL4, or amino acids 27-524 of DLL4, (see, e.g., SEQ ID NO: 9 for full-length sequence of DLL4). In some embodiments, the extracellular domain of human DLL4 comprises SEQ ID NO: 10 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 5, and that maintains the same functions as SEQ ID NO: 10 (e.g., binding and/or activating a Notch receptor).

SEQ ID NO: 10, human DLL4 extracellular domain,
526 amino acids
(SEQ ID NO: 10)
MAAASRSASGWALLLLVALWQQRAAGSGVFQLQLQEFINERGVLASGRP
CEPGCRTFFRVCLKHFQAVVSPGPCTFGTVSTPVLGTNSFAVRDDSSGG
GRNPLQLPFNFTWPGTFSLIIEAWHAPGDDLRPEALPPDALISKIAIQG
SLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGW
QGRLCNECIPHNGCRHGTCSTPWQCTCDEGWGGLFCDQDLNYCTHHSPC
KNGATCSNSGQRSYTCTCRPGYTGVDCELELSECDSNPCRNGGSCKDQE
DGYHCLCPPGYYGLHCEHSTLSCADSPCFNGGSCRERNQGANYACECPP
NFTGSNCEKKVDRCTSNPCANGGQCLNRGPSRMCRCRPGFTGTYCELHV
SDCARNPCAHGGTCHDLENGLMCTCPAGFSGRRCEVRTSIDACASSPCF
NRATCYTDLSTDTFVCNCPYGFVGSRCEFPVGLPPS

In some embodiments, the Notch ligand (e.g., Delta1ext-IgG) comprises SEQ ID NO: 42 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 42, and that maintains the same functions as SEQ ID NO: 42 (e.g., binding and/or activating a Notch receptor).

SEQ ID NO: 42, Recombinant Human DLL1 Fc Chimera
Protein, R&D SYSTEMS 10184-DL: Human DLL1
(Ser22-Glu537) Accession #O00548 + IEGRMDP
(SEQ ID NO: 49) + Human IgG1 Fc (Pro100-Lys330)
(SEQ ID NO: 42)
SGVFELKLQEFVNKKGLLGNRNCCRGGAGPPPCACRTFFRVCLKHYQAS
VSPEPPCTYGSAVTPVLGVDSFSLPDGGGADSAFSNPIRFPFGFTWPGT
FSLIIEALHTDSPDDLATENPERLISRLATQRHLTVGEEWSQDLHSSGR
TDLKYSYRFVCDEHYYGEGCSVFCRPRDDAFGHFTCGERGEKVCNPGWK
GPYCTEPICLPGCDEQHGFCDKPGECKCRVGWQGRYCDECIRYPGCLHG
TCQQPWQCNCQEGWGGLFCNQDLNYCTHHKPCKNGATCTNTGQGSYTCS
CRPGYTGATCELGIDECDPSPCKNGGSCTDLENSYSCTCPPGFYGKICE
LSAMTCADGPCFNGGRCSDSPDGGYSCRCPVGYSGFNCEKKIDYCSSSP
CSNGAKCVDLGDAYLCRCQAGFSGRHCDDNVDDCASSPCANGGTCRDGV
NDFSCTCPPGYTGRNCSAPVSRCEHAPCHNGATCHERGHRYVCECARGY
GGPNCQFLLPELPPGPAVVDLTEKLEIEGRMDPPKSCDKTHTCPPCPAP
ELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVM
HEALHNHYTQKSLSLSPGK

In some embodiments, the Notch ligand (e.g., Delta4ext-IgG) comprises SEQ ID NO: 43 or an amino acid sequence that is at least 85%, at least 87%, at least 90%, at least 91%, at least 92%, at least 93%, at least 94%, at least 95%, at least 96%, at least 97%, at least 98%, or at least 99%) identical to the sequence of SEQ ID NO: 43, and that maintains the same functions as SEQ ID NO: 43 (e.g., binding and/or activating a Notch receptor).

SEQ ID NO: 43, Human DLL4 Protein Fc Tag, ACRO
BIOSYSTEMS DLL4-H5259: Human DLL4 (Ser27-Pro524) +
Human IgG1 Fc (Pro100-Lys330)
(SEQ ID NO: 43)
SGVFQLQLQEFINERGVLASGRPCEPGCRTFFRVCLKHFQAVVSPGPCTF
GTVSTPVLGTNSFAVRDDSSGGGRNPLQLPFNFTWPGTFSLIIEAWHAPG
DDLRPEALPPDALISKIAIQGSLAVGQNWLLDEQTSTLTRLRYSYRVICS
DNYYGDNCSRLCKKRNDHFGHYVCQPDGNLSCLPGWTGEYCQQPICLSGC
HEQNGYCSKPAECLCRPGWQGRLCNECIPHNGCRHGTCSTPWQCTCDEGW
GGLFCDQDLNYCTHHSPCKNGATCSNSGQRSYTCTCRPGYTGVDCELELS
ECDSNPCRNGGSCKDQEDGYHCLCPPGYYGLHCEHSTLSCADSPCFNGGS
CRERNQGANYACECPPNFTGSNCEKKVDRCTSNPCANGGQCLNRGPSRMC
RCRPGFTGTYCELHVSDCARNPCAHGGTCHDLENGLMCTCPAGFSGRRCE
VRTSIDACASSPCFNRATCYTDLSTDTFVCNCPYGFVGSRCEFPVGLPPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

In some embodiments, the Notch ligand comprises an extracellular domain of a Notch ligand as described herein linked (e.g., through an optional linker sequence) to the Fc domain of human IgG1. In some embodiments, the human IgG1 Fc domain comprises SEQ ID NO: 44 or an amino acid sequence that is at least 85%, at least 87%, at least 90%, at least 91%, at least 92%, at least 93%, at least 94%, at least 95%, at least 96%, at least 97%, at least 98%, or at least 99% identical to the sequence of SEQ ID NO: 44, and that maintains the same functions as SEQ ID NO: 44.

SEQ ID NO: 44, Pro 100-Lys330 of P01857
(IGHG1_HUMAN)
(SEQ ID NO: 44)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK

There are several ways to provide a Notch ligand, for example by providing a purified recombinant form of a Notch ligand or a Notch receptor-binding fragment, the receptor-binding fragment being sufficient to elicit cell signaling events in vivo upon contact and binding with the extracellular Notch receptors on these cells. In some embodiments, the Notch ligand is attached to a solid substrate, for example using a covalent or non-covalent bond or linkage. In some embodiments, the Notch ligand is attached to a cell culture dish.

In some embodiments, the Notch ligand further comprises a domain to immobilize the Notch ligand to a solid substrate. As a non-limiting example, the Notch ligand comprises a first member of an affinity pair, and the solid substrate comprises a second member of an affinity pair. In some embodiments, the first and second members of the affinity pair are selected from the group consisting of: a haptenic or antigenic compound in combination with a corresponding antibody or binding portion or fragment thereof (e.g., FLAG and anti-FLAG monoclonal antibody, the sequence of which are known in the art); digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof; IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

In some embodiments, the population of hemogenic endothelium is differentiated into a population of CD56+NK cells by culturing in a non-tissue culture treated culture vessel; said another way, the culture vessel is not exposed to a plasma gas in order to modify the hydrophobic plastic surface to make it more hydrophilic. As used herein, the term “culture vessel” includes dishes, flasks, plates, multi-well plates, and the like. In some embodiments, the culture vessel is coated with recombinant human DLL1-Fc protein (e.g., commercially available via R&D SYSTEMS, item number 10184-DL), recombinant human DLL4-Fc protein (e.g., commercially available via ACRO BIOSYSTEMS, item number DL4-H5259), or a mixture of both Notch ligands, or any Notch ligand as described herein. In some embodiments, the culture vessel is coated with Notch ligand for at least 0.5 hour, at least 1.0 hour, at least 1.5 hours, at least 2.0 hours, at least 2.5 hours, at least 3.0 hours, at least 3.5 hours, at least 4.0 hours, at least 4.5 hours, or at least 5.0 hours. In some embodiments, the culture vessel is coated with Notch ligand at room temperature.

In some embodiments, the non-stromal-derived Notch ligand (e.g., the Notch ligand immobilized on a tissue culture plate) is provided at a concentration of 1 μg/mL to 100 μg/mL or a concentration of 5 μg/mL to 15 μg/mL. In some embodiments, the non-stromal-derived Notch ligand is provided at a concentration of at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 11 μg/mL, at least 12 μg/mL, at least 13 μg/mL, at least 14 μg/mL, at least 15 μg/mL, at least 16 μg/mL, at least 17 μg/mL, at least 18 μg/mL, at least 19 μg/mL, at least 20 μg/mL, at least 25 μg/mL, at least 30 μg/mL, at least 35 μg/mL, at least 40 μg/mL, at least 45 μg/mL, at least 50 μg/mL, at least 55 μg/mL, at least 60 μg/mL, at least 65 μg/mL, at least 70 μg/mL, at least 75 μg/mL, at least 80 μg/mL, at least 85 μg/mL, at least 90 μg/mL, at least 95 μg/mL, or at least 100 μg/mL. In a preferred embodiment, the non-stromal-derived Notch ligand is provided at a concentration of at most 5 μg/mL. In a preferred embodiment, the non-stromal-derived Notch ligand is not provided at a concentration of 10 μg/mL.

In some embodiments, the cells are cultured exposed to a non-stromal-derived Notch ligand (e.g., a Notch ligand immobilized on a tissue culture plate) for at least 1 day, at least 2 days, at least 3 days, at least 4 days, at least 5 days, at least 6 days, at least 7 days, at least 8 days, at least 9 days, at least 10 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, or more. In some embodiments, the cells are cultured exposed to a non-stromal-derived Notch ligand for at least 4 weeks.

### Stroma Free Differentiation

The method described herein is a stroma-free NK cell differentiation method, i.e., a method that does not comprise co-culturing with stromal cells or any other type of supporting cell. Co-culture with stromal cells such as mouse stromal cells limits the translational potential of iPSC-derived NK cells; for example, there can be fears of transplantation rejection due to the presence of stromal cells. Additionally, as described herein, stroma-free NK cell differentiation methods result in increased numbers or percentages of CD56+NK cells and decreased numbers or percentages of CD3+ T cells compared to differentiation methods comprising stromal co-culture.

Accordingly, NK cells differentiated using stromal-free methods, and in one embodiment, in combination with inhibition of an epigenetic regulator (e.g., an HMT; e.g., EZH1, G9a/GLP), exhibit at least one of the following unexpected benefits compared to stromal co-culture methods: (1) increased potential for transplantation in humans; (2) increased number and/or percentage of resultant CD56+NK cells; (3) decreased number or percentage of total CD3+ T cells; (4) increased expression of NK cell receptors; or (5) increased expression of genes that are responsible for lymphoid differentiation/function (see, e.g., Example 1, FIG. 2, FIG. 3A-3B, Example 3, FIG. 4, FIGS. 5A-5C, FIGS. 6A-6C).

As used herein, the term “supporting cell or stromal cell” when used in the context of cell differentiation refers to any cells that are capable of creating, promoting, or supporting a microenvironment for the growth, proliferation, differentiation, or expansion of multipotent hematopoietic progenitor cells or NK cells. Non-limiting examples of supporting cells that are not comprised by the differentiation methods described herein include, but are not limited to, stromal cells and fibroblast cells.

Supporting cells used previously in co-cultures for cell differentiation purposes are typically stromal cells. However, the methods described herein do not comprise co-cultures comprising stromal cells. Examples of stromal cell lines that are not comprised by the differentiation methods described herein include, but are not limited to, murine MS5 stromal cell line; murine bone marrow-derived stromal cell lines, such as S10, S17, OP9 (e.g., OP9-DLL1 cells or OP9-DLL4 cells) and BMS2 cell lines; human marrow stromal cell lines such as those described in U.S. Pat. No. 5,879,940, which is incorporated herein by reference in its entirety; or any other similar cells that express and display extracellular or secretes a Notch ligand. OP9-DLL1 cells are a bone-marrow-derived stromal cell line that ectopically expresses the Notch ligand, Delta-like 1 (DLL1). Method of differentiating pluripotent stem cells to NK-cells using OP9-Notch ligand expressing cells are known in the art. See, e.g., U.S. Pat. Nos. 7,575,925, 8,772,028, 8,871,510, and 9,206,394 and US Patent Publication Nos: 20090217403, 20110123502, 20110052554 20110027881, 20110236363, 20120149100, 20130281304, 20140322808, 20140248248, and 20140037599; Beck et al. The Notch ligands Jagged2, Delta1, and Delta4 induce differentiation and expansion of functional human NK cells from CD34+ cord blood hematopoietic progenitor cells, Biol Blood Marrow Transplant. 2009 September; 15(9): 1026-1037. These references are incorporated herein by reference in their entirety.

Described herein are methods of differentiating NK cells from pluripotent stem cells, wherein the methods do not comprise a step of co-culturing the cells with supporting cells or stromal cells. In some embodiments, the Notch ligand used herein is not derived from a stromal cell. In some embodiments, differentiating the hemogenic endothelium in the presence of a Notch ligand does not comprise co-culturing with a stromal cell expressing a Notch ligand. In some embodiments, differentiating the hemogenic endothelium in the presence of a Notch ligand does not comprise co-culturing with OP9-DLL1 cells or OP9-DLL4 cells.

NK-Cell-Differentiation Media

In some embodiments, the differentiation method comprises differentiating the resultant population of CD34⁺ hemogenic endothelium in NK-cell-differentiation media for a sufficient time to promote differentiation into a population of CD56+NK cells. In some embodiments, the differentiation method comprises differentiating the resultant population of CD34+ hemogenic endothelium in a single type of NK-cell-differentiation medium for a sufficient time to promote differentiation into a population of CD56+NK cells. In some embodiments, the differentiation method comprises differentiating the resultant population of CD34+ hemogenic endothelium in multiple types of NK-cell-differentiation media, e.g., by altering the components of the medium over time, for a sufficient time to promote differentiation into a population of CD56+NK cells (see, e.g., Table 1).

In some embodiments, the NK-cell-differentiation media comprises Serum-Free Expansion Medium II (SFEM II). In some embodiments, the NK-cell-differentiation media (e.g., SFEM II) comprises: Iscove's Modified Dulbecco's Medium (IMDM); bovine serum albumin; recombinant human insulin; human transferrin (iron-saturated); and/or 2-mercaptoethanol. In some embodiments, the NK-cell-differentiation media comprises alpha Minimum Essential Medium (MEM). In some embodiments, the NK-cell-differentiation media (e.g., alpha MEM) comprises non-essential amino acids, sodium pyruvate, lipoic acid, vitamin B12, biotin, ascorbic acid, ribonucleosides, deoxyribonucleosides, phenol red, and/or L-glutamine. In some embodiments, the sufficient time to promote differentiation into a population of CD56+NK cells is at least 3 weeks, at least 3.5 weeks, at least 4 weeks, at least 4.5 weeks, at least 5 weeks, at least 5.5 weeks, at least 6 weeks, or more. In some embodiments, the sufficient time to promote differentiation into a population of CD56+NK cells is at most 6 weeks. In some embodiments, the sufficient time to promote differentiation into a population of CD56+NK cells is at most 4 weeks.

In some embodiments, a polypeptide (e.g., growth or differentiation factors) that can be expressed by the supporting cell or stromal cell can be provided in the cell culture medium. Non limiting examples of polypeptides that support the differentiation of NK cells that can be included in the cell culture medium include IL-7, SCF, Flt3, TPO, IL-15, and IL-3. Interleukin-7 (IL-7 or IL7) is a hematopoietic growth factor secreted by stromal cells in the bone marrow and thymus, and it is involved in NK, B and T cell development. Stem cell factor (also known as SCF, KIT-ligand, KL, or steel factor) is a cytokine that binds to the c-KIT receptor (CD117) and is involved in NK and T cell differentiation. FLT3 (also referred to as Flit3 or Fms-Like Tyrosine Kinase 3) is a class III receptor tyrosine kinase that regulates hematopoiesis. In some embodiments, the NK-cell-differentiation media can further comprise thrombopoietin (TPO or THPO), which is a cytokine that is chiefly responsible for megakaryocyte production but also has a role in maintaining hematopoietic stem cells (HSCs). In some embodiments, the NK-cell-differentiation media does not comprise TPO. See, e.g., Wang et al., Distinct roles of IL-7 and stem cell factor in the OP9-DLL1 T cell differentiation culture system. Exp Hematol. 2006 December; 34(12):1730-40. Interleukin-15 (IL-15 or IL15) induces the activation and proliferation of NK and T cells. IL-15 binds to and signals through a complex composed of IL-2/IL-15 receptor beta chain (CD122) and the common gamma chain (CD132). Interleukin-3 (IL-3 or IL3) is also referred to as colony-stimulating factor, multi-CSF, mast cell growth factor, MULTI-CSF, MCGF; MGC79398, or MGC79399. The major function of IL-3 cytokine is to regulate the concentrations of various blood-cell types. IL-3 induces proliferation and differentiation in both early pluripotent stem cells and committed progenitors.

In some embodiments, the NK-cell-differentiation media is serum-free. In some embodiments, the NK-cell-differentiation media comprises at least one of SCF, FLT3, and/or IL7. In some embodiments, the NK-cell-differentiation media comprises SCF, FLT3, and IL7. In some embodiments, the NK-cell-differentiation media comprises 50 ng/ml SCF, 50 ng/ml FLT3, and 10 ng/ml IL7. In some embodiments, the NK-cell-differentiation media comprises 100 ng/ml SCF, 100 ng/ml FLT3, and 5 ng/ml IL7. In some embodiments, the NK-cell-differentiation media comprises 30 ng/ml SCF, 15 ng/ml FLT3, and 20 ng/ml IL7. In some embodiments, the NK-cell-differentiation media comprises 30 ng/ml SCF, 20 ng/ml FLT3, and 25 ng/ml IL7. In some embodiments, the concentrations of SCF, FLT3, and IL7 are selected from Table 7 or Table 8.

TABLE 7
Exemplary SCF, FLT3, and IL7 concentrations in NK-cell-differentiation
media; each row shows an exemplary combination.

| SCF | FLT3 | IL-7 |
|---|---|---|
| (ng/mL) | (ng/mL) | (ng/mL) |
| 30 | 15 | 5 |
| 30 | 15 | 10 |
| 30 | 15 | 20 |
| 30 | 15 | 25 |
| 30 | 20 | 5 |
| 30 | 20 | 10 |
| 30 | 20 | 20 |
| 30 | 20 | 25 |
| 30 | 50 | 5 |
| 30 | 50 | 10 |
| 30 | 50 | 20 |
| 30 | 50 | 25 |
| 30 | 100 | 5 |
| 30 | 100 | 10 |
| 30 | 100 | 20 |
| 30 | 100 | 25 |
| 50 | 15 | 5 |
| 50 | 15 | 10 |
| 50 | 15 | 20 |
| 50 | 15 | 25 |
| 50 | 20 | 5 |
| 50 | 20 | 10 |
| 50 | 20 | 20 |
| 50 | 20 | 25 |
| 50 | 50 | 5 |
| 50 | 50 | 10 |
| 50 | 50 | 20 |
| 50 | 50 | 25 |
| 50 | 100 | 5 |
| 50 | 100 | 10 |
| 50 | 100 | 20 |
| 50 | 100 | 25 |
| 100 | 15 | 5 |
| 100 | 15 | 10 |
| 100 | 15 | 20 |
| 100 | 15 | 25 |
| 100 | 20 | 5 |
| 100 | 20 | 10 |
| 100 | 20 | 20 |
| 100 | 20 | 25 |
| 100 | 50 | 5 |
| 100 | 50 | 10 |
| 100 | 50 | 20 |
| 100 | 50 | 25 |
| 100 | 100 | 5 |
| 100 | 100 | 10 |
| 100 | 100 | 20 |
| 100 | 100 | 25 |

TABLE 8
Exemplary SCF, FLT3, and IL7 concentration ranges
in NK-cell-differentiation media; each row shows
an exemplary combination; each range is inclusive
of the indicated minimum and maximum values.

| SCF | FLT3 | IL-7 |
|---|---|---|
| (ng/mL) | (ng/mL) | (ng/mL) |
| 1-30 | 1-15 | 1-5 |
| 1-30 | 1-15 | 5-10 |
| 1-30 | 1-15 | 10-20 |
| 1-30 | 1-15 | 20-25 |
| 1-30 | 1-15 | 25-200 |
| 1-30 | 15-20 | 1-5 |
| 1-30 | 15-20 | 5-10 |
| 1-30 | 15-20 | 10-20 |
| 1-30 | 15-20 | 20-25 |
| 1-30 | 15-20 | 25-200 |
| 1-30 | 20-50 | 1-5 |
| 1-30 | 20-50 | 5-10 |
| 1-30 | 20-50 | 10-20 |
| 1-30 | 20-50 | 20-25 |
| 1-30 | 20-50 | 25-200 |
| 1-30 | 50-100 | 1-5 |
| 1-30 | 50-100 | 5-10 |
| 1-30 | 50-100 | 10-20 |
| 1-30 | 50-100 | 20-25 |
| 1-30 | 50-100 | 25-200 |
| 1-30 | 100-200 | 1-5 |
| 1-30 | 100-200 | 5-10 |
| 1-30 | 100-200 | 10-20 |
| 1-30 | 100-200 | 20-25 |
| 1-30 | 100-200 | 25-200 |
| 30-50 | 1-15 | 1-5 |
| 30-50 | 1-15 | 5-10 |
| 30-50 | 1-15 | 10-20 |
| 30-50 | 1-15 | 20-25 |
| 30-50 | 1-15 | 25-200 |
| 30-50 | 15-20 | 1-5 |
| 30-50 | 15-20 | 5-10 |
| 30-50 | 15-20 | 10-20 |
| 30-50 | 15-20 | 20-25 |
| 30-50 | 15-20 | 25-200 |
| 30-50 | 20-50 | 1-5 |
| 30-50 | 20-50 | 5-10 |
| 30-50 | 20-50 | 10-20 |
| 30-50 | 20-50 | 20-25 |
| 30-50 | 20-50 | 25-200 |
| 30-50 | 50-100 | 1-5 |
| 30-50 | 50-100 | 5-10 |
| 30-50 | 50-100 | 10-20 |
| 30-50 | 50-100 | 20-25 |
| 30-50 | 50-100 | 25-200 |
| 30-50 | 100-200 | 1-5 |
| 30-50 | 100-200 | 5-10 |
| 30-50 | 100-200 | 10-20 |
| 30-50 | 100-200 | 20-25 |
| 30-50 | 100-200 | 25-200 |
| 50-100 | 1-15 | 1-5 |
| 50-100 | 1-15 | 5-10 |
| 50-100 | 1-15 | 10-20 |
| 50-100 | 1-15 | 20-25 |
| 50-100 | 1-15 | 25-200 |
| 50-100 | 15-20 | 1-5 |
| 50-100 | 15-20 | 5-10 |
| 50-100 | 15-20 | 10-20 |
| 50-100 | 15-20 | 20-25 |
| 50-100 | 15-20 | 25-200 |
| 50-100 | 20-50 | 1-5 |
| 50-100 | 20-50 | 5-10 |
| 50-100 | 20-50 | 10-20 |
| 50-100 | 20-50 | 20-25 |
| 50-100 | 20-50 | 25-200 |
| 50-100 | 50-100 | 1-5 |
| 50-100 | 50-100 | 5-10 |
| 50-100 | 50-100 | 10-20 |
| 50-100 | 50-100 | 20-25 |
| 50-100 | 50-100 | 25-200 |
| 50-100 | 100-200 | 1-5 |
| 50-100 | 100-200 | 5-10 |
| 50-100 | 100-200 | 10-20 |
| 50-100 | 100-200 | 20-25 |
| 50-100 | 100-200 | 25-200 |
| 100-200 | 1-15 | 1-5 |
| 100-200 | 1-15 | 5-10 |
| 100-200 | 1-15 | 10-20 |
| 100-200 | 1-15 | 20-25 |
| 100-200 | 1-15 | 25-200 |
| 100-200 | 15-20 | 1-5 |
| 100-200 | 15-20 | 5-10 |
| 100-200 | 15-20 | 10-20 |
| 100-200 | 15-20 | 20-25 |
| 100-200 | 15-20 | 25-200 |
| 100-200 | 20-50 | 1-5 |
| 100-200 | 20-50 | 5-10 |
| 100-200 | 20-50 | 10-20 |
| 100-200 | 20-50 | 20-25 |
| 100-200 | 20-50 | 25-200 |
| 100-200 | 50-100 | 1-5 |
| 100-200 | 50-100 | 5-10 |
| 100-200 | 50-100 | 10-20 |
| 100-200 | 50-100 | 20-25 |
| 100-200 | 50-100 | 25-200 |
| 100-200 | 100-200 | 1-5 |
| 100-200 | 100-200 | 5-10 |
| 100-200 | 100-200 | 10-20 |
| 100-200 | 100-200 | 20-25 |
| 100-200 | 100-200 | 25-200 |

In some embodiments, the NK-cell-differentiation media comprises FLT3 and IL7. In some embodiments, the NK-cell-differentiation media comprises 50 ng/ml FLT3 and 10 ng/ml IL7. In some embodiments, the NK-cell-differentiation media comprises 100 ng/ml FLT3 and 5 ng/ml IL7. In some embodiments, the NK-cell-differentiation media comprises 15 ng/ml FLT3 and 20 ng/ml IL7. In some embodiments, the NK-cell-differentiation media comprises 20 ng/ml FLT3 and 25 ng/ml IL7.

The concentrations of SCF, FLT3, and/or IL7 should be used such that they promote the differentiation of hemogenic endothelium into a population of CD56⁺ NK cells. The concentration of SCF can range from 1 ng/mL to 200 ng/mL. In some embodiments, the concertation of SCF (e.g., in the NK-cell-differentiation media) is 30 ng/ml. In some embodiments, the concertation of SCF (e.g., in the NK-cell-differentiation media) is 50 ng/mL. In some embodiments, the concertation of SCF (e.g., in the NK-cell-differentiation media) is 100 ng/ml. In some embodiments, the concertation of SCF (e.g., in the NK-cell-differentiation media) ranges from 30 ng/ml to 100 ng/ml.

The concentration of FLT3 can range from 1 ng/mL to 200 ng/mL. In some embodiments, the concertation of FLT3 (e.g., in the NK-cell-differentiation media) is 15 ng/ml. In some embodiments, the concertation of FLT3 (e.g., in the NK-cell-differentiation media) is 20 ng/ml. In some embodiments, the concertation of FLT3 (e.g., in the NK-cell-differentiation media) is 50 ng/ml. In some embodiments, the concertation of FLT3 (e.g., in the NK-cell-differentiation media) is 100 ng/ml. In some embodiments, the concertation of FLT3 (e.g., in the NK-cell-differentiation media) ranges from 15 ng/ml to 100 ng/ml.

The concentration of IL7 can range from 1 ng/mL to 200 ng/mL. In some embodiments, the concertation of IL7 (e.g., in the NK-cell-differentiation media) is 5 ng/ml. In some embodiments, the concertation of IL7 (e.g., in the NK-cell-differentiation media) is 10 ng/ml. In some embodiments, the concertation of IL7 (e.g., in the NK-cell-differentiation media) is 20 ng/ml. In some embodiments, the concertation of IL7 (e.g., in the NK-cell-differentiation media) ranges from 5 ng/ml to 20 ng/ml. In some embodiments, the concertation of IL7 (e.g., in the NK-cell-differentiation media) is 25 ng/ml. In some embodiments, the concertation of IL7 (e.g., in the NK-cell-differentiation media) ranges from 5 ng/ml to 25 ng/ml.

In some embodiments, the NK-cell-differentiation media further comprises thrombopoietin (TPO), e.g., for at least the first 2 weeks of differentiating in the NK-cell-differentiation media. As a non-limiting example, the NK-cell-differentiation media further comprises thrombopoietin (TPO) for at least the first 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of differentiating in the NK-cell-differentiation media.

In some embodiments, the concentration of TPO should be used such that it promotes the differentiation of hemogenic endothelium into a population of CD56+NK cells. In some embodiments, the concentration of TPO can range from 1 ng/mL to 200 ng/mL. In some embodiments, the concertation of TPO (e.g., in the NK-cell-differentiation media, for at least the first 2 weeks) is 5 ng/mL. In some embodiments, the NK-cell-differentiation media does not comprise or is substantially free of TPO.

In some embodiments, the NK-cell-differentiation media (e.g., comprising IL-7 and/or FLT3) further comprises SCF for at least the first 2 weeks of differentiating in the NK-cell-differentiation media. As a non-limiting example, the NK-cell-differentiation media further comprises SCF for at least the first 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of differentiating in the NK-cell-differentiation media.

In some embodiments, the NK-cell-differentiation media further comprises IL-15, e.g., starting after at least the first 2 weeks of differentiating in the NK-cell-differentiation media. As a non-limiting example, the NK-cell-differentiation media further comprises IL-15 starting after at least the first 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of differentiating in the NK-cell-differentiation media. In some embodiments, NK-cell-differentiation media (e.g., starting after at least the first 2 weeks of differentiating in the NK-cell-differentiation media) comprises IL-15 for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, NK-cell-differentiation media comprises IL-15 for week 2 to week 4 of differentiation in the NK-cell-differentiation media. In some embodiments, NK-cell-differentiation media comprises IL-15 for week 2 to week 5 of differentiation in the NK-cell-differentiation media.

In some embodiments, the concentration of IL-15 should be used such that it promotes the differentiation of hemogenic endothelium into a population of CD56+NK cells. In some embodiments, the concentration of IL-15 ranges from 1 ng/mL to 200 ng/mL. In some embodiments, the concertation of IL-15 (e.g., in the NK-cell-differentiation media, starting after at least the first 2 weeks) is 10 ng/mL.

In some embodiments, the NK-cell-differentiation media further comprises IL-3, e.g., for at least the first week of differentiating in the NK-cell-differentiation media. As a non-limiting example, the NK-cell-differentiation media further comprises IL-3 for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, NK-cell-differentiation media comprises IL-3 for week 1 to week 2, or week 1 to week 3, or week 1 to week 4 of differentiation in the NK-cell-differentiation media. In some embodiments, the concentration of IL-3 should be used such that it promotes the differentiation of hemogenic endothelium into a population of CD56+NK cells. In some embodiments, the concentration of IL-3 ranges from 1 ng/mL to 200 ng/mL. In some embodiments, the concertation of IL-3 (e.g., in the NK-cell-differentiation media, for at least the first one) is 5 ng/mL.

In some embodiments, SCF, FLT3, IL7, TPO, IL-15, and/or IL-3 are provided in the NK-cell-differentiation media at a concentration of at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 16 ng/mL, at least 17 ng/mL, at least 18 ng/mL, at least 19 ng/mL, at least 20 ng/mL, at least 25 ng/mL, at least 30 ng/mL, at least 35 ng/mL, at least 40 ng/mL, at least 45 ng/mL, at least 50 ng/mL, at least 55 ng/mL, at least 60 ng/mL, at least 65 ng/mL, at least 70 ng/mL, at least 75 ng/mL, at least 80 ng/mL, at least 85 ng/mL, at least 90 ng/mL, at least 95 ng/mL, at least 100 ng/mL, at least 105 ng/mL, at least 110 ng/mL, at least 115 ng/mL, at least 120 ng/mL, at least 125 ng/mL, at least 130 ng/mL, at least 135 ng/mL, at least 140 ng/mL, at least 145 ng/mL, at least 150 ng/mL, at least 155 ng/mL, at least 160 ng/mL, at least 165 ng/mL, at least 170 ng/mL, at least 175 ng/mL, at least 180 ng/mL, at least 185 ng/mL, at least 190 ng/mL, at least 195 ng/mL, or at least 200 ng/mL. The concentration of SCF, FLT3, IL7, TPO, IL-15, and/or IL-3 can be the same or different.

In some embodiments, CD56+NK cells can be detected after at least 5.0 weeks of differentiating in the NK-cell-differentiation media. In some embodiments, CD56+NK cells can be detected after at least 1.5 weeks, 2 weeks, 2.5 weeks, 3.0 weeks, 3.5 weeks, 4.0 weeks, 4.5 weeks, or 5.0 weeks of differentiating in the NK-cell-differentiation media.

In some embodiments, the method further comprises, after at least 1 week (e.g., in the NK-cell-differentiation media), a step of CD56+NK cell enrichment. In some embodiments, a step of CD56+NK cell enrichment can occur at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 1.5 weeks, at least 2 weeks, at least 2.5 weeks, at least 3 weeks, at least 3.5 weeks, at least 4 weeks, at least 4.5 weeks, or at least 5 weeks of culturing in the NK-cell-differentiation media.

Methods of enriching for CD56+NK cells are known in the art. As non-limiting examples, the CD56+NK cells can be enriched using magnetic-activated cell sorting (MACS) and fluorescence-activated cell sorting (FACS) with anti-CD56 antibodies. Additional non-limiting examples of NK markers that can be used to detect or enrich for NK (e.g., human) cells include: CD3−; CD94+, Fc gamma RIII/CD16+; CD57; NK1.1+; NK1.2+; CD122/IL-2 beta+; CD217/IL-7R alpha−; KIR family receptors+; NKG2A+; NKG2D+; NKp30+; NKp44+; NKp46+; or NKp80+.

In some embodiments, the entire NK cell differentiation protocol described herein occurs in a stromal-free environment, e.g., the cells are cultured exposed to a non-stromal-derived Notch ligand (e.g., Notch ligand immobilized on a tissue culture plate). In some embodiments, at least a portion of the NK cell differentiation protocol (e.g., comprising culturing in the NK-cell-differentiation media) occurs in a stromal-free environment, e.g., the cells are cultured exposed to a non-stromal-derived Notch ligand (e.g., Notch ligand immobilized on a tissue culture plate).

Derived NK Cell Population

As described herein, the population of NK cells derived using stromal-free methods as described herein, and in one embodiment, in combination with inhibition of an epigenetic regulator (e.g., an HMT; e.g., EZH1, G9a/GLP), exhibits at least one of the following unexpected benefits compared to stromal co-culture methods: (1) increased potential for transplantation in humans; (2) increased number and/or percentage of resultant NK cells; (3) decreased number or percentage of total CD3+ T cells; (4) increased expression of NK cell receptors; or (5) increased expression of genes that are responsible for lymphoid differentiation/function (see, e.g., Example 1, FIG. 2, FIGS. 3A-3B, Example 3, FIG. 4, FIGS. 5A-5C, FIGS. 6A-6C).

In some embodiments, the population of NK cells (e.g., CD56+NK cells) derived using stromal-free methods and/or inhibition of an epigenetic regulator (e.g., an HMT; e.g., EZH1, G9a/GLP) as described herein exhibits at least a 10% higher transplantation or engraftment rate than a population of NK cells derived using a stromal method. In some embodiments, the population of NK cells derived using stromal-free methods and/or inhibition of an epigenetic regulator as described herein exhibits at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% or more, or at least 10×, 20×, 30×, 40×, 50×, 60×, 70×, 80×, 90×, 100×, 500×, 1,000×, or more higher transplantation or engraftment rate than a population of NK cells derived using a stromal method or without inhibition of an epigenetic regulator.

In some embodiments, the population of NK cells (e.g., CD56⁺ NK cells) derived using stromal-free methods and/or inhibition of an epigenetic regulator as described herein comprises at least 10% more NK cells than a population of NK cells derived using a stromal method or without inhibition of an epigenetic regulator. In some embodiments, the population of NK cells derived using stromal-free methods and/or inhibition of an epigenetic regulator as described herein comprises at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% or more, or at least 10×, 20×, 30×, 40×, 50×, 60×, 70×, 80×, 90×, 100×, 500×, 1,000×, or more NK cells than a population of NK cells derived using a stromal method or without inhibition of an epigenetic regulator.

In some embodiments, the population of cells derived using stromal-free methods and/or inhibition of an epigenetic regulator as described herein comprises at least 10% fewer T cells (e.g., CD3+ T cells) than a population of cells derived using a stromal method or without inhibition of an epigenetic regulator. In some embodiments, the population of cells derived using stromal-free methods and/or inhibition of an epigenetic regulator as described herein comprises at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% or more, or at least 10×, 20×, 30×, 40×, 50×, 60×, 70×, 80×, 90×, 100×, 500×, 1,000×, or more, fewer T cells (e.g., CD3+ T cells) than a population of cells derived using a stromal method or without inhibition of an epigenetic regulator.

In some embodiments, the population of cells derived using stromal-free methods and/or inhibition of an epigenetic regulator as described herein comprises at least 40% NK cells (e.g., CD56+NK cells). In some embodiments, the population of cells derived using stromal-free methods and/or inhibition of an epigenetic regulator as described herein comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more NK cells (e.g., CD56+NK cells).

In some embodiments, the population of cells derived using stromal-free methods and/or inhibition of an epigenetic regulator as described herein comprises at most 0.2% T cells (e.g., CD3⁺ T cells). In some embodiments, the population of cells derived using stromal-free methods and/or inhibition of an epigenetic regulator as described herein comprises at most 0.1%, at most 0.2%, at most 0.3%, at most 0.4%, at most 0.5%, at most 0.6%, at most 0.7%, at most 0.8%, at most 0.9%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, or at most 60% T cells (e.g., CD3+ T cells).

In some embodiments, the population of cells derived using stromal-free methods and/or inhibition of an epigenetic regulator as described herein comprises at least 30% of the NK cells (e.g., CD56+NK cells) expressing NK cell receptors (e.g., NCR1, CD16, NKG2A; see, e.g., FIG. 4). In some embodiments, the population of cells derived using stromal-free methods and/or inhibition of an epigenetic regulator as described herein comprises at least 15%, at least 20%, at least 25%, at least 30%, at least 35%, at least 40%, at least 45%, at least 50%, or more, of the NK cells (e.g., CD56+NK cells) expressing NK cell receptors.

In some embodiments, the population of cells derived using stromal-free methods and/or inhibition of an epigenetic regulator as described herein comprises an increase of at least 135% of the NK cells (e.g., CD56+NK cells) expressing NK cell receptors (e.g., NCR1, CD16, NKG2A; see, e.g., FIG. 4) as compared to NK cell receptor expression on NK cells derived without stromal-free methods or inhibition of an epigenetic regulator. In some embodiments, the population of cells derived using stromal-free methods and/or inhibition of an epigenetic regulator as described herein comprises an increase of at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, 100%-200%, 200%-300%, or more, of the NK cells (e.g., CD56+NK cells) expressing NK cell receptors (e.g., NCR1, CD16, NKG2A; see, e.g., FIG. 4) as compared to NK cell receptor expression on NK cells derived without stromal-free methods or inhibition of an epigenetic regulator.

Genetic Modifications of NK Cells

In some embodiments, the resultant population of CD34+ hemogenic endothelium or another population as described herein (e.g., ESCs; iPSCs; HSCs; CD56+NK cells) are genetically modified. In some embodiments, a native NK cell receptor locus can be removed and/or replaced to enhance targeted specificity. Non-limiting examples of NK cell receptors include NKp46, CD16, hNKp30, hNKp44, hNKp80, mNKR-P1C, NKG2D, mNKG2D-S, hKIR-S, mAct Ly49, CD94/NKG2C, CRACC, Ly9, CD84, NTBA, 2B4, hKIR-L, hLILRB1, CD94/NKG2A, mNKR-P1B, mNKR-P1D, KLRG-1, TIGIT, CEACAM-1, or LAIR-1; see, e.g., Vivier et al. Science 331(6013):44-9 (2011), the content of which is incorporated herein by reference in its entirety. In some embodiments, a native inhibitory NK cell receptor locus can be removed or inhibited (e.g., knocked down using nucleic acid inhibitors) to enhance NK cell activity. Non-limiting examples of inhibitory NK cell receptors include hKIR-L, hLILRB1, CD94/NKG2A, mNKR-P1B, mNKR-P1D, KLRG-1, TIGIT, CEACAM-1, or LAIR-1

In some embodiments, the cells are engineered to reduce immunogenicity in the resultant population of NK cells. As used herein, the term “immunogenicity” refers to the ability of cells to provoke an immune response. In some embodiments, the resultant NK cells are hypoimmunogenic. As used herein, the term “hypoimmunogenic,” also referred to as “universal,” describes cells with reduced immunogenicity compared to unengineered or normal cells, such that the cells can evade a host's immune system following transplantation. In some embodiments, an endogenous HLA (e.g., class I and/or class II major histocompatibility complexes) can be edited or removed to reduce immunogenicity. HLA class Ia and class II mismatch results in a cytotoxic CD8+ T cell and CD4+T helper cell response, respectively; thus editing or removing class I and/or class II MHC can reduce T cell responses to the transplanted cells. In some embodiments, the genetic modification to reduce immunogenicity can comprise introduction and expression of tolerance-promoting immunomodulatory molecules, including but not limited to HLA-G, HLA-E, CD47, and/or PD-L1. In some embodiments, the genetic modification can comprise introduction and expression of non-canonical HLA-G and HLA-E to prevent NK cell-mediated lysis (see, e.g., Riolobos L et al. 2013), which can provide a source of universal NK cells for immunotherapy, e.g., cancer immune therapy. CD47 and PD-L1 are immune checkpoints, which are inhibitory immune pathways that can help maintain tolerance. In some embodiments, the genetic modification to reduce immunogenicity can comprise introduction and expression of at least one immunomodulatory molecule selected from the group consisting of: CCL21, PD-L1, FasL, SERPINB9, H2-M3, CD47, CD200 and MFGE8. For further details about generating hypoimmunogenic cells, see, e.g., Malik et al., Engineering strategies for generating hypoimmunogenic cells with high clinical and commercial value, Regenerative Medicine vol. 14, no. 11 (2019).

In some embodiments, the genetic modification comprises expressing a chimeric antigen receptor (CAR). Chimeric antigen receptors (CARS, also known as chimeric immunoreceptors, chimeric NK cell receptors or artificial NK cell receptors) are receptor proteins that have been engineered to give NK cells the new ability to target a specific protein. The receptors are chimeric because they combine both antigen-binding and NK-cell activating functions into a single receptor. Methods of engineering chimeric antigen receptor cells (e.g., CAR NK cells) are known in the art. See, e.g., U.S. Pat. Nos. 7,446,190, 8,399,645, 8,822,647, 9,212,229, 9,273,283, 9,447,194, 9,587,020, 9,932,405, U.S. Ser. No. 10/125,193, U.S. Ser. No. 10/221,245, U.S. Ser. No. 10/273,300, U.S. Ser. No. 10/287,354, U.S. Ser. No. 10/640,570; US patent publications US20160152723, US20190336533; PCT publication WO2009091826, WO2012079000, WO2014165707, WO2015164740, WO2016168595A1, WO2017040945, WO2017100428, WO2017117112, WO2017149515, WO2018067992, WO2018102787, WO2018102786, WO2018165228, WO2019084288; the contents of each of which are incorporated herein by reference in their entireties.

In some embodiments, methods of genetically modifying a cell to express a CAR can comprise but are not limited to: transfection or electroporation of a cell with a vector encoding a CAR; transduction with a viral vector (e.g., retrovirus, lentivirus) encoding a CAR; gene editing using zinc finger nucleases (ZFNs), transcription activator-like effector nucleases (TALENs), meganuclease-TALENs, or CRISPR-Cas; or any other methods known in the art of genetically modifying a cell to express a CAR.

Preferably, a population of cells at an early stage of differentiation (e.g., ESCs; PSCs; iPSCs; hemogenic endothelium; HSCs) is genetically modified with the CAR. In some embodiments, the antigen-binding region of the CAR is directed against an antigen involved in a disease or disorder, such as but not limited to cancer, autoimmune disease, or heart disease (e.g., cardiac fibrosis).

### Cancer

Described herein are methods of treating of cancer. In one aspect, the method of treating cancer comprises administering an effective amount of cells derived using stromal-free differentiation methods and/or inhibition of an epigenetic regulator (e.g., an HMT; e.g., EZH1, G9a/GLP) as described herein or population thereof, or a composition of comprising said cell or population, or a pharmaceutical composition comprising said cell or population to a recipient subject in need thereof. In some embodiments the cell is a CD56+NK cell.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments, the cancer is a primary cancer. In some embodiments, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, e.g., in the case of CAR NK therapy, the cancer is a blood cancer such as a leukemia or lymphoma.

Immunotherapy with chimeric antigen receptor (CAR) NK cells offers a promising method to improve cure rates and decrease morbidities for patients with cancer. In this regard, CD19-specific CAR NK cell therapies have achieved dramatic objective responses for a high percent of patients with CD19-positive leukemia or lymphoma. Accordingly, in some embodiments, the antigen-binding region of the CAR is directed against CD19; see, e.g., US patents U.S. Ser. No. 10/221,245, U.S. Ser. No. 10/357,514; US patent publication US20160152723; PCT publication WO2016033570; Liu et al., N Engl J Med. 2020 Feb. 6, 382(6): 545-553; the contents of each of which are incorporated herein by reference in their entireties.

Tumor antigens are proteins that are produced by tumor cells that elicit an immune response. The selection of the antigen binding domain of the invention will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, B7H3, Kit, CA-IX, CS-1, MUC1, BCMA, bcr-abl, HER2, β-human chorionic gonadotropin, alphafetoprotein (AFP), ALK, CD19, CD123, cyclin B 1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGSS, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYP1B1, BORIS, prostase, prostate-specific antigen (PSA), PAX3, PAP, NY-ESO-1, LAGE-1a, LMP2, NCAM, p53, p53 mutant, Ras mutant, gp100, prostein, OR51E2, PANX3, PSMA, PSCA, Her2/neu, hTERT, HMWMAA, HAVCR1, VEGFR2, PDGFR-beta, legumain, HPV E6, E7, survivin and telomerase, sperm protein 17, SSEA-4, tyrosinase, TARP, WT1, prostate-carcinoma tumor antigen-1 (PCTA-1), ML-IAP, MAGE, MAGE-AL MAD-CT-1, MAD-CT-2, MelanA/MART1, XAGE1, ELF2M, ERG (TMPRSS2 ETS fusion gene), NA17, neutrophil elastase, sarcoma translocation breakpoints, NY-BR-1, ephrinB2, CD20, CD22, CD24, CD30, CD33, CD38, CD44v6, CD97, CD171, CD179a, androgen receptor, insulin growth factor (IGF)-I, IGF-II, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, CD33, BCMA, GD2, CLL-1, CA-IX, MUC1, HER2, and any combination thereof; see, e.g., US Patent publications 20170209492 and 20180022795, the contents of each of which are incorporated herein by reference in their entireties.

### Cellular Replacement Therapy

In one embodiment, provided herein is a population of engineered cells produced by a method described herein, wherein the cell population is produced using a stroma-free differentiation method as described herein. In some embodiments, the cell population comprises immune cells. In some embodiments, the population of engineered cells comprises a cell differentiated using methods described herein, including but not limited to: hemogenic endothelium; HSCs; or CD56+NK cells.

In one embodiment, the population of cells further comprises a pharmaceutically acceptable carrier. These engineered cells can be culture expanded to increase the number of cells for use.

The engineered cells described herein are useful in the laboratory for biological studies. For examples, these cells can be derived from an individual having a genetic disease or defect, and used in the laboratory to study the biological aspects of the disease or defect, and to screen and test for potential remedy for that disease or defect.

Alternatively, the engineered cells described herein are useful in cellular replacement therapy and other medical treatment in subjects having the need. For example, patients who have undergone chemotherapy or irradiation or both, and manifest deficiencies in immune function and/or lymphocyte reconstitution, or in cancer immune therapy.

In various aspects and embodiments, the engineered cells described herein are administered (i.e., implanted or transplanted) to a subject in need of cellular replacement therapy.

In one aspect, provided herein is a method of cellular replacement therapy, or for the treatment of cancer, autoimmune disorders, hematological diseases, or other genetic diseases and disorders in a subject, comprising (a) providing a somatic cell from a donor subject, (b) generating hemogenic endothelium from pluripotent stem cells derived from the somatic cell as described herein; (c) optionally inhibiting a histone methyltransferase in the resultant population of hemogenic endothelium as described herein; (d) differentiating the resultant population of hemogenic endothelium in the presence of a notch ligand to promote differentiation into the lymphoid lineage (e.g., NK cells) as described herein, and (e) implanting or administering the resultant differentiated lymphoid cells (e.g., NK cells) into a recipient subject.

In another aspect, provided herein is a method of cellular replacement therapy, or for the treatment of cancer, autoimmune disorders, hematological diseases, or other genetic diseases and disorders in a subject, comprising (a) generating hemogenic endothelium from pluripotent stem cells; (b) optionally inhibiting a histone methyltransferase in the resultant population of hemogenic endothelium as described herein; (c) differentiating the resultant population of hemogenic endothelium in the presence of a notch ligand to promote differentiation into the lymphoid lineage (e.g., NK cells) as described herein, and (d) implanting or administering the resultant differentiated lymphoid cells (e.g., NK cells) into a recipient subject.

In another aspect, provided herein is a method of cellular replacement therapy, or for the treatment of cancer, autoimmune disorders, hematological diseases, or other genetic diseases and disorders in a subject, comprising (a) optionally inhibiting a histone methyltransferase in a population of hemogenic endothelium; (b) differentiating the resultant population of hemogenic endothelium in the presence of a notch ligand to promote differentiation into the lymphoid lineage (e.g., NK cells) as described herein, and (c) implanting or administering the resultant differentiated lymphoid cells (e.g., NK cells) into a recipient subject.

In one embodiment, the host subject and the recipient subject are the same individual. Alternatively, the host subject and the recipient subject are not the same individual, but are at least HLA compatible. In some embodiments, the PSC (e.g., iPSC), hemogenic endothelium, HSPC, and/or differentiated lymphoid cell (e.g., NK cell) is hypoimmunogenic. In other embodiments, hypoimmunogenecity od the PSC (e.g., iPSC), hemogenic endothelium, HSPC, and/or differentiated lymphoid cell (e.g., NK cell) is achieved by modifying gene expression (e.g., knocking in or knocking out certain genes).

Hematological diseases are disorders which primarily affect the blood. Non-limiting such diseases or disorders include myeloid derived disorders such as hemoglobinopathies (congenital abnormality of the hemoglobin molecule or of the rate of hemoglobin synthesis), examples, sickle-cell disease, thalassemia, and methemoglobinemia; Anemias (lack of red blood cells or hemoglobin), Pernicious anemia; disorders resulting in decreased numbers of cells, such as myelodysplastic syndrome, neutropenia (decrease in the number of neutrophils), and thrombotic thrombocytopenic purpura (TTP), thrombocytosis, hematological malignancies such as lymphomas, myelomas, and leukemia. Lymphomas such as Hodgkin's disease, Non-Hodgkin's lymphoma, Burkitt's lymphoma, Anaplastic large cell lymphoma, Splenic marginal zone lymphoma, Hepatosplenic T-cell lymphoma, and Angioimmunoblastic T-cell lymphoma (AILT); myelomas such as Multiple myeloma, Waldenstrom macroglobulinemia, Plasmacytoma; leukemias that increases defect WBC such as Acute lymphocytic leukemia (ALL), Chronic lymphocytic leukemia (CLL), Acute myelogenous leukemia (AML), Chronic Idiopathic Myelofibrosis (MF), Chronic myelogenous leukemia (CML), T-cell prolymphocytic leukemia (T-PLL), B-cell prolymphocytic leukemia (B-PLL), Chronic neutrophilic leukemia (CNL), Hairy cell leukemia (HCL), T-cell large granular lymphocyte leukemia (T-LGL), and Aggressive NK-cell leukemia.

Provided herein is a method of treating an autoimmune disease, which comprises administering an effective amount of a cell or population thereof, or a composition, or a pharmaceutical composition as described herein to a patient in need thereof “Autoimmune disease” refers to a class of diseases in which a subject's own antibodies react with host tissue or in which immune effector T cells are autoreactive to endogenous self-peptides and cause destruction of tissue. Thus an immune response is mounted against a subject's own antigens, referred to as self-antigens. A “self-antigen” as used herein refers to an antigen of a normal host tissue. Normal host tissue does not include neoplastic cells.

Non-limiting examples of autoimmune diseases that can be treated include pemphigus (pemphigus vulgaris, pemphigus foliaceus or paraneoplastic pemphigus), Crohn's disease, idiopathic thrombocytopenic purpura (ITP), heparin induced thrombocytopenia (HIT), thrombotic thrombocytopenic purpura (TTP), Myasthenia Gravis (MG), and Chronic Inflammatory Demyelinating Polyneuropathy (CIDP). Additional non-limiting autoimmune diseases include autoimmune thrombocytopenia, immune neutropenia, antihemophilic FVIII inhibitor, antiphospholipid syndrome, Kawasaki Syndrome, ANCA-associated disease, polymyositis, bullous pemphigoid, multiple sclerosis (MS), Guillain-Barre Syndrome, chronic polyneuropathy, ulcerative colitis, diabetes mellitus, autoimmune thyroiditis, Graves' opthalmopathy, rheumatoid arthritis, ulcerative colitis, primary sclerosing cholangitis, systemic lupus erythematosus (SLE), autoimmune encephalomyelitis, Hashimoto's thyroiditis, Goodpasture's syndrome, autoimmune hemolytic anemia, scleroderma with anticollagen antibodies, mixed connective tissue disease, pernicious anemia, idiopathic Addison's disease, autoimmune-associated infertility, glomerulonephritis (e.g., crescentic glomerulonephritis, proliferative glomerulonephritis), insulin resistance, and autoimmune diabetes mellitus (type 1 diabetes mellitus; insulin dependent diabetes mellitus). Autoimmune disease has been recognized also to encompass atherosclerosis and Alzheimer's disease. In another embodiment, the autoimmune diseases include hepatitis, autoimmune hemophilia, autoimmune lymphoproliferative syndrome (ALPS), autoimmune uveoretinitis, glomerulonephritis, agammaglobulinemia, alopecia areata, amyloidosis, ankylosing spondylitis, autoimmune angioedema, autoimmune aplastic anemia, autoimmune dysautonomia, autoimmune hyperlipidemia, autoimmune immunodeficiency, autoimmune inner ear disease (AIED), autoimmune myocarditis, autoimmune pancreatitis, autoimmune retinopathy, autoimmune urticaria, autoimmune urticarial neuropathy, autoimmune axonal neuropathy, Balo disease, Behcet's disease, Castleman disease, celiac disease, Chagas disease, chronic recurrent multifocal osteomyelitis (CRMO), Churg-Strauss syndrome, cicatricial pemphigoid, benign mucosal pemphigoid, Cogan's syndrome, cold agglutinin disease, coxsackie myocarditis, CREST disease, essential mixed cryoglobulinemia, dermatitis herpetiformis, dermatomyositis, Devic's disease (neuromyelitis optica), dilated cardiomyopathy, discoid lupus, Dressler's syndrome, endometriosis, eosinophilic angiocentric fibrosis, Eosinophilic fasciitis, Erythema nodosum, Evans syndrome, Fibrosing alveolitis, Giant cell arteritis (temporal arteritis), Hashimoto's encephalitis, Henoch-Schonlein purpura, Herpes gestationis, Idiopathic hypocomplementemic tubulointerstitial nephritis, multiple myeloma, multifocal motor neuropathy, NMDA receptor antibody encephalitis, IgG4-related disease, IgG4-related sclerosing disease, inflammatory aortic aneurysm, inflammatory pseudotumour, inclusion body myositis, interstitial cystitis, juvenile arthritis, Kuttner's tumour, Lambert-Eaton syndrome, leukocytoclastic vasculitis, lichen planus, lichen sclerosus, Ligneous conjunctivitis, Linear IgA disease (LAD), Lyme disease, chronic, mediastinal fibrosis, Meniere's disease, Microscopic polyangiitis, Mikulicz's syndrome, Mooren's ulcer, Mucha-Habermann disease, multifocal fibrosclerosis, narcolepsy, optic neuritis, Ormond's disease (retroperitoneal fibrosis), palindromic rheumatism, PANDAS (pediatric autoimmune neuropsychiatric disorders associated with Streptococcus), paraneoplastic cerebellar degeneration, paraproteinemic polyneuropathies, paroxysmal nocturnal hemoglobinuria (PNH), Parry Romberg syndrome, Parsonnage-Turner syndrome, periaortitis, periarteritis, peripheral neuropathy, perivenous encephalomyelitis, POEMS syndrome, polyarteritis nodosa, Type I, II, & III autoimmune polyglandular syndromes, polymyalgia rheumatic, postpericardiotomy syndrome, progesterone dermatitis, primary biliary cirrhosis, psoriasis, psoriatic arthritis, idiopathic pulmonary fibrosis, pyoderma gangrenosum, pure red cell aplasia, Raynaud's phenomenon, reflex sympathetic dystrophy, Reiter's syndrome, relapsing polychondritis, restless legs syndrome, rheumatic fever, Riede's thyroiditis, sarcoidosis, Schmidt syndrome, scleritis, Sjogren's syndrome, sperm and testicular autoimmunity, stiff person syndrome, subacute bacterial endocarditis (SBE), Susac's syndrome, sympathetic ophthalmia, Takayasu's arteritis, Tolosa-Hunt syndrome, transverse myelitis, undifferentiated connective tissue disease (UCTD), vesiculobullous dermatosis, vitiligo, Rasmussen's encephalitis, Waldenstrom's macroglobulinaemia.

As used herein, the terms “administering,” “introducing” and “transplanting” are used interchangeably in the context of the placement of described cells, e.g. hematopoietic progenitor cells, into a subject, by a method or route which results in at least partial localization of the introduced cells at a desired site, such as a site of injury or repair, such that a desired effect(s) is produced. The cells e.g. hematopoietic progenitor cells, or their differentiated progeny (e.g., NK cells) can be administered by any appropriate route which results in delivery to a desired location in the subject where at least a portion of the implanted cells or components of the cells remain viable.

In various embodiments, the engineered cells described herein are optionally expanded ex vivo prior to administration to a subject. In other embodiments, the engineered cells are optionally cryopreserved for a period, then thawed prior to administration to a subject.

The engineered cells used for cellular replacement therapy can be autologous/autogenic (“self”) or non-autologous (“non-self,” e.g., allogeneic, syngeneic or xenogeneic) in relation to the recipient of the cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic.

In various embodiments, the engineered cell described herein that is to be implanted into a subject in need thereof is autologous or allogeneic to the subject.

In various embodiments, the engineered cell described herein can be derived from one or more donors, or can be obtained from an autologous source. In some embodiments, the engineered cells are expanded in culture prior to administration to a subject in need thereof.

In various embodiments, prior to implantation, the recipient subject is treated with chemotherapy and/or radiation.

In one embodiment, the chemotherapy and/or radiation is to reduce endogenous stem cells to facilitate engraftment of the implanted cells.

In various embodiments, prior to implantation, the engineered cells or the histone methyltransferase inhibited, multilineage hematopoietic progenitor cells or NK cells differentiated using a stroma-free method as described herein are treated ex vivo with prostaglandin E2 and/or antioxidant N-acetyl-L-cysteine (NAC) to promote subsequent engraftment in a recipient subject.

In various embodiments, the recipient subject is a human.

In various embodiments, the subject has been previously diagnosed with HIV or other viral disease, a hematological disease, or undergoing a cancer treatment.

In one embodiment, a subject is selected to donate a somatic cell which would be used to produce iPSCs and an engineered cell described herein. In one embodiment, the selected subject has a genetic disease or defect.

In various embodiments, the donor subject is a human, non-human animal, rodent or non-rodent. For example, the subject can be any mammal, e.g., a human, other primate, pig, rodent such as mouse or rat, rabbit, guinea pig, hamster, cow, horse, cat, dog, sheep or goat, or a non-mammal such as a bird.

In various embodiments, the donor has been previously diagnosed with HIV, a hematological disease or cancer.

In one embodiment, a biological sample, a population of embryonic stem cells, somatic stem cells, progenitor cells, bone marrow cells, hematopoietic stem cells, or hematopoietic progenitor cells is obtained from the donor subject.

In various embodiments, the biological sample, a population of embryonic stem cells, somatic stem cells, progenitor cells, bone marrow cells, hematopoietic stem cells, or hematopoietic progenitor cells described herein can be derived from one or more donors, or can be obtained from an autologous source.

In one embodiment, the embryonic stem cells, somatic stem cells, progenitor cells, bone marrow cells, hematopoietic stem cells, hematopoietic progenitor cells are isolated from the donor subject, transfected, cultured (optional), and transplanted back into the same subject, i.e. an autologous cell transplant. Here, the donor and the recipient subject is the same individual. In another embodiment, the embryonic stem cells, somatic stem cells, progenitor cells, bone marrow cells, hematopoietic stem cells, or hematopoietic progenitor cells are isolated from a donor who is an HLA-type match with a subject (recipient). Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different subject, i.e., allogeneic to the recipient host subject. The donor's or subject's embryonic stem cells, somatic stem cells, progenitor cells, bone marrow cells, hematopoietic stem cells, or hematopoietic progenitor cells can be transfected with a vector or nucleic acid comprising the nucleic acid molecule(s) described herein, the transfected cells are cultured, inhibited, and differentiated as disclosed, optionally expanded, and then transplanted into the recipient subject. In one embodiment, the transplanted engineered cells engraft in the recipient subject. In one embodiment, the transplanted engineered cells reconstitute the immune system in the recipient subject. The transfected cells can also be cryopreserved after transfected and stored, or cryopreserved after cell expansion and stored.

The engineered cells or the histone methyltransferase inhibited, multilineage hematopoietic progenitor cells or NK cells differentiated using a stroma-free method as described herein may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment, a dose of cells is delivered to a subject intravenously. In one embodiment, the cells are intravenously administered to a subject.

In particular embodiments, patients receive a dose of the modified cells described herein, e.g., engineered cells or the histone methyltransferase inhibited, multilineage hematopoietic progenitor cells or NK cells differentiated using a stroma-free method as described herein, of about 1×10⁵ cells/kg, about 5×10⁵ cells/kg, about 1×10⁶ cells/kg, about 2×10 6 cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose.

In certain embodiments, patients receive a dose of the modified cells described herein, e.g., engineered cells or the histone methyltransferase inhibited, multilineage hematopoietic progenitor cells or NK cells differentiated using a stroma-free method as described herein, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of the modified cells described herein, e.g., engineered cells or the histone methyltransferase inhibited, multilineage hematopoietic progenitor cells or NK cells differentiated using a stroma-free method as described herein, of about 1×10⁵ cells/kg to about 1×10 8 cells/kg, about 1×10⁶ cells/kg to about 1×10 8 cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In general, the engineered cells or the histone methyltransferase inhibited, multilineage hematopoietic progenitor cell described herein or NK cells differentiated using a stroma-free method as described herein are administered as a suspension with a pharmaceutically acceptable carrier, for example, as therapeutic compositions. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include, e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

In some embodiments, the composition of engineered cells described further comprises a pharmaceutically acceptable carrier.

In various embodiments, at least a second or subsequent dose of cells is administered to the recipient subject. For example, a second administration can be given between about one day to 30 weeks from the previous administration. Two, three, four or more total subsequent administrations can be delivered to the individual, as needed, e.g., determined by a skilled clinician.

A cell composition can be administered by any appropriate route which results in effective cellular replacement treatment in the subject, i.e. administration results in delivery to a desired location in the subject where at least a portion of the composition delivered, i.e. at least 1×10⁴ cells are delivered to the desired site for a period of time. Modes of administration include injection, infusion, or instillation, “Injection” includes, without limitation, intravenous, intra-arterial, intraventricular, intracardiac injection and infusion. For the delivery of cells, administration by injection or infusion is generally preferred.

Efficacy testing can be performed during the course of treatment using the methods described herein. Measurements of the degree of severity of a number of symptoms associated with a particular ailment are noted prior to the start of a treatment and then at later specific time period after the start of the treatment. In some embodiments, a pharmaceutical composition comprising an immune as described herein or a population thereof can be used for cellular replacement therapy in a subject.

Accordingly, it is also the objective of this the present disclosure to provide compositions of modified (also referred to as engineered) cells for use in in vivo cellular replacement therapy, medical therapy such as cancer immune therapy, and for the in vitro studies of disease modeling, drug screening, and hematological diseases.

The advantage of the disclosed protocols is that the methods permit semi-permanent bulk production of desired cells or other types of hematopoietic cells (e.g., cells differentiated from multipotent HSCs) from a variety of types of cell source, from stem cells, hematopoietic progenitor cells, and mature and differentiated somatic cells, all of which can be readily collected from the patient's body.

The produced engineered cells or engineered histone methyltransferase-inhibited, CD34⁺/CD 38^{lo/-} hemogenic endothelium or NK cells differentiated using a stroma-free method as described herein can be transplanted into a patient for various medical treatments such as immune system reconstruction therapy (e.g., after bone marrow ablation) or immunotherapy (e.g., in cancer therapy or autoimmune diseases). One added advantage is that if the donor of the source cells and recipient of the engineered cells are the same person, the produced engineered cells have HLA that are identical to the recipient and this avoids host-graft immune rejection after the transplantation. For recipient patients that are HLA allogeneic to the donor person of the source cells, host-graft immune rejection is greatly reduced.

The produced engineered cells or engineered histone methyltransferase-inhibited, CD34⁺/CD38^{lo/-} hemogenic endothelium or NK cells differentiated using a stroma-free method as described herein can also be cryopreserved until needed in the future.

Currently, bone marrow transplantation is the most established cellular replacement therapy for a variety of hematological disorders. The functional unit of a bone marrow transplant is the hematopoietic stem cell (HSC), which resides at the apex of a complex cellular hierarchy and replenishes blood development throughout life. The scarcity of HLA-matched HSCs severely limits the ability to carry out transplantation, disease modeling and drug screening. As such, many studies have aimed to generate HSCs from alternative sources. Advances in reprogramming to induced pluripotent stem cells (iPSCs) has provided access to a wide array of patient-specific pluripotent cells, a promising source for disease modeling, drug screens and cellular therapies. However, the inability to derive engraftable hematopoietic stem and progenitor cells from human pluripotent stem cells (hPSCs) has limited the characterization of hematological diseases to in vitro assays. Generation of HSCs by directed differentiation has remained elusive, and there is a need for novel approaches to this problem.

Accordingly, in one aspect described herein is a method of cellular replacement therapy, the method comprising administering an cell as described herein (e.g., a CD56+NK cell) or population thereof, or a composition comprising said cell or population thereof, or a pharmaceutical composition comprising said cell or population thereof to a recipient subject in need thereof.

In some embodiments, the recipient subject has undergone chemotherapy and/or irradiation. In some embodiments, the recipient subject has deficiencies in immune function and/or lymphocyte reconstitution. In some embodiments, prior to transplanting, the cell or population thereof is treated ex vivo with prostaglandin E2 and/or antioxidant N-acetyl-L-cysteine (NAC) to promote subsequent engraftment in a recipient subject.

### Kits

Another aspect of the technology described herein relates to kits for differentiating NK cells using a stroma-free method as described herein, among others. Described herein are kit components that can be included in one or more of the kits described herein.

In some embodiments, the kit comprises an effective amount of NK-cell-differentiation factors (e.g., IL-7, SCF, FLT3, TPO, IL-15, and/or IL-3); or an effective amount of iPSC reprogramming factors (e.g., OCT4, SOX2, KLF4, c-MYC, nanog, and/or LIN28); or an effective amount of hemogenic endothelium differentiation factors (e.g., BMP4, SB-431542, CHIR99021, bFGF, VEGF, IL-6, IL-11, IGF-1, SCF, and EPO); or an effective amount of an inhibitor of an epigenetic regulator (e.g., MC1568; CAY10591; UNC0224; UNC0638; A366; BRD4770; BIX01294; UNC0642; UNC0631; UNC0646; UNC0321; E72; BIX-01338; BRD9539; Chaetocin; or DCG066; e.g., an EZH1 RNA interference agent). As will be appreciated by one of skill in the art, such cell differentiation factors can be supplied in a lyophilized form or a concentrated form that can diluted prior to use with cultured cells. Preferred formulations include those that are non-toxic to the cells and/or does not affect growth rate or viability etc. NK-cell-differentiation factors can be supplied in aliquots or in unit doses.

In some embodiments, the kit comprises a cell culture vessel comprising an immobilized Notch ligand. In some embodiments, the kit comprises a cell culture vessel and a Notch ligand that can be immobilized to the cell culture vessel using reagents and/or instructions provided therein. In some embodiments, the kit does not comprise stromal cells as described herein.

In some embodiments, the kit further comprises a vector comprising a nucleic acid encoding a CAR.

In some embodiments, the components described herein can be provided singularly or in any combination as a kit. The kit includes the components described herein, e.g., a composition comprising Notch ligand that does not comprise stromal cells, a composition(s) comprising differentiation factor(s), a composition(s) that includes a vector comprising e.g., CAR as described throughout the specification. Such kits can optionally include one or more agents that permit the detection of markers for NK cell maturation (e.g., CD3−; CD56+; Fc gamma RIII/CD16+; CD57+; NK1.1+; NK1.2+; CD94+, CD122/IL-2 beta+; CD217/IL-7R alpha−; KIR family receptors+; NKG2A+; NKG2D+; NKp30+; NKp44+; NKp46+; or NKp80+, etc.) or a set thereof. Such kits can optionally include one or more agents that permit the detection of markers for NK cell activation (e.g., TRAIL, IFNg, TNFa, granzyme B, perforin, etc.) or a set thereof. Such kits can optionally include one or more agents that permit the detection of markers for hemogenic endothelium (e.g., CD34, CD38, CD45, KDR, CD235, CD43, etc.). In addition, the kit optionally comprises informational material. The kit can also contain a substrate for coating culture dishes, such as laminin, fibronectin, Poly-L-Lysine, or methylcellulose.

In some embodiments, the compositions in the kit can be provided in a watertight or gas tight container which in some embodiments is substantially free of other components of the kit. For example, a cell differentiation reagent can be supplied in more than one container, e.g., it can be supplied in a container having sufficient reagent for a predetermined number of differentiation assays, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of a cell culture vessel comprising immobilized Notch ligand; or the production of NK cells differentiated using a stroma-free method as described herein; or the concentration, date of expiration, batch or production site information, and so forth of reagents used herein such as cell differentiation factors. In one embodiment, the informational material relates to methods for using or administering the components of the kit.

The kit can include a component for the detection of a marker for cell differentiation. In addition, the kit can include one or more antibodies that bind a cell marker, or primers for an RT-PCR or PCR reaction, e.g., a semi-quantitative or quantitative RT-PCR or PCR reaction. Such components can be used to assess the activation of cell maturation markers or the loss of undifferentiated or immature cell markers. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “cell” refers to a single cell as well as to a population of (i.e., more than one) cells. The population may be a pure population comprising one cell type, such as a population of pluripotent stem cells or a population of differentiated NK cells. As used herein, the term “population” refers to a pure population or to a population comprising a majority (e.g., at least 50%, at least 60%, at least 70%, at least 80%, at least 90%, at least 95%, at least 99%) of one cell type. Alternatively, the population may comprise more than one cell type, for example a mixed cell population. It is not meant to limit the number of cells in a population; for example, a mixed population of cells may comprise at least one differentiated cell. In the present invention, there is no limit on the number of cell types that a mixed cell population may comprise.

As used herein, the term “immune cell” (which can be used interchangeably with the terms “white blood cell” or “leukocyte”) refers to a single cell as well as to a population of (i.e., more than one) cells that is part of the immune system and fights infections and other diseases. Immune cells differentiate from stem cells in the bone marrow. Non-limiting examples of immune cells include T-cells, B-cells, NK-cells, dendritic cells, monocytes, macrophages, neutrophils, basophils, and eosinophils.

As used herein, in one embodiment, the term “hematopoietic stem cell” or “HSC” refers to a stem cell that has self-renewal capacity and also gives rise to all the blood cell types of the three hematopoietic lineages, erythroid, lymphoid, and myeloid. These cell types include the lymphoid lineages (T-cells, B-cells, NK-cells) and the myeloid lineages, which include dendritic cell lineages, granulocyte—monocyte lineages (e.g., monocytes, macrophages, neutrophils, basophils, eosinophils) and megakaryocyte—erythroid lineages (e.g., erythrocytes, megakaryocytes/platelets). Human HSCs can express a combination of the following markers: CD34⁺, CD59⁺, CD90/Thy1⁺, CD38^{low/-}, c-kit/CD117^{-/low}, and/or Lin⁻. Mouse HSC are considered CD34^{low/-}, SCA-1⁺, CD90/Thy1^{+/low}, CD38⁺, c-Kit/CD117⁺, and Lin⁻. Detecting the expression of these marker panels allows separation of specific cell populations via techniques like fluorescence-activated cell sorting (FACS). In one embodiment, the term “hematopoietic stem cell” or “HSC” refers to a stem cell that has self-renewal capacity and that have the following cell surface markers: CD34+, CD59+, Thy1/CD90⁺, CD38^{lo/-}, CD133+, c-Kit/CD117^{-/lo}, and Lin⁻. In one embodiment, the term “hematopoietic stem cell” or “HSC” refers to a stem cell that is at least CD34+. In one embodiment, the term “hematopoietic stem cell” or “HSC” refers to a stem cell that has self-renewal capacity and that is at least CD34⁺ and c-kit/CD117^{lo/-}. In one embodiment, the term “hematopoietic stem cell” or “HSC” refers to a stem cell that has self-renewal capacity and that is at least CD38^{low/-}, c-kit/CD117^{-/low}. The term HSC can be used interchangeably with the term “hematopoietic stem and progenitor cell” (HSPC) or “hematopoietic progenitor cell” (HPC).

As used herein, the terms “iPS cell”, “iPSC”, and “induced pluripotent stem cell” are used interchangeably and refers to an induced pluripotent cell that can self-renew indefinitely and differentiate to cell types characteristic of all three germ cell layers. In some embodiments, the induced pluripotent stem cells are produced by introducing into mature cells at least one reprogramming factor selected from the group consisting of: OCT4, SOX2, KLF4, c-MYC, nanog, and LIN28, or any combination thereof (see, e.g., Table 9). Such reprogramming factors can be introduced into mature cells using transfection with DNA or RNA encoding the reprogramming factor(s). In some embodiments, the iPSC can be artificially derived by the introduction of the following reprogramming factors OCT4, SOX2, KLF4, and optionally c-MYC or nanog and LIN28, to a differentiated cell, e.g., a somatic cell. The term “hPSC” refers to a human pluripotent stem cell.

As used herein, the term “lineage” when used in the context of stem and progenitor cell differentiation and development refers to the cell differentiation and development pathway, which the cell can take to becoming a fully differentiated cell. For example, a HSC has three hematopoietic lineages, erythroid, lymphoid, and myeloid; the HSC has the potential, i.e., the ability, to differentiate and develop into those terminally differentiated cell types known for all these three lineages. When the term “multilineage” used, it means the cell is able to, in the future, differentiate and develop into those terminally differentiated cell types known for more than one lineage. For example, the HSC has multilineage potential and can also be referred to as be as multilineage hematopoietic stem cells (MHSC) or multilineage hematopoietic progenitor cells (MHPC). When the term “limited lineage” used, it means the cell can differentiate and develop into those terminally differentiated cell types known for one lineage. For example, a common myeloid progenitor cell (CMP) or a megakaryocyte-erythroid progenitor (MEP) has a limited lineage because the cell can only differentiate and develop into those terminally differentiated cell types of the myeloid lineage and not that of the lymphoid lineage. Terminally differentiated cells of the myeloid lineage include erythrocytes, monocytes, macrophages, megakaryocytes, myeloblasts, dendritic cells, and granulocytes (basophils, neutrophils, eosinophils, and mast cells); and terminally differentiated cells of the lymphoid lineage include T lymphocytes/T cells, B lymphocytes/B cells, dendritic cells, and natural killer cells.

As used herein, the term “a progenitor cell” refers to an immature or undifferentiated cell that has the potential later on to mature (differentiate) into a specific cell type (a fully differentiated or terminally differentiated cell), for example, a blood cell, a skin cell, a bone cell, or hair cells. Progenitor cells have a cellular phenotype that is more primitive (e.g., is at an earlier step along a developmental pathway or progression than is a fully differentiated cell) relative to a cell, which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. A progenitor cell also can proliferate to make more progenitor cells that are similarly immature or undifferentiated.

As used herein, the term “aggregation media” refers to a series of cell culture media that are used to differentiate a population of pluripotent stem cells, or a derivative cell population thereof such as embryoid bodies, into a population of CD34+ hemogenic endothelium. In some embodiments, the aggregation media comprises at least BMP4, SB-431542, CHIR99021, bFGF, VEGF, IL-6, IL-11, IGF-1, SCF, and EPO, each of which can be added into the aggregation media at designated timepoints and concentrations (see, e.g., Example 2; see, e.g., Table 2).

As used herein, the term “Natural Killer-cell-differentiation media” or “NK-cell-differentiation media” refers to a series of cell culture media that are used to differentiate a population of CD34⁺ hemogenic endothelium into a population of CD56⁺ NK cells. In some embodiments, “NK-cell-differentiation medium” refers to a single type of cell culture medium that is used to differentiate a population of CD34+ hemogenic endothelium into a population of CD56⁺ NK cells. In some embodiments, the NK-cell-differentiation media comprises at least SCF, FLT3, and IL7, and optionally TPO, IL-15, and/or IL-3, each of which can be added into the NK-cell-differentiation media at designated timepoints and concentrations (see, e.g., Example 1; see, e.g., Table 1).

The term “differentiated cell” is meant any primary cell that is not, in its native form, pluripotent as that term is defined herein. The term a “differentiated cell” also encompasses cells that are partially differentiated, such as multipotent cells (e.g., adult somatic stem cells). In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell or a endodermal stem cell), which in turn can differentiate into other types of precursor cells further down the pathway (such as an tissue specific precursor, for example, a cardiomyocyte precursor, or a pancreatic precursor), and then to an end-stage differentiated cell, which plays a characteristic role in a certain tissue type, and may or may not retain the capacity to proliferate further.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult somatic stem cells, such as for example, hematopoietic stem cells and neural stem cells, hair follicle stem cells, liver stem cells etc. Multipotent means a stem cell may form many types of cells in a given lineage, but not cells of other lineages. For example, a multipotent blood stem cell can form the many different types of blood cells (red, white, platelets, etc.), but it cannot form neurons; cardiovascular progenitor cell (MICP) differentiation into specific mature cardiac, pacemaker, smooth muscle, and endothelial cell types; pancreas-derived multipotent progenitor (PMP) colonies produce cell types of pancreatic lineage (cells that produces insulin, glucagon, amylase or somatostatin) and neural lineage (cells that are morphologically neuron-like, astrocytes-like or oligodendrocyte-like).

The term a “reprogramming gene”, as used herein, refers to a gene whose expression, contributes to the reprogramming of a differentiated cell, e.g. a somatic cell to an undifferentiated cell (e.g. a cell of a pluripotent state or partially pluripotent state, multipotent state). A reprogramming gene can be, for example, genes encoding master transcription factors Sox2, Oct3/4, Klf4, Nanog, Lin-28, c-myc and the like. The term “reprogramming factor” refers to the protein encoded by the reprogramming gene.

The term “exogenous” refers to a substance present in a cell other than its native source. The terms “exogenous” when used herein refers to a nucleic acid (e.g. a nucleic acid encoding a reprogramming transcription factor, e.g. Sox2, Oct3/4, Klf4, Nanog, Lin-28, c-myc and the like) or a protein (e.g., a transcription factor polypeptide) that has been introduced by a process involving the hand of man into a biological system such as a cell or organism in which it is not normally found or in which it is found in lower amounts. A substance (e.g. a nucleic acid encoding a sox2 transcription factor, or a protein, e.g., a SOX2 polypeptide) will be considered exogenous if it is introduced into a cell or an ancestor of the cell that inherits the substance.

The term “isolated” as used herein signifies that the cells are placed into conditions other than their natural environment. The term “isolated” does not preclude the later use of these cells thereafter in combinations or mixtures with other cells.

As used herein, the term “expanding” refers to increasing the number of like cells through cell division (mitosis). The term “proliferating” and “expanding” are used interchangeably.

As used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. A cell-surface marker of particular relevance to the methods described herein is CD34. The useful hematopoietic progenitor cells (e.g., hemogenic endothelium) according to the present disclosure preferably express CD34 or in other words, they are CD34 positive.

A cell can be designated “positive” or “negative” for any cell-surface marker, and both such designations are useful for the practice of the methods described herein. A cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the methods described herein, the cell must express it on its surface. Similarly, a cell is considered “negative” or “negative/low” (abbreviated as “−/lo” or “lo/−”) for a cell-surface marker if the cell does not express the marker on its cell surface in amounts sufficient to be detected using methods known to those of skill in the art, such as contacting a cell with an antibody that binds specifically to that marker and subsequently performing flow cytometric analysis of such a contacted cell to determine whether the antibody is bound the cell. In some embodiments, where agents specific for cell-surface lineage markers used, the agents can all comprise the same label or tag, such as fluorescent tag, and thus all cells positive for that label or tag can be excluded or removed, to leave uncontacted hematopoietic stem or progenitor cells for use in the methods described herein.

As used herein, the term “a histone methyltransferase inhibitor” is any molecule that inhibits expression of a histone methyltransferase (e.g., G9a, GLP, EZH1), or inhibits the catalytic activity of the enzyme to methylate lysine resides on the substrate histone protein. For example, a histone methyltransferase inhibitor can be an siRNA or dsRNA that inhibits expression of G9a, GLP, or EZH1 in the inhibited cell, or a gRNA, together with for example Cas or dead Cas9 (dCas), that promotes the degradation of the mRNA or decreases the transcription of mRNA of G9a, GLP, or EZH1 in the inhibited cell. In some embodiments, a histone methyltransferase inhibitor is a small molecule that antagonizes the enzyme activity. Examples include but are not limited to small molecules AMI-1, A-366, BIX-01294, BIX01338, BRD4770, chaetocin, UNC0224, UNC0631, UNC0638, UNC0642, UNC0646, EPZ5676, EPZ005687, GSK343, EPZ-6438, 3-deazaneplanocin A (DZNeP) HCl, UNC1999, MM-102, SGC 0946, Entacapone, EPZ015666, UNC0379, EI1, MI-2 (Menin-MLL Inhibitor), MI-3 (Menin-MLL Inhibitor), PFI-2, GSK126, EPZ004777, BRD4770, and EPZ-6438 as described herein.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the small molecule is a heterorganic compound or an organometallic compound.

The term “nucleic acid inhibitor” is meant to include a nucleic acid molecule that contains a sequence that is complementary to a target nucleic acid (e.g., a target mRNA) that mediates a decrease in the level or activity of the target nucleic acid. Non-limiting examples of nucleic acid inhibitors include interfering RNA, microRNA, shRNA, siRNA, ribozymes, antagomirs, and antisense oligonucleotides or gRNA in combination with Cas enzyme. Methods of making nucleic acid inhibitors are described herein. Additional methods of making nucleic acid inhibitors are known in the art. In one embodiment, the G9a/GLP or EZH1 nucleic acid inhibitor causes a decrease in the activity of G9a/GLP or EZH1 mRNA or decreased transcription of G9a/GLP or EZH1 mRNA.

As used herein, “an interfering RNA” refers to any double stranded or single stranded RNA sequence, capable, either directly or indirectly (i.e., upon conversion), of inhibiting or down-regulating gene expression by mediating RNA interference. Interfering RNA includes, but is not limited to, small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family. As used herein, the phrase “post-transcriptional processing” refers to mRNA processing that occurs after transcription and is mediated, for example, by the enzymes Dicer and/or Drosha.

A “small interfering RNA” or “siRNA” as used herein refers to any small RNA molecule capable of inhibiting or down regulating gene expression by mediating RNA interference in a sequence specific manner. The small RNA can be, for example, about 18 to 21 nucleotides long. Each siRNA duplex is formed by a guide strand and a passenger strand. The endonuclease Argonaute 2 (Ago 2) catalyzes the unwinding of the siRNA duplex. Once unwound, the guide strand is incorporated into the RNA Interference Specificity Complex (RISC), while the passenger strand is released. RISC uses the guide strand to find the mRNA that has a complementary sequence leading to the endonucleolytic cleavage of the target mRNA.

Retroviruses are RNA viruses that utilize reverse transcriptase during their replication cycle. The term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus.

The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules, which encode the structural proteins and enzymes needed to produce new viral particles.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R, and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R, and U5 regions, appears at both the both the 5′ and 3′ ends of the viral genome. In one embodiment of the invention, the promoter within the LTR, including the 5′ LTR, is replaced with a heterologous promoter. Examples of heterologous promoters that can be used include, for example, a spleen focus-forming virus (SFFV) promoter, a tetracycline-inducible (TET) promoter, a 0-globin locus control region and a 0-globin promoter (LCR), and a cytomegalovirus (CMV) promoter.

The term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells.

The term “R region” refers to the region within retroviral LTRs beginning at the start of the capping group (i.e., the start of transcription) and ending immediately prior to the start of the poly A tract. The R region is also defined as being flanked by the U3 and U5 regions. The R region plays an important role during reverse transcription in permitting the transfer of nascent DNA from one end of the genome to the other.

The term “promoter/enhancer” refers to a segment of DNA which contains sequences capable of providing both promoter and enhancer functions. For example, the long terminal repeats of retroviruses contain both promoter and enhancer functions. The enhancer/promoter may be “endogenous,” “exogenous,” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can take any shapes, for example, linear or circular. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, iRNA, miRNA, siRNA, etc.

The nucleic acid can be selected, for example, from a group including: nucleic acid encoding a protein of interest, oligonucleotides, nucleic acid analogues, for example peptide-nucleic acid (PNA), pseudo-complementary PNA (pc-PNA), and locked nucleic acid (LNA). Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, microRNAi (miRNA), and antisense oligonucleotides.

As used herein, the term “engraftment” in reference to a recipient host is when the new blood-forming cells start to grow and which are derived from the implanted cells and make healthy blood stem cells that show up in recipient's blood after a minimum period of 10 days after implantation. Engraftment can occur as early as 10 days after transplant but is more common around 14-20 days.

As used herein, the term “reconstitution” with respect to the immune system or the blood system in a recipient host refers to the rebuilding the innate reservoir or working system, or part thereof within the body of recipient host to a natural or a functionally state. For example, such as bone marrow after chemotherapy had obliterated the bone marrow stem cells.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cellular replacement therapy. A subject can be male or female.

A subject can be one who has been previously diagnosed with or identified as suffering from or having a condition in need of treatment (e.g. hematologic disease, cancer, etc.) or one or more complications related to such a condition, and optionally, have already undergone treatment for a hematologic disease or the one or more complications related to a hematologic disease. Alternatively, a subject can also be one who has not been previously diagnosed as having a hematologic disease or one or more complications related to a hematologic disease. For example, a subject can be one who exhibits one or more risk factors for a hematologic disease or one or more complications related to a hematologic disease or a subject who does not exhibit risk factors.

A “subject in need” of treatment for a particular condition can be a subject having that condition, diagnosed as having that condition, or at risk of developing that condition.

A variant amino acid or DNA sequence can be at least 85%, at least 87%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

In some embodiments, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, the differentiated and/or engineered NK cell described herein is exogenous. In some embodiments, the differentiated and/or engineered NK cell described herein is ectopic. In some embodiments, the differentiated and/or engineered NK cell described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

Nucleic acids encoding a polypeptide as described herein (e.g., a CAR polypeptide) can be comprised by a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

The vector can be recombinant, e.g., it comprises sequences originating from at least two different sources. In some embodiments, the vector comprises sequences originating from at least two different species. In some embodiments, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. For example, the use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a hematological disease or cancer. The term “treating” includes reducing or alleviating at least one adverse effect or symptom of a condition, disease or disorder associated with a hematological disease or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method comprising:
   a) inhibiting a histone methyltransferase in a population of CD34⁺ hemogenic endothelium; and
   b) differentiating the population of CD34⁺ hemogenic endothelium in Natural Killer (NK)-cell-differentiation media in the presence of a Notch ligand for a sufficient time to promote differentiation into a population of CD56⁺ NK cells.
2. A method comprising:
   a) differentiating a population of pluripotent stem cells in aggregation media for a sufficient time to promote differentiation into a population of CD34⁺ hemogenic endothelium;
   b) inhibiting a histone methyltransferase in the resultant population of CD34⁺ hemogenic endothelium; and
   c) differentiating the resultant population of CD34⁺ hemogenic endothelium in Natural Killer (NK)-cell-differentiation media in the presence of a Notch ligand for a sufficient time to promote differentiation into a population of CD56⁺ NK cells.
3. A method comprising:
   a) inhibiting an epigenetic regulator in a population of CD34⁺ hemogenic endothelium; and
   b) differentiating the population of CD34⁺ hemogenic endothelium in NK-cell-differentiation media in the presence of a Notch ligand for a sufficient time to promote differentiation into a population of CD56⁺ NK cells.
4. A method comprising:
   a) differentiating a population of pluripotent stem cells in aggregation media for a sufficient time to promote differentiation into a population of CD34⁺ hemogenic endothelium;
   b) inhibiting an epigenetic regulator in the resultant population of CD34⁺ hemogenic endothelium; and
   c) differentiating the resultant population of CD34⁺ hemogenic endothelium in NK-cell-differentiation media in the presence of a Notch ligand for a sufficient time to promote differentiation into a population of CD56⁺ NK cells.
5. A method comprising:
   a) inhibiting G9a and/or GLP in a population of CD34⁺ hemogenic endothelium; and
   b) differentiating the population of CD34⁺ hemogenic endothelium in NK-cell-differentiation media in the presence of a Notch ligand for a sufficient time to promote differentiation into a population of CD56⁺ NK cells.
6. A method comprising:
   a) differentiating a population of pluripotent stem cells in aggregation media for a sufficient time to promote differentiation into a population of CD34⁺ hemogenic endothelium;
   b) inhibiting G9a and/or GLP in the resultant population of CD34⁺ hemogenic endothelium; and
   c) differentiating the resultant population of CD34⁺ hemogenic endothelium in NK-cell-differentiation media in the presence of a Notch ligand for a sufficient time to promote differentiation into a population of CD56⁺ NK cells.
7. A method comprising differentiating a population of CD34⁺ hemogenic endothelium in NK-cell-differentiation media in the presence of a Notch ligand for a sufficient time to promote differentiation into a population of CD56⁺ NK cells.
8. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media in the presence of a Notch ligand for a sufficient time to promote differentiation into a population of CD56⁺ NK cells.
9. The method of any one of paragraphs 1-8, wherein the Notch ligand is attached to a solid substrate.
10. The method of any one of paragraphs 1-9, wherein the Notch ligand is attached to a cell culture dish.
11. The method of any one of paragraphs 1-10, wherein the Notch ligand is not derived from a stromal cell.
12. The method of any one of paragraphs 1-11, wherein differentiating the hemogenic endothelium in the presence of a Notch ligand does not comprise co-culturing with a stromal cell expressing a Notch ligand.
13. The method of any one of paragraphs 1-12, wherein differentiating the hemogenic endothelium in the presence of a Notch ligand does not comprise co-culturing with OP9-DLL1 cells or OP9-DLL4 cells.
14. The method of any one of paragraphs 1-13, wherein the Notch ligand is selected from the group consisting of Delta-like-1 (DLL1), Delta-like-4 (DLL4), immobilized Delta1^{ext-IgG}, and immobilized Delta4^{ext-IgG}.
15. The method of paragraph 14, wherein immobilized Delta1^{ext-IgG} consists of an extracellular domain of human Delta-like-1 fused to the Fc domain of human IgG1.
16. The method of any one of paragraphs 1-15, wherein the Notch ligand is DLL4.
17. The method of any one of paragraphs 1-16, wherein the Notch ligand is provided at a concentration of at most 5 μg/ml.
18. The method of any one of paragraphs 1-17, wherein the sufficient time to promote differentiation into a population of CD56⁺ NK cells is at least 4 weeks.
19. The method of any one of paragraphs 1-18, wherein the NK-cell-differentiation media is serum-free.
20. The method of any one of paragraphs 1-19, wherein the NK-cell-differentiation media comprises SCF, FLT3, and IL7.
21. The method of any one of paragraphs 1-20, wherein the NK-cell-differentiation media comprises 30 ng/ml-100 ng/mL SCF; 15 ng/ml-100 ng/mL FLT3; and 5 ng/ml-20 ng/ml IL7.
22. The method of any one of paragraphs 1-21, wherein the NK-cell-differentiation media comprises 30 ng/mL SCF, 15 ng/ml FLT3, and 20 ng/ml IL7.
23. The method of any one of paragraphs 1-22, wherein the NK-cell-differentiation media comprises 50 ng/mL SCF, 50 ng/ml FLT3, and 10 ng/ml IL7.
24. The method of any one of paragraphs 1-23, wherein the NK-cell-differentiation media comprises 100 ng/mL SCF, 100 ng/ml FLT3, and 5 ng/ml IL7.
25. The method of any one of paragraphs 1-24, wherein the NK-cell-differentiation media comprises 30 ng/ml SCF, 20 ng/ml FLT3, and 25 ng/ml IL7
26. The method of any one of paragraphs 1-25, wherein the NK-cell-differentiation media further comprises thrombopoietin (TPO).
27. The method of any one of paragraphs 1-26, wherein the NK-cell-differentiation media further comprises 5 ng/mL thrombopoietin (TPO) for at least the first 2 weeks of differentiating in the NK-cell-differentiation media.
28. The method of any one of paragraphs 1-27, wherein the NK-cell-differentiation media further comprises interleukin-15 (IL-15).
29. The method of any one of paragraphs 1-28, wherein the NK-cell-differentiation media further comprises 10 ng/mL interleukin-15 (IL-15) starting after at least the first 2 weeks of differentiating in the NK-cell-differentiation media.
30. The method of any one of paragraphs 1-29, wherein the NK-cell-differentiation media further comprises interleukin-3 (IL-3).
31. The method of any one of paragraphs 1-30, wherein the NK-cell-differentiation media further comprises 5 ng/mL interleukin-3 (IL-3) for at least the first week of differentiating in the NK-cell-differentiation media.
32. The method of any one of paragraphs 1-31, further comprising a step of CD56⁺ NK cell enrichment.
33. The method of any one of paragraphs 2, 4, 6, or 8, wherein the population of pluripotent stem cells comprises induced pluripotent stem cells (iPS cells) or embryonic stem cells (ESC).
34. The method of paragraph 33, wherein the induced pluripotent stem cells are produced by introducing into mature cells at least one reprogramming factor selected from the group consisting of: OCT4, SOX2, KLF4, c-MYC, nanog, and LIN28, or any combination thereof
35. The method of paragraph 34, wherein the induced pluripotent stem cells are produced by introducing only reprogramming factors OCT4, SOX2, KLF4 and optionally c-MYC or nanog and LIN28 into mature cells.
36. The method of any one of paragraphs 33-35, wherein the induced pluripotent stem cells are produced by introducing the reprogramming factors two or more times into the mature cells.
37. The method of any one of paragraphs 2, 4, 6, or 8, wherein the population of pluripotent stem cells is differentiated into a population of CD34⁺ hemogenic endothelium using embryoid bodies or 2D adherent cultures.
38. The method of any one of paragraphs 2, 4, 6, or 8, wherein the sufficient time to promote differentiation into a population of CD34⁺ hemogenic endothelium is at least 8 days.
39. The method of any one of paragraphs 2, 4, 6, or 8, wherein the aggregation media comprises BMP4, SB-431542, CHIR99021, bFGF, VEGF, IL-6, IL-11, IGF-1, SCF, and EPO.
40. The method of any one of paragraphs 2, 4, 6, or 8, wherein the aggregation media comprises:
   a) BMP4 for at least days 0 and 2;
   b) SB-431542 for at least day 2;
   c) CHIR99021 for at least day 2;
   d) bFGF for at least day 1, 2, 3, and 6;
   e) VEGF for at least days 3 and 6;
   f) IL-6 for at least day 6;
   g) IL-11 for at least day 6;
   h) IGF-1 for at least day 6;
   i) SCF for at least day 6; and/or
   j) EPO for at least day 6.
41. The method of any one of paragraphs 2, 4, 6, 9, or 40, wherein the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO.
42. The method of any one of paragraphs 39-41, wherein BMP4 is at a concentration of about 10 ng/ml in the aggregation media.
43. The method of any one of paragraphs 39-41, wherein SB-431542 is at a concentration of about 6 mM in the aggregation media.
44. The method of any one of paragraphs 39-41, wherein CHIR99021 is at a concentration of about 3 mM in the aggregation media.
45. The method of any one of paragraphs 39-41, wherein bFGF is at a concentration of about 5 ng/ml in the aggregation media.
46. The method of any one of paragraphs 39-41, wherein VEGF is at a concentration of about 15 ng/ml in the aggregation media.
47. The method of any one of paragraphs 39-41, wherein IL-6 is at a concentration of about 10 ng/ml in the aggregation media.
48. The method of any one of paragraphs 39-41, wherein IL-11 is at a concentration of about 5 ng/mL in the aggregation media.
49. The method of any one of paragraphs 39-41, wherein IGF-1 is at a concentration of about 25 ng/mL in the aggregation media.
50. The method of any one of paragraphs 39-41, wherein SCF is at a concentration of about 50 ng/mL in the aggregation media.
51. The method of any one of paragraphs 39-41, wherein EPO is at a concentration of about 2 U/ml in the aggregation media.
52. The method of any one of paragraphs 1-51, further comprising selecting or isolating the resultant population of CD34⁺ hemogenic endothelium using expression of surface markers on the population of CD34⁺ hemogenic endothelium.
53. The method of any one of paragraphs 1-52, wherein the population of CD34⁺ hemogenic endothelium is CD45 negative/low.
54. The method of any one of paragraphs 1-53, wherein the population of CD34⁺ hemogenic endothelium is CD38 negative/low.
55. The method of any one of paragraphs 1-54, further comprising the step of genetically modifying the pluripotent stem cells, the resultant population of CD34⁺ hemogenic endothelium, or the resultant population of CD56⁺ NK cells.
56. The method of paragraph 55, wherein the genetic modification is removing an endogenous NK cell receptor and/or expressing a chimeric antigen receptor (CAR).
57. The method of paragraph 56, wherein the genetic modification reduces immunogenicity in the cell.
58. The method of paragraph 57, wherein the genetic modification that reduces immunogenicity is editing an endogenous HLA.
59. The method of paragraph 58, wherein the genetic modification that reduces immunogenicity comprises removing or editing HLA class I or HLA class II.
60. The method of paragraph 59, wherein the genetic modification that reduces immunogenicity comprises expressing at least one tolerance-promoting immunomodulatory molecule selected from the group consisting of: HLA-G, HLA-E, CD47, and PD-L1.
61. The method of paragraph 60, wherein the genetic modification that reduces immunogenicity comprises expressing at least one immunomodulatory molecule selected from the group consisting of: CCL21, PD-L1, FasL, SERPINB9, H2-M3, CD47, CD200 and MFGE8.
62. The method of paragraph 1 or 2, wherein the histone methyltransferase catalyzes the addition of methyl group to the histone 3 lysine residue 9 (H3K9) and/or histone 3 lysine residue 27 (H3K27).
63. The method of paragraph 62, wherein the histone methyltransferase H3K9 and/or H3K27 is inhibited by a small molecule inhibitor or a nucleic acid inhibitor.
64. The method of paragraph 63, wherein the histone methyltransferase H3K9 and/or H3K27 small molecule inhibitor is a heterorganic compound or an organometallic compound.
65. The method of paragraph 63 or 64, wherein the histone methyltransferase H3K9 and/or H3K27 small molecule inhibitor is selected from the group consisting of BIX-01294, UNC0638, E72, BRD4770, A-366, chaetocin, UNC0224, UNC0631, UNC0646, EPZ005687, EPZ-6438 (E7438), 3-deazaneplanocin A (DZNep), EI1, GSK343, GSK126, and UNC1999.
66. The method of paragraph 63, wherein the nucleic acid inhibitor is a nucleic acid targeting the expression of histone methyltransferase.
67. The method of paragraph 63, wherein the nucleic acid inhibitor is an RNA interference inhibitor or agent.
68. The method of paragraph 63, wherein the nucleic acid inhibitor is an EZH1-specific nucleic acid inhibitor.
69. The method of paragraph 63, wherein the nucleic acid inhibitor is an aptamer that binds EZH1.
70. The method of paragraph 63, wherein the nucleic acid inhibitor is an EZH1-specific RNA interference agent or a vector encoding an EZH1-specific RNA interference agent, wherein the RNA interference agent comprises one or more of the nucleotide sequences selected from SEQ ID NOS: 11-19.
71. The method of paragraph 63, wherein the nucleic acid inhibitor is an EZH1-specific CRISPR guide RNA in combination with a Cas enzyme, or a vector encoding an EZH1-specific CRISPR guide RNA and a Cas enzyme, wherein the CRISPR guide RNA comprises one or more of the nucleotide sequences selected from SEQ ID NOS: 20-41.
72. The method of paragraph 63, wherein the nucleic acid inhibitor is an EZH1-specific CRISPRi guide RNA in combination with a dCas enzyme, or a vector encoding an EZH1-specific CRISPRi guide RNA and a dCas enzyme, wherein the CRISPRi guide RNA comprises one or more of the nucleotide sequences selected from SEQ ID NOS: 51-53.
73. The method of paragraph 3 or 4, wherein the epigenetic regulator is a DNA-methyltransferase (DNMT); a methyl-CpG-binding domain (MBD) protein; a DNA demethylase; a histone methyl transferase (HMT); a methyl-histone binding protein; a histone demethylase; a histone acetyl transferase (HAT); an acetyl-binding protein; or a histone deacetylase (HDAC).
74. The method of paragraph 3 or 4, wherein the epigenetic regulator is inhibited by a small molecule inhibitor or a nucleic acid inhibitor.
75. The method of paragraph 74, wherein the inhibitor of the epigenetic regulator is selected from the group consisting of: UNC0224; MC1568; and CAY10591.
76. The method of paragraph 74, wherein the inhibitor of the epigenetic regulator is UNC0224.
77. The method of paragraph 74, wherein the inhibitor of the epigenetic regulator is MC1568.
78. The method of paragraph 74, wherein the inhibitor of the epigenetic regulator is CAY10591.
79. The method of any one of paragraphs 74-78, wherein the inhibitor of the epigenetic regulator is provided at a concentration of at least 500 nM.
80. The method of paragraph 5 or 6, wherein G9a and/or GLP is inhibited by a small molecule inhibitor.
81. The method of paragraph 5 or 6, wherein G9a and/or GLP is inhibited by a nucleic acid inhibitor.
82. The method of paragraph 80, wherein the G9a and/or GLP inhibitor is selected from the group consisting of: UNC0224; UNC0638; A366; BRD4770; BIX01294; UNC0642; UNC0631; UNC0646; UNC0321; E72; BIX-01338; BRD9539; Chaetocin; and DCG066.
83. The method of paragraph 80, wherein the G9a and/or GLP inhibitor is UNC0224.
84. The method of paragraph 80, wherein the G9a and/or GLP inhibitor is UNC0638.
85. The method of paragraph 80, wherein the G9a and/or GLP inhibitor is A366
86. The method of paragraph 80, wherein the G9a and/or GLP inhibitor is BRD4770
87. The method of paragraph 80, wherein the G9a and/or GLP inhibitor is BIX01294
88. The method of paragraph 80, wherein the G9a and/or GLP inhibitor is UNC0642
89. The method of paragraph 80, wherein the G9a and/or GLP inhibitor is UNC0631
90. The method of paragraph 80, wherein the G9a and/or GLP inhibitor is UNC0646
91. The method of paragraph 80, wherein the G9a and/or GLP inhibitor is UNC0321
92. The method of paragraph 80, wherein the G9a and/or GLP inhibitor is E72
93. The method of paragraph 80, wherein the G9a and/or GLP inhibitor is BIX-01338
94. The method of paragraph 80, wherein the G9a and/or GLP inhibitor is BRD9539
95. The method of paragraph 80, wherein the G9a and/or GLP inhibitor is Chaetocin
96. The method of paragraph 80, wherein the G9a and/or GLP inhibitor is DCG066.
97. The method of any one of paragraphs 80-96, wherein the G9a and/or GLP inhibitor is provided at a concentration of 300 nM-5 μM.
98. The method of paragraph 81, wherein the G9a inhibitor comprises SEQ ID NO: 50, or a nucleic acid sequence that is at least 95% identical and maintains the same function.
99. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.
100. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.
101. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.
102. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells;
      wherein the NK-cell-differentiation media further comprises 5 ng/mL thrombopoietin (TPO) for at least the first two weeks; and
      wherein the NK-cell-differentiation media further comprises 10 ng/mL interleukin-15 (IL-15) starting after at least the first two weeks.
103. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells;
      wherein the NK-cell-differentiation media further comprises 5 ng/mL thrombopoietin (TPO) for at least the first two weeks; and
      wherein the NK-cell-differentiation media further comprises 10 ng/mL interleukin-15 (IL-15) starting after at least the first two weeks.
104. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells;
      wherein the NK-cell-differentiation media further comprises 5 ng/mL interleukin-3 (IL-3) for at least the first week;
      wherein the NK-cell-differentiation media further comprises 5 ng/mL thrombopoietin (TPO) for at least the first two weeks; and
      wherein the NK-cell-differentiation media further comprises 10 ng/mL interleukin-15 (IL-15) starting after at least the first two weeks.
105. A method comprising:
   a) differentiating a population of pluripotent stem cells in aggregation media for a sufficient time to promote differentiation into a population of CD34⁺ hemogenic endothelium; and
   b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells;
      wherein the NK-cell-differentiation media further comprises 5 ng/mL interleukin-3 (IL-3) for at least the first week;
      wherein the NK-cell-differentiation media further comprises 5 ng/mL thrombopoietin (TPO) for at least the first two weeks; and
      wherein the NK-cell-differentiation media further comprises 10 ng/mL interleukin-15 (IL-15) starting after at least the first two weeks.
106. A method comprising: differentiating a population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.
107. A method comprising: differentiating a population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.
108. A method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.
109. A method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.
110. A method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.
111. A method comprising: differentiating a population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.
112. A method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.
113. The method of any one of paragraphs 99-112, further comprising inhibiting G9a and/or GLP in the population of CD34⁺ hemogenic endothelium.
114. The method of any one of paragraphs 99-112, further comprising inhibiting EZH1 in the population of CD34⁺ hemogenic endothelium.
115. The method of any one of paragraphs 99-112, further comprising inhibiting EZH1 and G9a in the population of CD34⁺ hemogenic endothelium.
116. The method of any one of paragraphs 1-115, wherein the population of CD56⁺ NK cells comprises an at least 2-fold higher number of CD56⁺ NK cells than the number of CD56⁺ NK cells produced by a NK differentiation method comprising stroma cells.
117. The method of any one of paragraphs 1-116, wherein the population of CD56⁺ NK cells comprises an at least 2-fold higher percentage of CD56⁺ NK cells than the percentage of CD56⁺ NK cells produced by a NK differentiation method comprising stroma cells.
118. A cell produced by the method of any one of paragraphs 1-117.
119. A composition comprising the cell of paragraph 118 or population thereof
120. A pharmaceutical composition comprising the cell of paragraph 118 or population thereof, and a pharmaceutically acceptable carrier.
121. The pharmaceutical composition of paragraph 120 for use in cellular replacement therapy in a subject.
122. A method of cellular replacement therapy, the method comprising administering a cell of paragraph 118 or population thereof, or a composition of paragraph 119, or a pharmaceutical composition of paragraph 120 to a recipient subject in need thereof
123. The method of cellular replacement therapy of paragraph 122, wherein the recipient subject has undergone chemotherapy and/or irradiation.
124. The method of cellular replacement therapy of paragraph 122, wherein the recipient subject has cancer.
125. The method of cellular replacement therapy of paragraph 122, wherein the recipient subject has deficiencies in immune function and/or immune cell reconstitution.
126. The method of cellular replacement therapy of any one of paragraphs 122-125, wherein prior to transplanting, the cell or population thereof is treated ex vivo with prostaglandin E2 and/or antioxidant N-acetyl-L-cysteine (NAC) to promote subsequent engraftment in a recipient subject.
127. The method of cellular replacement therapy of any one of paragraphs 122-126, wherein the cell or population thereof is autologous to the recipient subject.
128. The method of cellular replacement therapy of any one of paragraphs 122-127, wherein the cell or population thereof is HLA type matched with the recipient subject.
129. The method of cellular replacement therapy of any one of paragraphs 122-128, wherein the cell or population thereof is hypoimmunogenic.
130. A method of treating cancer, comprising administering an effective amount of a cell of paragraph 118 or population thereof, or a composition of paragraph 119, or a pharmaceutical composition of paragraph 120 to a recipient subject in need thereof
131. The method of paragraph 130, wherein the cell is a CD56⁺ NK cell.

The technology described herein is further illustrated by the following examples which in no way should be construed as being further limiting.

### EXAMPLES

### Example 1: Stroma-Free NK Cell Differentiation from Human Pluripotent Stem Cells

NK cells have shown great therapeutic potential for cancer immunotherapy. IPSC-derived NK cells provide a source for NK cell based immunotherapy. However, current protocols for the generation of NK cells from iPSC rely on mouse stroma cells, which limits the translational potential of the iPSC-derived NK cells. Described herein is a serum-free, stromal-free differentiation protocol for NK cell differentiation. Such a system can generate NK cells in vitro.

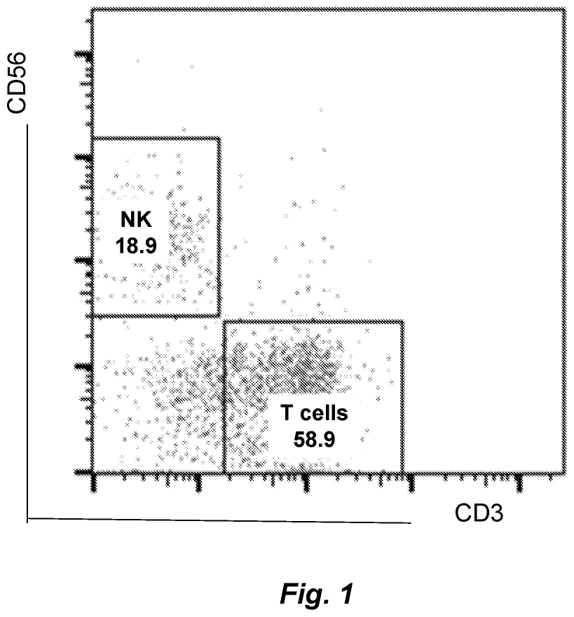

Hemogenic CD34⁺ endothelial cells were first derived from iPS (Example 2). The iPS-derived CD34⁺ hemogenic endothelial cells were cultured on tissue culture plates coated with Notch ligands (e.g., DLL4; e.g., 5 μg/ml or 10 μg/ml in PBS, 3 hours in room temperature) using media containing a cocktail of growth factors (e.g., Flt3, SCF, IL7). A higher concentration of DLL4 (e.g., 10 μg/ml) promotes T cell differentiation, while a lower concentration (e.g., 5 μg/ml) of DLL4 facilitates the generation of CD56⁺ NK cells. A traditional stromal-dependent differentiation protocol yielded both NK (CD56⁺) and T cells (CD3⁺) at a low efficiency (FIG. 1), whereas the stromal-free NK cell differentiation method described herein specifically generated a high percentage of NK cells (second panel of FIG. 2). EZH-1 knockdown increased the amount of differentiated CD56⁺ NK cells by approximately 3-6 fold (FIGS. 3A-3B). See, e.g., Table 1 for a summary of different differentiation protocols. For more details concerning the stroma-free T cell differentiation protocol, see, e.g., International PCT publication WO 2021/150919, the content of which is incorporated herein by reference in its entirety. The concentrations of cytokines are not affected by the basal media, but by the presence of stromal cells (e.g., OP9-DLL1 or OP9-DLL4 cells); in some embodiments, the concentrations of cytokines are lower in the presence of OP9 cells, and the concentrations of cytokines are higher in stromal-free differentiation methods.

TABLE 1
Comparison of traditional differentiation protocol and stroma-free T
cell and NK cell differentiation protocols; * alpha Minimum Essential
Medium (MEM) was used as the basal cell culture medium; ** StemSpan ™ SFEM
II medium was used as the basal cell culture medium.

| | | DLL4 | IL-7 | SCF | Flt3 | TPO | IL-15 | IL-3 |
|---|---|---|---|---|---|---|---|---|
| Protocol | Stroma | (ug/ml) | (ng/ml) | (ng/ml) | (ng/ml) | (ng/ml) | (ng/ml) | (ng/ml) |
| Traditional; | OP9- | N/A | 5 | 30 | 5 | | | |
| T and NK | DLL1 | | | | | | | |
| cells | | | | | | | | |
| (FIG. 1) * | | | | | | | | |
| T cell | Stroma- | 10 | 50 | 100 | 100 | 50 (first | | |
| (FIG. 2) ** | free | | | | | 2 weeks) | | |
| NK cell | Stroma- | 5 | 10 | 50 | 50 | | | |
| (FIGS. 2-3) ** | free | | | | | | | |
| NK cell ** | Stroma- | 5 | 5 | 100 | 100 | | | |
| | free | | | | | | | |
| NK cell ** | Stroma- | 5 | 20 | 30 | 15 | 5 (first | 10 (after | |
| | free | | | | | two | first 2 | |
| | | | | | | weeks) | weeks) | |
| NK cell | Stroma- | 5 | 25 | 30 | 20 | 5 (first | 10 (after | 5 (first |
| (FIG. 4-5) ** | free | | | | | two | first | week |
| | | | | | | weeks) | week) | only) |

### Example 2: Method for Producing Hemogenic Endothelium

Described herein is an 8-day protocol for producing hemogenic (CD34⁺) endothelium from induced pluripotent stem (iPS) cells; see, e.g., Sturgeon et al., Wnt Signaling Controls the Specification of Definitive and Primitive Hematopoiesis From Human Pluripotent Stem Cells, Nat Biotechnol. 2014 June; 32(6): 554-561, the content of which is incorporated herein by reference in its entirety.

Day 0: Formation of EBs from iPSCs on MEFs

Generally, after three days to one week of culture on murine embryonic fibroblasts (MEFs), iPS cells are ready to make embryoid bodies (EBs). The following protocol is followed for DO.
1. Wash the iPS cells grown on MEFs with 5 mL of DMEM/F12 media.
2. Aspirate the media from each dish. Replace with 5 mL of 1× collagenase IV diluted in 0.22 μM filtered DMEM/F12.
3. Incubate at 37° C. for 5-10 minutes and check periodically on the microscope that the iPS colonies are detaching.
4. Aspirate collagenase IV and replace with 5 mL of filtered DMEM/F12.
5. Using a sterile cell scrapper, scrape colonies first around the edges of the dish then left to right then top to bottom.
6. Gently and slowly transfer the cells to a conical tube using a 10 mL serological pipet. If there are residual colonies, wash plates with additional 5 mL and add to same tube.
7. Spin down 1100 rpm for 1 min
8. While cells are spinning, add 9 mL of Aggregation media with BMP4 (see, e.g., Table 2) to Corning Ultra Low Adherent 10 cm dishes.
9. Aspirate the media from the pelleted iPS colonies and resuspend in 1 mL of Aggregation media with BMP4.
10. Gently transfer the 1 mL of cells to each Ultra Low Adherent 10 cm containing the Aggregation media. Using the same pipette, pipet up 1 mL from an area of the plate without any cells and wash the conical. Add back the 1 mL to the conical; final volume of each plate now containing 3-4 starting plates of iPS cells is 10 mL.
11. Transfer to hypoxic incubator (5% O₂) at 37° C. 4-5 plates can be stacked on top of one another, with one plate filled with PBS at the bottom of the stack to prevent evaporation. This is Day 0 of EB culture.
   Day 1: Add bFGF

On Day 1 the following protocol is followed: 1. Directly add bFGF to each 10 cm dish of EBs for a final concentration of 5 ng/mL. Shake plate to distribute in media.

Day 2: Complete media change D2 Media

On Day 2, the D2 media is introduced. The following protocol is followed for D2.
1. The D2 Aggregation Media comprises the following (see, e.g., Table 2): BMP4, bFGF, CHIR99021 (StemCell Technologies Inc. #72054), and SB431542 (StemCell Technologies Inc. #72234). Once SB and CHIR are thaw it is not recommendable to freeze them again.
2. Collect EBs using a 10 mL serological pipet and place into a conical tube.
3. Let EBs settle for ˜15 minutes.
4. Aspirate the media and resuspend in D2 media (10 mL/10-cm dish) then gently transfer back to the Ultra-Low Adherent dishes.
   Day 3: Complete Media Change with D3 Media

On Day 3, the D3 media is introduced. The following protocol is followed for D3.
1. The D3 Aggregation Media comprises the following (see, e.g., Table 2): VEGF and bFGF.
2. Collect EBs using a 10 mL serological pipet and place into a conical tube.
3. Let EBs settle for ˜15 minutes.
4. Aspirate the media and resuspend in D2 media (10 mL/10-cm dish) then gently transfer back to the Ultra-Low Adherent dishes.

### Days 4-5: No Media Change

No media change is made on Days 4-5.

Day 6: Complete media change with D6 Media

On Day 6, the D6 media is introduced. The following protocol is followed for D6.
1. The D6 Aggregation Media comprises the following (see, e.g., Table 2): VEGF Recombinant Human VEGF 165 (VEGF-A) (R & D Systems (R&D) #293-VE-500), bFGF, SCF, EPO, IL-6 Recombinant Human IL-6 (20 μg) (Peprotech #200-06), IL-11, and IGF-1.
2. Collect EBs using a 10 mL serological pipet and place into a conical tube
3. Let EBs settle for ˜15 minutes
4. Aspirate the media and resuspend in D6 media (10 mL/10-cm dish) then gently transfer back to the Ultra-Low Adherent dishes

### Day 7: No Media Change

No media change is made on Day 7.

### Day 8: Isolation of Hemogenic Endothelium by MAC Sorting for CD34⁺ Cells

On Day 8, the hemogenic endothelium is isolated using magnetic-activated cell sorting (MACS) for CD34⁺ cells. The population of CD34⁺ hemogenic endothelium can then be used to differentiate NK cells using the stroma-free NK cell differentiation method as described herein (see, e.g., Example 1).

TABLE 2
Cytokines for EB culture

| | | | | | | | For |
|---|---|---|---|---|---|---|---|
| | | | | | | | 10 mL |
| Product | [Final] | | [Stock] | | Dilution | Days | media |
| BMP4 | 10 | ng/ml | 100 | ug/ml | 10000 | 0, 2 | 1 |
| SB-431542 | 6 | mM | 100 | mM | 16666 | 2 | 0.6 |
| CHIR99021 | 3 | mM | 50 | mM | 16666 | 2 | 0.6 |
| bFGF | 5 | ng/ml | 50 | ug/ml | 2000 | 1, 2, 3, 6 | 1 |
| VEGF | 15 | ng/ml | 150 | ug/ml | 10000 | 3, 6 | 1 |
| IL-6 | 10 | ng/ml | 100 | ug/ml | 10000 | 6 | 1 |
| IL-11 | 5 | ng/ml | 50 | ug/ml | 10000 | 6 | 1 |
| IGF-1 | 25 | ng/ml | 250 | ug/ml | 10000 | 6 | 1 |
| SCF | 50 | ng/ml | 100 | ug/ml | 2000 | 6 | 5 |
| EPO | 2 | U/ml | 10000 | U/ml | 5000 | 6 | 2 |

The basal media for the aggregation media are different for DO-D2 and D3-D8 during EB formation (see e.g., Tables 10-11).

TABLE 10
D 0-D 2 basal medium for aggregation media

| Component | Volume | |
|---|---|---|
| IMDM | 375 | mL |
| HAM'S F-12 (LIFETECH 11765-054) | 125 | mL |
| Pen/Strep (10 ng/mL) | 5 | mL |
| N2 (1%, LIFETECH 17502-048) | 5 | mL |
| B27 (0.5%, LIFETECH 17504-044) | 10 | mL |
| BSA (0.05%; stock 7.5%, stored at +4° C.) | 3.3 | mL |
| L-glutamin (2 mM) (usually supplemented in the IMDM) | 5 | mL |
| Ascorbic Acid (1 mM; 100 mg/mL stock (0.57M) in H2O, | 250 | μL |
| stored at −20° C.) | | |
| Holo-Transferrin (150 ug/ml; SIGMA T0665-1G; 100 mg/ml | 750 | μL |
| s tock in IMDM, stored at −20° C.) | | |

TABLE 11
D 3-D 8 basal medium for aggregation media

| Component | Volume | |
|---|---|---|
| STEMPRO-34 with Supplement (INVITROGEN 10639-011) | 500 | mL |
| L-glutamin (2 mM) | 5 | mL |
| Ascorbic Acid (1 mM; 100 mg/mL stock (0.57M) in H₂O, | 250 | μL |
| stored at −20° C.) | | |
| Holo-Transferrin (150 ug/ml; SIGMA T0665-1G; 100 mg/ml | 750 | μL |
| stock in IMDM, stored at −20° C.) | | |
| Pen/Strep (10 ng/mL) | 5 | mL |

### Example 3

FIG. 4 shows that the stroma-free differentiation protocol generated NK cells that expressed higher levels of NK cell receptors (e.g., NCR1, CD16, NKG2A), indicating increased developmental maturation and functionality of the iPSC-derived NK cells produced by the methods described herein.

FIGS. 5A-5C demonstrate the effect of G9a (EHMT2) knockdown on NK cells differentiation. iPSC lines were engineered with a Dox-inducible G9a (EHMT2) clustered regularly interspaced short palindromic repeats interference (CRISPRi) machinery; CRISPRi is similar to CRISPR, but instead of knocking out genes, CRISPRi uses catalytically dead Cas9 (usually denoted as dCas9) protein that lacks endonuclease activity to transcriptionally repress gene expression and achieve gene knockdown. The iPSCs were then differentiated into CD34+HE cells and treated with Dox to induce G9a knockdown. The control or Dox treated cells were then differentiated into CD7+ lymphoid progenitors and eventually CD5-CD56+NK cells using the stroma-free methods described herein (see, e.g., Table 1). The result shows that repression of G9a in CD34+HE cells promoted NK cell differentiation from iPSCs.

FIGS. 6A-6C show the mechanism by which G9a (EHMT2) regulates lymphoid fate decision in iPSC-derived HSPCs (iPSC-HSPCs). The iPSCs were first differentiated into CD34+ HEs, and the cells were then treated with DMSO or G9a inhibitor UNC0224 during the EHT stage, which allowed the HEs to specify into CD34+CD45+ HSPCs. ATAC-seq analysis was then performed on these iPSC-HSPCs to detect ATAC peaks that indicated accessible chromatin regions (as indicated by higher ATAC-seq scores). The peaks upregulated in UNC0224 treated cells were associated with genes that are responsible for lymphoid differentiation/function, indicating that repression of G9a unlocks the gene expression program that determines a lymphoid fate. These data demonstrate how repression of G9a (EHMT2) promotes NK cell differentiation from iPSCs.

FIG. 6C shows results form ATAC-seq experiment, which provides information on how G9a inhibitor affects chromatin accessibility. RNA-seq analysis was performed on the same samples as in FIG. 6A. Compared to control EHT cells, G9a-inhibitor (UNC0224) treated cells showed upregulation of genes associated with lymphoid development and function. Exemplary lymphoid-related genes identified by the RNA-seq analysis include, but are not limited to: RUNX3, CD1A, TNF, GZMA, GNLY, and IKZF5. Table 6 below shows the biological processes that were enriched in the G9a-inhibitor (UNC0224) treated cells, identified by RNA-seq analysis.

TABLE 6
RNA-seq data of pathways up-regulated by G9a-inhibition; analysis type: PANTHER overrepresentation
test; annotation version: PANTHER version 17.0; analyzed list: client text box input (Homo sapiens);
reference list: Homo sapiens (all genes in database); test type: FISHER; correction: FDR.

| | Homo | | | | Client Text | Client Text | |
|---|---|---|---|---|---|---|---|
| | sapiens - | Client Text | Client Text | Client Text | Box Input | Box Input | Client Text |
| PANTHER GO-Slim Biological | REFLIST | Box Input | Box Input | Box Input | (fold | (raw P- | Box Input |
| Process | (20589) | (239) | (expected) | (over/under) | Enrichment) | value) | (FDR) |
| antigen processing and | 41 | 14 | 0.48 | + | 29.42 | 1.34E−15 | 2.98E−12 |
| presentation (GO:0019882) | | | | | | | |
| positive regulation of leukocyte | 39 | 10 | 0.45 | + | 22.09 | 1.82E−10 | 1.35E−07 |
| cell-cell adhesion (GO:1903039) | | | | | | | |
| positive regulation of T cell | 39 | 10 | 0.45 | + | 22.09 | 1.82E−10 | 1.01E−07 |
| activation (GO:0050870) | | | | | | | |
| positive regulation of cell-cell | 40 | 10 | 0.46 | + | 21.54 | 2.25E−10 | 1.00E−07 |
| adhesion (GO:0022409) | | | | | | | |
| T cell differentiation | 14 | 3 | 0.16 | + | 18.46 | 9.01E−04 | 4.65E−02 |
| (GO:0030217) | | | | | | | |
| positive regulation of cell adhesion | 48 | 10 | 0.56 | + | 17.95 | 1.06E−09 | 3.91E−07 |
| (GO:0045785) | | | | | | | |
| regulation of leukocyte cell-cell | 51 | 10 | 0.59 | + | 16.89 | 1.77E−09 | 5.62E−07 |
| adhesion (GO:1903037) | | | | | | | |
| regulation of T cell activation | 53 | 10 | 0.62 | + | 16.25 | 2.46E−09 | 6.83E−07 |
| (GO:0050863) | | | | | | | |
| leukocyte cell-cell adhesion | 57 | 10 | 0.66 | + | 15.11 | 4.59E−09 | 1.13E−06 |
| (GO:0007159) | | | | | | | |
| regulation of cell-cell adhesion | 57 | 10 | 0.66 | + | 15.11 | 4.59E−09 | 1.02E−06 |
| (GO:0022407) | | | | | | | |
| T cell activation (GO:0042110) | 83 | 14 | 0.96 | + | 14.53 | 5.64E−12 | 6.26E−09 |
| regulation of cell adhesion | 80 | 10 | 0.93 | + | 10.77 | 8.40E−08 | 1.24E−05 |
| (GO:0030155) | | | | | | | |
| adaptive immune response based | 142 | 13 | 1.65 | + | 7.89 | 2.92E−08 | 5.89E−06 |
| on somatic recombination of | | | | | | | |
| immune receptors built from | | | | | | | |
| immunoglobulin superfamily | | | | | | | |
| domains (GO:0002460) | | | | | | | |
| lymphocyte mediated immunity | 147 | 13 | 1.71 | + | 7.62 | 4.26E−08 | 7.88E−06 |
| (GO:0002449) | | | | | | | |
| production of molecular mediator | 125 | 11 | 1.45 | + | 7.58 | 4.99E−07 | 5.27E−05 |
| of immune response | | | | | | | |
| (GO:0002440) | | | | | | | |
| B cell mediated immunity | 118 | 10 | 1.37 | + | 7.3 | 2.28E−06 | 2.20E−04 |
| (GO:0019724) | | | | | | | |
| immunoglobulin mediated | 118 | 10 | 1.37 | + | 7.3 | 2.28E−06 | 2.11E−04 |
| immune response (GO:0016064) | | | | | | | |
| leukocyte mediated immunity | 154 | 13 | 1.79 | + | 7.27 | 7.08E−08 | 1.12E−05 |
| (GO:0002443) | | | | | | | |
| adaptive immune response | 165 | 13 | 1.92 | + | 6.79 | 1.50E−07 | 1.85E−05 |
| (GO:0002250) | | | | | | | |
| immune effector process | 182 | 14 | 2.11 | + | 6.63 | 6.51E−08 | 1.11E−05 |
| (GO:0002252) | | | | | | | |
| positive regulation of lymphocyte | 132 | 10 | 1.53 | + | 6.53 | 5.80E−06 | 4.77E−04 |
| activation (GO:0051251) | | | | | | | |
| lymphocyte activation | 188 | 14 | 2.18 | + | 6.42 | 9.48E−08 | 1.31E−05 |
| (GO:0046649) | | | | | | | |
| positive regulation of leukocyte | 137 | 10 | 1.59 | + | 6.29 | 7.89E−06 | 6.25E−04 |
| activation (GO:0002696) | | | | | | | |
| positive regulation of cell | 137 | 10 | 1.59 | + | 6.29 | 7.89E−06 | 6.04E−04 |
| activation (GO:0050867) | | | | | | | |
| leukocyte activation | 198 | 14 | 2.3 | + | 6.09 | 1.72E−07 | 2.01E−05 |
| (GO:0045321) | | | | | | | |
| regulation of lymphocyte | 148 | 10 | 1.72 | + | 5.82 | 1.49E−05 | 1.03E−03 |
| activation (GO:0051249) | | | | | | | |
| cell activation (GO:0001775) | 212 | 14 | 2.46 | + | 5.69 | 3.76E−07 | 4.17E−05 |
| regulation of leukocyte activation | 157 | 10 | 1.82 | + | 5.49 | 2.40E−05 | 1.57E−03 |
| (GO:0002694) | | | | | | | |
| positive regulation of immune | 254 | 16 | 2.95 | + | 5.43 | 1.00E−07 | 1.31E−05 |
| system process (GO:0002684) | | | | | | | |
| regulation of cell activation | 159 | 10 | 1.85 | + | 5.42 | 2.66E−05 | 1.69E−03 |
| (GO:0050865) | | | | | | | |
| cell-cell adhesion (GO:0098609) | 214 | 12 | 2.48 | + | 4.83 | 1.25E−05 | 8.98E−04 |
| cellular protein-containing | 348 | 17 | 4.04 | + | 4.21 | 1.19E−06 | 1.20E−04 |
| complex assembly (GO:0034622) | | | | | | | |
| regulation of immune system | 339 | 16 | 3.94 | + | 4.07 | 3.77E−06 | 3.22E−04 |
| process (GO:0002682) | | | | | | | |
| protein-containing complex | 367 | 17 | 4.26 | + | 3.99 | 2.37E−06 | 2.11E−04 |
| assembly (GO:0065003) | | | | | | | |
| protein-containing complex | 452 | 18 | 5.25 | + | 3.43 | 8.94E−06 | 6.62E−04 |
| subunit organization | | | | | | | |
| (GO:0043933) | | | | | | | |
| immune response (GO:0006955) | 541 | 18 | 6.28 | + | 2.87 | 8.66E−05 | 5.20E−03 |
| immune system process | 694 | 21 | 8.06 | + | 2.61 | 8.31E−05 | 5.12E−03 |
| (GO:0002376) | | | | | | | |
| cellular component biogenesis | 1024 | 29 | 11.89 | + | 2.44 | 1.56E−05 | 1.05E−03 |
| (GO:0044085) | | | | | | | |
| cellular component assembly | 896 | 23 | 10.4 | + | 2.21 | 6.32E−04 | 3.42E−02 |
| (GO:0022607) | | | | | | | |
| positive regulation of cellular | 1227 | 30 | 14.24 | + | 2.11 | 1.60E−04 | 9.35E−03 |
| process (GO:0048522) | | | | | | | |
| positive regulation of biological | 1365 | 32 | 15.85 | + | 2.02 | 2.03E−04 | 1.16E−02 |
| process (GO:0048518) | | | | | | | |
| cellular component organization or | 2644 | 50 | 30.69 | + | 1.63 | 6.18E−04 | 3.43E−02 |
| biogenesis (GO:0071840) | | | | | | | |
| gene expression (GO:0010467) | 2894 | 53 | 33.59 | + | 1.58 | 7.21E−04 | 3.81E−02 |

What is claimed herein is:

1. A method comprising:
a) inhibiting a histone methyltransferase in a population of CD34⁺ hemogenic endothelium; and
b) differentiating the population of CD34⁺ hemogenic endothelium in Natural Killer (NK)-cell-differentiation media in the presence of a Notch ligand for a sufficient time to promote differentiation into a population of CD56⁺ NK cells.

2. A method comprising:
a) differentiating a population of pluripotent stem cells in aggregation media for a sufficient time to promote differentiation into a population of CD34⁺ hemogenic endothelium;
b) inhibiting a histone methyltransferase in the resultant population of CD34⁺ hemogenic endothelium; and
c) differentiating the resultant population of CD34⁺ hemogenic endothelium in Natural Killer (NK)-cell-differentiation media in the presence of a Notch ligand for a sufficient time to promote differentiation into a population of CD56⁺ NK cells.

3. A method comprising:
a) inhibiting an epigenetic regulator in a population of CD34⁺ hemogenic endothelium; and
b) differentiating the population of CD34⁺ hemogenic endothelium in NK-cell-differentiation media in the presence of a Notch ligand for a sufficient time to promote differentiation into a population of CD56⁺ NK cells.

4. A method comprising:
a) differentiating a population of pluripotent stem cells in aggregation media for a sufficient time to promote differentiation into a population of CD34⁺ hemogenic endothelium;
b) inhibiting an epigenetic regulator in the resultant population of CD34⁺ hemogenic endothelium; and
c) differentiating the resultant population of CD34⁺ hemogenic endothelium in NK-cell-differentiation media in the presence of a Notch ligand for a sufficient time to promote differentiation into a population of CD56⁺ NK cells.

5. A method comprising:
a) inhibiting G9a and/or GLP in a population of CD34⁺ hemogenic endothelium; and
b) differentiating the population of CD34⁺ hemogenic endothelium in NK-cell-differentiation media in the presence of a Notch ligand for a sufficient time to promote differentiation into a population of CD56⁺ NK cells.

6. A method comprising:
a) differentiating a population of pluripotent stem cells in aggregation media for a sufficient time to promote differentiation into a population of CD34⁺ hemogenic endothelium;
b) inhibiting G9a and/or GLP in the resultant population of CD34⁺ hemogenic endothelium; and
c) differentiating the resultant population of CD34⁺ hemogenic endothelium in NK-cell-differentiation media in the presence of a Notch ligand for a sufficient time to promote differentiation into a population of CD56⁺ NK cells.

7. A method comprising differentiating a population of CD34⁺ hemogenic endothelium in NK-cell-differentiation media in the presence of a Notch ligand for a sufficient time to promote differentiation into a population of CD56⁺ NK cells.

8. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media in the presence of a Notch ligand for a sufficient time to promote differentiation into a population of CD56⁺ NK cells.

9. The method of any one of claims 1-8, wherein the Notch ligand is attached to a solid substrate.

10. The method of any one of claims 1-9, wherein the Notch ligand is attached to a cell culture dish.

11. The method of any one of claims 1-10, wherein the Notch ligand is not derived from a stromal cell.

12. The method of any one of claims 1-11, wherein differentiating the hemogenic endothelium in the presence of a Notch ligand does not comprise co-culturing with a stromal cell expressing a Notch ligand.

13. The method of any one of claims 1-12, wherein differentiating the hemogenic endothelium in the presence of a Notch ligand does not comprise co-culturing with OP9-DLL1 cells or OP9-DLL4 cells.

14. The method of any one of claims 1-13, wherein the Notch ligand is selected from the group consisting of Delta-like-1 (DLL1), Delta-like-4 (DLL4), immobilized Delta1^{ext-IgG}, and immobilized Delta4^{ext-IgG}.

15. The method of claim 14, wherein immobilized Delta1^{ext-IgG} consists of an extracellular domain of human Delta-like-1 fused to the Fc domain of human IgG1.

16. The method of any one of claims 1-15, wherein the Notch ligand is DLL4.

17. The method of any one of claims 1-16, wherein the Notch ligand is provided at a concentration of at most 5 μg/ml.

18. The method of any one of claims 1-17, wherein the sufficient time to promote differentiation into a population of CD56⁺ NK cells is at least 4 weeks.

19. The method of any one of claims 1-18, wherein the NK-cell-differentiation media is serum-free.

20. The method of any one of claims 1-19, wherein the NK-cell-differentiation media comprises SCF, FLT3, and IL7.

21. The method of any one of claims 1-20, wherein the NK-cell-differentiation media comprises 30 ng/ml-100 ng/mL SCF; 15 ng/ml-100 ng/mL FLT3; and 5 ng/ml-20 ng/ml IL7.

22. The method of any one of claims 1-21, wherein the NK-cell-differentiation media comprises 30 ng/mL SCF, 15 ng/ml FLT3, and 20 ng/ml IL7.

23. The method of any one of claims 1-22, wherein the NK-cell-differentiation media comprises 50 ng/mL SCF, 50 ng/ml FLT3, and 10 ng/ml IL7.

24. The method of any one of claims 1-23, wherein the NK-cell-differentiation media comprises 100 ng/mL SCF, 100 ng/ml FLT3, and 5 ng/ml IL7.

25. The method of any one of claims 1-24, wherein the NK-cell-differentiation media comprises 30 ng/ml SCF, 20 ng/ml FLT3, and 25 ng/ml IL7.

26. The method of any one of claims 1-25, wherein the NK-cell-differentiation media further comprises thrombopoietin (TPO).

27. The method of any one of claims 1-26, wherein the NK-cell-differentiation media further comprises 5 ng/mL thrombopoietin (TPO) for at least the first 2 weeks of differentiating in the NK-cell-differentiation media.

28. The method of any one of claims 1-27, wherein the NK-cell-differentiation media further comprises interleukin-15 (IL-15).

29. The method of any one of claims 1-28, wherein the NK-cell-differentiation media further comprises 10 ng/mL interleukin-15 (IL-15) starting after at least the first 2 weeks of differentiating in the NK-cell-differentiation media.

30. The method of any one of claims 1-29, wherein the NK-cell-differentiation media further comprises interleukin-3 (IL-3).

31. The method of any one of claims 1-30, wherein the NK-cell-differentiation media further comprises 5 ng/mL interleukin-3 (IL-3) for at least the first week of differentiating in the NK-cell-differentiation media.

32. The method of any one of claims 1-31, further comprising a step of CD56⁺ NK cell enrichment.

33. The method of any one of claim 2, 4, 6, or 8, wherein the population of pluripotent stem cells comprises induced pluripotent stem cells (iPS cells) or embryonic stem cells (ESC).

34. The method of claim 33, wherein the induced pluripotent stem cells are produced by introducing into mature cells at least one reprogramming factor selected from the group consisting of: OCT4, SOX2, KLF4, c-MYC, nanog, and LIN28, or any combination thereof.

35. The method of claim 34, wherein the induced pluripotent stem cells are produced by introducing only reprogramming factors OCT4, SOX2, KLF4 and optionally c-MYC or nanog and LIN28 into mature cells.

36. The method of any one of claims 33-35, wherein the induced pluripotent stem cells are produced by introducing the reprogramming factors two or more times into the mature cells.

37. The method of any one of claim 2, 4, 6, or 8, wherein the population of pluripotent stem cells is differentiated into a population of CD34⁺ hemogenic endothelium using embryoid bodies or 2D adherent cultures.

38. The method of any one of claim 2, 4, 6, or 8, wherein the sufficient time to promote differentiation into a population of CD34⁺ hemogenic endothelium is at least 8 days.

39. The method of any one of claim 2, 4, 6, or 8, wherein the aggregation media comprises BMP4, SB-431542, CHIR99021, bFGF, VEGF, IL-6, IL-11, IGF-1, SCF, and EPO.

40. The method of any one of claim 2, 4, 6, or 8, wherein the aggregation media comprises:
a) BMP4 for at least days 0 and 2;
b) SB-431542 for at least day 2;
c) CHIR99021 for at least day 2;
d) bFGF for at least day 1, 2, 3, and 6;
e) VEGF for at least days 3 and 6;
f) IL-6 for at least day 6;
g) IL-11 for at least day 6;
h) IGF-1 for at least day 6;
i) SCF for at least day 6; and/or
j) EPO for at least day 6.

41. The method of any one of claim 2, 4, 6, 9, or 40, wherein the aggregation media comprises 10 ng/ml BMP4, 6 mM SB-431542, 3 mM CHIR99021, 5 ng/ml bFGF, 15 ng/ml VEGF, 10 ng/ml IL-6, 5 ng/mL IL-11, 25 ng/mL IGF-1, 50 ng/mL SCF, and 2 U/ml EPO.

42. The method of any one of claims 39-41, wherein BMP4 is at a concentration of about 10 ng/ml in the aggregation media.

43. The method of any one of claims 39-41, wherein SB-431542 is at a concentration of about 6 mM in the aggregation media.

44. The method of any one of claims 39-41, wherein CHIR99021 is at a concentration of about 3 mM in the aggregation media.

45. The method of any one of claims 39-41, wherein bFGF is at a concentration of about 5 ng/ml in the aggregation media.

46. The method of any one of claims 39-41, wherein VEGF is at a concentration of about 15 ng/ml in the aggregation media.

47. The method of any one of claims 39-41, wherein IL-6 is at a concentration of about 10 ng/ml in the aggregation media.

48. The method of any one of claims 39-41, wherein IL-11 is at a concentration of about 5 ng/mL in the aggregation media.

49. The method of any one of claims 39-41, wherein IGF-1 is at a concentration of about 25 ng/mL in the aggregation media.

50. The method of any one of claims 39-41, wherein SCF is at a concentration of about 50 ng/mL in the aggregation media.

51. The method of any one of claims 39-41, wherein EPO is at a concentration of about 2 U/ml in the aggregation media.

52. The method of any one of claims 1-51, further comprising selecting or isolating the resultant population of CD34⁺ hemogenic endothelium using expression of surface markers on the population of CD34⁺ hemogenic endothelium.

53. The method of any one of claims 1-52, wherein the population of CD34⁺ hemogenic endothelium is CD45 negative/low.

54. The method of any one of claims 1-53, wherein the population of CD34⁺ hemogenic endothelium is CD38 negative/low.

55. The method of any one of claims 1-54, further comprising the step of genetically modifying the pluripotent stem cells, the resultant population of CD34⁺ hemogenic endothelium, or the resultant population of CD56⁺ NK cells.

56. The method of claim 55, wherein the genetic modification is removing an endogenous NK cell receptor and/or expressing a chimeric antigen receptor (CAR).

57. The method of claim 56, wherein the genetic modification reduces immunogenicity in the cell.

58. The method of claim 57, wherein the genetic modification that reduces immunogenicity is editing an endogenous HLA.

59. The method of claim 58, wherein the genetic modification that reduces immunogenicity comprises removing or editing HLA class I or HLA class II.

60. The method of claim 59, wherein the genetic modification that reduces immunogenicity comprises expressing at least one tolerance-promoting immunomodulatory molecule selected from the group consisting of: HLA-G, HLA-E, CD47, and PD-L1.

61. The method of claim 60, wherein the genetic modification that reduces immunogenicity comprises expressing at least one immunomodulatory molecule selected from the group consisting of: CCL21, PD-L1, FasL, SERPINB9, H2-M3, CD47, CD200 and MFGE8.

62. The method of claim 1 or 2, wherein the histone methyltransferase catalyzes the addition of methyl group to the histone 3 lysine residue 9 (H3K9) and/or histone 3 lysine residue 27 (H3K27).

63. The method of claim 62, wherein the histone methyltransferase H3K9 and/or H3K27 is inhibited by a small molecule inhibitor or a nucleic acid inhibitor.

64. The method of claim 63, wherein the histone methyltransferase H3K9 and/or H3K27 small molecule inhibitor is a heterorganic compound or an organometallic compound.

65. The method of claim 63 or 64, wherein the histone methyltransferase H3K9 and/or H3K27 small molecule inhibitor is selected from the group consisting of BIX-01294, UNC0638, E72, BRD4770, A-366, chaetocin, UNC0224, UNC0631, UNC0646, EPZ005687, EPZ-6438 (E7438), 3-deazaneplanocin A (DZNep), EI1, GSK343, GSK126, and UNC1999.

66. The method of claim 63, wherein the nucleic acid inhibitor is a nucleic acid targeting the expression of histone methyltransferase.

67. The method of claim 63, wherein the nucleic acid inhibitor is an RNA interference inhibitor or agent.

68. The method of claim 63, wherein the nucleic acid inhibitor is an EZH1-specific nucleic acid inhibitor.

69. The method of claim 63, wherein the nucleic acid inhibitor is an aptamer that binds EZH1.

70. The method of claim 63, wherein the nucleic acid inhibitor is an EZH1-specific RNA interference agent or a vector encoding an EZH1-specific RNA interference agent, wherein the RNA interference agent comprises one or more of the nucleotide sequences selected from SEQ ID NOS: 11-19.

71. The method of claim 63, wherein the nucleic acid inhibitor is an EZH1-specific CRISPR guide RNA in combination with a Cas enzyme, or a vector encoding an EZH1-specific CRISPR guide RNA and a Cas enzyme, wherein the CRISPR guide RNA comprises one or more of the nucleotide sequences selected from SEQ ID NOS: 20-41.

72. The method of claim 63, wherein the nucleic acid inhibitor is an EZH1-specific CRISPRi guide RNA in combination with a dCas enzyme, or a vector encoding an EZH1-specific CRISPRi guide RNA and a dCas enzyme, wherein the CRISPRi guide RNA comprises one or more of the nucleotide sequences selected from SEQ ID NOS: 51-53.

73. The method of claim 3 or 4, wherein the epigenetic regulator is a DNA-methyltransferase (DNMT); a methyl-CpG-binding domain (MBD) protein; a DNA demethylase; a histone methyl transferase (HMT); a methyl-histone binding protein; a histone demethylase; a histone acetyl transferase (HAT);
an acetyl-binding protein; or a histone deacetylase (HDAC).

74. The method of claim 3 or 4, wherein the epigenetic regulator is inhibited by a small molecule inhibitor or a nucleic acid inhibitor.

75. The method of claim 74, wherein the inhibitor of the epigenetic regulator is selected from the group consisting of: UNC0224; MC1568; and CAY10591.

76. The method of claim 74, wherein the inhibitor of the epigenetic regulator is UNC0224.

77. The method of claim 74, wherein the inhibitor of the epigenetic regulator is MC1568.

78. The method of claim 74, wherein the inhibitor of the epigenetic regulator is CAY10591.

79. The method of any one of claims 74-78, wherein the inhibitor of the epigenetic regulator is provided at a concentration of at least 500 nM.

80. The method of claim 5 or 6, wherein G9a and/or GLP is inhibited by a small molecule inhibitor.

81. The method of claim 5 or 6, wherein G9a and/or GLP is inhibited by a nucleic acid inhibitor.

82. The method of claim 80, wherein the G9a and/or GLP inhibitor is selected from the group consisting of: UNC0224; UNC0638; A366; BRD4770; BIX01294; UNC0642; UNC0631; UNC0646;
UNC0321; E72; BIX-01338; BRD9539; Chaetocin; and DCG066.

83. The method of claim 80, wherein the G9a and/or GLP inhibitor is UNC0224.

84. The method of claim 80, wherein the G9a and/or GLP inhibitor is UNC0638.

85. The method of claim 80, wherein the G9a and/or GLP inhibitor is A366

86. The method of claim 80, wherein the G9a and/or GLP inhibitor is BRD4770

87. The method of claim 80, wherein the G9a and/or GLP inhibitor is BIX01294

88. The method of claim 80, wherein the G9a and/or GLP inhibitor is UNC0642

89. The method of claim 80, wherein the G9a and/or GLP inhibitor is UNC0631

90. The method of claim 80, wherein the G9a and/or GLP inhibitor is UNC0646

91. The method of claim 80, wherein the G9a and/or GLP inhibitor is UNC0321

92. The method of claim 80, wherein the G9a and/or GLP inhibitor is E72

93. The method of claim 80, wherein the G9a and/or GLP inhibitor is BIX-01338

94. The method of claim 80, wherein the G9a and/or GLP inhibitor is BRD9539

95. The method of claim 80, wherein the G9a and/or GLP inhibitor is Chaetocin

96. The method of claim 80, wherein the G9a and/or GLP inhibitor is DCG066.

97. The method of any one of claims 80-96, wherein the G9a and/or GLP inhibitor is provided at a concentration of 300 nM-5 μM.

98. The method of claim 81, wherein the G9a inhibitor comprises SEQ ID NO: 50, or a nucleic acid sequence that is at least 95% identical and maintains the same function.

99. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

100. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.

101. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

102. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells;
wherein the NK-cell-differentiation media further comprises 5 ng/mL thrombopoietin (TPO) for at least the first two weeks; and
wherein the NK-cell-differentiation media further comprises 10 ng/mL interleukin-15 (IL-15) starting after at least the first two weeks.

103. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells;
wherein the NK-cell-differentiation media further comprises 5 ng/mL thrombopoietin (TPO) for at least the first two weeks; and
wherein the NK-cell-differentiation media further comprises 10 ng/mL interleukin-15 (IL-15) starting after at least the first two weeks.

104. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells;
wherein the NK-cell-differentiation media further comprises 5 ng/mL interleukin-3 (IL-3) for at least the first week;
wherein the NK-cell-differentiation media further comprises 5 ng/mL thrombopoietin (TPO) for at least the first two weeks; and
wherein the NK-cell-differentiation media further comprises 10 ng/mL interleukin-15 (IL-15) starting after at least the first two weeks.

105. A method comprising:
a) differentiating a population of pluripotent stem cells in aggregation media for a sufficient time to promote differentiation into a population of CD34⁺ hemogenic endothelium; and
b) differentiating the resultant population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells;
wherein the NK-cell-differentiation media further comprises 5 ng/mL interleukin-3 (IL-3) for at least the first week;
wherein the NK-cell-differentiation media further comprises 5 ng/mL thrombopoietin (TPO) for at least the first two weeks; and
wherein the NK-cell-differentiation media further comprises 10 ng/mL interleukin-15 (IL-15) starting after at least the first two weeks.

106. A method comprising: differentiating a population of CD34⁺ hemogenic endothelium in NK-cell-differentiation media comprising 50 ng/mL SCF, 50 ng/ml FLT3, and 10 ng/ml IL7 in the presence of 5 μg/mL Notch ligand for at least 4 weeks to promote differentiation into a population of CD56⁺ NK cells.

107. A method comprising: differentiating a population of CD34⁺ hemogenic endothelium in NK-cell-differentiation media comprising 100 ng/mL SCF, 100 ng/ml FLT3, and 5 ng/ml IL7 in the presence of 5 μg/mL Notch ligand for at least 4 weeks to promote differentiation into a population of CD56⁺ NK cells.

108. A method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells.

109. A method comprising: differentiating a population of CD34⁺ hemogenic endothelium in NK-cell-differentiation media comprising 30 ng/mL SCF, 15 ng/ml FLT3, and 20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

110. A method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-20 ng/ml IL7 in the presence of 5 μg/mL Notch ligand for at least 4 weeks to promote differentiation into a population of CD56⁺ NK cells; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

111. A method comprising: differentiating a population of CD34⁺ hemogenic endothelium in NK-cell-differentiation media comprising 30 ng/mL SCF, 20 ng/ml FLT3, and 25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

112. A method comprising: differentiating a population of CD34⁺ hemogenic endothelium in NK-cell-differentiation media comprising 30 ng/ml-100 ng/ml SCF, 15 ng/ml-100 ng/ml FLT3, and 5 ng/ml-25 ng/ml IL7 in the presence of 5 μg/mL Notch ligand for at least 4 weeks to promote differentiation into a population of CD56⁺ NK cells; wherein the NK-cell-differentiation media further comprises 5 ng/mL interleukin-3 (IL-3) for at least the first week; wherein the NK-cell-differentiation media further comprises 5 ng/mL thrombopoietin (TPO) for at least the first two weeks; and wherein the NK-cell-differentiation media further comprises 10 ng/mL interleukin-15 (IL-15) starting after at least the first two weeks.

113. The method of any one of claims 99-112, further comprising inhibiting G9a and/or GLP in the population of CD34⁺ hemogenic endothelium.

114. The method of any one of claims 99-112, further comprising inhibiting EZH1 in the population of CD34⁺ hemogenic endothelium.

115. The method of any one of claims 99-112, further comprising inhibiting EZH1 and G9a in the population of CD34⁺ hemogenic endothelium.

116. The method of any one of claims 1-115, wherein the population of CD56⁺ NK cells comprises an at least 2-fold higher number of CD56+NK cells than the number of CD56+NK cells produced by a NK differentiation method comprising stroma cells.

117. The method of any one of claims 1-116, wherein the population of CD56⁺ NK cells comprises an at least 2-fold higher percentage of CD56+NK cells than the percentage of CD56+NK cells produced by a NK differentiation method comprising stroma cells.

118. A cell produced by the method of any one of claims 1-117.

119. A composition comprising the cell of claim 118 or population thereof.

120. A pharmaceutical composition comprising the cell of claim 118 or population thereof, and a pharmaceutically acceptable carrier.

121. The pharmaceutical composition of claim 120 for use in cellular replacement therapy in a subject.

122. A method of cellular replacement therapy, the method comprising administering a cell of claim 118 or population thereof, or a composition of claim 119, or a pharmaceutical composition of claim 120 to a recipient subject in need thereof.

123. The method of cellular replacement therapy of claim 122, wherein the recipient subject has undergone chemotherapy and/or irradiation.

124. The method of cellular replacement therapy of claim 122, wherein the recipient subject has cancer.

125. The method of cellular replacement therapy of claim 122, wherein the recipient subject has deficiencies in immune function and/or immune cell reconstitution.

126. The method of cellular replacement therapy of any one of claims 122-125, wherein prior to transplanting, the cell or population thereof is treated ex vivo with prostaglandin E2 and/or antioxidant N-acetyl-L-cysteine (NAC) to promote subsequent engraftment in a recipient subject.

127. The method of cellular replacement therapy of any one of claims 122-126, wherein the cell or population thereof is autologous to the recipient subject.

128. The method of cellular replacement therapy of any one of claims 122-127, wherein the cell or population thereof is HLA type matched with the recipient subject.

129. The method of cellular replacement therapy of any one of claims 122-128, wherein the cell or population thereof is hypoimmunogenic.

130. A method of treating cancer, comprising administering an effective amount of a cell of claim 118 or population thereof, or a composition of claim 119, or a pharmaceutical composition of claim 120 to a recipient subject in need thereof.

131. The method of claim 130, wherein the cell is a CD56+ NK cell.
